(19) United States
(12) Patent Application Publication
(10) Pub. No.: US 2025/0179490 A1
(43) Pub. Date: Jun. 5, 2025

(54) METHODS OF TREATING GLAUCOMA

(71) Applicant: PYC Therapeutics Limited, Nedlands (AU)

(72) Inventors: Janya Grainok, Nedlands (AU); Lee Chai, Nedlands (AU); Quanqing Gao, Nedlands (AU)

(21) Appl. No.: 18/844,484

(22) PCT Filed: Mar. 23, 2023
(86) PCT No.: PCT/AU2023/050213

§ 371 Date: Sep. 6, 2024

(30) Foreign Application Priority Data
Mar. 23, 2022 (AU) 2022900727

(51) Int. Cl. C12N 15/113

(52) U.S. Cl. CPC C12N 15/113; C12N 2310/11; C12N 2310/321; C12N 2310/3233; C12N 2310/3513; C12N 2320/33

(57) ABSTRACT

The present disclosure generally relates to methods of treating, preventing and/or delaying progression of glaucoma in a subject, the method comprising administering an antisense oligonucleotide that modulates mRNA productive transcript, stability and/or translation of OPA1 gene transcript or part thereof.

### RELATED APPLICATION DATA

The present application claims priority from Australian Patent Application No. 2022900727 filed on 23 Mar. 2022 entitled “Methods of Treating Glaucoma”, the entire contents of which is hereby incorporated by reference.

### SEQUENCE LISTING

The present application is filed together with a Sequence Listing in electronic form. The entire contents of the Sequence Listing are hereby incorporated by reference.

### TECHNICAL FIELD

The present disclosure generally relates to methods of treating, preventing and/or delaying progression of glaucoma in a subject, the method comprising administering an antisense oligonucleotide that modulates mRNA translation, stability, and productive transcript of OPA1 gene transcript or part thereof.

### BACKGROUND

Glaucoma, a progressive optic neuropathy and the leading cause of blindness, is characterized by impairment or degeneration of retinal ganglion cells (RGCs), which transmit visual information to the brain. Currently, about 80 million people are affected by glaucoma worldwide, and this number is expected to increase to over 120 million by 2040. The prevalence of glaucoma increases with aging, and this increase is strongly affected by the African and Asian populations.

Glaucoma can be triggered when the aqueous humour builds up in the front part of the eye. Excess production or reduced draining of the aqueous humour increases the intraocular pressure (IOP), which irreversible damages the optic nerve and RGCs. Glaucoma can be classified as either primary or secondary, with secondary glaucoma attributable to another disorder or problem within the eye, such as injury, surgery, drugs, or other ocular diseases. Primary glaucoma is classified as open-angle glaucoma (POAG), normal-tension glaucoma (NTG), angle-closure glaucoma and congenital glaucoma. Secondary glaucoma is classified into neovascular glaucoma, pigmentary glaucoma, exfoliation glaucoma and uveitic glaucoma. In all subtypes of glaucoma, the gradual loss of RGCs is the hallmark. RGC dysfunction and death lead to vision impairment and ultimately blindness.

There is no approved treatment for glaucoma that directly targets RGCs. Of the drugs that have been clinically studied for neuroprotective activity and to reduce vision loss in POAG patients, e.g. brimonidine and memantine, none have conclusively proven effective thus far. Instead, the only available treatments to reduce IOP levels are indirectly protective for RGCs. Further, it has been reported that in about one-third of cases of glaucoma the characteristic optic nerve changes and visual field loss can develop in an eye with normal IOP levels. Therefore, there is an urgent need to identify therapeutic strategies for RGC neuroprotection to limit the projected burden of vision impairment and blindness from glaucoma. The use of neurotrophic factors such as brain derived-, ciliary derived-, glial cell derived, and nerve growth factor has been a focus of recent research for it is known to prevent uncontrolled RGCs loss and aid to the cell viability. However, their effectiveness is limited by a relatively short half-life, insufficient permeability, and poor concentrations in target RGCs.

Therefore, there remains a need for new interventions for treating, preventing and/or delaying progression of glaucoma.

### SUMMARY

In producing the present invention, the inventors identified optic atrophy gene 1 (OPA1) as a potential target for pharmacological intervention for treating or preventing glaucoma. The inventors have identified antisense oligonucleotides (ASOs) that increase expression of OPA1 expression that are useful for the treatment or prevention of glaucoma. The inventors have identified ASOs that rely on any of a variety of mechanisms of action to upregulate OPA1 expression. For example, an ASO identified by the inventors increase OPA1 expression by:
Binding to an OPA1 gene pre-mRNA in a cell to promote exclusion of a nonsense-mediated RNA decay-inducing (NMD) exon during splicing of the OPA1 pre-mRNA to increase the level of OPA1 mRNA transcripts encoding full length, functional OPA1
Binding to the 5′ untranslated region (UTR) of an OPA1 gene transcript in a cell to increase translation efficiency and/or transcript stability of an OPA1 mRNA
Binding to the 3′ UTR of an OPA1 gene transcript in a cell to increase transcript stability of an OPA1 mRNA

These findings additionally provide the basis for methods of treating, preventing and/or delaying progression of glaucoma.

Accordingly, the present disclosure provides a method of treating, preventing and/or delaying progression of glaucoma in a subject, the method comprising administering an antisense oligonucleotide that increases functional OPA1 protein levels in the subject. For example, the level of OPA1 protein is increased in the subject compared to the level in the subject prior to administration of the OPA1.

In one example, the ASO increases the level of OPA1 mRNA and the amount of functional OPA1 protein in a cell and/or a tissue of the subject. For example, the ASO increases the level of OPA1 mRNA in a cell and/or a tissue of the subject. In another example, the ASO increases the amount of functional OPA1 protein in a cell and/or a tissue of the subject.

In one example, the amount of functional OPA1 protein in the cell and/or the tissue is increased by about 1.1 to about 10-fold. For example, the amount of functional OPA1 protein in the cell and/or the tissue is increased by about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold. For example, the amount of functional OPA1 protein in the cell and/or the tissue is increased by at least about 1.5-fold, at least about 2-fold, at least about 2.5-fold, at least about 3-fold, at least about 3.5-fold, at least about 4-fold, at least about 5-fold, or at least about 10-fold. For example, the amount of functional OPA1 protein in the cell and/or the tissue is increased compared to the level in the tissue prior to the administration or contact. In one example, administration to a subject or contact with cells with any of the ASOs or pharmaceutical compositions disclosed herein increases the level of OPA1 protein about 1.1 to about 2.5-fold compared to the level in the tissue prior to the administration or contact.

In one example, the cell and/or tissue is selected from the group consisting of an ocular tissue, retinal pigment epithelium (RPE) cells, Müller glial cells, endothelial cells, glial cells, astrocytes, photoreceptors. For example, the cell and/or tissue is selected from the group consisting of the retina, RPE cells and combinations thereof.

In one example, the ASOs bind to a targeted portion of:
(i) an OPA1 gene pre-mRNA in a cell to promote exclusion of a nonsense-mediated RNA decay-inducing (NMD) exon during splicing of the OPA1 pre-mRNA to increase the level of OPA1 mRNA transcripts encoding full length, functional OPA1;
(ii) the 5′ untranslated region (UTR) of an OPA1 gene transcript in a cell to increase translation efficiency of an OPA1 mRNA;
(iii) the 5′ UTR of an OPA1 gene transcript in a cell to increase transcript stability, e.g., by inhibiting the activity of a decapping enzyme; and/or
(iv) the 3′ UTR of an OPA1 gene transcript in a cell to increase transcript stability, e.g., by preventing binding of a miRNA to the 3′ UTR.

In one example, the ASOs binds to a targeted portion of an OPA1 pre-mRNA in a cell to promote exclusion of a NMD exon during splicing of the OPA1 pre-mRNA to increase the level of OPA1 mRNA transcripts encoding full length, functional OPA1.

In one example, the ASO binds to a targeted portion of intron 7 OPA1 pre-mRNA. Exemplary ASOs bind within a targeted portion of OPA1 pre-mRNA nucleotide sequence corresponding to one or more of SEQ ID NO:1.

In one example, the ASO binds to intron 7 of an OPA1 gene pre-mRNA in a cell and increases the level of OPA1 gene transcripts encoding full length, functional OPA1 by exclusion of NMD exon 7×. For example, the ASO is within sufficient proximity to an acceptor site of exon 7× to promote exclusion of exon 7× in splicing of OPA1 mRNA.

In one example, the ASO that binds to a targeted portion of intron 7 OPA1 pre-mRNA comprises or consists of any one of SEQ ID NOs: 2-54.

In one example, the ASO that binds to a targeted portion of intron 7 OPA1 pre-mRNA comprises or consists of any one of SEQ ID NOs: 2-54 or SEQ ID NOs: 2491-2503.

In one example, the ASO that binds to a targeted portion of intron 7 OPA1 pre-mRNA comprises or consists of any one of SEQ ID NOs: 2491-2503 In one example, the ASO binds to a targeted portion of the 5′ UTR of an OPA1 gene transcript in a cell to increase translation efficiency or transcript stability of an OPA1 mRNA. For example, the ASO increases expression of OPA1 protein. Without being bound by theory or mode of action, such ASOs may sterically inhibit translation from upstream Open Reading Frames (uORF) start site and/or sterically inhibit secondary structure in the 5′ UTR and/or inhibiting the activity of a decapping enzyme.

In one example, the ASO binds within a targeted portion of the 5′ UTR of OPA1 mRNA, wherein the targeted portion is within the nucleotide sequence corresponding to SEQ ID NO: 55.

In one example, the ASO that binds to a targeted portion of the 5′ UTR of OPA1 mRNA comprises or consists of any one of SEQ ID NOs: 56-138.

In one example, the ASO that binds to a targeted portion of the 5′ UTR of OPA1 mRNA comprises or consists of SEQ ID NO: 112.

In one example, the ASO binds to a targeted portion of the 3′ UTR of an OPA1 gene transcript in a cell to increase transcript stability of an OPA1 mRNA. For example, the ASO increases expression of OPA1 protein. Without being bound by theory or mode of action, such ASOs may sterically inhibit binding of a miRNA to the 3′ UTR.

In one example, the ASO binds within a targeted portion of the 3′ UTR of OPA1 mRNA, wherein the targeted portion is within the nucleotide sequence corresponding to SEQ ID NO: 139.

In one example, the ASO that binds to a targeted portion of the 3′ UTR of OPA1 mRNA comprises or consists of any one of SEQ ID NOs: 140-2488.

In one example, the nucleotide sequence of the ASO is at least 60%, 65%, 70%, 75%, 80%, 85%, 90%, 95%, or 100% complementary to the nucleotide sequence of the targeted portion over the length of the ASO.

In one example, the ASO comprises a backbone modification. For example, the backbone modification comprises a phosphorothioate linkage or a phosphorodiamidate linkage. In one example, the ASO comprises a phosphorothioate linkage. In another example, the ASO comprises a phosphorodiamidate linkage.

In one example, the ASO comprises a phosphorodiamidate morpholino, a locked nucleic acid, a peptide nucleic acid, a 2-O-methyl, a 2′-Fluoro, or a 2′-O-methoxyethyl moiety. For example, the ASO comprises a phosphorodiamidate morpholino moiety. In another example, the ASO comprises a locked nucleic acid. In a further example, the ASO comprises a 2-O-methyl moiety. In one example, the ASO comprises a 2′-Fluoro moiety. In another example, the ASO comprises a 2′-O-methoxyethyl moiety.

In one example, the ASO comprises at least one modified sugar moiety. For example, each sugar moiety in the antisense oligonucleotide is a modified sugar moiety.

In one example, the ASO comprises a 2′-O-methoxyethyl moiety. For example, each nucleotide of the ASO comprises a 2′-O-methoxyethyl moiety.

In one example, the nucleotide sequence of the ASO consists of 10 to 50 nucleotides, 15 to 40 nucleotides, 18 to 40 nucleotides, 17 to 25 nucleotides, 20 to 35 nucleotides, 20 to 30 nucleotides, 22 to 30 nucleotides, 22 to 28 nucleotides, 24 to 30 nucleotides, 25 to 30 nucleotides, or 26 to 30 nucleotides. In one example, the nucleotide sequence of the ASO consists of 20 to 30 nucleotides. For example, the nucleotide sequence of the ASO consists of 17 nucleotides. In one example, the nucleotide sequence of the ASO consists of 19 nucleotides. In another example, the nucleotide sequence of the ASO consists of 21 nucleotides. In a further example, the nucleotide sequence of the ASO consists of 22 nucleotides. In one example, the nucleotide sequence of the ASO consists of 23 nucleotides. In another example, the nucleotide sequence of the ASO consists of 24 nucleotides. In another example, the nucleotide sequence of the ASO consists of 25 nucleotides. In another example, the nucleotide sequence of the ASO consists of 26 nucleotides. In another example, the nucleotide sequence of the ASO consists of 27 nucleotides. In another example, the nucleotide sequence of the ASO consists of 28 nucleotides. In another example, the nucleotide sequence of the ASO consists of 29 nucleotides. In another example, the nucleotide sequence of the ASO consists of 30 nucleotides.

In one example, the ASO comprises one or more phosphorodiamidate morpholino moieties.

In one example of any method described herein, the ASO is linked to a functional moiety. The functional moiety can be covalently linked or non-covalently linked to the ASO. The functional moiety can be at the 5′ end and/or 3′ end of the ASO.

In some examples, the functional moiety comprises a delivery moiety. For example, the delivery moiety is selected from the group consisting of lipids, peptides, carbohydrates, and antibodies. An exemplary delivery moiety comprises a cell-penetrating peptide (CPP). The present disclosure additionally contemplates delivery moieties such as a N-acetylgalactosamine (GalNAc) moiety, a fatty acid moiety, or a lipid moiety.

In some examples, the functional moiety comprises a stabilising moiety.

The present disclosure additionally provides a pharmaceutical composition comprising an ASO of the disclosure, and a pharmaceutically acceptable excipient, for use in any method of the disclosure.

In one example, the ASO is complexed with a delivery nanocarrier. For example, the delivery nanocarrier is selected from the group consisting of: lipoplexes, liposomes, exosomes, inorganic nanoparticles, and DNA nanostructures. In one example, the delivery nanocarrier comprises a lipid nanoparticle (LNP) encapsulating the antisense oligonucleotide.

In one example of any method described herein, the ASO is formulated for a route of administration selected from the group consisting of intravitreal, suprachoroidal, subretinal, ciliary intramuscular, intravenous, intra-arterial, subcutaneous, and topical routes.

The present disclosure also provides use of an ASO in the manufacture of a medicament for treating, preventing and/or delaying progression of glaucoma in a subject, wherein the ASO modulates mRNA translation of the OPA1 gene transcript or part thereof.

The disclosure also provides a modified cell comprising an ASO of the disclosure for use in any method described herein. For example, the modified cell is a mammalian cell, such as a human cell.

The disclosure additionally provides an ASO that binds to a targeted portion of the intron 7× of an OPA1 gene transcript in a cell and increases the level of OPA1 gene transcripts encoding full length, functional OPA1 by exclusion of NMD exon 7×. For example, the ASO comprises or consists of any one of SEQ ID NOs: 2-54. In one example, the ASO comprises or consists of any one of SEQ ID NOs: 2-54 or SEQ ID NOs: 2491-2503. In another example, the ASO comprises or consists of any one of SEQ ID NOs: 2491-2503.

The disclosure additionally provides an ASO that binds to a targeted portion of the 5′ UTR of an OPA1 gene transcript in a cell and increases transcript stability of an OPA1 mRNA, e.g., by inhibiting the activity of a decapping enzyme.

In one example, the ASO comprises or consists of any one of SEQ ID NOs: 56-138.

The disclosure additionally provides an ASO that binds to a targeted portion of the 3′ UTR of an OPA1 gene transcript in a cell and increases transcript stability of an OPA1 mRNA, e.g., sterically inhibiting binding of a miRNA to the 3′ UTR.

In one example, the ASO binds within a targeted portion of the 3′ UTR of OPA1 mRNA, wherein the targeted portion is within the nucleotide sequence corresponding to SEQ ID NO: 139.

In one example, the ASO that binds to a targeted portion of the 3′ UTR of OPA1 mRNA comprises or consists of any one of SEQ ID NOs: 140-2488.

The present disclosure additionally provides a method of treating a condition, the method comprising administering an ASO of the disclosure. In one example, the condition is associated with OPA1 expression, e.g., reduced OPA1 expression. In one example, the condition is glaucoma. In another example, the condition is autosomal dominant optic atrophy.

### BRIEF DESCRIPTION OF THE DRAWINGS

FIG. 1 includes a series of graphical representations showing the binding sites for ASOs that increase OPA1 protein levels. (A) Schematic illustration of exon structure of OPA1 (Transcript ID: ENST00000361510), indicating start and stop codons and the regions of 5′ UTR, NMD exon 7× (in case of unspliced) and 3′ UTR. (B) Prediction for secondary structure of the 5′ UTR of OPA1 transcript (corresponding to SEQ ID NO: 55) using RNAfold web tool (http://ma.tbi.univie.ac.at/cgi-bin/RNAWebSuite/RNAfold.cgi). The free energy of the thermodynamic ensemble is −137.26 kcal/mol. ASOs 56-138 were designed, which target start codons of upstream open reading frames (uORFs), regulatory binding sites, inhibitory 5′ UTR secondary structures and/or G-quadruplexes. (C) Exon 7× (black box) containing the premature termination codon (PTC), is located between exons 7 and 8 (not drawn to scale). ASOs (SEQ ID NOs: 2-54) were designed to target splicing regulatory elements within intron 7 (dash line, corresponding to SEQ ID NO: 1) to mediate exclusion of exon 7× during pre-mRNA splicing to increase productive OPA1 transcript. (D) Schematic illustration of the 3′ UTR (not drawn to scale) located in exon 31. ASOs were designed to hybridize with the transcript and mask/inhibit binding of miRNA(s) to prevent mRNA degradation and increase in OPA1 protein levels.

FIG. 2 shows screening of PMOs (25 and 50 μM) in ADOA patient fibroblasts. Patient fibroblasts were transfected for 48 hr with PMOs targeting removal of the OPA1 exon 7× as indicated. OPA1 transcript expression was assessed by digital droplet PCR (ddPCR) and normalised to GAPDH, RPL27 and SCL25A3 transcript levels. The OPA1 expression in untreated cells was set to 1.

FIG. 3 shows screening of PMOs (50 and 100 μM) in ADOA patient fibroblasts. (A) The western blot gel image shows expression of long and short OPA1 isoforms in patient fibroblasts transfected with PMOs targeting intron 7 of the OPA1 transcript at 48 hr. (B) The band intensity of OPA1 expression was normalised to beta-actin (assessed by ImageJ™). The OPA1 expression in untreated cells was set to 1.

FIG. 4 is a schematic of the refinement of antisense oligonucleotides to improve OPA1 upregulation. (A) Illustration of OPA1 exons and the location of exon7× exists in the transcript. (B) Binding region of parental PMOs on OPA1 transcript upstream of exon7×. Exon 7× is not drawn to scale. (C) Binding region of daughter sequences with microwalk, nucleotide base substitution and lengthening to improve the efficacy of PMOs.

FIG. 5 shows screening of cell penetrating peptide-conjugated PMOs (PPMOs) (5, 10 and 20 μM) in ADOA patient fibroblasts. ADOA patient fibroblasts were transfected for 5 days with PPMOs targeting intron 7 of the OPA1 transcript as indicated. OPA1 transcript expression was assessed by ddPCR and normalised to HPRTL. The OPA1 expression in untreated cells was set to 1.

FIG. 6 shows screening of PMOs targeting exon 7× exclusion (25 and 50 μM) in ADOA patient fibroblasts. Patient fibroblasts were transfected in triplicates for 48 hr with PMOs targeting removal of the OPA1 exon 7× as indicated. Experiments were performed in 1-4 biological replicates as indicated with the number of data points within a bar graph. OPA1 transcript expression was assessed by ddPCR and normalised to the HPRT1 transcript level. The OPA1 expression in untreated cells was set to 1.

FIG. 7 shows screening of 5′ UTR PMOs (25 and 50 μM) in ADOA patient fibroblasts. PMOs with SEQ ID NOs: 78, 112 and 2500-2503 were transfected into ADOA patient fibroblasts in triplicates for 72 hr with PMOs targeting the 5′ UTR of an OPA1 mRNA. Western blot analysis was used to determine the upregulation of OPA1 protein in PMO-treated cells. The band intensity of OPA1 expression was normalised to HPRT1 (assessed by ImageJ™). The OPA1 expression in untreated cells was set to 1. PMOs SEQ ID NOs: 78, 112 and 2502 significantly increased OPA1 protein upregulation (greater than 1.3 fold) in patient fibroblasts. Student's t test was used for statistical analysis.

FIG. 8 shows the PMO OPA1 H1A(+10+32)1 mm10C>T (SEQ ID NO: 112) was conjugated with CPP for enhanced cell penetrating ability. The CPP-PMO (or PPMO) was incubated for 7 days to dermal skin fibroblasts derived from ADOA patients containing OPA1 mutations c. 2708_2711delTTAG (patient 1) and c.985-1G>A (patient 2). The efficacy of PPMO-induced OPA1 upregulation was assessed using western blot assay. The results showed significant OPA1 protein upregulation in a dose dependent manner in 2 patients with distinct OPA1 mutations. Student's t test was used for statistical analysis.

FIG. 9 shows the mitochondrial functional improvement following PPMO treatment in ADOA patient-derived fibroblasts. A PMO OPA1 H1A(+10+32)1 mm10C>T (SEQ ID NO: 112) was incubated to fibroblasts for 7 days in a 6-well plate format. Upon day 7, cells were trysinised and reseeded into a 96-well plate at 8,000 cells/well and incubated in glucose-depleted DMEM cell culture media supplemented with 2.5 mM 2-deoxy-D-glucose and 5 mM pyruvate for 18 hrs. A CellTiter-Glo® assay was used to assess mitochondrial ATP and calculate the concentration of ATP according to a standard curve using (14.7-10,000 nM of ATP standard dilutions (ThermoFisher). Student's t test was used for statistical analysis.

FIG. 10 shows the PMO OPA1 H1A(+10+32)1 mm10C>T (SEQ ID NO: 112) enhance OPA1 protein upregulation in enriched iPSC-derived RGCs obtained from an ADOA patient harbouring OPA1 c.985-1G>A mutation. iPSC-RGCs were incubated with PPMO for 5 days prior to protein harvest. The efficacy of PPMO-induced OPA1 upregulation was assessed using western blot assay and normalised to beta-actin expression. The results showed significant OPA1 protein upregulation at 10 μM. Student's t test was used for statistical analysis.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, molecular biology, microbiology, virology, recombinant DNA technology, peptide synthesis in solution, solid phase peptide synthesis, and immunology).

Unless otherwise indicated, the conventional techniques of molecular biology, microbiology, virology, recombinant DNA technology, peptide synthesis in solution, solid phase peptide synthesis, and immunology utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

The term “about”, unless stated to the contrary, refers to +/−20%, more preferably +/−10%, of the designated value. For the avoidance of doubt, the term “about” followed by a designated value is to be interpreted as also encompassing the exact designated value itself (for example, “about 10” also encompasses 10 exactly).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

The term “antisense oligonucleotide” “antisense oligomer” or “ASO,” as used herein, encompasses oligonucleotides and any other oligomeric molecule that comprises nucleobases capable of hybridizing to a complementary sequence on a target RNA transcript, but does not comprise a sugar moiety, such as in the case of a peptide nucleic acid (PNA). Preferably, the ASO is an ASO that is resistant to nuclease cleavage or degradation.

The phrase “binds to a targeted portion” or “binds within a targeted portion,” in reference to an ASO, as used herein, refers to specific hybridization between the ASO nucleotide sequence and a target nucleotide sequence that is complementary within the ranges set forth herein. In some examples, specific hybridization occurs where, under ex vivo conditions, the hybridization occurs under high stringency conditions. By “high stringency conditions” is meant that the ASO, under such ex vivo conditions, hybridize to a target sequence in an amount that is detectably stronger than non-specific hybridization. High stringency conditions, then, are conditions that distinguish a polynucleotide with an exact complementary sequence, or one containing only a few scattered mismatches from a random sequence that happened to have a few small regions (e.g., 1-5 bases) that matched the probe. Such small regions of complementarity are more easily melted than a full-length complement of 12-17 or more bases, and moderate stringency hybridization makes them easily distinguishable. In one example, high stringency conditions include, for example, low salt and/or high temperature conditions, such as provided by about 0.02-0.1 M NaCl or the equivalent, at temperatures of about 50-70° C. The skilled person will appreciate that under in vivo conditions, the specificity of hybridization between an ASO and its target sequence is defined in terms of the level of complementarity between the ASO and the target sequence to which it hybridizes within a cell.

The term “nonsense-mediated RNA decay-inducing (NMD) exon” or “NMD exon” refers to an exon or a pseudo-exon that is a region within an intron and can activate the NMD pathway if included in a mature RNA transcript. In the constitutive splicing events, the intron containing an NMD exon is usually spliced out, but the intron or a portion of it can be retained during alternative or aberrant splicing events. Mature mRNA transcripts containing such an NMD exon can be non-productive due to a frame shift which induces the NMD pathway. Inclusion of an NMD exon in mature OPA1 RNA transcripts can downregulate overall OPA1 mRNA and OPA1 protein expression.

The term “precursor mRNA” or “pre-mRNA” refers to the primary transcript is the single-stranded RNA product synthesized by transcription of the genomic DNA sequence of the transcription unit for a particular gene, which generally encompasses the nucleotide sequence between a transcription start site and a termination signal.

The term “peptide” is intended to include compounds composed of amino acid residues linked by amide bonds. A peptide may be natural or unnatural, ribosome encoded or synthetically derived. Typically, a peptide will consist of between 2 and 200 amino acids. For example, the peptide may have a length in the range of 10 to 20 amino acids or 10 to 30 amino acids or 10 to 40 amino acids or 10 to 50 amino acids or 10 to 60 amino acids or 10 to 70 amino acids or 10 to 80 amino acids or 10 to 90 amino acids or 10 to 100 amino acids, including any length within said range(s). The peptide may comprise or consist of fewer than about 150 amino acids or fewer than about 125 amino acids or fewer than about 100 amino acids or fewer than about 90 amino acids or fewer than about 80 amino acids or fewer than about 70 amino acids or fewer than about 60 amino acids or fewer than about 50 amino acids.

Peptides, as referred to herein, include “inverso” peptides in which all L-amino acids are substituted with the corresponding D-amino acids, “retro-inverso” peptides in which the sequence of amino acids is reversed and all L-amino acids are replaced with D-amino acids.

Peptides may comprise amino acids in both L- and/or D-form. For example, both L- and D-forms may be used for different amino acids within the same peptide sequence. In some examples the amino acids within the peptide sequence are in L-form, such as natural amino acids. In some examples the amino acids within the peptide sequence are a combination of L- and D-form. Further, peptides may comprise unusual, but naturally occurring, amino acids including, but not limited to, hydroxyproline (Hyp), beta-alanine, citrulline (Cit), ornithine (Orn), norleucine (Nle), 3-nitrotyrosine, nitroarginine, pyroglutamic acid (Pyr). Peptides may also incorporate unnatural amino acids including, but not limited to, homo amino acids, N-methyl amino acids, alpha-methyl amino acids, beta (homo) amino acids, gamma amino acids, and N-substituted glycines. Peptides may be linear peptides or cyclic peptides.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical bond or a disulfide bond. Examples of non-covalent bonds include hydrogen bonds, ionic bonds, Van der Waals forces, and hydrophobic interactions.

Percentage amino acid sequence identity with respect to a given amino acid sequence is defined as the percentage of amino acid residues in a candidate sequence that are identical to the amino acid residues in the reference sequence, after aligning the sequences and introducing gaps, if necessary, to achieve the maximum percent sequence identity, and not considering any conservative substitutions as part of the sequence identity. Amino acid sequence identity may be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term “cell penetrating peptide” (CPP) refers to a peptide that is capable of crossing a cellular membrane. In one example, a CPP is capable of translocating across a mammalian cell membrane and entering into a cell. In another example, a CPP may direct a conjugate to a desired subcellular compartment. Thus, a CPP may direct or facilitate penetration of a molecule of interest across a phospholipid, mitochondrial, endosomal, lysosomal, vesicular, or nuclear membrane. A CPP may be translocated across the membrane with its amino acid sequence complete and intact, or alternatively partially degraded.

A CPP may direct a molecule of interest, such as an antisense oligonucleotide disclosed herein, from outside a cell through the plasma membrane, and into the cytoplasm or a desired subcellular compartment. Alternatively, or in addition, a CPP may direct a molecule of interest across the blood-brain, trans-mucosal, hematoretinal, skin, gastrointestinal and/or pulmonary barriers.

The term “peptide ligand” or “receptor binding domain” refers to a peptide that is capable of binding to a membrane surface receptor to enable translocation of the peptide across a cellular membrane. In one example a peptide ligand may enable translocation across the cellular membrane via the natural endocytosis of the targeted receptor. In another example the peptide ligand may utilise a complementary mechanism of translocation across the cellular membrane including utilising a conjugated CPP. In one example, a peptide ligand is capable of translocating across a mammalian cell membrane and to enter a cell. In another example, a peptide ligand may direct a conjugate to a desired subcellular compartment. Thus, a peptide ligand may direct or facilitate cellular uptake of a molecule of interest across a phospholipid, mitochondrial, endosomal, lysosomal, vesicular, or nuclear membrane. A peptide ligand may be translocated across the membrane with its amino acid sequence complete and intact, or alternatively partially degraded.

A peptide ligand via its binding to a target receptor may direct a molecule of interest, such as an ASO disclosed herein, from outside a cell through the plasma membrane, and into the cytoplasm or a desired subcellular compartment. Alternatively, or in addition, a peptide ligand via its binding to a target receptor may direct a molecule of interest across a relevant biological barrier, e.g., the blood-brain, trans-mucosal, hematoretinal, skin, gastrointestinal, and/or pulmonary barriers.

### Methods of Treating or Preventing Glaucoma

The present disclosure provides, for example, a method of treating, preventing and/or delaying progression of glaucoma in a subject. The methods described herein include a method for treating, preventing and/or delaying progression of glaucoma in a subject in need thereof by administering to the subject a therapeutically effective amount of a pharmaceutical composition comprising any of the ASOs disclosed herein. Likewise, in some examples, any of the ASOs herein are used in the manufacture of a medicament for treating, preventing and/or delaying progression of glaucoma.

Glaucoma is a group of eye diseases that result in vision loss. It is typically caused by an increase in intraocular pressure (IOP) which can result in damage to the optic nerve. Glaucoma can be classified as either primary or secondary, with secondary glaucoma attributable to another disorder or problem within the eye, such as injury, surgery, drugs, or other ocular diseases. Primary glaucoma is classified as open-angle glaucoma (POAG), normal-tension glaucoma (NTG), angle-closure glaucoma and congenital glaucoma. Secondary glaucoma is classified into neovascular glaucoma, pigmentary glaucoma, exfoliation glaucoma and uveitic glaucoma.

In one example of the methods of the present disclosure, the glaucoma is primary glaucoma. For example, the primary glaucoma is open-angle glaucoma (POAG), normal-tension glaucoma (NTG), angle-closure glaucoma or congenital glaucoma.

In one example, the primary glaucoma is open-angle glaucoma (POAG).

In one example, the primary glaucoma is normal-tension glaucoma (NTG).

In one example, the primary glaucoma is angle-closure glaucoma.

In one example, the primary glaucoma is congenital glaucoma.

In one example of the methods of the present disclosure, the glaucoma is secondary glaucoma. For example, the secondary glaucoma is neovascular glaucoma, pigmentary glaucoma, exfoliation glaucoma or uveitic glaucoma.

In one example, the secondary glaucoma is neovascular glaucoma.

In one example, the secondary glaucoma is pigmentary glaucoma.

In one example, the secondary glaucoma is exfoliation glaucoma.

In one example, the secondary glaucoma is uveitic glaucoma.

In one example, the subject to be treated is suffering from glaucoma. For example, the subject has been diagnosed as having or suffering from glaucoma. In one example, the subject suffers from glaucoma. For example, the subject is in need of treatment. Such subjects can be administered the ASOs as described here to treat or prevent the progression of glaucoma.

In one example, administration of an ASO as described herein slows progression of glaucoma.

In one example, the subject is at risk of developing glaucoma. Such subjects can be administered the ASOs as described here to prevent onset of glaucoma.

As used herein, the term “at risk” means that the subject has an increased chance of developing glaucoma compared to a normal individual. Subjects can be identified as at risk of developing glaucoma using any method known in the art and/or those described herein. For example, the subject may be identified at risk of developing glaucoma if that subject has one or more common risk factors including family history, high eye pressure, diabetes, high or low blood pressure and prolonged use of steroidal medication.

Also provided herein is a method for increasing the OPA1 protein in a cell, the method comprising contacting the cell with a composition or pharmaceutical composition, as disclosed herein, whereby the amount of OPA1 protein in the cell is increased. Also provided herein is a method for increasing the level of OPA1 protein in a cell, ex vivo or in a tissue in vivo, the method comprising contacting the cell with an ASO or pharmaceutical composition, as disclosed herein, whereby the amount of OPA1 protein in the cell is increased. In some examples, the cell is a retinal cell. In some examples, the tissue is a retinal tissue, e.g., retina and/or retinal pigment epithelium.

In some examples, administration to a subject or contact with cells with any of the ASOs or pharmaceutical compositions disclosed herein increases the level of OPA1 protein about 1.1 to about 10-fold, e.g., 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the level in the tissue prior to the administration or contact.

Suitable routes of administration for treatment with the compositions, pharmaceutical compositions, or medicaments disclosed herein include, but are not limited to, intravitreal, suprachoroidal, subretinal, ciliary intramuscular, intravenous, intra-arterial, subcutaneous, and topical.

In some examples administration is into the eye by an intravitreal, suprachoroidal, or sub-retinal route. For example, administration to the eye is by intravitreal administration. In another example, administration to the eye is by suprachoroidal administration. In a further example, administration to the eye is by sub-retinal administration. In one example, administration to the eye is by a topical administration.

As the skilled person will understand, the treatment methods disclosed herein include administration of the compositions and pharmaceutical compositions disclosed herein in a therapeutically effective amount to a subject (e.g., a human subject). The terms “effective amount” or “therapeutically effective amount,” as used herein, refer to a sufficient amount of a disclosed ASO being administered to relieve to some extent one or more of the symptoms and/or clinical indicia associated with pathological inflammation in a particular disease or health condition. In some examples, an “effective amount” for therapeutic uses is the amount of one of the foregoing agents required to provide a clinically significant decrease in disease symptoms and/or inflammatory markers or to prevent disease symptoms without undue adverse side effects. An appropriate “effective amount” in any individual case may be determined using techniques, such as a dose escalation study. The term “therapeutically effective amount” includes, for example, a prophylactically effective amount. It is understood that “an effective amount” or “a therapeutically effective amount” can vary from subject to subject, due to variation in metabolism of the compound of any age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial. Where more than one therapeutic agent is used in combination, a “therapeutically effective amount” of each therapeutic agent can refer to an amount of the therapeutic agent that would be therapeutically effective when used on its own, or may refer to a reduced amount that is therapeutically effective by virtue of its combination with one or more additional therapeutic agents.

### Compositions for Increasing OPA1 Protein Levels

OPA1 mitochondrial dynamin like GTPase gene (also known as OPA1, FLJ12460, KIAA0567, MGM1, NPG and NTG; referred to herein as OPA1) is composed of 30 coding exons distributed across more than 90 kb of genomic DNA. It is located on chromosome 3q29 and encodes for a ubiquitously expressed dynamic-related GTPase, which is imported into mitochondria by an N-terminal import sequence and localizes to the inner membrane facing the intermembrane space. OPA1 contains a highly conserved functional GTPase domain shared by members of the dynamin superfamily of mechanoenzymes and regulates several important cellular processes including the stability of the mitochondrial network. In humans, OPA1 generates at least eight isoforms via differential splicing of exons 4, 4b and 5b. For the purposes of nomenclature only and not limitation the sequence of the entire human OPA1 gene sequence and known transcript maps and sequences are publicly available through the online ensembl database under record ENSG00000198836. An exemplary gene sequence of human OPA1 is set out in NCBI Reference Sequence NM_130837, or SEQ ID NO: 2489, and UniProt ID 060313, or SEQ ID NO: 2490.

The OPA1 gene contains an intron with a premature termination codon (PTC) in intron 7 (located between exons 7 and 8). In some subjects, a proportion of the OPA1 RNA transcripts from wild-type OPA1 genes retain a section of intron 7 containing this PTC; this retained intron section is called exon 7× in the transcribed RNA. The RNA transcripts that contain exon 7× (the retained intron segment containing the PTC) are subject to nonsense-mediated RNA decay. Therefore, a proportion of OPA1 RNA that is translated to mature wild-type protein, and a portion of OPA1 RNA that is degraded by RNase almost immediately due to the presence of the PTC.

As described herein, the ASOs according to any example bind to a targeted portion of human OPA1 pre-mRNA and which increase expression of OPA1 protein by promoting the exclusion of exon 7× in splicing of OPA1 in mammalian cells.

Without being bound by theory or mode of action, the ASOs that bind to targeted portions of human OPA1 pre-mRNA in mammalian cells and which result in the exclusion of NMD exon 7×, are thought to increase expression of OPA1 protein by preventing the translation of NMD exon 7×.

Also described herein, the ASOs according to any example bind to the 5′ UTR or 3′ UTR of OPA1 mRNA and increase expression of OPA1 protein.

Without being bound by theory or mode of action, the ASOs that bind to the 5′ UTR are thought to increase expression of OPA1 protein through steric inhibition of translation from upstream Open Reading Frames (uORF) start site and/or steric inhibition of secondary structure in the UTR and/or inhibiting the binding and/or activity of a decapping enzyme.

Also described herein, the ASOs according to any example bind to the 3′ UTR or 3′ UTR of OPA1 mRNA and increase expression of OPA1 protein. Without being bound by theory or mode of action, such ASOs may sterically inhibit binding of a miRNA to the 3′ UTR.

### Antisense Oligonucleotides (ASOs)

In some examples of the compositions and methods described herein, ASOs have a sequence that is completely complementary across its length to the target sequence or a sequence near complementarity (e.g., sufficient complementarity to bind the target sequence to promote exon splicing). ASOs are designed so that they bind (hybridize) to a target RNA sequence (e.g., a targeted portion of a pre-mRNA transcript) and remain hybridized under physiological conditions. Selection of suitable sequences for ASOs generally avoids, where possible, similar nucleic acid sequences in other (i.e., off-target) locations in the genome or in cellular mRNAs or miRNAs, such that the likelihood the ASO will hybridize at such sites is limited.

In some examples, ASOs “specifically hybridize” to or are “specific” to a target nucleic acid or a targeted portion of the OPA1 mRNA 5′ UTR. In some examples, ASOs “specifically hybridize” to or are “specific” to a target nucleic acid or a targeted portion of the OPA1 pre-mRNA. At a given ionic strength and pH, the Tₘ is the temperature at which 50% of a target sequence hybridizes to a complementary oligonucleotide.

ASO sequences are “complementary” to their target sequences when hybridization occurs in an antiparallel configuration between two single-stranded polynucleotides. Complementarity is quantifiable in terms of the proportion (e.g., the percentage) of bases in opposing strands that are expected to form hydrogen bonds with each other, according to generally accepted base-pairing rules. The nucleotide sequence of an ASO need not be 100% complementary to that of its target nucleic acid to hybridize. In certain examples, the nucleotide sequences of ASOs in the compositions disclosed herein can be at least 60%, at least 65%, at least 70%, at least 75%, at least 80%, at least 85%, at least 90%, at least 95%, or 100% complementary to the nucleotide sequence of the targeted portion of an RNA transcript over the length of the ASO nucleotide sequence. For example, an ASO in which 18 of 20 nucleotides of ASO sequence are complementary to a target region, and would therefore specifically hybridize, would represent 90 percent complementarity. In such an example, the remaining non-complementary nucleotides of the ASO could be clustered together or interspersed with complementary nucleotides and need not be contiguous. Complementarity of an ASO sequence to a target nucleotide sequence (expressed as “percent complementarity” to its target sequence; or “percent identity” to its reverse complement sequence) can be determined routinely using algorithms known in the art, as exemplified in the BLAST programs (basic local alignment search tools) and PowerBLAST programs (Altschul, et al., 1990, J Mol. Biol., 215:403-410; Zhang et al., 1997, Genome Res., 7:649-656).

In some examples, an ASO does not hybridize to all nucleotides in a target sequence and the nucleotide positions at which it does hybridize may be contiguous or noncontiguous. ASOs may hybridize over one or more segments of a 5′ UTR region of a mRNA or over one or more segments of intron 7 of the OPA1 pre-mRNA, such that intervening or adjacent segments are not involved in the hybridization event (e.g., a loop structure or hairpin structure may be formed).

In some examples the nucleotide sequences of ASOs described herein are complementary to a targeted portion of OPA1 mRNA 5′ UTR. For example, the ASOs are complementary to a targeted portion of the 5′ UTR of an OPA1 mRNA corresponding to SEQ ID NO:55. In some examples, the ASOs are complementary to a targeted portion of an OPA1 mRNA corresponding to SEQ ID NO:55, which encompasses the 5′ UTR. In some examples the nucleotide sequence of the ASO is at least 60%, 65%, 70%, 75%, 80%, 85%, 90%, 95%, or 100% complementary to the nucleotide sequence of the targeted portion of the OPA1 5′ UTR over the length of the ASO.

In some examples, the nucleotide sequences of ASOs that bind to targeted portions of the 5′ UTR of OPA1 mRNA comprise or consist of any one of SEQ ID NOs: 56-138.

In some examples the nucleotide sequences of ASOs described herein are complementary to a targeted portion of intron 7 of the OPA1 pre-mRNA. In some examples, the ASOs are complementary to a targeted portion within sufficient proximity to an acceptor site of exon 7× to promote exclusion of exon 7× in splicing of OPA1 mRNA e.g. the antisense oligonucleotide comprises any one of SEQ ID NOs: 2-54. In some examples, the ASOs are complementary to a targeted portion within sufficient proximity to an acceptor site of exon 7× to promote exclusion of exon 7× in splicing of OPA1 mRNA e.g. the antisense oligonucleotide comprises any one of SEQ ID NOs: 2-54 or SEQ ID NOs: 2491-2503 In some examples, the ASOs are complementary to a targeted portion within sufficient proximity to an acceptor site of exon 7× to promote exclusion of exon 7× in splicing of OPA1 mRNA e.g. the antisense oligonucleotide comprises any one of SEQ ID NOs: 2491-2503 In some examples the nucleotide sequence of the ASO is at least 60%, 65%, 70%, 75%, 80%, 85%, 90%, 95%, or 100% complementary to the nucleotide sequence of the targeted portion of intron 7 of the OPA1 pre-mRNA over the length of the ASO.

In some examples the nucleotide sequences of ASOs described herein are complementary to a targeted portion of OPA1 mRNA 3′ UTR. For example, the ASOs are complementary to a targeted portion of the 3′ UTR of an OPA1 mRNA corresponding to SEQ ID NO: 139. In some examples, the ASOs are complementary to a targeted portion of an OPA1 mRNA corresponding to SEQ ID NO: 139, which encompasses the 3′ UTR. In some examples the nucleotide sequence of the ASO is at least 60%, 65%, 70%, 75%, 80%, 85%, 90%, 95%, or 100% complementary to the nucleotide sequence of the targeted portion of the OPA1 3′ UTR over the length of the ASO.

The ASOs described herein may be of any length suitable for specific hybridization to a target sequence. In some examples, the nucleotide sequence of the ASOs consist of 8 to 50 nucleotides. For example, the ASO sequence can be 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleotides in length. In some examples, the ASOs consist of more than 50 nucleotides, but no more than 100 nucleotides in length.

In some examples, the ASO nucleotide sequence is from 8 to 50 nucleotides, 8 to 40 nucleotides, 8 to 35 nucleotides, 8 to 30 nucleotides, 8 to 25 nucleotides, 8 to 20 nucleotides, 8 to 15 nucleotides, 9 to 50 nucleotides, 9 to 40 nucleotides, 9 to 35 nucleotides, 9 to 30 nucleotides, 9 to 25 nucleotides, 9 to 20 nucleotides, 9 to 15 nucleotides, 10 to 50 nucleotides, 10 to 40 nucleotides, 10 to 35 nucleotides, 10 to 30 nucleotides, 10 to 25 nucleotides, 10 to 20 nucleotides, 10 to 15 nucleotides, 11 to 50 nucleotides, 11 to 40 nucleotides, 11 to 35 nucleotides, 11 to 30 nucleotides, 11 to 25 nucleotides, 11 to 20 nucleotides, 11 to 15 nucleotides, 12 to 50 nucleotides, 12 to 40 nucleotides, 12 to 35 nucleotides, 12 to 30 nucleotides, 12 to 25 nucleotides, 12 to 20 nucleotides, 12 to 15 nucleotides, 13 to 50 nucleotides, 13 to 40 nucleotides, 13 to 35 nucleotides, 13 to 30 nucleotides, 13 to 25 nucleotides, 13 to 20 nucleotides, 14 to 50 nucleotides, 14 to 40 nucleotides, 14 to 35 nucleotides, 14 to 30 nucleotides, 14 to 25 nucleotides, 14 to 20 nucleotides, 15 to 50 nucleotides, 15 to 40 nucleotides, 15 to 35 nucleotides, 15 to 30 nucleotides, 15 to 25 nucleotides, 15 to 20 nucleotides, 20 to 50 nucleotides, 20 to 40 nucleotides, 20 to 35 nucleotides, 20 to 30 nucleotides, 20 to 25 nucleotides, 25 to 50 nucleotides, 25 to 40 nucleotides, 25 to 35 nucleotides, or 25 to 30 nucleotides in length. In some examples, the ASOs are 17 nucleotides in length. In some preferred examples, the nucleotide sequence of the ASO nucleotide is 25 nucleotides in length.

### ASO Chemistry and Modifications

The ASOs used in the compositions described herein may comprise naturally-occurring nucleotides, nucleotide analogues, modified nucleotides, or any combination thereof. The term “naturally occurring nucleotides” includes deoxyribonucleotides and ribonucleotides. The term “modified nucleotides” includes nucleotides with modified or substituted sugar groups and/or having a modified backbone. In some examples, all the nucleotides of an ASO are modified nucleotides. Chemical modifications of ASOs or components of ASOs that are compatible with the compositions and methods described herein are known in the art as disclosed in, e.g., in U.S. Pat. Nos. 8,258,109, 5,656,612, U.S. Patent Publication No. 2012/0190728, and Roberts et al., 2020, Nature Rev. Drug Disc., 19:673-694.

One or more nucleotides of an ASO may be any naturally occurring, unmodified nucleobase such as adenine, guanine, cytosine, thymine, uracil and inosine, or any synthetic or modified nucleobase that is sufficiently similar to an unmodified nucleobase such that it is capable of hydrogen bonding with a nucleobase present on a target RNA transcript. Examples of suitable modified nucleobases include, but are not limited to, hypoxanthine, xanthine, 7-methylguanine, 5, 6-dihydrouracil, 5-methylcytosine, and 5 hydroxymethoylcytosine.

ASOs include a “backbone” structure that refers to the connection between nucleotides/monomers of the ASO. In naturally occurring oligonucleotides, the backbone comprises a 3′-5′ phosphodiester linkage connecting sugar moieties of adjacent nucleotides. Suitable types of backbone linkages for the ASOs described herein include, but are not limited to, phosphodiester, phosphorothioate, phosphorodithioate, phosphorodiamidate, phosphoroselenoate, phosphorodiselenoate, phosphoroanilothioate, phosphoraniladate, phosphoramidate, and the like. In some examples, the backbone modification is a phosphorothioate linkage. In other examples, the backbone modification is a phosphorodiamidate linkage. See, e.g., Roberts et al. supra; and Agrawal (2021), Biomedicines, 9:503. In some examples, the backbone structure of the ASO does not contain phosphorous-based linkages, but rather contains peptide bonds, for example in a peptide nucleic acid (PNA), or linking groups including carbamate, amides, and linear and cyclic hydrocarbon groups.

In some examples, the stereochemistry at each of the phosphorus internucleotide linkages of the ASO backbone is random. In other examples, the stereochemistry at each of the phosphorus internucleotide linkages of the ASO backbone is controlled and is not random. For example, U.S. Pat. No. 9,605,019 describes methods for independently selecting the handedness of chirality at each phosphorous atom in an oligonucleotide. In some examples, a composition or composition used in the methods disclosed herein comprises a pure diastereomeric ASO. In other examples, the composition comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some examples, the ASO has a non-random mixture of Rp and Sp configurations at its phosphorus internucleotide linkages. In some examples, an ASO used in the compositions and methods disclosed herein, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp.

In some examples, the ASOs described herein contain a sugar moiety that comprises ribose or deoxyribose, or a modified sugar moiety or sugar analog, including a morpholine ring. Suitable examples of modified sugar moieties include, but are not limited to, 2′ substitutions such as 2′-O-modifications, 2′-O-methyl (2′-O-Me), 2′-O-methoxyethyl (2′MOE), 2′-O-aminoethyl, 2′F, N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some examples, the sugar moiety modification is selected from among 2′-O-Me, 2′F, and 2′MOE. In other examples, the sugar moiety modification is an extra bridge bond, such as in a locked nucleic acid (LNA). In some examples the sugar analogue contains a morpholine ring, such as phosphorodiamidate morpholino (PMO). In some examples, the sugar moiety comprises a ribofuransyl or 2′deoxyribofuransyl modification. In some examples, the sugar moiety comprises 2′4′-constrained 2′-O-methyloxyethyl (cMOE) modifications. In some examples, the sugar moiety comprises cEt 2′, 4′ constrained 2′-0 ethyl BNA modifications. In other examples, the sugar moiety comprises tricycloDNA (tcDNA) modifications. In some examples, the sugar moiety comprises ethylene nucleic acid (ENA) modifications. In some examples, the sugar moiety comprises 2′-O-(2-N-methylcarbamoylethyl) (MCE). Modifications are known in the art as exemplified in Jarver, et al., 2014, Nucleic Acid Therapeutics, 24(1): 37 47.

In some examples, each constituent nucleotide of the ASO is modified in the same way, e.g., every linkage of the backbone of the ASO comprises a phosphorothioate linkage, or each ribose sugar moiety comprises a 2-O-methyl modification. In other examples, a combination of different modifications is used, e.g., an ASO comprising a combination of phosphorodiamidate linkages and sugar moieties comprising morpholine rings (morpholinos).

In some examples, the ASO comprises one or more backbone modifications. In some examples, the ASO comprises one or more sugar moiety modification. In some examples, the ASO comprises one or more backbone modifications and one or more sugar moiety modifications. In some examples, the ASO comprises a 2′MOE modification and a phosphorothioate backbone. In some examples, the ASO comprises a peptide nucleic acid (PNA).

In some examples, the ASO comprises a phosphorodiamidate morpholino (PMO).

The skilled person in the art will appreciate that ASOs may be modified in order to achieve desired properties or activities of the ASO or reduce undesired properties or activities of the ASO. In some examples, an ASO is modified to alter one or more properties. For example, such modifications can: enhance binding affinity to a target sequence on a pre-mRNA transcript; reduce binding to any non-target sequence; reduce degradation by cellular nucleases (e.g., RNase H); improve uptake of an ASO into a cell and/or particular subcellular compartments; alter the pharmacokinetics or pharmacodynamics of the ASO; and/or modulate the half-life of the ASO in vivo.

In some examples, the ASOs comprise one or more 2′-O-(2-methoxyethyl) (MOE) phosphorothioate-modified nucleotides, which have been shown to confer significantly enhanced resistance of ASOs to nuclease degradation and increased bioavailability.

Methods for synthesis and chemical modification of ASOs, as well as synthesis of ASO conjugates is well known in the art, and such ASOs are available commercially.

In some examples, a composition (e.g., a pharmaceutical composition) provided here includes two or more ASOs with different chemistries but complementary to the same targeted portion of the OPA1 mRNA 5′ UTR. In other examples, two or more ASOs that are complementary to different targeted portions of the OPA1 mRNA 5′ UTR.

In some examples, a composition (e.g., a pharmaceutical composition) provided here includes two or more ASOs with different chemistries but complementary to the same targeted portion of intron 7 of the OPA1 pre-mRNA. In other examples, two or more ASOs that are complementary to different targeted portions of intron 7 of the OPA1 pre-mRNA.

In some examples, a composition (e.g., a pharmaceutical composition) provided here includes two or more ASOs with different chemistries but complementary to the same targeted portion of the OPA1 mRNA 3′ UTR. In other examples, two or more ASOs that are complementary to different targeted portions of the OPA1 mRNA 3′ UTR.

In some examples, the compositions disclosed herein include ASOs that are linked to a functional moiety. In some examples, the functional moiety is a delivery moiety, a targeting moiety, a detection moiety, a stabilizing moiety, or a therapeutic moiety. In some examples the functional moiety includes a delivery moiety or a targeting moiety. In some examples the functional moiety includes a stabilizing moiety. In some examples the functional moiety is a delivery moiety.

Suitable delivery moieties include, but are not limited to, lipids, peptides, carbohydrates, and antibodies.

In some examples, the delivery moiety includes a cell-penetrating peptide (CPP). Suitable examples of CPPs are described in, e.g., PCT/AU2020/051397. In some examples the amino acid sequence of the CPP comprises or consists of: RRSRTARAGRPGRNSSRPSAPRGASGGASG (SEQ ID NO: 2504). In one example, the CPP comprises the sequence RRSRTARAGRPGRNSSRPSAPRGASGGASG (SEQ ID NO: 2504), optionally wherein any amino acid other than glycine is a D amino acid. In other examples, the delivery moiety includes a receptor binding domain. In other examples, the delivery moiety includes a carbohydrate. In some examples, a carbohydrate delivery moiety is selected from among N acetylgalactosamine (GalNAc), N-Ac-Glucosamine (GluNAc), and a mannose. In one example, the carbohydrate delivery moiety is GalNac.

In other examples, the delivery moiety includes a lipid. Examples of suitable lipids as delivery moieties include, but are not limited to, cholesterol moiety, a cholesteryl moiety, and aliphatic lipids. In some examples the delivery moiety includes a fatty acid or lipid moiety. In some embodiments the fatty acid chain length is about C8 to C20. Examples of suitable fatty acid moieties and their conjugation to oligonucleotides are found in, e.g., International Patent Publication WO 2019232255 and in Prakash et al., (2019).

In further examples, the delivery moiety includes an antibody, as described in, e.g., Dugal-Tessier et al., (2021), J Clin Med., 10(4):838.

Suitable examples of stabilizing moieties include, but are not limited to, polyethylene glycol (PEG), poly(oligo(ethylene glycol) methyl ether methacrylate) (POEGMA), and Poly(2-oxazoline)s (POx).

In some examples, where an ASO is linked to a functional moiety, the functional moiety is covalently linked to the ASO. In other examples, the functional moiety is non-covalently linked to the ASO.

Functional moieties can be linked to one or more of any nucleotides in an ASO at any of several positions on the sugar, base or phosphate group, as understood in the art and described in the literature, e.g., using a linker. Linkers can include a bivalent or trivalent branched linker. In some examples, the functional moiety is linked to the 5′ end of the ASO. In other examples, the functional moiety is linked to the 3′ end of the ASO. In further examples, the functional moiety is linked to the 5′ end and the 3′ of the ASO.

In some examples compositions comprising any of the ASOs disclosed herein also include a delivery nanocarrier complexed with ASO. In some examples, a delivery nanocarrier is selected from among lipoplexes, liposomes, exosomes, inorganic nanoparticles, and DNA nanostructures. In other examples the delivery nanocarrier includes a lipid nanoparticle encapsulating the ASO. Various delivery ASO-nanocarrier complex formats are known in the art, as reviewed in, e.g., Roberts et al., supra.

### Pharmaceutical Compositions

Also provided herein are pharmaceutical compositions comprising any of the foregoing ASOs, and modified messenger RNAs (mmRNAs) disclosed herein, and formulated with at least a pharmaceutically acceptable excipient, including a carrier, filler, preservative, adjuvant, solubilizer and/or diluent.

Pharmaceutical compositions containing any of the ASOs compositions described herein, for use in the methods disclosed herein, can be prepared according to conventional techniques well known in the pharmaceutical industry and described in the published literature. In some examples, a pharmaceutical composition for treating a subject comprises a therapeutically effective amount of any ASO disclosed herein.

Pharmaceutically acceptable salts are suitable for use in contact with the tissues of humans and lower animals without undue toxicity, irritation, allergic response, etc., and are commensurate with a reasonable benefit/risk ratio. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Exemplary salts useful in a composition of the present disclosure include calcium chloride, magnesium chloride or sodium chloride.

In one example, a composition comprises a buffer. Exemplary buffers useful in a composition of the present disclosure include sodium phosphate.

In some examples, pharmaceutical compositions are formulated into any of a number of possible dosage forms including, but not limited to, ocular emulsions, topical ointments, solutions for intravitreal injection, intravenous administration, intrathecal administration, intracisterna magna administration, tablets, capsules, gel capsules, liquid syrups, and soft gels. In some examples, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some examples, a pharmaceutical formulation disclosed herein is provided in a form including, but not limited to, a solution, emulsion, microemulsion, foam or liposome-containing formulation (e.g., cationic or noncationic liposomes).

In some examples, pharmaceutical formulations comprising any of the ASOs described herein may comprise one or more penetration enhancers, carriers, excipients or other active or inactive ingredients as appropriate and known to the skilled person. In some examples, where a pharmaceutical composition includes liposomes, such liposomes can also include sterically stabilized liposomes, e.g., liposomes comprising one or more specialized lipids. These specialized lipids result in liposomes with enhanced circulation lifetimes. In some examples, a sterically stabilized liposome comprises one or more glycolipids or is derivatized with one or more hydrophilic polymers, such as PEG moiety. In some examples, a surfactant is included in the pharmaceutical formulation.

In some examples, a pharmaceutical composition also includes a penetration enhancer to enhance the delivery of ASOs, e.g., to aid diffusion across cell membranes and/or enhance the permeability of a lipophilic drug. In some examples, the penetration enhancers include a surfactant, a fatty acid, a bile salt, or a chelating agent.

In some examples, a pharmaceutical composition comprises a dose of ASOs ranging from about 0.01 mg/kg to 20 mg/kg, e.g., 0.05 mg/kg, 0.1 mg/kg, 0.2 mg/kg, 0.5 mg/kg, 1 mg/kg, 3 mg/kg, 5 mg/kg, 8 mg/kg, 10 mg/kg, 15 mg/kg, or another dose ranging from about 0.01 mg/kg to 20 mg/kg.

In some examples, a pharmaceutical composition comprises multiple ASOs. In some examples, a pharmaceutical composition comprises, in addition to ASOs, another drug or therapeutic agent suitable for treatment of a subject suffering from glaucoma.

### Combination Therapies

The pharmaceutical compositions comprising any of the ASOs disclosed herein, can also be used in combination with other agents of therapeutic value in the treatment of glaucoma. In general, other agents do not necessarily have to be administered in the same pharmaceutical composition, and may, because of different physical and chemical characteristics, be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

Compositions and pharmaceutical compositions comprising ASOs and an additional therapeutic agent may be administered concurrently (e.g., simultaneously, essentially simultaneously or within the same treatment protocol) or sequentially, depending upon the stage and progression of the glaucoma to be treated, the condition of the patient, and the choice of specific therapeutic agents used. The determination of the order of administration, and the number of repetitions of administration of each therapeutic agent during a treatment protocol, is well within the knowledge of the skilled physician after evaluation of the glaucoma being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies, dosages of co-administered therapeutic agents will of course vary depending on the type of co-agents employed, ASO, and the disease stage of the patient to be treated.

Pharmaceutical compositions comprising ASOs and an additional therapeutic agent which make up a combination therapy disclosed herein may be a combined dosage form or in separate dosage forms intended for substantially simultaneous administration. The pharmaceutical compositions that make up the combination therapy may also be administered sequentially, with either therapeutic agent being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of various physiological parameters may also be evaluated to determine the optimal dose interval.

Examples of suitable therapeutic agents for co-administration with a composition or a pharmaceutical composition disclosed herein include, but are not limited to, prostaglandins (e.g., latanoprost (Xalatan®), travoprost (Travatan Z®), tafluprost (Zioptan®), bimatoprost (Lumigan®) and latanoprostene bunod (Vyzulta®)), beta blockers (e.g., timolol (Betimol®, Istalol®, Timoptic®) and betaxolol (Betoptic®)), alpha-adrenergic agonists (e.g., apraclonidine (Iopidine®) and brimonidine (Alphagan P, Qoliana®)), carbonic anhydrase inhibitors (e.g., dorzolamide (Trusopt®) and brinzolamide (Azopt®)), a rho kinase inhibitor (e.g., netarsudil (Rhopressa®)) and miotic or cholinergic agents (e.g., pilocarpine (Isopto Carpine®)).

The present disclosure is not to be limited by the following non-limiting examples.

### EXAMPLES

### Example 1: ASO Design to Target Exclusion of an OPA1 NMD Exon 7×

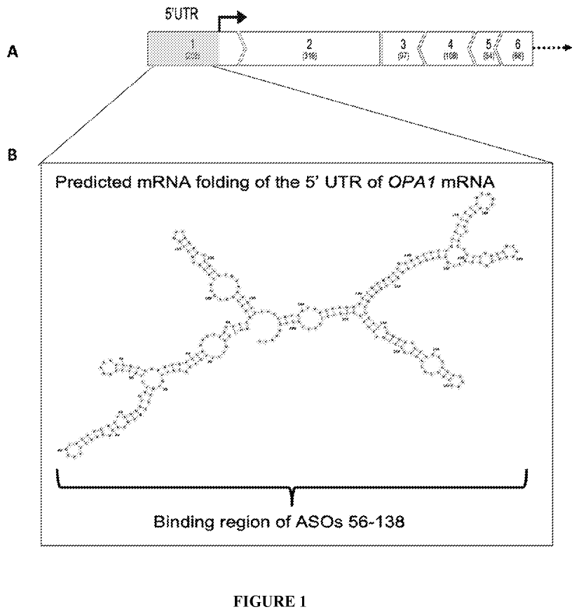

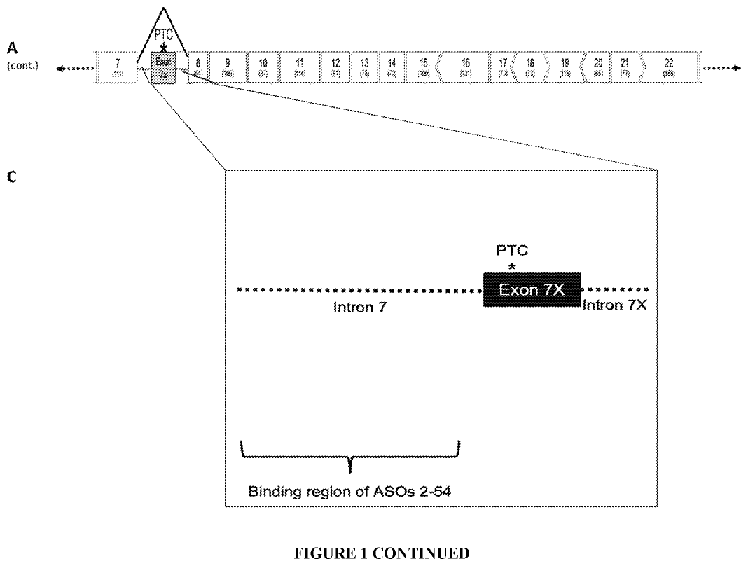

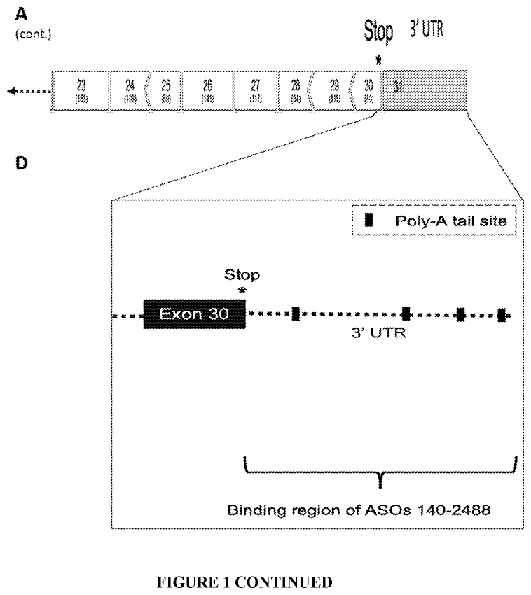

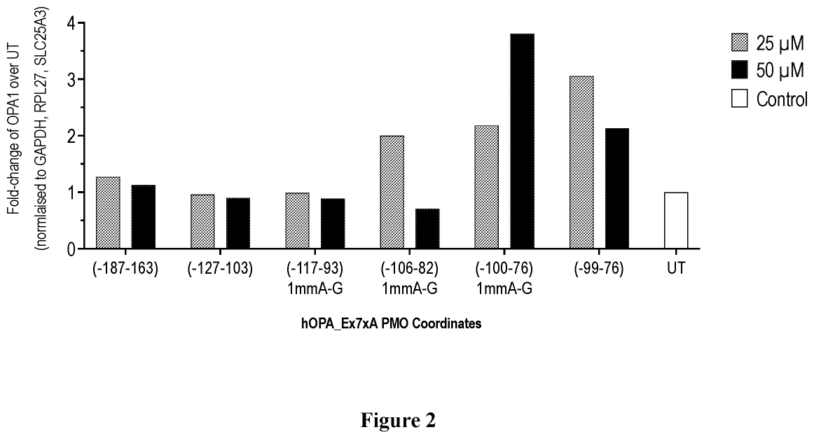

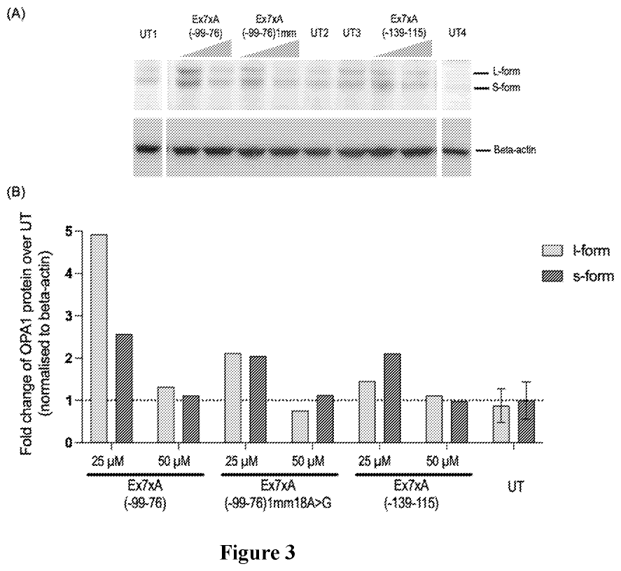

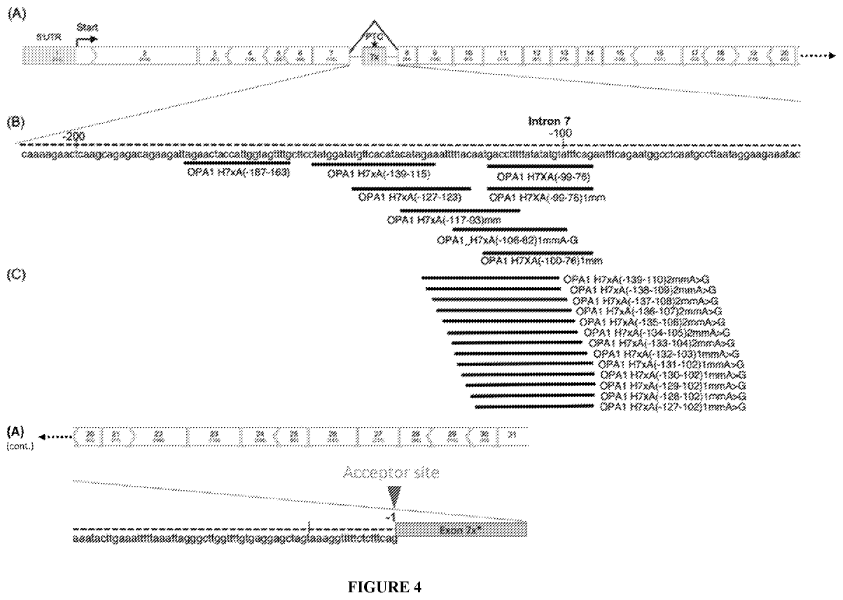

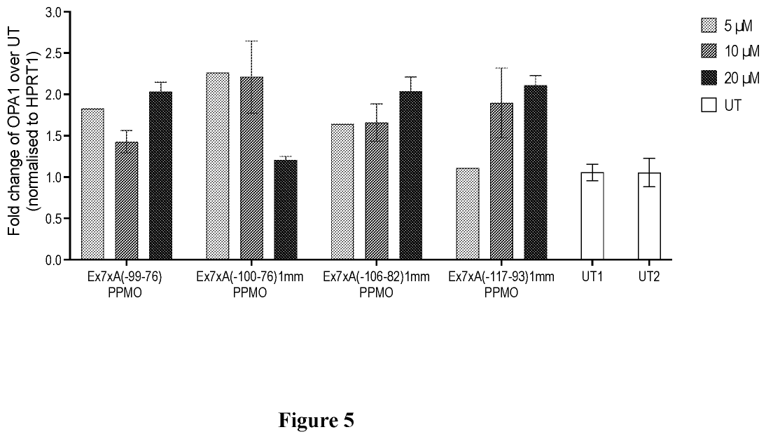

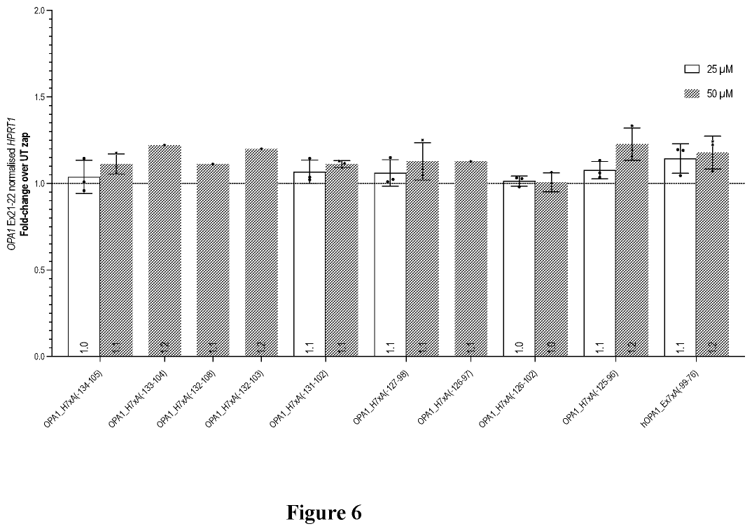

The ASO target region of OPA1 intron 7 and exon 7× is shown in FIGS. 1A and C. ASOs with 24-25 nucleotides in length (Table 1, SEQ ID NOs: 2-9) are designed to target the intronic splice enhancer motifs (prediction using SpliceAid online tool) in intron 7 to mediate exclusion of exon 7× and generate productive OPA1 transcripts. The identified ASO sequences are synthesized as PMOs and/or 2′MOE chemistry) and nucleofected into HEK293 cells or ADOA patient fibroblasts carrying the OPA1 mutation (c.2708_2711delTTAG) using the NEON® electroporation system (ThermoFisher) at 25 μM and 50 μM and the nucleofected cells are cultured for 48 hr. Total RNA was extracted using the MagMAX™—96 Total RNA Isolation kit and the level of OPA1 transcript is assessed by digital droplet PCR (Qiagen; probe catalogue number: dHsaCPE5043545). OPA1 transcript expression is normalized to GAPDH, RPL27 and SCL25A3 transcript levels (Qiagen; probe catalogue number: dHsaCPE5031596, dHsaCPE5036407, dHsaCPE5032926 respectively). The result of ASO screening is shown in FIG. 2. PMOs that show induced OPA1 mRNA levels were further validated for the ability to increase OPA1 protein upregulation using a western blot assay as shown in FIG. 3. Further refinement of ASO sequences (FIGS. 4, Table 1; SEQ ID NOs: 10-31 and Table 2; SEQ ID NOs: 32-54) is performed to reduce or extend ASO length and micro-walk or engineered mismatch oligos and re-validated by ddPCR and protein assays. The efficacy of refined ASOs in inducing OPA1 upregulation in shown in FIGS. 5 and 6.

### Example 2 ASO Design to Target the 5′ UTR of an OPA1 Transcript

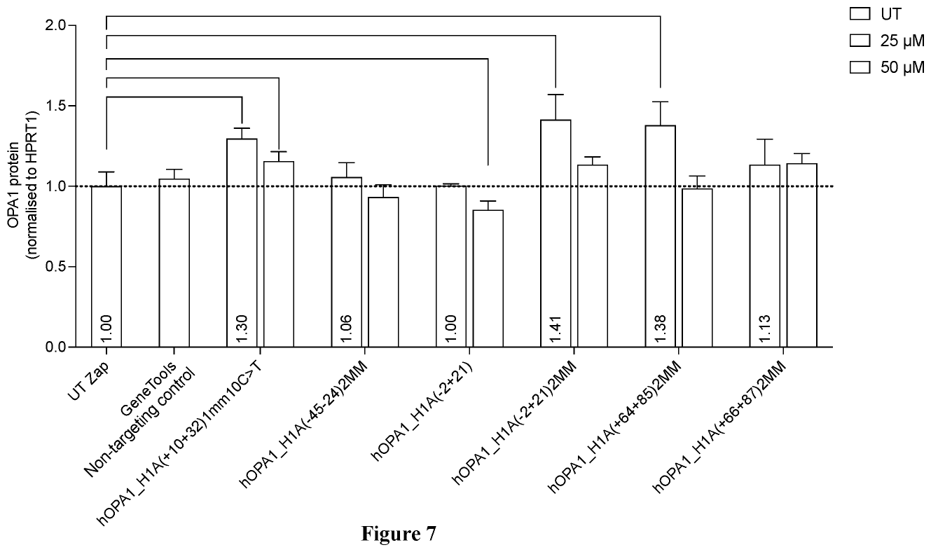

ASOs with 18-25 nucleotides in length (Table 3, SEQ ID NOs: 56-116) are designed to sterically inhibit a uORF or reduce the complexity of RNA secondary structure in the 5′ UTR. The secondary structure of RNA is predicted using RNAfold online tool. The identified ASO sequences are synthesized as PMO and or 2′MOE chemistry) and nucleofected into HEK293 cells or ADOA patient fibroblasts carrying OPA1 mutation (c.2708_2711delTTAG) using the NEON® electroporation system (ThermoFisher) at 25 μM and 50 μM and the nucleofected cells were cultured for 48 hr. Total protein is harvested from the transfected cells using the CytoBuster protein extraction reagent (Merck Millipore) following the manufacturer's instruction and assessed by Western blot assay using rabbit anti-OPA1 monoclonal antibody (Cell Signaling, catalogue number 67589) at a dilution of 1:250 in 5% BSA in TBST buffer followed by goat anti-rabbit IgG H&L antibody (Abcam, catalogue number ab216773, IRDye® 800CW). Beta-actin serves as loading control and is detected using monoclonal mouse anti-beta actin antibody (Sigma-Aldrich, catalogue number A5441) followed by goat anti-mouse IgG H&L antibody (Abcam, catalogue number ab216776, IRDye® 680RD). ASO sequences are further refined by micro-walk or engineered mismatch oligos and/or extended up to 30 nucleotides (Table 3; SEQ ID NOs: 117-138). ASOs was subsequently re-validated using a western blot assay and the results is shown in FIG. 7.

### Example 3 ASO Design to Target the 3′ UTR to Increase OPA1 Expression Levels

An ASO sequence “micro-walk” of 25-mers (Table 4; SEQ ID NOs: 140-1312) or 17-mers (Table 5; SEQ ID NOs: 1313-2488) in 3 bp increments of distance is performed over the sequences of the 3′ UTR of the ENST00000361510 transcript to mediate improvement in RNA stability. ASOs are screened to guide the ASO selection for OPA1 expression upregulation using ddPCR and western blot assays described in Examples 1 and 2.

### Example 4 PPMO-Mediated OPA1 Upregulation to Improve Mitochondrial ATP Production

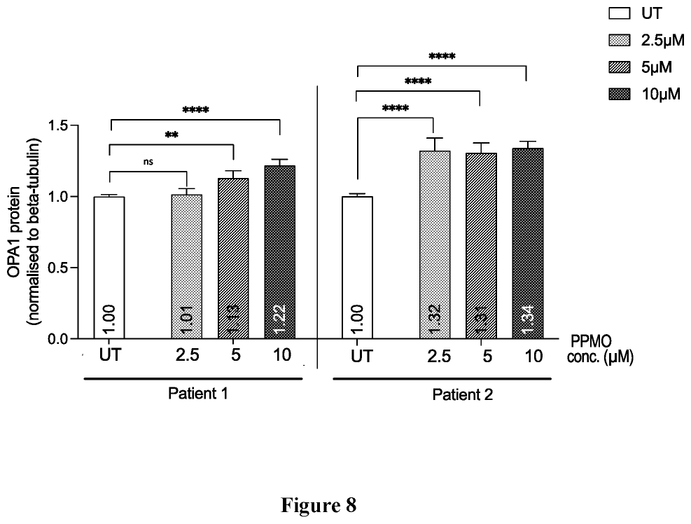

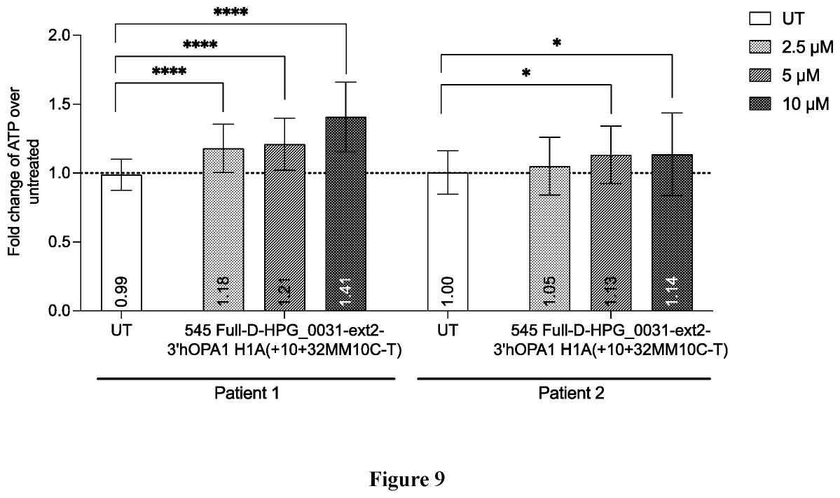

PMO OPA1 H1A(+10+32)1 mm10C>T (SEQ ID NO: 112) was conjugated with CPP for enhanced delivery into cells. The CPP-PMO (or PPMO) was tested for the ability to improve OPA1 protein upregulation in fibroblasts derived from ADOA patients with distinct OPA1 mutations. PPMO was incubated to patient fibroblasts and the efficacy of PPMO-induced OPA1 upregulation was assessed using a western blot assay. Total protein was harvested from the transfected cells using RIPA buffer (ThermoFisher) following the manufacturer's instruction and assessed by western blot assay using rabbit anti-OPA1 monoclonal antibody (Cell Signaling Technology, catalogue number 67589) at a dilution of 1:250 in 5% BSA in TBST buffer followed by goat anti-rabbit IgG H&L antibody (Abcam, catalogue number ab216773, IRDye® 800CW). HPRT1 served as the loading control and was detected using HPRT1 Polyclonal antibody (ProteinTech, catalogue number 15059-1-AP). Expression levels of OPA1 protein were compared between no PPMO-transfected cells (UT) and OPA1 PPMO-incubated cells. FIG. 8 shows the PPMO exhibited upregulation of OPA1 protein expression compared to untreated patient fibroblasts (n=3 biological replicates). In addition, PPMO-treated cells were evaluated for the improvement of mitochondrial function using a CellTiter-Glo® assay to assess ATP levels. PPMO-treated cells were culture in a glucose starvation condition supplemented with 5 mM pyruvate (cat #11360070, ThermoFisher) to continuously supply a substrate for mitochondrial respiratory chain reaction while glycolysis was inhibited using 2.5 mM D-deoxy glucose (catalogue number D8375, Sigma-Aldrich). An ATP standard curve was analysed using 14.7-10,000 nM ATP (catalogue number R0441, ThermoFisher). Results in FIG. 9 show improvement in mitochondrial ATP production in patient fibroblasts treated with the PPMO (SEQ ID NO: 112) targeting the 5′ UTR of OPA1 transcript.

### Example 5 PPMO Treatment Induces Total OPA1 Protein in RGC Enriched Culture Derived from an ADOA Patient

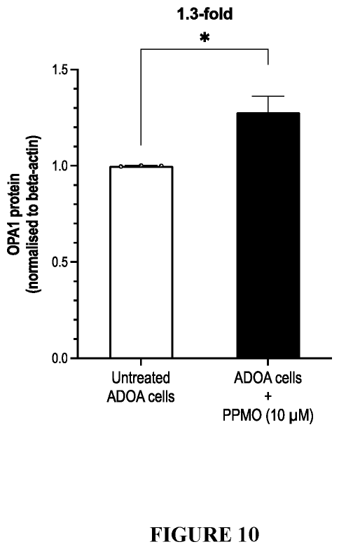

PPMO OPA1 H1A(+10+32)1 mm10C>T (SEQ ID NO: 112) was incubated to iPSC-RGCs derived from an ADOA patient carrying the OPA1 mutation (c.985-1G>A) for 120 hr in triplicates. Total protein was harvested from the transfected cells using the CytoBuster protein extraction reagent (Merck Millipore) following the manufacturer's instruction and assessed by western blot assay using rabbit anti-OPA1 monoclonal antibody (Cell Signaling Technology, catalogue number 67589) at a dilution of 1:250 in 5% BSA in TBST buffer followed by goat anti-rabbit IgG H&L antibody (Abcam, catalogue number ab216773, IRDye® 800CW). Beta-actin served as loading control and was detected using monoclonal mouse anti-beta actin antibody (Sigma-Aldrich, catalogue number A5441) followed by goat anti-mouse IgG H&L antibody (Abcam, catalogue number ab216776, IRDye® 680RD). FIG. 10 shows the PPMO mediated upregulation of total OPA1 protein by up to 1.3-fold at 10 μM as compared to untreated patient fibroblasts. Student's t test was used for statistical analysis.

### Sequences

SEQ ID NO: 1: OPA1 intron 7 (lowercase) and exon7× (uppercase) cDNA sequence (GRCh38/hg38: chr3 193626203-193628616)

gtgatggatggtttaagggggctaccgatacattcacactaatcagccatttctgccaagatcatgtcacctcaa
tctgttcatggactccaaatacaagaaattaatttgacaaagtgaaaatataaaagatgcatcatataaatatgt
aacttttctggagtgggtagtataggtaaagccaaaagaaacaaattcaagcagaggaattttggtttctgaaaa
ttaggttgtctgtagggtccctgtatttatacttagaacaaaattaggaatttctgtttatgtggtccagttatt
gagtcaccctaagtttgtaggcatcttacctacctacttgctccccaagtttttatttctaaaatgaaaagcatt
gctgtagatgaccagtttacactaaagaataacatttatttatttgttttagctaaagtatatggacagggaaca
ttcatattcttgtagaagaaaattattttgacttttgggcaaaagcatgtagttcttatacactttgacaaactc
attgcgtacatttttcacattaatcaaagtcagcacaaataaattttcaccttggaccacggagggtttgaacac
tggaaatttgatataattctggttgctaaagaacaagttctaataaaagcttaagtgtataccaatatgtggctg
ttggtgcaatcagcaggtccgtaaaaatatgattttaatggttaggtaatcccacaacggagatcccaaagttca
tgtttggaagagacttttgggtcaaagtgaaatcagtgtaatgaatttaaaattatactctgagatcttgaaatc
agctaattatgttacatcttattagctcagaaaagttttgaagttatatacaaatgctagtcaggaaaaaagatt
cagtcatgtaattcttgtacattctactatttaaatcaaccaatattatagattatgatttagtgcagtaattct
gctggctaaccttatctcatttggtggtggttagtacttcagagtactcaccatagtttcatttatgttttcagc
atcacttcctggtttttctcaattccatggctgtggaatcaattcatatgtatatttagcttcggtgagcaaaaa
catagctagaaaaagaaaagaagtgagtttcctacctggttaaattaaagtcgatgtgttaagccaaggaggact
tcttttgaatggtactttaacaatccctgttctgtatactgtgaatatatcatttaaatagcctaataaattgga
tgcttaggctgagccacctatactttagttttgttatggaaagaagggagaggagcaagtatgttcttatatgtt
acttagaaataagaatgtagctgtagttacacattgttcttaagtttttttcgtaagacaacttgaaatgagtcc
cataggcctgctatttaacattctaagatatgacttaaggttaatgatgagcttttgaatctgacaattcaagag
atatccataatgaatactgattcattttctacattgctgaaagctaatgttcattttaagcctactttagtagcc
tttatttgggcttagagatgttattcctctttctgatatttattgggttatctgtttaacccttttatatctccc
tttcccgatttgtaaattagagactggcaagactttttaccctgagtagagcaccaaacatggcttgtttctgcc
cacactgtagttaccttgaggggaagtaaatgggactttaaaagcaatttatgctcttttatagtgaaattatcc
ctcttactatcccgaaagactgttaccttacaatatcctccactcctttccccctgtagttactatagagatgac
ttttcggttcttcactgccataatgatcaaaatcctaattcatgagatttttatcattccaggcatgtgaggttt
acttgatgcataaaaccgcaagtactttttgttgttttttaattgttttttctctcttatcttcttgaaagtcta
agtagatcatcatttttgatgtcttattagtagcaactaataaattttccctgtatcttctcagcaaaagaactc
aagcagagacagaagattagaactaccattggtagttttgcttcctatggatatgttcacatacatagaaatttt
tacaatgacctttttatatatgtatttcagaatttcagaatggcctcaatgccttaataggaagaaatacttgaa
atttttaaattagggcttggttttgtgaggagctagtaaaggtttttctctttcagCTTTAGCTTGTTTCTGCGG
AGGATTCCGCTCTTTCTCCATCAGTTTCATAGCCCTGGAATTGTAGAAAAGCTCTGGTTTCAAGACCATTGATAT
CCATTTCTGTCAGG

TABLE 1
List of antisense oligonucleotide sequences targeting
removal of NMD exon 7X of an OPA1 transcript

| SEQ | | |
|---|---|---|
| ID | ASO Coordinates | Sequence 5′>3′ |
| 2 | hOPA1_Ex7xA(−99 − 76) | CTGAAATACATATATAAAAAGGTC |
| 3 | hOPA1_Ex7xA(−99 − 76) | CTGAAATACATATATAAGAAGGTC |
| | 1mm | |
| 4 | hOPA1_Ex7xA(−100 − 76) | CTGAAATACATATATAAGAAGGTC |
| | 1mmA-G | A |
| 5 | hOPA1_Ex7xA(−106 − 82) | TACATATATAAGAAGGTCATTGTA |
| | 1mmA-G | A |
| 6 | hOPA1_Ex7xA(−117 − 93) | AAAGGTCATTGTAAGAATTTCTATG |
| | 1mmA-G | |
| 7 | hOPA1_Ex7xA(−127 − 103) | GTAAAAATTTCTATGTATGTGAACA |
| 8 | hOPA1_Ex7xA(−139 − 115) | TCTATGTATGTGAACATATCCATAG |
| 9 | hOPA1_Ex7xA(−187 − 163) | AATCTTCTGTCTCTGCTTGAGTTCT |
| 10 | hOPA1_Ex7xA(−113 − 84) | TGAGGCCATTCTGAAATTCTGAAAT |
| | | ACATA |
| 11 | hOPA1_Ex7xA(−113 − 84) | TGAGGCCATTCTGGAATTCTGAAAT |
| | 14mmA>G | ACATA |
| 12 | hOPA1_Ex7xA(−112 − 83) | TTGAGGCCATTCTGAAATTCTGAAA |
| | | TACAT |
| 13 | hOPA1_Ex7xA(−111 − 82) | ATTGAGGCCATTCTGAAATTCTGAA |
| | | ATACA |
| 14 | hOPA1_Ex7xA(−110 − 81) | CATTGAGGCCATTCTGAAATTCTGA |
| | | AATAC |
| 15 | hOPA1_Ex7xA(−110 − 81) | CATTGAGGCCATTCTGAAATTCTGG |
| | 25mmA>G | AATAC |
| 16 | hOPA1_Ex7xA(−109 − 80) | GCATTGAGGCCATTCTGAAATTCTG |
| | | AAATA |
| 17 | hOPA1_Ex7xA(−108 − 79) | GGCATTGAGGCCATTCTGAAATTCT |
| | | GAAAT |
| 18 | hOPA1_Ex7xA(−107 − 78) | AGGCATTGAGGCCATTCTGAAATTC |
| | | TGAAA |
| 19 | hOPA1_Ex7xA(−106 − 77) | AAGGCATTGAGGCCATTCTGAAATT |
| | | CTGAA |
| 20 | hOPA1_Ex7xA(−105 − 76) | TAAGGCATTGAGGCCATTCTGAAAT |
| | | TCTGA |
| 21 | hOPA1_Ex7xA(−104 − 76) | TAAGGCATTGAGGCCATTCTGAAAT |
| | | TCTG |
| 22 | hOPA1_Ex7xA(−103 − 76) | TAAGGCATTGAGGCCATTCTGAAAT |
| | | TCT |
| 23 | hOPA1_Ex7xA(−102 − 76) | TAAGGCATTGAGGCCATTCTGAAAT |
| | | TC |
| 24 | hOPA1_Ex7xA(−101 − 76) | TAAGGCATTGAGGCCATTCTGAAAT |
| | | T |
| 25 | hOPA1_Ex7xA(−100 − 76) | TAAGGCATTGAGGCCATTCTGGAA |
| | 22mmA>G | T |
| 26 | hOPA1_Ex7xA(−104 − 75) | TTAAGGCATTGAGGCCATTCTGAAA |
| | | TTCTG |
| 27 | hOPA1_Ex7xA(−103 − 74) | ATTAAGGCATTGAGGCCATTCTGAA |
| | | ATTCT |
| 28 | hOPA1_Ex7xA(−102 − 73) | TATTAAGGCATTGAGGCCATTCTGA |
| | | AATTC |
| 29 | hOPA1_Ex7xA(−101 − 72) | CTATTAAGGCATTGAGGCCATTCTG |
| | | AAATT |
| 30 | hOPA1_Ex7xA(−100 − 71) | CCTATTAAGGCATTGAGGCCATTCT |
| | | GAAAT |
| 31 | hOPA1_Ex7xA(−99 − 70) | TCCTATTAAGGCATTGAGGCCATTC |
| | | TGAAA |

TABLE 2
List of antisense oligonucleotide micro-walked sequences
with 17 nucleotides targeting intron 7 of an OPA1 transcript.

| SEQ | | |
|---|---|---|
| ID | ASO Coordinates | Sequence 5′>3′ |
| 32 | OPA1 H7XA(−215 − 199) | CTCTGCTTGAGTTCTTT |
| 33 | OPA1 H7XA(−212 − 196) | TGTCTCTGCTTGAGTTC |
| 34 | OPA1 H7XA(−209 − 193) | TTCTGTCTCTGCTTGAG |
| 35 | OPA1 H7XA(−206 − 190) | ATCTTCTGTCTCTGCTT |
| 36 | OPA1 H7XA(−203 − 187) | CTAATCTTCTGTCTCTG |
| 37 | OPA1 H7XA(−164 − 148) | TGAACATATCCATAGGA |
| 38 | OPA1 H7XA(−161 − 145) | ATGTGAACATATCCATA |
| 39 | OPA1 H7XA(−158 − 142) | TGTATGTGAACATATCC |
| 40 | OPA1 H7XA(−155 − 139) | CTATGTATGTGAACATA |
| 41 | OPA1 H7XA(−152 − 136) | TTTCTATGTATGTGAAC |
| 42 | OPA1 H7XA(−149 − 133) | AAATTTCTATGTATGTG |
| 43 | OPA1 H7XA(−146 − 130) | TAAAAATTTCTATGTAT |
| 44 | OPA1 H7XA(−143 − 127) | TTGTAAAAATTTCTATG |
| 45 | OPA1 H7XA(−140 − 124) | TCATTGTAAAAATTTCT |
| 46 | OPA1 H7XA(−137 − 121) | AGGTCATTGTAAAAATT |
| 47 | OPA1 H7XA(−134 − 118) | AAAAGGTCATTGTAAAA |
| 48 | OPA1 H7XA(−131 − 115) | ATAAAAAGGTCATTGTA |
| 49 | OPA1 H7XA(−128 − 112) | TATATAAAAAGGTCATT |
| 50 | OPA1 H7XA(−125 − 109) | ACATATATAAAAAGGTC |
| 51 | OPA1 H7XA(−122 − 106) | AATACATATATAAAAAG |
| 52 | OPA1 H7XA(−119 − 103) | TGAAATACATATATAAA |
| 53 | OPA1 H7XA(−116 − 100) | TTCTGAAATACATATAT |
| 54 | OPA1 H7XA(−113 − 97) | AAATTCTGAAATACATA |

SEQ ID: 55: cDNA sequence of the 5′ UTR of an OPA transcript (GRCh38/hg38: chr3 193593064-193593380).
GTCCGTTCCCGACGCACTGTGCGCATGCGCTGGTCCTCCGCGGACCGTTCGTGCTGCCCGCCTAGAAAGGGTGAAGTGGTTGTTTCCGTGACGGACTGAGTACGGGTGCCTGTCAGGCTCTTGCGGAAGTCCATGCGCCATTGGGAGGGCCTCGGCCGCGGCTCTGTGCCCTTGCTGCTGAGGGCCACTTCCTGGGTCATTCCTGGACCGGGAGCCGGGCTGGGGCTCACACGGGGGCTCCCGCGTGGCCGTCTCGGCGCCTGCGTGACCTCCCCGCCGGCGGG

TABLE 3
List of antisense oligonucleotide sequences targeting
the 5′ UTR of an OPA1 transcript.

| SEQ | | |
|---|---|---|
| ID | ASO Coordinates | Sequence 5′>3′ |
| 56 | OPA1_H1A(−39 − 17) | ACCAGCGCATGCGCACAGTGCGT |
| 57 | OPA1_H1A(−35 − 14) | AGGACCAGCGCATGCGCACAGT |
| 58 | OPA1_H1A(−26 − 6)1mm | GTCCGCTGAGGACCAGCGCAT |
| 59 | OPA1_H1A(−3 + 18) | TTTCTAGGGGGCAGCACGAA |
| 60 | OPA1_H1A(+10 + 32) | CAACCACTTCACCCTTTCTAGGC |
| 61 | OPA1_H1A(+13 + 31) | AACCACTTCACCCTTTCTA |
| 62 | OPA1_H1A(+33 + 57) | ACCCGTACTCAGTCCGTCACGGAAA |
| 63 | OPA1_H1A(+33 + 54) | CGTACTCAGTCCGTCACGGAAA |
| 64 | OPA1_H1A(+56 + 80) | ACTTCCGCAAGAGCCTGACAGGCAC |
| 65 | hOPA1_H1A(+63 + 82) | GGACTTCCGCAAGAGCCTGA |
| 66 | OPA1_H1A(+68 + 92) | CAATGGCGCATGGACTTCCGCAAGA |
| 67 | OPA1_H1A(+78 + 98) | CTCCCAATGGCGCATGGACT |
| 68 | OPA1_H1A(+128 + 146)1mm | CCCAGGAAGTGGTCCTCAG |
| 69 | OPA1_H1A(+148 + 166) | GGCTCCCGGTCCAGGAATG |
| 70 | OPA1_H1A(+200 + 218)2mm | TCACGCAGGTGCTGAGACG |
| 71 | hOPA1_H1A(−30 − 9) | CGTGGAGGACCAGTGCATGCGC |
| | 2mmT(C>T) | |
| 72 | hOPA1_H1A(−22 − 2) | AACGGTCCGCGGAGGACCAGC |
| 73 | hOPA1_H1A(−18 + 4) | GCACGAACGGTCCGCGGAGGAC |
| 74 | hOPA1_H1A(−14 + 8) | GGCAGTACGAACGGTCTGCGGA |
| | 2mmT(C>T) | |
| 75 | hOPA1_H1A(−10 + 12) | GGTGGGCAGCACGAATGGTCCG |
| | 2mmT(C>T) | |
| 76 | hOPA1_H1A(−6 + 16) | TCTAGGCGGGCAGCACGAACGG |
| 77 | hOPA1_H1A(−2 + 21) | CCTTTCTAGGCGGGCAGCACGA |
| 78 | hOPA1_H1A(+2 + 25) | TCACCCTTTCTAGGCGGGCAGC |
| 79 | hOPA1_H1A(+7 + 31) | AACCACTTCACCCTTTCTAGGCGGG |
| 80 | hOPA1_H1A(+12 + 36) | GAAACAACCACTTCACCCTTTCTAG |
| 81 | hOPA1_H1A(+17 + 41) | TCACGGAAACAACCACTTCACCCTT |
| 82 | hOPA1_H1A(+22 + 46) | GTCCGTCACGGAAACAACCACTTCA |
| 83 | hOPA1_H1A(+27 + 51) | ACTCAGTCCGTCACGGAAACAACCA |
| 84 | hOPA1_H1A(+38 + 62) | CAGGCACCCGTACTCAGTCCGTCAC |
| 85 | hOPA1_H1A(+44 + 67) | CCTGACAGGCACCCGTACTCAGTCC |
| 86 | hOPA1_H1A(+50 + 71) | GAGCCTGACAGGCACCCGTACTC |
| 87 | hOPA1_H1A(+56 + 75) | GCAAGAGCCTGACAGGCACCCG |
| 88 | hOPA1_H1A(+120 + 141) | AAGTGGCCCTCAGCAGCAAGGG |
| 89 | hOPA1_H1A(+125 + 148)2mmT | ACCTAGGAAGTGGTCCTCAGCAGC |
| | (C>T) | |
| 90 | hOPA1_H1A(+129 + 154) | GGAATGACCCAGGAAGTGGCCCTCA |
| 91 | hOPA1_H1A(+134 + 159) | GTCCAGGAATGACCCAGGAAGTGGC |
| 92 | hOPA1_H1A(+42 + 66) | CTGACAGGCACCCGTACTCAGTCCG |
| 93 | hOPA1_H1A(+44 + 68) | GCCTGACAGGCACCCGTACTCAGTC |
| 94 | hOPA1_H1A(+45 + 69) | AGCCTGACAGGCACCCGTACTCAGT |
| 95 | hOPA1_H1A(+46 + 70) | GAGCCTGACAGGCACCCGTACTCAG |
| 96 | hOPA1_H1A(+47 + 71) | AGAGCCTGACAGGCACCCGTACTCA |
| 97 | hOPA1_H1A(+48 + 72) | AAGAGCCTGACAGGCACCCGTACTC |
| 98 | hOPA1_H1A(+49 + 73) | CAAGAGCCTGACAGGCACCCGTACT |
| 99 | hOPA1_H1A(+50 + 74) | GCAAGAGCCTGACAGGCACCCGTAC |
| 100 | hOPA1_H1A(+42 + 66)2mmC>T | CTGACAGGCATTCGTACTCAGTCCG |
| 101 | hOPA1_H1A(+44 + 68)2mmC>T | GCCTGACAGGCACTTGTACTCAGTC |
| 102 | hOPA1_H1A(+45 + 69)2mmC>T | AGCCTGACAGGCATTCGTACTCAGT |
| 103 | hOPA1_H1A(+46 + 70)2mmC>T | GAGCTTGACAGGCACCTGTACTCAG |
| 104 | hOPA1_H1A(+47 + 71)2mmC>T | AGAGCTTGACAGGCACTCGTACTCA |
| 105 | hOPA1_H1A(+48 + 72)2mmC>T | AAGAGCTTGACAGGCACTCGTACTC |
| 106 | hOPA1_H1A(+49 + 73)2mmC>T | CAAGAGCTTGACAGGCATCCGTACT |
| 107 | hOPA1_H1A(+50 + 74)2mmC>T | GCAAGAGCTTGACAGGCATCCGTAC |
| 108 | OPA1_H1A(+10 + 34) | AACAACCACTTCACCCTTTCTAGGC |
| 109 | OPA1_H1A(+10 + 33) | ACAACCACTTCACCCTTTCTAGGC |
| 110 | OPA1 H1A(+9 + 33) | ACAACCACTTCACCCTTTCTAGGCG |
| 111 | OPA1_H1A(+11 + 35) | AAACAACCACTTCACCCTTTCTAGG |
| 112 | OPA1 H1A(+10 + 32) | CAACCACTTTACCCTTTCTAGGC |
| | 1mm10C>T | |
| 113 | OPA1 H1A(+10 + 32) | CAACCACTTCATCCTTTCTAGGC |
| | 1mm12C>T | |
| 114 | hOPA1_H1A(+63 + 80) | ACTTCCGCAAGAGCCTGA |
| 115 | hOPA1_H1A(+79 + 96) | TCCCAATGGCGCATGGAC |
| 116 | hOPA1_H1A(−50 − 21) | GCGCATGCGCACAGTGCGTCGGGA |
| | | ACGGAC |
| 117 | hOPA1_H1A(−49 − 20) | AGCGCATGCGCACAGTGCGTCGGG |
| | | AACGGA |
| 118 | hOPA1_H1A(−48 − 19) | CAGCGCATGCGCACAGTGCGTCGG |
| | | GAACGG |
| 119 | hOPA1_H1A(−47 − 18) | CCAGCGCATGCGCACAGTGCGTCG |
| | | GGAACG |
| 120 | hOPA1_H1A(−46 − 17) | ACCAGCGCATGCGCACAGTGCGTC |
| | | GGGAAC |
| 121 | hOPA1_H1A(−45 − 16) | GACCAGCGCATGCGCACAGTGCGT |
| | | CGGGAA |
| 122 | hOPA1_H1A(−44 − 15) | GGACCAGCGCATGCGCACAGTGCG |
| | | TCGGGA |
| 123 | hOPA1_H1A(−43 − 14) | AGGACCAGCGCATGCGCACAGTGC |
| | | GTCGGG |
| 124 | hOPA1_H1A(−42 − 13) | GAGGACCAGCGCATGCGCACAGTG |
| | | CGTCGG |
| 125 | hOPA1_H1A(−41 − 12) | GGAGGACCAGCGCATGCGCACAGT |
| | | GCGTCG |
| 126 | hOPA1_H1A(−40 − 11) | CGGAGGACCAGCGCATGCGCACAG |
| | | TGCGTC |
| 127 | hOPA1_H1A(−2 + 28)mm | CACTTTACCCTTTCTAGGCGGGCAG |
| | | CACGA |
| 128 | hOPA1_H1A(−1 + 29)mm | CCACTTTACCCTTTCTAGGCGGGCA |
| | | GCACG |
| 129 | hOPA1_H1A(+1 + 30)mm | ACCACTTTACCCTTTCTAGGCGGGC |
| | | AGCAC |
| 130 | hOPA1_H1A(+2 + 31)mm | AACCACTTTACCCTTTCTAGGCGG |
| | | GCAGCA |
| 131 | hOPA1_H1A(+3 + 32)mm | CAACCACTTTACCCTTTCTAGGCG |
| | | GGCAGC |
| 132 | hOPA1_H1A(+4 + 32)mm | CAACCACTTTACCCTTTCTAGGCG |
| | | GGCAG |
| 133 | hOPA1_H1A(+5 + 32)mm | CAACCACTTTACCCTTTCTAGGCG |
| | | GGCA |
| 134 | hOPA1_H1A(+6 + 32)mm | CAACCACTTTACCCTTTCTAGGCGGGC |
| 135 | hOPAI_H1A(+7 + 32)mm | CAACCACTTTACCCTTTCTAGGCGGG |
| 136 | hOPA1_H1A(+8 + 32)mm | CAACCACTTTACCCTTTCTAGGCGG |
| 137 | hOPA1_H1A(+9 + 32)mm | CAACCACTTTACCCTTTCTAGGCG |
| 138 | hOPA1_H1A(+10 + 34)mm | AACAACCACTTTACCCTTTCTAGGC |

SEQ ID: 139: cDNA sequence of the 3′ UTR located within exon 30 of an OPA transcript (GRCh38/hg38: chr3 193694606-193697811)
GTCCGTTCCCGACGCACTGTGCGCATGCGCTGGTCCTCCGCGGACCGTTCGTGCTGCCCGCCTAGAAAGGGTGAAGAATCGTACTCATAATCAGCTCTGCATACATCTGAAGAACAAAAACATCAACGTCTTTTGTCCAGCCTCTTTTTCTTCTGCTGTTCCACCTTTCTAAACATACAATAAAGTCATGGGATAAAAATAATCGATGTATGTTACGGGCGCTTTAACCATCAGCTGCCTCTCGAATGGAAGAACAGTGGTAATGGATTAACATCCTATTTTGTTGTACTAAAGTGACAAATCGGAATAATATAATTGGTATGGCCATTAGGTTCAGTCCTTGAAGATAAGAAACTTGTTCTCTGTTTGTTGTCTTATTTGTGGTGGCACTCGTTTAATGGATTAACTGAGGTTGCTCAATGTTCAGTTTCTTTTCCAGAAATACAATGCTAGGTGTTTTGAAATAAAACTTATATAGCAATTGTTTAAAGTTATCAATTGTATATAAAATCACAGTAGCCTGCTAAATCATTGTATGTGTCTGTAGTATTCTATTCCCAGAAACTATTTGACCATGATAATTCAGTTTATATTCACCACATGAAAGAAAAATGGGTAACAGAAGAACCCTTAAAACAGGTTAATTTGGATTGTAACGTTCAGTGAAAGAAATTTCAACCCTTCATAGCCAGCGAAGAAATTTGCCTTGGAAGCCAAGTCAGTACCAGCTTACCTATTTGATTCAGTTGCTGTTTTCTCACTCTCTATATCCATTTGAAATTGATTTATTTTAGATGTTGTATACTTACGTTAGGCTTTCTGTTAATAGTGGTTTTTCTCCTGTTGACAGAGCCACCGGATTATGACACAGGATGAGGAAGATTAAGGATAATCAATTGACTAATTTCATTTAGAATATTATCAAACATTTCAACTAGGTATCAGAAAAAGGCTTTCTTTCATAAGACTATTTTAAATAGAAATTATTTCAACAATTAAAGTAATGTTGACCATCCCCCTCTCAGCTGAATAAAGAAAAATTTAGTTCAATTTATTGCAATTTAATTACAATACTACCTTCACAACATTTTCATGTGTTTTAAATAAATATTTTTTAATTGGCTAAAGGACATTCAAGCAAAGAAATGCTTTCTTTACTTAAAATGTCTATCTCATTTGCTGCCTTTTCACTAAGCCTTTACTTTGTTAATAAAAGTGTCCATTGTGTGATGTTTTTGATTTTACAGTTTGCTAAATCTTATTTTCTTGGAGTTGCTTTTTGGTAACAGCCCCATTGCTACTCCCCATTTTATTGTTTTACATCAATGCATGCTTCGTTGTGATCCCTCAAGATGTAACACTTGGTATGCTCGGTTGAGGATATGAAAAAATACTTCCGAAACCAGGAATTCAATGTATGTTTGTTTTATACTGTTTGATAAGAAAAGTAGGTCCAGCCTTAAGCAGCACAGATGCGCTGGTAGATGCATAGTCAGGAACTTTTTTTATTTCTTTTAGGTCTAGGGACAGGAGTGAATAGAAAGGGAGGAGAGCTCTATTATGTTCTATACACAGATTAGGAGATGACCTTACTGGGTACACCCCTCTAACCAGTGCTTACAGGTTAATGCATGTTAATGAATATTTTTGCAGTTGTAAAGCATAACAATTACAACTACACATCTATTTCTAAAGAATAAAACAGGACCATATTTATTTACTTCTGTCAACTATAGAAAGAAAGACCTTCAGCTGTATTTCCACAGATTTCTCCCAAGGAAAAGGCTAATATTAGTCACTACTGTTATCACATCCCTTTGTATAAGTTTTAAAAAGAGATGGAGGGAGATCTTCATTTCTTTGAGGAGATCAGTATTGTAACGTATGTGAATAGATGATAACAATTAATATTACTAAAAGTCCCACATGAGAGTCCTGACGCCCTCTCCATGCCCCACAGTAATGTGGCTTCTTTCATGGGTTTTTTTTTCTTCTTTTTAGCTGATCTCATCCTAAGCATGCTTTATTTTTCCTTGAAAGCTAGGTATTTATCAACTGCAGATGTTATTGAAAGAAAATAAAATTCAGTCTCAAGAGTAAACCCTGTGTCTTGTGTCTGTAGTTCAAAAGTCAGAAATGATTCTAATTTAAACAAAAAGATACTAAATATACAGAAGTTAAATTCGAACTAGCCACAGAATCATTTGTTTTTATGTCAGAATTTGCAAAGAGTGGAGTGGACAAAGCTCTGTATGGAAGACTGAACAACTGTAAATAGATGATATCCAAACTTAATTTGGCTAGGACTTCAATTTTAAAAATCAGTGTACCTAGGCAGTGCACAGCACGAAATAAGTGGCCCTTGCAGCTTCCCCGTTTAACCCACTGTGCTATAGTTGCGGGTGGAACAGTCAACCTTTCTAGTAGTTTATGATATTGCCCTCTTTGTATTCCCATTTTCTACAGTTTTTTCCGCAGACTTCTTTCTGCAAATTATTCAGCCTCCAAATGCAAATGAATGATATAAAAATAAGTAGGGAACATGGCAGAGAGTGGTGCTTCCCAGCCTCACAATGTGGGAATTTGACATAGGATGAGAGTCAGAGTATAGGTTTAAAAGATAAAATCTTTAGTTAATAATTTTGTATTTATTTATTCTAGATGTATGTATCTGAGGAAAGAAATCTGGTATTTTTGCTTTCCAATAAAGGGGATCAAAGTAATGGTTTTTCTCTCAGTTCTCTAAGCTGGTCTATGTTATAGCTCTAGCAGTATGGAAATGTGCTTTAAAATATGCTTACCTTTTGAATGATCATGGCTATATGTTGTTGAGATATTTGAAACTTACCTTGTTTTCACTTGTGCACTGTGAATGAACTTTGTATTATTTTTTTAAAACCTTCACATTACGTGTAGATATTATTGCAACTTATATTTTGCCTGAGCTTGATCAAAGGTCATTTGTGTAGATGAGTAATTAAAAAATATTTAAATCACATTATAATTCTATTATTGGAGAGCATCTTTTAAATTTTTTTCTGTTTTAACGAGGGAAAGAGAAACCTGTATACCTAGGGTCATTATTTGACCCCATAGTATAACCAGATTCATGGTCTAACAAGCTCTCAGTGTGGCTTTTCTCTGAATGCTTGAATTTCACATGCCTTGCATTTCACAGTTGTACTCCATGGTCAACCGGTGCTTTTTTTCACATCGTGGTACTTGTCAAAACATTTTGTTATTTTCCTTGGTAAAATATATAAAAAAGGTTTTCTAATTTCA

TABLE 4
List of antisense oligonucleotide sequences with 25 nucleotides in
length that target the 3′ UTR of an OPA1 transcript.

| SEQ | | | SEQ | | |
|---|---|---|---|---|---|
| ID | ASO Coordinates | Sequence 5′>3′ | ID | ASO Coordinates | Sequence 5′>3′ |
| 140 | OPA1 H30:31 | AGCTGATTATGAGTACGATTTTAAT | 156 | OPA1 H313UTR | GAGGCTGGACAAAAGACGTTGATGT |
| | 3UTR(+1+25) | | | (+49+73) | |
| 141 | OPA1 H30:31 | CAGAGCTGATTATGAGTACGATTTT | 157 | OPA1 H313UTR | AAAGAGGCTGGACAAAAGACGTTGA |
| | 3UTR(+4+28) | | | (+52+76) | |
| 142 | OPA1 H31 | ATGCAGAGCTGATTATGAGTACGAT | 158 | OPA1 H313 | GAAAAAGAGGCTGGACAAAAGACGT |
| | 3UTR(+7+31) | | | UTR(+55+79) | |
| 143 | OPA1 H31 | TGTATGCAGAGCTGATTATGAGTAC | 159 | OPA1 H31 | GAAGAAAAAGAGGCTGGACAAAAGA |
| | 3UTR(+10+34) | | | 3UTR(+58+82) | |
| 144 | OPA1 H31 | AGATGTATGCAGAGCTGATTATGAG | 160 | OPA1 H31 | GCAGAAGAAAAAGAGGCTGGACAAA |
| | 3UTR(+13+37) | | | 3UTR(+61+85) | |
| 145 | OPA1 H31 | TTCAGATGTATGCAGAGCTGATTAT | 161 | OPA1 H31 | ACAGCAGAAGAAAAAGAGGCTGGAC |
| | 3UTR(+16+40) | | | 3UTR(+64+88) | |
| 146 | OPA1 H31 | TTCTTCAGATGTATGCAGAGCTGAT | 162 | OPA1 H31 | GGAACAGCAGAAGAAAAAGAGGCTG |
| | 3UTR(+19+43) | | | 3UTR(+67+91) | |
| 147 | OPA1 H31 | TTGTTCTTCAGATGTATGCAGAGCT | 163 | OPA1 H31 | GGTGGAACAGCAGAAGAAAAAGAGG |
| | 3UTR(+22+46) | | | 3UTR(+70+94) | |
| 148 | OPA1 H31 | TTTTTGTTCTTCAGATGTATGCAGA | 164 | OPA1 H31 | AAAGGTGGAACAGCAGAAGAAAAAG |
| | 3UTR(+25+49) | | | 3UTR(+73+97) | |
| 149 | OPA1 H31 | ATGTTTTTGTTCTTCAGATGTATGC | 165 | OPA1 H31 | TAGAAAGGTGGAACAGCAGAAGAAA |
| | 3UTR(+28+52) | | | 3UTR(+76+100) | |
| 150 | OPA1 H31 | TTGATGTTTTTGTTCTTCAGATGTA | 166 | OPA1 H31 | GTTTAGAAAGGTGGAACAGCAGAAG |
| | 3UTR(+31+55) | | | 3UTR(+79+103) | |
| 151 | OPA1 H31 | ACGTTGATGTTTTTGTTCTTCAGAT | 167 | OPA1 H31 | TATGTTTAGAAAGGTGGAACAGCAG |
| | 3UTR(+34+58) | | | 3UTR(+82+106) | |
| 152 | OPA1 H31 | AAGACGTTGATGTTTTTGTTCTTCA | 168 | OPA1 H31 | TTGTATGTTTAGAAAGGTGGAACAG |
| | 3UTR(+37+61) | | | 3UTR(+85+109) | |
| 153 | OPA1 H31 | CAAAAGACGTTGATGTTTTTGTTCT | 169 | OPA1 H31 | TTATTGTATGTTTAGAAAGGTGGAA |
| | 3UTR(+40+64) | | | 3UTR(+88+112) | |
| 154 | OPA1 H31 | GGACAAAAGACGTTGATGTTTTTGT | 170 | OPA1 H31 | ACTTTATTGTATGTTTAGAAAGGTG |
| | 3UTR(+43+67) | | | 3UTR(+91+115) | |
| 155 | OPA1 H31 | GCTGGACAAAAGACGTTGATGTTTT | | | |
| | 3UTR(+46+70) | | | | |
| 171 | OPA1 H31 | ATGACTTTATTGTATGTTTAGAAAG | 189 | OPA1 H31 | GAGAGGCAGCTGATGGTTAAAGCGC |
| | 3UTR(+94+118) | | | 3UTR(+148+172) | |
| 172 | OPA1 H31 | CCCATGACTTTATTGTATGTTTAGA | 190 | OPA1 H31 | TTCGAGAGGCAGCTGATGGTTAAAG |
| | 3UTR(+97+121) | | | 3UTR(+151+175) | |
| 173 | OPA1 H31 | TATCCCATGACTTTATTGTATGTTT | 191 | OPA1 H31 | CCATTCGAGAGGCAGCTGATGGTTA |
| | 3UTR(+100+124) | | | 3UTR(+154+178) | |
| 174 | OPA1 H31 | TTTTATCCCATGACTTTATTGTATG | 192 | OPA1 H31 | CTTCCATTCGAGAGGCAGCTGATGG |
| | 3UTR(+103+127) | | | 3UTR(+157+181) | |
| 175 | OPA1 H31 | TATTTTTATCCCATGACTTTATTGT | 193 | OPA1 H31 | GTTCTTCCATTCGAGAGGCAGCTGA |
| | 3UTR(+106+130) | | | 3UTR(+160+184) | |
| 176 | OPA1 H31 | GATTATTTTTATCCCATGACTTTAT | 194 | OPA1 H31 | ACTGTTCTTCCATTCGAGAGGCAGC |
| | 3UTR(+109+133) | | | 3UTR(+163+187) | |
| 177 | OPA1 H31 | ATCGATTATTTTTATCCCATGACTT | 195 | OPA1 H31 | ACCACTGTTCTTCCATTCGAGAGGC |
| | 3UTR(+112+136) | | | 3UTR(+166+190) | |
| 178 | OPA1 H31 | TACATCGATTATTTTTATCCCATGA | 196 | OPA1 H31 | ATTACCACTGTTCTTCCATTCGAGA |
| | 3UTR(+115+139) | | | 3UTR(+169+193) | |
| 179 | OPA1 H31 | ACATACATCGATTATTTTTATCCCA | 197 | OPA1 H31 | TCCATTACCACTGTTCTTCCATTCG |
| | 3UTR(+118+142) | | | 3UTR(+172+196) | |
| 180 | OPA1 H31 | GTAACATACATCGATTATTTTTATC | 198 | OPA1 H31 | TAATCCATTACCACTGTTCTTCCAT |
| | 3UTR(+121+145) | | | 3UTR(+175+199) | |
| 181 | OPA1 H31 | CCCGTAACATACATCGATTATTTTT | 199 | OPA1 H31 | TGTTAATCCATTACCACTGTTCTTC |
| | 3UTR(+124+148) | | | 3UTR(+178+202) | |
| 182 | OPA1 H31 | GCGCCCGTAACATACATCGATTATT | 200 | OPA1 H31 | GGATGTTAATCCATTACCACTGTTC |
| | 3UTR(+127+151) | | | 3UTR(+181+205) | |
| 183 | OPA1 H31 | AAAGCGCCCGTAACATACATCGATT | 201 | OPA1 H31 | ATAGGATGTTAATCCATTACCACTG |
| | 3UTR(+130+154) | | | 3UTR(+184+208) | |
| 184 | OPA1 H31 | GTTAAAGCGCCCGTAACATACATCG | 202 | OPA1 H31 | AAAATAGGATGTTAATCCATTACCA |
| | 3UTR(+133+157) | | | 3UTR(+187+211) | |
| 185 | OPA1 H31 | ATGGTTAAAGCGCCCGTAACATACA | 203 | OPA1 H31 | AACAAAATAGGATGTTAATCCATTA |
| | 3UTR(+136+160) | | | 3UTR(+190+214) | |
| 186 | OPA1 H31 | CTGATGGTTAAAGCGCCCGTAACAT | 204 | OPA1 H31 | TACAACAAAATAGGATGTTAATCCA |
| | 3UTR(+139+163) | | | 3UTR(+193+217) | |
| 187 | OPA1 H31 | CAGCTGATGGTTAAAGCGCCCGTAA | 205 | OPA1 H31 | TAGTACAACAAAATAGGATGTTAAT |
| | 3UTR(+142+166) | | | 3UTR(+196+220) | |
| 188 | OPA1 H31 | AGGCAGCTGATGGTTAAAGCGCCCG | 206 | OPA1 H31 | CTTTAGTACAACAAAATAGGATGTT |
| | 3UTR(+145+169) | | | 3UTR(+199+223) | |
| 207 | OPA1 H31 | TCACTTTAGTACAACAAAATAGGAT | 225 | OPA1 H31 | CTTATCTTCAAGGACTGAACCTAAT |
| | 3UTR(+202+226) | | | 3UTR(+256+280) | |
| 208 | OPA1 H31 | TTGTCACTTTAGTACAACAAAATAG | 226 | OPA1 H31 | TTTCTTATCTTCAAGGACTGAACCT |
| | 3UTR(+205+229) | | | 3UTR(+259+283) | |
| 209 | OPA1 H31 | GATTTGTCACTTTAGTACAACAAAA | 227 | OPA1 H31 | AAGTTTCTTATCTTCAAGGACTGAA |
| | 3UTR(+208+232) | | | 3UTR(+262+286) | |
| 210 | OPA1 H31 | TCCGATTTGTCACTTTAGTACAACA | 228 | OPA1 H31 | AACAAGTTTCTTATCTTCAAGGACT |
| | 3UTR(+211+235) | | | 3UTR(+265+289) | |
| 211 | OPA1 H31 | TATTCCGATTTGTCACTTTAGTACA | 229 | OPA1 H31 | GAGAACAAGTTTCTTATCTTCAAGG |
| | 3UTR(+214+238) | | | 3UTR(+268+292) | |
| 212 | OPA1 H31 | TATTATTCCGATTTGTCACTTTAGT | 230 | OPA1 H31 | ACAGAGAACAAGTTTCTTATCTTCA |
| | 3UTR(+217+241) | | | 3UTR(+271+295) | |
| 213 | OPA1 H31 | TTATATTATTCCGATTTGTCACTTT | 231 | OPA1 H31 | CAAACAGAGAACAAGTTTCTTATCT |
| | 3UTR(+220+244) | | | 3UTR(+274+298) | |
| 214 | OPA1 H31 | CAATTATATTATTCCGATTTGTCAC | 232 | OPA1 H31 | CAACAAACAGAGAACAAGTTTCTTA |
| | 3UTR(+223+247) | | | 3UTR(+277+301) | |
| 215 | OPA1 H31 | TACCAATTATATTATTCCGATTTGT | 233 | OPA1 H31 | AGACAACAAACAGAGAACAAGTTTC |
| | 3UTR(+226+250) | | | 3UTR(+280+304) | |
| 216 | OPA1 H31 | CCATACCAATTATATTATTCCGATT | 234 | OPA1 H31 | ATAAGACAACAAACAGAGAACAAGT |
| | 3UTR(+229+253) | | | 3UTR(+283+307) | |
| 217 | OPA1 H31 | TGGCCATACCAATTATATTATTCCG | 235 | OPA1 H31 | CAAATAAGACAACAAACAGAGAACA |
| | 3UTR(+232+256) | | | 3UTR(+286+310) | |
| 218 | OPA1 H31 | TAATGGCCATACCAATTATATTATT | 236 | OPA1 H31 | CCACAAATAAGACAACAAACAGAGA |
| | 3UTR(+235+259) | | | 3UTR(+289+313) | |
| 219 | OPA1 H31 | ACCTAATGGCCATACCAATTATATT | 237 | OPA1 H31 | CCACCACAAATAAGACAACAAACAG |
| | 3UTR(+238+262) | | | 3UTR(+292+316) | |
| 220 | OPA1 H31 | TGAACCTAATGGCCATACCAATTAT | 238 | OPA1 H31 | GTGCCACCACAAATAAGACAACAAA |
| | 3UTR(+241+265) | | | 3UTR(+295+319) | |
| 221 | OPA1 H31 | GACTGAACCTAATGGCCATACCAAT | 239 | OPA1 H31 | CGAGTGCCACCACAAATAAGACAAC |
| | 3UTR(+244+268) | | | 3UTR(+298+322) | |
| 222 | OPA1 H31 | AAGGACTGAACCTAATGGCCATACC | 240 | OPA1 H31 | AAACGAGTGCCACCACAAATAAGAC |
| | 3UTR(+247+271) | | | 3UTR(+301+325) | |
| 223 | OPA1 H31 | TTCAAGGACTGAACCTAATGGCCAT | 241 | OPA1 H31 | ATTAAACGAGTGCCACCACAAATAA |
| | 3UTR(+250+274) | | | 3UTR(+304+328) | |
| 224 | OPA1 H31 | ATCTTCAAGGACTGAACCTAATGGC | 242 | OPA1 H31 | TCCATTAAACGAGTGCCACCACAAA |
| | 3UTR(+253+277) | | | 3UTR(+307+331) | |
| 243 | OPA1 H31 | TAATCCATTAAACGAGTGCCACCAC | 261 | OPA1 H31 | AAACACCTAGCATTGTATTTCTGGA |
| | 3UTR(+310+334) | | | 3UTR(+364+388) | |
| 244 | OPA1 H31 | AGTTAATCCATTAAACGAGTGCCAC | 262 | OPA1 H31 | TCAAAACACCTAGCATTGTATTTCT |
| | 3UTR(+313+337) | | | 3UTR(+367+391) | |
| 245 | OPA1 H31 | CTCAGTTAATCCATTAAACGAGTGC | 263 | OPA1 H31 | ATTTCAAAACACCTAGCATTGTATT |
| | 3UTR(+316+340) | | | 3UTR(+370+394) | |
| 246 | OPA1 H31 | AACCTCAGTTAATCCATTAAACGAG | 264 | OPA1 H31 | TTTATTTCAAAACACCTAGCATTGT |
| | 3UTR(+319+343) | | | 3UTR(+373+397) | |
| 247 | OPA1 H31 | AGCAACCTCAGTTAATCCATTAAAC | 265 | OPA1 H31 | AGTTTTATTTCAAAACACCTAGCAT |
| | 3UTR(+322+346) | | | 3UTR(+376+400) | |
| 248 | OPA1 H31 | TTGAGCAACCTCAGTTAATCCATTA | 266 | OPA1 H31 | ATAAGTTTTATTTCAAAACACCTAG |
| | 3UTR(+325+349) | | | 3UTR(+379+403) | |
| 249 | OPA1 H31 | ACATTGAGCAACCTCAGTTAATCCA | 267 | OPA1 H31 | TATATAAGTTTTATTTCAAAACACC |
| | 3UTR(+328+352) | | | 3UTR(+382+406) | |
| 250 | OPA1 H31 | TGAACATTGAGCAACCTCAGTTAAT | 268 | OPA1 H31 | TGCTATATAAGTTTTATTTCAAAAC |
| | 3UTR(+331+355) | | | 3UTR(+385+409) | |
| 251 | OPA1 H31 | AACTGAACATTGAGCAACCTCAGTT | 269 | OPA1 H31 | AATTGCTATATAAGTTTTATTTCAA |
| | 3UTR(+334+358) | | | 3UTR(+388+412) | |
| 252 | OPA1 H31 | AGAAACTGAACATTGAGCAACCTCA | 270 | OPA1 H31 | AACAATTGCTATATAAGTTTTATTT |
| | 3UTR(+337+361) | | | 3UTR(+391+415) | |
| 253 | OPA1 H31 | AAAAGAAACTGAACATTGAGCAACC | 271 | OPA1 H31 | TTAAACAATTGCTATATAAGTTTTA |
| | 3UTR(+340+364) | | | 3UTR(+394+418) | |
| 254 | OPA1 H31 | TGGAAAAGAAACTGAACATTGAGCA | 272 | OPA1 H31 | ACTTTAAACAATTGCTATATAAGTT |
| | 3UTR(+343+367) | | | 3UTR(+397+421) | |
| 255 | OPA1 H31 | TTCTGGAAAAGAAACTGAACATTGA | 273 | OPA1 H31 | ATAACTTTAAACAATTGCTATATAA |
| | 3UTR(+346+370) | | | 3UTR(+400+424) | |
| 256 | OPA1 H31 | TATTTCTGGAAAAGAAACTGAACAT | 274 | OPA1 H31 | TTGATAACTTTAAACAATTGCTATA |
| | 3UTR(+349+373) | | | 3UTR(+403+427) | |
| 257 | OPA1 H31 | TTGTATTTCTGGAAAAGAAACTGAA | 275 | OPA1 H31 | CAATTGATAACTTTAAACAATTGCT |
| | 3UTR(+352+376) | | | 3UTR(+406+430) | |
| 258 | OPA1 H31 | GCATTGTATTTCTGGAAAAGAAACT | 276 | OPA1 H31 | ATACAATTGATAACTTTAAACAATT |
| | 3UTR(+355+379) | | | 3UTR(+409+433) | |
| 259 | OPA1 H31 | CTAGCATTGTATTTCTGGAAAAGAA | 277 | OPA1 H31 | TATATACAATTGATAACTTTAAACA |
| | 3UTR(+358+382) | | | 3UTR(+412+436) | |
| 260 | OPA1 H31 | CACCTAGCATTGTATTTCTGGAAAA | 278 | OPA1 H31 | TTTTATATACAATTGATAACTTTAA |
| | 3UTR(+361+385) | | | 3UTR(+415+439) | |
| 279 | OPA1 H31 | TGATTTTATATACAATTGATAACTT | 297 | OPA1 H31 | TAGTTTCTGGGAATAGAATACTACA |
| | 3UTR(+418+442) | | | 3UTR(+472+496) | |
| 280 | OPA1 H31 | CTGTGATTTTATATACAATTGATAA | 298 | OPA1 H31 | AAATAGTTTCTGGGAATAGAATACT |
| | 3UTR(+421+445) | | | 3UTR(+475+499) | |
| 281 | OPA1 H31 | CTACTGTGATTTTATATACAATTGA | 299 | OPA1 H31 | GTCAAATAGTTTCTGGGAATAGAAT |
| | 3UTR(+424+448) | | | 3UTR(+478+502) | |
| 282 | OPA1 H31 | AGGCTACTGTGATTTTATATACAAT | 300 | OPA1 H31 | ATGGTCAAATAGTTTCTGGGAATAG |
| | 3UTR(+427+451) | | | 3UTR(+481+505) | |
| 283 | OPA1 H31 | AGCAGGCTACTGTGATTTTATATAC | 301 | OPA1 H31 | ATCATGGTCAAATAGTTTCTGGGAA |
| | 3UTR(+430+454) | | | 3UTR(+484+508) | |
| 284 | OPA1 H31 | TTTAGCAGGCTACTGTGATTTTATA | 302 | OPA1 H31 | ATTATCATGGTCAAATAGTTTCTGG |
| | 3UTR(+433+457) | | | 3UTR(+487+511) | |
| 285 | OPA1 H31 | TGATTTAGCAGGCTACTGTGATTTT | 303 | OPA1 H31 | TGAATTATCATGGTCAAATAGTTTC |
| | 3UTR(+436+460) | | | 3UTR(+490+514) | |
| 286 | OPA1 H31 | CAATGATTTAGCAGGCTACTGTGAT | 304 | OPA1 H31 | AACTGAATTATCATGGTCAAATAGT |
| | 3UTR(+439+463) | | | 3UTR(+493+517) | |
| 287 | OPA1 H31 | ATACAATGATTTAGCAGGCTACTGT | 305 | OPA1 H31 | ATAAACTGAATTATCATGGTCAAAT |
| | 3UTR(+442+466) | | | 3UTR(+496+520) | |
| 288 | OPA1 H31 | CACATACAATGATTTAGCAGGCTAC | 306 | OPA1 H31 | AATATAAACTGAATTATCATGGTCA |
| | 3UTR(+445+469) | | | 3UTR(+499+523) | |
| 289 | OPA1 H31 | AGACACATACAATGATTTAGCAGGC | 307 | OPA1 H31 | GTGAATATAAACTGAATTATCATGG |
| | 3UTR(+448+472) | | | 3UTR(+502+526) | |
| 290 | OPA1 H31 | TACAGACACATACAATGATTTAGCA | 308 | OPA1 H31 | GTGGTGAATATAAACTGAATTATCA |
| | 3UTR(+451+475) | | | 3UTR(+505+529) | |
| 291 | OPA1 H31 | TACTACAGACACATACAATGATTTA | 309 | OPA1 H31 | CATGTGGTGAATATAAACTGAATTA |
| | 3UTR(+454+478) | | | 3UTR(+508+532) | |
| 292 | OPA1 H31 | GAATACTACAGACACATACAATGAT | 310 | OPA1 H31 | TTTCATGTGGTGAATATAAACTGAA |
| | 3UTR(+457+481) | | | 3UTR(+511+535) | |
| 293 | OPA1 H31 | ATAGAATACTACAGACACATACAAT | 311 | OPA1 H31 | TTCTTTCATGTGGTGAATATAAACT |
| | 3UTR(+460+484) | | | 3UTR(+514+538) | |
| 294 | OPA1 H31 | GGAATAGAATACTACAGACACATAC | 312 | OPA1 H31 | TTTTTCTTTCATGTGGTGAATATAA |
| | 3UTR(+463+487) | | | 3UTR(+517+541) | |
| 295 | OPA1 H31 | CTGGGAATAGAATACTACAGACACA | 313 | OPA1 H31 | CCATTTTTCTTTCATGTGGTGAATA |
| | 3UTR(+466+490) | | | 3UTR(+520+544) | |
| 296 | OPA1 H31 | TTTCTGGGAATAGAATACTACAGAC | 314 | OPA1 H31 | TACCCATTTTTCTTTCATGTGGTGA |
| | 3UTR(+469+493) | | | 3UTR(+523+547) | |
| 315 | OPA1 H31 | TGTTACCCATTTTTCTTTCATGTGG | 333 | OPA1 H31 | AAATTTCTTTCACTGAACGTTACAA |
| | 3UTR(+526+550) | | | 3UTR(+580+604) | |
| 316 | OPA1 H31 | TTCTGTTACCCATTTTTCTTTCATG | 334 | OPA1 H31 | TTGAAATTTCTTTCACTGAACGTTA |
| | 3UTR(+529+553) | | | 3UTR(+583+607) | |
| 317 | OPA1 H31 | TTCTTCTGTTACCCATTTTTCTTTC | 335 | OPA1 H31 | GGGTTGAAATTTCTTTCACTGAACG |
| | 3UTR(+532+556) | | | 3UTR(+586+610) | |
| 318 | OPA1 H31 | GGGTTCTTCTGTTACCCATTTTTCT | 336 | OPA1 H31 | GAAGGGTTGAAATTTCTTTCACTGA |
| | 3UTR(+535+559) | | | 3UTR(+589+613) | |
| 319 | OPA1 H31 | TAAGGGTTCTTCTGTTACCCATTTT | 337 | OPA1 H31 | TATGAAGGGTTGAAATTTCTTTCAC |
| | 3UTR(+538+562) | | | 3UTR(+592+616) | |
| 320 | OPA1 H31 | TTTTAAGGGTTCTTCTGTTACCCAT | 338 | OPA1 H31 | GGCTATGAAGGGTTGAAATTTCTTT |
| | 3UTR(+541+565) | | | 3UTR(+595+619) | |
| 321 | OPA1 H31 | CTGTTTTAAGGGTTCTTCTGTTACC | 339 | OPA1 H31 | GCTGGCTATGAAGGGTTGAAATTTC |
| | 3UTR(+544+568) | | | 3UTR(+598+622) | |
| 322 | OPA1 H31 | AACCTGTTTTAAGGGTTCTTCTGTT | 340 | OPA1 H31 | TTCGCTGGCTATGAAGGGTTGAAAT |
| | 3UTR(+547+571) | | | 3UTR(+601+625) | |
| 323 | OPA1 H31 | ATTAACCTGTTTTAAGGGTTCTTCT | 341 | OPA1 H31 | TTCTTCGCTGGCTATGAAGGGTTGA |
| | 3UTR(+550+574) | | | 3UTR(+604+628) | |
| 324 | OPA1 H31 | CAAATTAACCTGTTTTAAGGGTTCT | 342 | OPA1 H31 | AATTTCTTCGCTGGCTATGAAGGGT |
| | 3UTR(+553+577) | | | 3UTR(+607+631) | |
| 325 | OPA1 H31 | ATCCAAATTAACCTGTTTTAAGGGT | 343 | OPA1 H31 | GCAAATTTCTTCGCTGGCTATGAAG |
| | 3UTR(+556+580) | | | 3UTR(+610+634) | |
| 326 | OPA1 H31 | ACAATCCAAATTAACCTGTTTTAAG | 344 | OPA1 H31 | AAGGCAAATTTCTTCGCTGGCTATG |
| | 3UTR(+559+583) | | | 3UTR(+613+637) | |
| 327 | OPA1 H31 | GTTACAATCCAAATTAACCTGTTTT | 345 | OPA1 H31 | TCCAAGGCAAATTTCTTCGCTGGCT |
| | 3UTR(+562+586) | | | 3UTR(+616+640) | |
| 328 | OPA1 H31 | AACGTTACAATCCAAATTAACCTGT | 346 | OPA1 H31 | GCTTCCAAGGCAAATTTCTTCGCTG |
| | 3UTR(+565+589) | | | 3UTR(+619+643) | |
| 329 | OPA1 H31 | CTGAACGTTACAATCCAAATTAACC | 347 | OPA1 H31 | TTGGCTTCCAAGGCAAATTTCTTCG |
| | 3UTR(+568+592) | | | 3UTR(+622+646) | |
| 330 | OPA1 H31 | TCACTGAACGTTACAATCCAAATTA | 348 | OPA1 H31 | GACTTGGCTTCCAAGGCAAATTTCT |
| | 3UTR(+571+595) | | | 3UTR(+625+649) | |
| 331 | OPA1 H31 | CTTTCACTGAACGTTACAATCCAAA | 349 | OPA1 H31 | ACTGACTTGGCTTCCAAGGCAAATT |
| | 3UTR(+574+598) | | | 3UTR(+628+652) | |
| 332 | OPA1 H31 | TTTCTTTCACTGAACGTTACAATCC | 350 | OPA1 H31 | GGTACTGACTTGGCTTCCAAGGCAA |
| | 3UTR(+577+601) | | | 3UTR(+631+655) | |
| 351 | OPA1 H31 | GCTGGTACTGACTTGGCTTCCAAGG | 369 | OPA1 H31 | CAATTTCAAATGGATATAGAGAGTG |
| | 3UTR(+634+658) | | | 3UTR(+688+712) | |
| 352 | OPA1 H31 | TAAGCTGGTACTGACTTGGCTTCCA | 370 | OPA1 H31 | AATCAATTTCAAATGGATATAGAGA |
| | 3UTR(+637+661) | | | 3UTR(+691+715) | |
| 353 | OPA1 H31 | AGGTAAGCTGGTACTGACTTGGCTT | 371 | OPA1 H31 | ATAAATCAATTTCAAATGGATATAG |
| | 3UTR(+640+664) | | | 3UTR(+694+718) | |
| 354 | OPA1 H31 | AATAGGTAAGCTGGTACTGACTTGG | 372 | OPA1 H31 | AAAATAAATCAATTTCAAATGGATA |
| | 3UTR(+643+667) | | | 3UTR(+697+721) | |
| 355 | OPA1 H31 | TCAAATAGGTAAGCTGGTACTGACT | 373 | OPA1 H31 | TCTAAAATAAATCAATTTCAAATGG |
| | 3UTR(+646+670) | | | 3UTR(+700+724) | |
| 356 | OPA1 H31 | GAATCAAATAGGTAAGCTGGTACTG | 374 | OPA1 H31 | ACATCTAAAATAAATCAATTTCAAA |
| | 3UTR(+649+673) | | | 3UTR(+703+727) | |
| 357 | OPA1 H31 | ACTGAATCAAATAGGTAAGCTGGTA | 375 | OPA1 H31 | ACAACATCTAAAATAAATCAATTTC |
| | 3UTR(+652+676) | | | 3UTR(+706+730) | |
| 358 | OPA1 H31 | GCAACTGAATCAAATAGGTAAGCTG | 376 | OPA1 H31 | TATACAACATCTAAAATAAATCAAT |
| | 3UTR(+655+679) | | | 3UTR(+709+733) | |
| 359 | OPA1 H31 | ACAGCAACTGAATCAAATAGGTAAG | 377 | OPA1 H31 | AAGTATACAACATCTAAAATAAATC |
| | 3UTR(+658+682) | | | 3UTR(+712+736) | |
| 360 | OPA1 H31 | AAAACAGCAACTGAATCAAATAGGT | 378 | OPA1 H31 | CGTAAGTATACAACATCTAAAATAA |
| | 3UTR(+661+685) | | | 3UTR(+715+739) | |
| 361 | OPA1 H31 | GAGAAAACAGCAACTGAATCAAATA | 379 | OPA1 H31 | TAACGTAAGTATACAACATCTAAAA |
| | 3UTR(+664+688) | | | 3UTR(+718+742) | |
| 362 | OPA1 H31 | AGTGAGAAAACAGCAACTGAATCAA | 380 | OPA1 H31 | GCCTAACGTAAGTATACAACATCTA |
| | 3UTR(+667+691) | | | 3UTR(+721+745) | |
| 363 | OPA1 H31 | GAGAGTGAGAAAACAGCAACTGAAT | 381 | OPA1 H31 | AAAGCCTAACGTAAGTATACAACAT |
| | 3UTR(+670+694) | | | 3UTR(+724+748) | |
| 364 | OPA1 H31 | ATAGAGAGTGAGAAAACAGCAACTG | 382 | OPA1 H31 | CAGAAAGCCTAACGTAAGTATACAA |
| | 3UTR(+673+697) | | | 3UTR(+727+751) | |
| 365 | OPA1 H31 | GATATAGAGAGTGAGAAAACAGCAA | 383 | OPA1 H31 | TAACAGAAAGCCTAACGTAAGTATA |
| | 3UTR(+676+700) | | | 3UTR(+730+754) | |
| 366 | OPA1 H31 | ATGGATATAGAGAGTGAGAAAACAG | 384 | OPA1 H31 | TATTAACAGAAAGCCTAACGTAAGT |
| | 3UTR(+679+703) | | | 3UTR(+733+757) | |
| 367 | OPA1 H31 | CAAATGGATATAGAGAGTGAGAAAA | 385 | OPA1 H31 | CACTATTAACAGAAAGCCTAACGTA |
| | 3UTR(+682+706) | | | 3UTR(+736+760) | |
| 368 | OPA1 H31 | TTTCAAATGGATATAGAGAGTGAGA | 386 | OPA1 H31 | AACCACTATTAACAGAAAGCCTAAC |
| | 3UTR(+685+709) | | | 3UTR(+739+763) | |
| 387 | OPA1 H31 | AAAAACCACTATTAACAGAAAGCCT | 405 | OPA1 H31 | TATCCTTAATCTTCCTCATCCTGTG |
| | 3UTR(+742+766) | | | 3UTR(+796+820) | |
| 388 | OPA1 H31 | GAGAAAAACCACTATTAACAGAAAG | 406 | OPA1 H31 | GATTATCCTTAATCTTCCTCATCCT |
| | 3UTR(+745+769) | | | 3UTR(+799+823) | |
| 389 | OPA1 H31 | CAGGAGAAAAACCACTATTAACAGA | 407 | OPA1 H31 | ATTGATTATCCTTAATCTTCCTCAT |
| | 3UTR(+748+772) | | | 3UTR(+802+826) | |
| 390 | OPA1 H31 | CAACAGGAGAAAAACCACTATTAAC | 408 | OPA1 H31 | TCAATTGATTATCCTTAATCTTCCT |
| | 3UTR(+751+775) | | | 3UTR(+805+829) | |
| 391 | OPA1 H31 | TGTCAACAGGAGAAAAACCACTATT | 409 | OPA1 H31 | TAGTCAATTGATTATCCTTAATCTT |
| | 3UTR(+754+778) | | | 3UTR(+808+832) | |
| 392 | OPA1 H31 | CTCTGTCAACAGGAGAAAAACCACT | 410 | OPA1 H31 | AATTAGTCAATTGATTATCCTTAAT |
| | 3UTR(+757+781) | | | 3UTR(+811+835) | |
| 393 | OPA1 H31 | TGGCTCTGTCAACAGGAGAAAAACC | 411 | OPA1 H31 | TGAAATTAGTCAATTGATTATCCTT |
| | 3UTR(+760+784) | | | 3UTR(+814+838) | |
| 394 | OPA1 H31 | CGGTGGCTCTGTCAACAGGAGAAAA | 412 | OPA1 H31 | AAATGAAATTAGTCAATTGATTATC |
| | 3UTR(+763+787) | | | 3UTR(+817+841) | |
| 395 | OPA1 H31 | ATCCGGTGGCTCTGTCAACAGGAGA | 413 | OPA1 H31 | TCTAAATGAAATTAGTCAATTGATT |
| | 3UTR(+766+790) | | | 3UTR(+820+844) | |
| 396 | OPA1 H31 | ATAATCCGGTGGCTCTGTCAACAGG | 414 | OPA1 H31 | TATTCTAAATGAAATTAGTCAATTG |
| | 3UTR(+769+793) | | | 3UTR(+823+847) | |
| 397 | OPA1 H31 | GTCATAATCCGGTGGCTCTGTCAAC | 415 | OPA1 H31 | TAATATTCTAAATGAAATTAGTCAA |
| | 3UTR(+772+796) | | | 3UTR(+826+850) | |
| 398 | OPA1 H31 | TGTGTCATAATCCGGTGGCTCTGTC | 416 | OPA1 H31 | TGATAATATTCTAAATGAAATTAGT |
| | 3UTR(+775+799) | | | 3UTR(+829+853) | |
| 399 | OPA1 H31 | TCCTGTGTCATAATCCGGTGGCTCT | 417 | OPA1 H31 | GTTTGATAATATTCTAAATGAAATT |
| | 3UTR(+778+802) | | | 3UTR(+832+856) | |
| 400 | OPA1 H31 | TCATCCTGTGTCATAATCCGGTGGC | 418 | OPA1 H31 | AATGTTTGATAATATTCTAAATGAA |
| | 3UTR(+781+805) | | | 3UTR(+835+859) | |
| 401 | OPA1 H31 | TCCTCATCCTGTGTCATAATCCGGT | 419 | OPA1 H31 | TGAAATGTTTGATAATATTCTAAAT |
| | 3UTR(+784+808) | | | 3UTR(+838+862) | |
| 402 | OPA1 H31 | TCTTCCTCATCCTGTGTCATAATCC | 420 | OPA1 H31 | AGTTGAAATGTTTGATAATATTCTA |
| | 3UTR(+787+811) | | | 3UTR(+841+865) | |
| 403 | OPA1 H31 | TAATCTTCCTCATCCTGTGTCATAA | 421 | OPA1 H31 | CCTAGTTGAAATGTTTGATAATATT |
| | 3UTR(+790+814) | | | 3UTR(+844+868) | |
| 404 | OPA1 H31 | CCTTAATCTTCCTCATCCTGTGTCA | 422 | OPA1 H31 | ATACCTAGTTGAAATGTTTGATAAT |
| | 3UTR(+793+817) | | | 3UTR(+847+871) | |
| 423 | OPA1 H31 | CTGATACCTAGTTGAAATGTTTGAT | 441 | OPA1 H31 | TTAATTGTTGAAATAATTTCTATTT |
| | 3UTR(+850+874) | | | 3UTR(+904+928) | |
| 424 | OPA1 H31 | TTTCTGATACCTAGTTGAAATGTTT | 442 | OPA1 H31 | ACTTTAATTGTTGAAATAATTTCTA |
| | 3UTR(+853+877) | | | 3UTR(+907+931) | |
| 425 | OPA1 H31 | CTTTTTCTGATACCTAGTTGAAATG | 443 | OPA1 H31 | ATTACTTTAATTGTTGAAATAATTT |
| | 3UTR(+856+880) | | | 3UTR(+910+934) | |
| 426 | OPA1 H31 | AGCCTTTTTCTGATACCTAGTTGAA | 444 | OPA1 H31 | AACATTACTTTAATTGTTGAAATAA |
| | 3UTR(+859+883) | | | 3UTR(+913+937) | |
| 427 | OPA1 H31 | GAAAGCCTTTTTCTGATACCTAGTT | 445 | OPA1 H31 | GTCAACATTACTTTAATTGTTGAAA |
| | 3UTR(+862+886) | | | 3UTR(+916+940) | |
| 428 | OPA1 H31 | AAAGAAAGCCTTTTTCTGATACCTA | 446 | OPA1 H31 | ATGGTCAACATTACTTTAATTGTTG |
| | 3UTR(+865+889) | | | 3UTR(+919+943) | |
| 429 | OPA1 H31 | ATGAAAGAAAGCCTTTTTCTGATAC | 447 | OPA1 H31 | GGGATGGTCAACATTACTTTAATTG |
| | 3UTR(+868+892) | | | 3UTR(+922+946) | |
| 430 | OPA1 H31 | CTTATGAAAGAAAGCCTTTTTCTGA | 448 | OPA1 H31 | AGGGGGATGGTCAACATTACTTTAA |
| | 3UTR(+871+895) | | | 3UTR(+925+949) | |
| 431 | OPA1 H31 | AGTCTTATGAAAGAAAGCCTTTTTC | 449 | OPA1 H31 | GAGAGGGGGATGGTCAACATTACTT |
| | 3UTR(+874+898) | | | 3UTR(+928+952) | |
| 432 | OPA1 H31 | AATAGTCTTATGAAAGAAAGCCTTT | 450 | OPA1 H31 | GCTGAGAGGGGGATGGTCAACATTA |
| | 3UTR(+877+901) | | | 3UTR(+931+955) | |
| 433 | OPA1 H31 | TAAAATAGTCTTATGAAAGAAAGCC | 451 | OPA1 H31 | TCAGCTGAGAGGGGGATGGTCAACA |
| | 3UTR(+880+904) | | | 3UTR(+934+958) | |
| 434 | OPA1 H31 | ATTTAAAATAGTCTTATGAAAGAAA | 452 | OPA1 H31 | TATTCAGCTGAGAGGGGGATGGTCA |
| | 3UTR(+883+907) | | | 3UTR(+937+961) | |
| 435 | OPA1 H31 | TCTATTTAAAATAGTCTTATGAAAG | 453 | OPA1 H31 | CTTTATTCAGCTGAGAGGGGGATGG |
| | 3UTR(+886+910) | | | 3UTR(+940+964) | |
| 436 | OPA1 H31 | ATTTCTATTTAAAATAGTCTTATGA | 454 | OPA1 H31 | TTTCTTTATTCAGCTGAGAGGGGGA |
| | 3UTR(+889+913) | | | 3UTR(+943+967) | |
| 437 | OPA1 H31 | ATAATTTCTATTTAAAATAGTCTTA | 455 | OPA1 H31 | ATTTTTCTTTATTCAGCTGAGAGGG |
| | 3UTR(+892+916) | | | 3UTR(+946+970) | |
| 438 | OPA1 H31 | GAAATAATTTCTATTTAAAATAGTC | 456 | OPA1 H31 | TAAATTTTTCTTTATTCAGCTGAGA |
| | 3UTR(+895+919) | | | 3UTR(+949+973) | |
| 439 | OPA1 H31 | GTTGAAATAATTTCTATTTAAAATA | 457 | OPA1 H31 | AACTAAATTTTTCTTTATTCAGCTG |
| | 3UTR(+898+922) | | | 3UTR(+952+976) | |
| 440 | OPA1 H31 | ATTGTTGAAATAATTTCTATTTAAA | 458 | OPA1 H31 | TTGAACTAAATTTTTCTTTATTCAG |
| | 3UTR(+901+925) | | | 3UTR(+955+979) | |
| 459 | OPA1 H31 | AAATTGAACTAAATTTTTCTTTATT | 477 | OPA1 H31 | TTATTTAAAACACATGAAAATGTTG |
| | 3UTR(+958+982) | | | 3UTR(+1012+1036) | |
| 460 | OPA1 H31 | AATAAATTGAACTAAATTTTTCTTT | 478 | OPA1 H31 | TATTTATTTAAAACACATGAAAATG |
| | 3UTR(+961+985) | | | 3UTR(+1015+1039) | |
| 461 | OPA1 H31 | TGCAATAAATTGAACTAAATTTTTC | 479 | OPA1 H31 | AAATATTTATTTAAAACACATGAAA |
| | 3UTR(+964+988) | | | 3UTR(+1018+1042) | |
| 462 | OPA1 H31 | AATTGCAATAAATTGAACTAAATTT | 480 | OPA1 H31 | AAAAAATATTTATTTAAAACACATG |
| | 3UTR(+967+991) | | | 3UTR(+1021+1045) | |
| 463 | OPA1 H31 | TTAAATTGCAATAAATTGAACTAAA | 481 | OPA1 H31 | ATTAAAAAATATTTATTTAAAACAC |
| | 3UTR(+970+994) | | | 3UTR(+1024+1048) | |
| 464 | OPA1 H31 | TAATTAAATTGCAATAAATTGAACT | 482 | OPA1 H31 | CCAATTAAAAAATATTTATTTAAAA |
| | 3UTR(+973+997) | | | 3UTR(+1027+1051) | |
| 465 | OPA1 H31 | TTGTAATTAAATTGCAATAAATTGA | 483 | OPA1 H31 | TAGCCAATTAAAAAATATTTATTTA |
| | 3UTR(+976+1000) | | | 3UTR(+1030+1054) | |
| 466 | OPA1 H31 | GTATTGTAATTAAATTGCAATAAAT | 484 | OPA1 H31 | CTTTAGCCAATTAAAAAATATTTAT |
| | 3UTR(+979+1003) | | | 3UTR(+1033+1057) | |
| 467 | OPA1 H31 | GTAGTATTGTAATTAAATTGCAATA | 485 | OPA1 H31 | GTCCTTTAGCCAATTAAAAAATATT |
| | 3UTR(+982+1006) | | | 3UTR(+1036+1060) | |
| 468 | OPA1 H31 | AAGGTAGTATTGTAATTAAATTGCA | 486 | OPA1 H31 | AATGTCCTTTAGCCAATTAAAAAAT |
| | 3UTR(+985+1009) | | | 3UTR(+1039+1063) | |
| 469 | OPA1 H31 | GTGAAGGTAGTATTGTAATTAAATT | 487 | OPA1 H31 | TTGAATGTCCTTTAGCCAATTAAAA |
| | 3UTR(+988+1012) | | | 3UTR(+1042+1066) | |
| 470 | OPA1 H31 | GTTGTGAAGGTAGTATTGTAATTAA | 488 | OPA1 H31 | TGCTTGAATGTCCTTTAGCCAATTA |
| | 3UTR(+991+1015) | | | 3UTR(+1045+1069) | |
| 471 | OPA1 H31 | AATGTTGTGAAGGTAGTATTGTAAT | 489 | OPA1 H31 | CTTTGCTTGAATGTCCTTTAGCCAA |
| | 3UTR(+994+1018) | | | 3UTR(+1048+1072) | |
| 472 | OPA1 H31 | GAAAATGTTGTGAAGGTAGTATTGT | 490 | OPA1 H31 | TTTCTTTGCTTGAATGTCCTTTAGC |
| | 3UTR(+997+1021) | | | 3UTR(+1051+1075) | |
| 473 | OPA1 H31 | CATGAAAATGTTGTGAAGGTAGTAT | 491 | OPA1 H31 | GCATTTCTTTGCTTGAATGTCCTTT |
| | 3UTR(+1000+1024) | | | 3UTR(+1054+1078) | |
| 474 | OPA1 H31 | ACACATGAAAATGTTGTGAAGGTAG | 492 | OPA1 H31 | AAAGCATTTCTTTGCTTGAATGTCC |
| | 3UTR(+1003+1027) | | | 3UTR(+1057+1081) | |
| 475 | OPA1 H31 | AAAACACATGAAAATGTTGTGAAGG | 493 | OPA1 H31 | AAGAAAGCATTTCTTTGCTTGAATG |
| | 3UTR(+1006+1030) | | | 3UTR(+1060+1084) | |
| 476 | OPA1 H31 | TTTAAAACACATGAAAATGTTGTGA | 494 | OPA1 H31 | GTAAAGAAAGCATTTCTTTGCTTGA |
| | 3UTR(+1009+1033) | | | 3UTR(+1063+1087) | |
| 495 | OPA1 H31 | TAAGTAAAGAAAGCATTTCTTTGCT | 513 | OPA1 H31 | TTTTATTAACAAAGTAAAGGCTTAG |
| | 3UTR(+1066+1090) | | | 3UTR(+1120+1144) | |
| 496 | OPA1 H31 | TTTTAAGTAAAGAAAGCATTTCTTT | 514 | OPA1 H31 | CACTTTTATTAACAAAGTAAAGGCT |
| | 3UTR(+1069+1093) | | | 3UTR(+1123+1147) | |
| 497 | OPA1 H31 | ACATTTTAAGTAAAGAAAGCATTTC | 515 | OPA1 H31 | GGACACTTTTATTAACAAAGTAAAG |
| | 3UTR(+1072+1096) | | | 3UTR(+1126+1150) | |
| 498 | OPA1 H31 | TAGACATTTTAAGTAAAGAAAGCAT | 516 | OPA1 H31 | AATGGACACTTTTATTAACAAAGTA |
| | 3UTR(+1075+1099) | | | 3UTR(+1129+1153) | |
| 499 | OPA1 H31 | AGATAGACATTTTAAGTAAAGAAAG | 517 | OPA1 H31 | CACAATGGACACTTTTATTAACAAA |
| | 3UTR(+1078+1102) | | | 3UTR(+1132+1156) | |
| 500 | OPA1 H31 | ATGAGATAGACATTTTAAGTAAAGA | 518 | OPA1 H31 | TCACACAATGGACACTTTTATTAAC |
| | 3UTR(+1081+1105) | | | 3UTR(+1135+1159) | |
| 501 | OPA1 H31 | CAAATGAGATAGACATTTTAAGTAA | 519 | OPA1 H31 | ACATCACACAATGGACACTTTTATT |
| | 3UTR(+1084+1108) | | | 3UTR(+1138+1162) | |
| 502 | OPA1 H31 | CAGCAAATGAGATAGACATTTTAAG | 520 | OPA1 H31 | AAAACATCACACAATGGACACTTTT |
| | 3UTR(+1087+1111) | | | 3UTR(+1141+1165) | |
| 503 | OPA1 H31 | AGGCAGCAAATGAGATAGACATTTT | 521 | OPA1 H31 | TCAAAAACATCACACAATGGACACT |
| | 3UTR(+1090+1114) | | | 3UTR(+1144+1168) | |
| 504 | OPA1 H31 | AAAAGGCAGCAAATGAGATAGACAT | 522 | OPA1 H31 | AAATCAAAAACATCACACAATGGAC |
| | 3UTR(+1093+1117) | | | 3UTR(+1147+1171) | |
| 505 | OPA1 H31 | GTGAAAAGGCAGCAAATGAGATAGA | 523 | OPA1 H31 | GTAAAATCAAAAACATCACACAATG |
| | 3UTR(+1096+1120) | | | 3UTR(+1150+1174) | |
| 506 | OPA1 H31 | TTAGTGAAAAGGCAGCAAATGAGAT | 524 | OPA1 H31 | ACTGTAAAATCAAAAACATCACACA |
| | 3UTR(+1099+1123) | | | 3UTR(+1153+1177) | |
| 507 | OPA1 H31 | GGCTTAGTGAAAAGGCAGCAAATGA | 525 | OPA1 H31 | CAAACTGTAAAATCAAAAACATCAC |
| | 3UTR(+1102+1126) | | | 3UTR(+1156+1180) | |
| 508 | OPA1 H31 | AAAGGCTTAGTGAAAAGGCAGCAAA | 526 | OPA1 H31 | TAGCAAACTGTAAAATCAAAAACAT |
| | 3UTR(+1105+1129) | | | 3UTR(+1159+1183) | |
| 509 | OPA1 H31 | AGTAAAGGCTTAGTGAAAAGGCAGC | 527 | OPA1 H31 | ATTTAGCAAACTGTAAAATCAAAAA |
| | 3UTR(+1108+1132) | | | 3UTR(+1162+1186) | |
| 510 | OPA1 H31 | CAAAGTAAAGGCTTAGTGAAAAGGC | 528 | OPA1 H31 | AAGATTTAGCAAACTGTAAAATCAA |
| | 3UTR(+1111+1135) | | | 3UTR(+1165+1189) | |
| 511 | OPA1 H31 | TAACAAAGTAAAGGCTTAGTGAAAA | 529 | OPA1 H31 | AATAAGATTTAGCAAACTGTAAAAT |
| | 3UTR(+1114+1138) | | | 3UTR(+1168+1192) | |
| 512 | OPA1 H31 | TATTAACAAAGTAAAGGCTTAGTGA | 530 | OPA1 H31 | GAAAATAAGATTTAGCAAACTGTAA |
| | 3UTR(+1117+1141) | | | 3UTR(+1171+1195) | |
| 531 | OPA1 H31 | CAAGAAAATAAGATTTAGCAAACTG | 549 | OPA1 H31 | ATGTAAAACAATAAAATGGGGAGTA |
| | 3UTR(+1174+1198) | | | 3UTR(+1228+1252) | |
| 532 | OPA1 H31 | CTCCAAGAAAATAAGATTTAGCAAA | 550 | OPA1 H31 | TTGATGTAAAACAATAAAATGGGGA |
| | 3UTR(+1177+1201) | | | 3UTR(+1231+1255) | |
| 533 | OPA1 H31 | CAACTCCAAGAAAATAAGATTTAGC | 551 | OPA1 H31 | GCATTGATGTAAAACAATAAAATGG |
| | 3UTR(+1180+1204) | | | 3UTR(+1234+1258) | |
| 534 | OPA1 H31 | AAGCAACTCCAAGAAAATAAGATTT | 552 | OPA1 H31 | CATGCATTGATGTAAAACAATAAAA |
| | 3UTR(+1183+1207) | | | 3UTR(+1237+1261) | |
| 535 | OPA1 H31 | AAAAAGCAACTCCAAGAAAATAAGA | 553 | OPA1 H31 | AAGCATGCATTGATGTAAAACAATA |
| | 3UTR(+1186+1210) | | | 3UTR(+1240+1264) | |
| 536 | OPA1 H31 | ACCAAAAAGCAACTCCAAGAAAATA | 554 | OPA1 H31 | ACGAAGCATGCATTGATGTAAAACA |
| | 3UTR(+1189+1213) | | | 3UTR(+1243+1267) | |
| 537 | OPA1 H31 | GTTACCAAAAAGCAACTCCAAGAAA | 555 | OPA1 H31 | ACAACGAAGCATGCATTGATGTAAA |
| | 3UTR(+1192+1216) | | | 3UTR(+1246+1270) | |
| 538 | OPA1 H31 | GCTGTTACCAAAAAGCAACTCCAAG | 556 | OPA1 H31 | ATCACAACGAAGCATGCATTGATGT |
| | 3UTR(+1195+1219) | | | 3UTR(+1249+1273) | |
| 539 | OPA1 H31 | GGGGCTGTTACCAAAAAGCAACTCC | 557 | OPA1 H31 | GGGATCACAACGAAGCATGCATTGA |
| | 3UTR(+1198+1222) | | | 3UTR(+1252+1276) | |
| 540 | OPA1 H31 | AATGGGGCTGTTACCAAAAAGCAAC | 558 | OPA1 H31 | TGAGGGATCACAACGAAGCATGCAT |
| | 3UTR(+1201+1225) | | | 3UTR(+1255+1279) | |
| 541 | OPA1 H31 | AGCAATGGGGCTGTTACCAAAAAGC | 559 | OPA1 H31 | TCTTGAGGGATCACAACGAAGCATG |
| | 3UTR(+1204+1228) | | | 3UTR(+1258+1282) | |
| 542 | OPA1 H31 | AGTAGCAATGGGGCTGTTACCAAAA | 560 | OPA1 H31 | ACATCTTGAGGGATCACAACGAAGC |
| | 3UTR(+1207+1231) | | | 3UTR(+1261+1285) | |
| 543 | OPA1 H31 | GGGAGTAGCAATGGGGCTGTTACCA | 561 | OPA1 H31 | GTTACATCTTGAGGGATCACAACGA |
| | 3UTR(+1210+1234) | | | 3UTR(+1264+1288) | |
| 544 | OPA1 H31 | ATGGGGAGTAGCAATGGGGCTGTTA | 562 | OPA1 H31 | AGTGTTACATCTTGAGGGATCACAA |
| | 3UTR(+1213+1237) | | | 3UTR(+1267+1291) | |
| 545 | OPA1 H31 | AAAATGGGGAGTAGCAATGGGGCTG | 563 | OPA1 H31 | CCAAGTGTTACATCTTGAGGGATCA |
| | 3UTR(+1216+1240) | | | 3UTR(+1270+1294) | |
| 546 | OPA1 H31 | AATAAAATGGGGAGTAGCAATGGGG | 564 | OPA1 H31 | ATACCAAGTGTTACATCTTGAGGGA |
| | 3UTR(+1219+1243) | | | 3UTR(+1273+1297) | |
| 547 | OPA1 H31 | AACAATAAAATGGGGAGTAGCAATG | 565 | OPA1 H31 | AGCATACCAAGTGTTACATCTTGAG |
| | 3UTR(+1222+1246) | | | 3UTR(+1276+1300) | |
| 548 | OPA1 H31 | TAAAACAATAAAATGGGGAGTAGCA | 566 | OPA1 H31 | CCGAGCATACCAAGTGTTACATCTT |
| | 3UTR(+1225+1249) | | | 3UTR(+1279+1303) | |
| 567 | OPA1 H31 | CAACCGAGCATACCAAGTGTTACAT | 585 | OPA1 H31 | TATAAAACAAACATACATTGAATTC |
| | 3UTR(+1282+1306) | | | 3UTR(+1336+1360) | |
| 568 | OPA1 H31 | CCTCAACCGAGCATACCAAGTGTTA | 586 | OPA1 H31 | CAGTATAAAACAAACATACATTGAA |
| | 3UTR(+1285+1309) | | | 3UTR(+1339+1363) | |
| 569 | OPA1 H31 | TATCCTCAACCGAGCATACCAAGTG | 587 | OPA1 H31 | AAACAGTATAAAACAAACATACATT |
| | 3UTR(+1288+1312) | | | 3UTR(+1342+1366) | |
| 570 | OPA1 H31 | TCATATCCTCAACCGAGCATACCAA | 588 | OPA1 H31 | ATCAAACAGTATAAAACAAACATAC |
| | 3UTR(+1291+1315) | | | 3UTR(+1345+1369) | |
| 571 | OPA1 H31 | TTTTCATATCCTCAACCGAGCATAC | 589 | OPA1 H31 | CTTATCAAACAGTATAAAACAAACA |
| | 3UTR(+1294+1318) | | | 3UTR(+1348+1372) | |
| 572 | OPA1 H31 | ATTTTTTCATATCCTCAACCGAGCA | 590 | OPA1 H31 | TTTCTTATCAAACAGTATAAAACAA |
| | 3UTR(+1297+1321) | | | 3UTR(+1351+1375) | |
| 573 | OPA1 H31 | AGTATTTTTTCATATCCTCAACCGA | 591 | OPA1 H31 | ACTTTTCTTATCAAACAGTATAAAA |
| | 3UTR(+1300+1324) | | | 3UTR(+1354+1378) | |
| 574 | OPA1 H31 | GGAAGTATTTTTTCATATCCTCAAC | 592 | OPA1 H31 | CCTACTTTTCTTATCAAACAGTATA |
| | 3UTR(+1303+1327) | | | 3UTR(+1357+1381) | |
| 575 | OPA1 H31 | TTCGGAAGTATTTTTTCATATCCTC | 593 | OPA1 H31 | GGACCTACTTTTCTTATCAAACAGT |
| | 3UTR(+1306+1330) | | | 3UTR(+1360+1384) | |
| 576 | OPA1 H31 | GGTTTCGGAAGTATTTTTTCATATC | 594 | OPA1 H31 | GCTGGACCTACTTTTCTTATCAAAC |
| | 3UTR(+1309+1333) | | | 3UTR(+1363+1387) | |
| 577 | OPA1 H31 | CCTGGTTTCGGAAGTATTTTTTCAT | 595 | OPA1 H31 | AAGGCTGGACCTACTTTTCTTATCA |
| | 3UTR(+1312+1336) | | | 3UTR(+1366+1390) | |
| 578 | OPA1 H31 | ATTCCTGGTTTCGGAAGTATTTTTT | 596 | OPA1 H31 | CTTAAGGCTGGACCTACTTTTCTTA |
| | 3UTR(+1315+1339) | | | 3UTR(+1369+1393) | |
| 579 | OPA1 H31 | TGAATTCCTGGTTTCGGAAGTATTT | 597 | OPA1 H31 | CTGCTTAAGGCTGGACCTACTTTTC |
| | 3UTR(+1318+1342) | | | 3UTR(+1372+1396) | |
| | OPA1 H31 | CATTGAATTCCTGGTTTCGGAAGTA | 598 | OPA1 H31 | GTGCTGCTTAAGGCTGGACCTACTT |
| 580 | 3UTR(+1321+1345) | | | 3UTR(+1375+1399) | |
| 581 | OPA1 H31 | ATACATTGAATTCCTGGTTTCGGAA | 599 | OPA1 H31 | TCTGTGCTGCTTAAGGCTGGACCTA |
| | 3UTR(+1324+1348) | | | 3UTR(+1378+1402) | |
| 582 | OPA1 H31 | AACATACATTGAATTCCTGGTTTCG | 600 | OPA1 H31 | GCATCTGTGCTGCTTAAGGCTGGAC |
| | 3UTR(+1327+1351) | | | 3UTR(+1381+1405) | |
| 583 | OPA1 H31 | ACAAACATACATTGAATTCCTGGTT | 601 | OPA1 H31 | AGCGCATCTGTGCTGCTTAAGGCTG |
| | 3UTR(+1330+1354) | | | 3UTR(+1384+1408) | |
| 584 | OPA1 H31 | AAAACAAACATACATTGAATTCCTG | 602 | OPA1 H31 | ACCAGCGCATCTGTGCTGCTTAAGG |
| | 3UTR(+1333+1357) | | | 3UTR(+1387+1411) | |
| 603 | OPA1 H31 | TCTACCAGCGCATCTGTGCTGCTTA | 621 | OPA1 H31 | TATTCACTCCTGTCCCTAGACCTAA |
| | 3UTR(+1390+1414) | | | 3UTR(+1444+1468) | |
| 604 | OPA1 H31 | GCATCTACCAGCGCATCTGTGCTGC | 622 | OPA1 H31 | TTCTATTCACTCCTGTCCCTAGACC |
| | 3UTR(+1393+1417) | | | 3UTR(+1447+1471) | |
| 605 | OPA1 H31 | TATGCATCTACCAGCGCATCTGTGC | 623 | OPA1 H31 | CCTTTCTATTCACTCCTGTCCCTAG |
| | 3UTR(+1396+1420) | | | 3UTR(+1450+1474) | |
| 606 | OPA1 H31 | GACTATGCATCTACCAGCGCATCTG | 624 | OPA1 H31 | CTCCCTTTCTATTCACTCCTGTCCC |
| | 3UTR(+1399+1423) | | | 3UTR(+1453+1477) | |
| | OPA1 H31 | CCTGACTATGCATCTACCAGCGCAT | 625 | OPA1 H31 | CTCCTCCCTTTCTATTCACTCCTGT |
| 607 | 3UTR(+1402+1426) | | | 3UTR(+1456+1480) | |
| 608 | OPA1 H31 | GTTCCTGACTATGCATCTACCAGCG | 626 | OPA1 H31 | GCTCTCCTCCCTTTCTATTCACTCC |
| | 3UTR(+1405+1429) | | | 3UTR(+1459+1483) | |
| 609 | OPA1 H31 | AAAGTTCCTGACTATGCATCTACCA | 627 | OPA1 H31 | AGAGCTCTCCTCCCTTTCTATTCAC |
| | 3UTR(+1408+1432) | | | 3UTR(+1462+1486) | |
| 610 | OPA1 H31 | AAAAAAGTTCCTGACTATGCATCTA | 628 | OPA1 H31 | AATAGAGCTCTCCTCCCTTTCTATT |
| | 3UTR(+1411+1435) | | | 3UTR(+1465+1489) | |
| 611 | OPA1 H31 | ATAAAAAAAGTTCCTGACTATGCAT | 629 | OPA1 H31 | CATAATAGAGCTCTCCTCCCTTTCT |
| | 3UTR(+1414+1438) | | | 3UTR(+1468+1492) | |
| 612 | OPA1 H31 | GAAATAAAAAAAGTTCCTGACTATG | 630 | OPA1 H31 | GAACATAATAGAGCTCTCCTCCCTT |
| | 3UTR(+1417+1441) | | | 3UTR(+1471+1495) | |
| 613 | OPA1 H31 | AAAGAAATAAAAAAAGTTCCTGACT | 631 | OPA1 H31 | ATAGAACATAATAGAGCTCTCCTCC |
| | 3UTR(+1420+1444) | | | 3UTR(+1474+1498) | |
| 614 | OPA1 H31 | CTAAAAGAAATAAAAAAAGTTCCTG | 632 | OPA1 H31 | TGTATAGAACATAATAGAGCTCTCC |
| | 3UTR(+1423+1447) | | | 3UTR(+1477+1501) | |
| 615 | OPA1 H31 | GACCTAAAAGAAATAAAAAAAGTTC | 633 | OPA1 H31 | CTGTGTATAGAACATAATAGAGCTC |
| | 3UTR(+1426+1450) | | | 3UTR(+1480+1504) | |
| 616 | OPA1 H31 | CTAGACCTAAAAGAAATAAAAAAAG | 634 | OPA1 H31 | AATCTGTGTATAGAACATAATAGAG |
| | 3UTR(+1429+1453) | | | 3UTR(+1483+1507) | |
| 617 | OPA1 H31 | TCCCTAGACCTAAAAGAAATAAAAA | 635 | OPA1 H31 | CCTAATCTGTGTATAGAACATAATA |
| | 3UTR(+1432+1456) | | | 3UTR(+1486+1510) | |
| 618 | OPA1 H31 | CTGTCCCTAGACCTAAAAGAAATAA | 636 | OPA1 H31 | TCTCCTAATCTGTGTATAGAACATA |
| | 3UTR(+1435+1459) | | | 3UTR(+1489+1513) | |
| 619 | OPA1 H31 | CTCCTGTCCCTAGACCTAAAAGAAA | 637 | OPA1 H31 | TCATCTCCTAATCTGTGTATAGAAC |
| | 3UTR(+1438+1462) | | | 3UTR(+1492+1516) | |
| 620 | OPA1 H31 | TCACTCCTGTCCCTAGACCTAAAAG | 638 | OPA1 H31 | AGGTCATCTCCTAATCTGTGTATAG |
| | 3UTR(+1441+1465) | | | 3UTR(+1495+1519) | |
| 639 | OPA1 H31 | GTAAGGTCATCTCCTAATCTGTGTA | 657 | OPA1 H31 | AAATATTCATTAACATGCATTAACC |
| | 3UTR(+1498+1522) | | | 3UTR(+1552+1576) | |
| 640 | OPA1 H31 | CCAGTAAGGTCATCTCCTAATCTGT | 658 | OPA1 H31 | CAAAAATATTCATTAACATGCATTA |
| | 3UTR(+1501+1525) | | | 3UTR(+1555+1579) | |
| 641 | OPA1 H31 | TACCCAGTAAGGTCATCTCCTAATC | 659 | OPA1 H31 | CTGCAAAAATATTCATTAACATGCA |
| | 3UTR(+1504+1528) | | | 3UTR(+1558+1582) | |
| 642 | OPA1 H31 | GTGTACCCAGTAAGGTCATCTCCTA | 660 | OPA1 H31 | CAACTGCAAAAATATTCATTAACAT |
| | 3UTR(+1507+1531) | | | 3UTR(+1561+1585) | |
| 643 | OPA1 H31 | GGGGTGTACCCAGTAAGGTCATCTC | 661 | OPA1 H31 | TTACAACTGCAAAAATATTCATTAA |
| | 3UTR(+1510+1534) | | | 3UTR(+1564+1588) | |
| 644 | OPA1 H31 | AGAGGGGTGTACCCAGTAAGGTCAT | 662 | OPA1 H31 | GCTTTACAACTGCAAAAATATTCAT |
| | 3UTR(+1513+1537) | | | 3UTR(+1567+1591) | |
| 645 | OPA1 H31 | GTTAGAGGGGTGTACCCAGTAAGGT | 663 | OPA1 H31 | TATGCTTTACAACTGCAAAAATATT |
| | 3UTR(+1516+1540) | | | 3UTR(+1570+1594) | |
| 646 | OPA1 H31 | CTGGTTAGAGGGGTGTACCCAGTAA | 664 | OPA1 H31 | TGTTATGCTTTACAACTGCAAAAAT |
| | 3UTR(+1519+1543) | | | 3UTR(+1573+1597) | |
| 647 | OPA1 H31 | GCACTGGTTAGAGGGGTGTACCCAG | 665 | OPA1 H31 | AATTGTTATGCTTTACAACTGCAAA |
| | 3UTR(+1522+1546) | | | 3UTR(+1576+1600) | |
| 648 | OPA1 H31 | TAAGCACTGGTTAGAGGGGTGTACC | 666 | OPA1 H31 | TGTAATTGTTATGCTTTACAACTGC |
| | 3UTR(+1525+1549) | | | 3UTR(+1579+1603) | |
| 649 | OPA1 H31 | CTGTAAGCACTGGTTAGAGGGGTGT | 667 | OPA1 H31 | AGTTGTAATTGTTATGCTTTACAAC |
| | 3UTR(+1528+1552) | | | 3UTR(+1582+1606) | |
| 650 | OPA1 H31 | AACCTGTAAGCACTGGTTAGAGGGG | 668 | OPA1 H31 | TGTAGTTGTAATTGTTATGCTTTAC |
| | 3UTR(+1531+1555) | | | 3UTR(+1585+1609) | |
| 651 | OPA1 H31 | ATTAACCTGTAAGCACTGGTTAGAG | 669 | OPA1 H31 | ATGTGTAGTTGTAATTGTTATGCTT |
| | 3UTR(+1534+1558) | | | 3UTR(+1588+1612) | |
| 652 | OPA1 H31 | TGCATTAACCTGTAAGCACTGGTTA | 670 | OPA1 H31 | TAGATGTGTAGTTGTAATTGTTATG |
| | 3UTR(+1537+1561) | | | 3UTR(+1591+1615) | |
| 653 | OPA1 H31 | ACATGCATTAACCTGTAAGCACTGG | 671 | OPA1 H31 | AAATAGATGTGTAGTTGTAATTGTT |
| | 3UTR(+1540+1564) | | | 3UTR(+1594+1618) | |
| 654 | OPA1 H31 | TTAACATGCATTAACCTGTAAGCAC | 672 | OPA1 H31 | TAGAAATAGATGTGTAGTTGTAATT |
| | 3UTR(+1543+1567) | | | 3UTR(+1597+1621) | |
| 655 | OPA1 H31 | TCATTAACATGCATTAACCTGTAAG | 673 | OPA1 H31 | CTTTAGAAATAGATGTGTAGTTGTA |
| | 3UTR(+1546+1570) | | | 3UTR(+1600+1624) | |
| 656 | OPA1 H31 | TATTCATTAACATGCATTAACCTGT | 674 | OPA1 H31 | ATTCTTTAGAAATAGATGTGTAGTT |
| | 3UTR(+1549+1573) | | | 3UTR(+1603+1627) | |
| 675 | OPA1 H31 | TTTATTCTTTAGAAATAGATGTGTA | 693 | OPA1 H31 | CAGCTGAAGGTCTTTCTTTCTATAG |
| | 3UTR(+1606+1630) | | | 3UTR(+1660+1684) | |
| 676 | OPA1 H31 | TGTTTTATTCTTTAGAAATAGATGT | 694 | OPA1 H31 | ATACAGCTGAAGGTCTTTCTTTCTA |
| | 3UTR(+1609+1633) | | | 3UTR(+1663+1687) | |
| 677 | OPA1 H31 | TCCTGTTTTATTCTTTAGAAATAGA | 695 | OPA1 H31 | GAAATACAGCTGAAGGTCTTTCTTT |
| | 3UTR(+1612+1636) | | | 3UTR(+1666+1690) | |
| 678 | OPA1 H31 | TGGTCCTGTTTTATTCTTTAGAAAT | 696 | OPA1 H31 | GTGGAAATACAGCTGAAGGTCTTTC |
| | 3UTR(+1615+1639) | | | 3UTR(+1669+1693) | |
| 679 | OPA1 H31 | ATATGGTCCTGTTTTATTCTTTAGA | 697 | OPA1 H31 | TCTGTGGAAATACAGCTGAAGGTCT |
| | 3UTR(+1618+1642) | | | 3UTR(+1672+1696) | |
| 680 | OPA1 H31 | TAAATATGGTCCTGTTTTATTCTTT | 698 | OPA1 H31 | AAATCTGTGGAAATACAGCTGAAGG |
| | 3UTR(+1621+1645) | | | 3UTR(+1675+1699) | |
| 681 | OPA1 H31 | AAATAAATATGGTCCTGTTTTATTC | 699 | OPA1 H31 | GAGAAATCTGTGGAAATACAGCTGA |
| | 3UTR(+1624+1648) | | | 3UTR(+1678+1702) | |
| 682 | OPA1 H31 | AGTAAATAAATATGGTCCTGTTTTA | 700 | OPA1 H31 | TGGGAGAAATCTGTGGAAATACAGC |
| | 3UTR(+1627+1651) | | | 3UTR(+1681+1705) | |
| 683 | OPA1 H31 | AGAAGTAAATAAATATGGTCCTGTT | 701 | OPA1 H31 | CCTTGGGAGAAATCTGTGGAAATAC |
| | 3UTR(+1630+1654) | | | 3UTR(+1684+1708) | |
| 684 | OPA1 H31 | GACAGAAGTAAATAAATATGGTCCT | 702 | OPA1 H31 | TTTCCTTGGGAGAAATCTGTGGAAA |
| | 3UTR(+1633+1657) | | | 3UTR(+1687+1711) | |
| 685 | OPA1 H31 | GTTGACAGAAGTAAATAAATATGGT | 703 | OPA1 H31 | CCTTTTCCTTGGGAGAAATCTGTGG |
| | 3UTR(+1636+1660) | | | 3UTR(+1690+1714) | |
| 686 | OPA1 H31 | ATAGTTGACAGAAGTAAATAAATAT | 704 | OPA1 H31 | TAGCCTTTTCCTTGGGAGAAATCTG |
| | 3UTR(+1639+1663) | | | 3UTR(+1693+1717) | |
| 687 | OPA1 H31 | TCTATAGTTGACAGAAGTAAATAAA | 705 | OPA1 H31 | TATTAGCCTTTTCCTTGGGAGAAAT |
| | 3UTR(+1642+1666) | | | 3UTR(+1696+1720) | |
| 688 | OPA1 H31 | CTTTCTATAGTTGACAGAAGTAAAT | 706 | OPA1 H31 | TAATATTAGCCTTTTCCTTGGGAGA |
| | 3UTR(+1645+1669) | | | 3UTR(+1699+1723) | |
| 689 | OPA1 H31 | TTTCTTTCTATAGTTGACAGAAGTA | 707 | OPA1 H31 | GACTAATATTAGCCTTTTCCTTGGG |
| | 3UTR(+1648+1672) | | | 3UTR(+1702+1726) | |
| 690 | OPA1 H31 | GTCTTTCTTTCTATAGTTGACAGAA | 708 | OPA1 H31 | AGTGACTAATATTAGCCTTTTCCTT |
| | 3UTR(+1651+1675) | | | 3UTR(+1705+1729) | |
| 691 | OPA1 H31 | AAGGTCTTTCTTTCTATAGTTGACA | 709 | OPA1 H31 | AGTAGTGACTAATATTAGCCTTTTC |
| | 3UTR(+1654+1678) | | | 3UTR(+1708+1732) | |
| 692 | OPA1 H31 | CTGAAGGTCTTTCTTTCTATAGTTG | 710 | OPA1 H31 | AACAGTAGTGACTAATATTAGCCTT |
| | 3UTR(+1657+1681) | | | 3UTR(+1711+1735) | |
| 711 | OPA1 H31 | GATAACAGTAGTGACTAATATTAGC | 729 | OPA1 H31 | CAAAGAAATGAAGATCTCCCTCCAT |
| | 3UTR(+1714+1738) | | | 3UTR(+1768+1792) | |
| 712 | OPA1 H31 | TGTGATAACAGTAGTGACTAATATT | 730 | OPA1 H31 | CCTCAAAGAAATGAAGATCTCCCTC |
| | 3UTR(+1717+1741) | | | 3UTR(+1771+1795) | |
| 713 | OPA1 H31 | GGATGTGATAACAGTAGTGACTAAT | 731 | OPA1 H31 | TCTCCTCAAAGAAATGAAGATCTCC |
| | 3UTR(+1720+1744) | | | 3UTR(+1774+1798) | |
| 714 | OPA1 H31 | AAGGGATGTGATAACAGTAGTGACT | 732 | OPA1 H31 | TGATCTCCTCAAAGAAATGAAGATC |
| | 3UTR(+1723+1747) | | | 3UTR(+1777+1801) | |
| 715 | OPA1 H31 | ACAAAGGGATGTGATAACAGTAGTG | 733 | OPA1 H31 | TACTGATCTCCTCAAAGAAATGAAG |
| | 3UTR(+1726+1750) | | | 3UTR(+1780+1804) | |
| 716 | OPA1 H31 | TATACAAAGGGATGTGATAACAGTA | 734 | OPA1 H31 | CAATACTGATCTCCTCAAAGAAATG |
| | 3UTR(+1729+1753) | | | 3UTR(+1783+1807) | |
| 717 | OPA1 H31 | ACTTATACAAAGGGATGTGATAACA | 735 | OPA1 H31 | TTACAATACTGATCTCCTCAAAGAA |
| | 3UTR(+1732+1756) | | | 3UTR(+1786+1810) | |
| 718 | OPA1 H31 | AAAACTTATACAAAGGGATGTGATA | 736 | OPA1 H31 | ACGTTACAATACTGATCTCCTCAAA |
| | 3UTR(+1735+1759) | | | 3UTR(+1789+1813) | |
| 719 | OPA1 H31 | TTTAAAACTTATACAAAGGGATGTG | 737 | OPA1 H31 | CATACGTTACAATACTGATCTCCTC |
| | 3UTR(+1738+1762) | | | 3UTR(+1792+1816) | |
| 720 | OPA1 H31 | CTTTTTAAAACTTATACAAAGGGAT | 738 | OPA1 H31 | TCACATACGTTACAATACTGATCTC |
| | 3UTR(+1741+1765) | | | 3UTR(+1795+1819) | |
| 721 | OPA1 H31 | TCTCTTTTTAAAACTTATACAAAGG | 739 | OPA1 H31 | TATTCACATACGTTACAATACTGAT |
| | 3UTR(+1744+1768) | | | 3UTR(+1798+1822) | |
| 722 | OPA1 H31 | CCATCTCTTTTTAAAACTTATACAA | 740 | OPA1 H31 | ATCTATTCACATACGTTACAATACT |
| | 3UTR(+1747+1771) | | | 3UTR(+1801+1825) | |
| 723 | OPA1 H31 | CCTCCATCTCTTTTTAAAACTTATA | 741 | OPA1 H31 | ATCATCTATTCACATACGTTACAAT |
| | 3UTR(+1750+1774) | | | 3UTR(+1804+1828) | |
| 724 | OPA1 H31 | CTCCCTCCATCTCTTTTTAAAACTT | 742 | OPA1 H31 | GTTATCATCTATTCACATACGTTAC |
| | 3UTR(+1753+1777) | | | 3UTR(+1807+1831) | |
| 725 | OPA1 H31 | GATCTCCCTCCATCTCTTTTTAAAA | 743 | OPA1 H31 | ATTGTTATCATCTATTCACATACGT |
| | 3UTR(+1756+1780) | | | 3UTR(+1810+1834) | |
| 726 | OPA1 H31 | GAAGATCTCCCTCCATCTCTTTTTA | 744 | OPA1 H31 | TTAATTGTTATCATCTATTCACATA |
| | 3UTR(+1759+1783) | | | 3UTR(+1813+1837) | |
| 727 | OPA1 H31 | AATGAAGATCTCCCTCCATCTCTTT | 745 | OPA1 H31 | ATATTAATTGTTATCATCTATTCAC |
| | 3UTR(+1762+1786) | | | 3UTR(+1816+1840) | |
| 728 | OPA1 H31 | AGAAATGAAGATCTCCCTCCATCTC | 746 | OPA1 H31 | GTAATATTAATTGTTATCATCTATT |
| | 3UTR(+1765+1789) | | | 3UTR(+1819+1843) | |
| 747 | OPA1 H31 | TTAGTAATATTAATTGTTATCATCT | 765 | OPA1 H31 | AAGCCACATTACTGTGGGGCATGGA |
| | 3UTR(+1822+1846) | | | 3UTR(+1876+1900) | |
| 748 | OPA1 H31 | CTTTTAGTAATATTAATTGTTATCA | 766 | OPA1 H31 | AAGAAGCCACATTACTGTGGGGCAT |
| | 3UTR(+1825+1849) | | | 3UTR(+1879+1903) | |
| 749 | OPA1 H31 | GGACTTTTAGTAATATTAATTGTTA | 767 | OPA1 H31 | TGAAAGAAGCCACATTACTGTGGGG |
| | 3UTR(+1828+1852) | | | 3UTR(+1882+1906) | |
| 750 | OPA1 H31 | GTGGGACTTTTAGTAATATTAATTG | 768 | OPA1 H31 | CCATGAAAGAAGCCACATTACTGTG |
| | 3UTR(+1831+1855) | | | 3UTR(+1885+1909) | |
| 751 | OPA1 H31 | CATGTGGGACTTTTAGTAATATTAA | 769 | OPA1 H31 | AACCCATGAAAGAAGCCACATTACT |
| | 3UTR(+1834+1858) | | | 3UTR(+1888+1912) | |
| 752 | OPA1 H31 | TCTCATGTGGGACTTTTAGTAATAT | 770 | OPA1 H31 | AAAAACCCATGAAAGAAGCCACATT |
| | 3UTR(+1837+1861) | | | 3UTR(+1891+1915) | |
| 753 | OPA1 H31 | GACTCTCATGTGGGACTTTTAGTAA | 771 | OPA1 H31 | AAAAAAAACCCATGAAAGAAGCCAC |
| | 3UTR(+1840+1864) | | | 3UTR(+1894+1918) | |
| 754 | OPA1 H31 | CAGGACTCTCATGTGGGACTTTTAG | 772 | OPA1 H31 | AGAAAAAAAAACCCATGAAAGAAGC |
| | 3UTR(+1843+1867) | | | 3UTR(+1897+1921) | |
| 755 | OPA1 H31 | CGTCAGGACTCTCATGTGGGACTTT | 773 | OPA1 H31 | AGAAGAAAAAAAAACCCATGAAAGA |
| | 3UTR(+1846+1870) | | | 3UTR(+1900+1924) | |
| 756 | OPA1 H31 | GGGCGTCAGGACTCTCATGTGGGAC | 774 | OPA1 H31 | AAAAGAAGAAAAAAAAACCCATGAA |
| | 3UTR(+1849+1873) | | | 3UTR(+1903+1927) | |
| 757 | OPA1 H31 | AGAGGGCGTCAGGACTCTCATGTGG | 775 | OPA1 H31 | CTAAAAAGAAGAAAAAAAAACCCAT |
| | 3UTR(+1852+1876) | | | 3UTR(+1906+1930) | |
| 758 | OPA1 H31 | TGGAGAGGGCGTCAGGACTCTCATG | 776 | OPA1 H31 | CAGCTAAAAAGAAGAAAAAAAAACC |
| | 3UTR(+1855+1879) | | | 3UTR(+1909+1933) | |
| 759 | OPA1 H31 | GCATGGAGAGGGCGTCAGGACTCTC | 777 | OPA1 H31 | GATCAGCTAAAAAGAAGAAAAAAAA |
| | 3UTR(+1858+1882) | | | 3UTR(+1912+1936) | |
| 760 | OPA1 H31 | GGGGCATGGAGAGGGCGTCAGGACT | 778 | OPA1 H31 | TGAGATCAGCTAAAAAGAAGAAAAA |
| | 3UTR(+1861+1885) | | | 3UTR(+1915+1939) | |
| 761 | OPA1 H31 | TGTGGGGCATGGAGAGGGCGTCAGG | 779 | OPA1 H31 | GGATGAGATCAGCTAAAAAGAAGAA |
| | 3UTR(+1864+1888) | | | 3UTR(+1918+1942) | |
| 762 | OPA1 H31 | TACTGTGGGGCATGGAGAGGGCGTC | 780 | OPA1 H31 | TTAGGATGAGATCAGCTAAAAAGAA |
| | 3UTR(+1867+1891) | | | 3UTR(+1921+1945) | |
| 763 | OPA1 H31 | CATTACTGTGGGGCATGGAGAGGGC | 781 | OPA1 H31 | TGCTTAGGATGAGATCAGCTAAAAA |
| | 3UTR(+1870+1894) | | | 3UTR(+1924+1948) | |
| 764 | OPA1 H31 | CCACATTACTGTGGGGCATGGAGAG | 782 | OPA1 H31 | GCATGCTTAGGATGAGATCAGCTAA |
| | 3UTR(+1873+1897) | | | 3UTR(+1927+1951) | |
| 783 | OPA1 H31 | AAAGCATGCTTAGGATGAGATCAGC | 801 | OPA1 H31 | ATTTTCTTTCAATAACATCTGCAGT |
| | 3UTR(+1930+1954) | | | 3UTR(+1984+2008) | |
| 784 | OPA1 H31 | AATAAAGCATGCTTAGGATGAGATC | 802 | OPA1 H31 | TTTATTTTCTTTCAATAACATCTGC |
| | 3UTR(+1933+1957) | | | 3UTR(+1987+2011) | |
| 785 | OPA1 H31 | AAAAATAAAGCATGCTTAGGATGAG | 803 | OPA1 H31 | AATTTTATTTTCTTTCAATAACATC |
| | 3UTR(+1936+1960) | | | 3UTR(+1990+2014) | |
| 786 | OPA1 H31 | AGGAAAAATAAAGCATGCTTAGGAT | 804 | OPA1 H31 | CTGAATTTTATTTTCTTTCAATAAC |
| | 3UTR(+1939+1963) | | | 3UTR(+1993+2017) | |
| 787 | OPA1 H31 | TCAAGGAAAAATAAAGCATGCTTAG | 805 | OPA1 H31 | AGACTGAATTTTATTTTCTTTCAAT |
| | 3UTR(+1942+1966) | | | 3UTR(+1996+2020) | |
| 788 | OPA1 H31 | CTTTCAAGGAAAAATAAAGCATGCT | 806 | OPA1 H31 | TTGAGACTGAATTTTATTTTCTTTC |
| | 3UTR(+1945+1969) | | | 3UTR(+1999+2023) | |
| 789 | OPA1 H31 | TAGCTTTCAAGGAAAAATAAAGCAT | 807 | OPA1 H31 | CTCTTGAGACTGAATTTTATTTTCT |
| | 3UTR(+1948+1972) | | | 3UTR(+2002+2026) | |
| 790 | OPA1 H31 | ACCTAGCTTTCAAGGAAAAATAAAG | 808 | OPA1 H31 | TTACTCTTGAGACTGAATTTTATTT |
| | 3UTR(+1951+1975) | | | 3UTR(+2005+2029) | |
| 791 | OPA1 H31 | AATACCTAGCTTTCAAGGAAAAATA | 809 | OPA1 H31 | GGTTTACTCTTGAGACTGAATTTTA |
| | 3UTR(+1954+1978) | | | 3UTR(+2008+2032) | |
| 792 | OPA1 H31 | ATAAATACCTAGCTTTCAAGGAAAA | 810 | OPA1 H31 | CAGGGTTTACTCTTGAGACTGAATT |
| | 3UTR(+1957+1981) | | | 3UTR(+2011+2035) | |
| 793 | OPA1 H31 | TTGATAAATACCTAGCTTTCAAGGA | 811 | OPA1 H31 | ACACAGGGTTTACTCTTGAGACTGA |
| | 3UTR(+1960+1984) | | | 3UTR(+2014+2038) | |
| 794 | OPA1 H31 | CAGTTGATAAATACCTAGCTTTCAA | 812 | OPA1 H31 | AAGACACAGGGTTTACTCTTGAGAC |
| | 3UTR(+1963+1987) | | | 3UTR(+2017+2041) | |
| 795 | OPA1 H31 | CTGCAGTTGATAAATACCTAGCTTT | 813 | OPA1 H31 | CACAAGACACAGGGTTTACTCTTGA |
| | 3UTR(+1966+1990) | | | 3UTR(+2020+2044) | |
| 796 | OPA1 H31 | CATCTGCAGTTGATAAATACCTAGC | 814 | OPA1 H31 | AGACACAAGACACAGGGTTTACTCT |
| | 3UTR(+1969+1993) | | | 3UTR(+2023+2047) | |
| 797 | OPA1 H31 | TAACATCTGCAGTTGATAAATACCT | 815 | OPA1 H31 | TACAGACACAAGACACAGGGTTTAC |
| | 3UTR(+1972+1996) | | | 3UTR(+2026+2050) | |
| 798 | OPA1 H31 | CAATAACATCTGCAGTTGATAAATA | 816 | OPA1 H31 | AACTACAGACACAAGACACAGGGTT |
| | 3UTR(+1975+1999) | | | 3UTR(+2029+2053) | |
| 799 | OPA1 H31 | TTTCAATAACATCTGCAGTTGATAA | 817 | OPA1 H31 | TTGAACTACAGACACAAGACACAGG |
| | 3UTR(+1978+2002) | | | 3UTR(+2032+2056) | |
| 800 | OPA1 H31 | TTCTTTCAATAACATCTGCAGTTGA | 818 | OPA1 H31 | CTTTTGAACTACAGACACAAGACAC |
| | 3UTR(+1981+2005) | | | 3UTR(+2035+2059) | |
| 819 | OPA1 H31 | TGACTTTTGAACTACAGACACAAGA | 837 | OPA1 H31 | TCGAATTTAACTTCTGTATATTTAG |
| | 3UTR(+2038+2062) | | | 3UTR(+2092+2116) | |
| 820 | OPA1 H31 | TTCTGACTTTTGAACTACAGACACA | 838 | OPA1 H31 | AGTTCGAATTTAACTTCTGTATATT |
| | 3UTR(+2041+2065) | | | 3UTR(+2095+2119) | |
| 821 | OPA1 H31 | CATTTCTGACTTTTGAACTACAGAC | 839 | OPA1 H31 | GCTAGTTCGAATTTAACTTCTGTAT |
| | 3UTR(+2044+2068) | | | 3UTR(+2098+2122) | |
| 822 | OPA1 H31 | AATCATTTCTGACTTTTGAACTACA | 840 | OPA1 H31 | GTGGCTAGTTCGAATTTAACTTCTG |
| | 3UTR(+2047+2071) | | | 3UTR(+2101+2125) | |
| 823 | OPA1 H31 | TAGAATCATTTCTGACTTTTGAACT | 841 | OPA1 H31 | TCTGTGGCTAGTTCGAATTTAACTT |
| | 3UTR(+2050+2074) | | | 3UTR(+2104+2128) | |
| 824 | OPA1 H31 | AATTAGAATCATTTCTGACTTTTGA | 842 | OPA1 H31 | GATTCTGTGGCTAGTTCGAATTTAA |
| | 3UTR(+2053+2077) | | | 3UTR(+2107+2131) | |
| 825 | OPA1 H31 | TTAAATTAGAATCATTTCTGACTTT | 843 | OPA1 H31 | AATGATTCTGTGGCTAGTTCGAATT |
| | 3UTR(+2056+2080) | | | 3UTR(+2110+2134) | |
| 826 | OPA1 H31 | TGTTTAAATTAGAATCATTTCTGAC | 844 | OPA1 H31 | ACAAATGATTCTGTGGCTAGTTCGA |
| | 3UTR(+2059+2083) | | | 3UTR(+2113+2137) | |
| 827 | OPA1 H31 | TTTTGTTTAAATTAGAATCATTTCT | 845 | OPA1 H31 | AAAACAAATGATTCTGTGGCTAGTT |
| | 3UTR(+2062+2086) | | | 3UTR(+2116+2140) | |
| 828 | OPA1 H31 | TCTTTTTGTTTAAATTAGAATCATT | 846 | OPA1 H31 | ATAAAAACAAATGATTCTGTGGCTA |
| | 3UTR(+2065+2089) | | | 3UTR(+2119+2143) | |
| 829 | OPA1 H31 | GTATCTTTTTGTTTAAATTAGAATC | 847 | OPA1 H31 | GACATAAAAACAAATGATTCTGTGG |
| | 3UTR(+2068+2092) | | | 3UTR(+2122+2146) | |
| 830 | OPA1 H31 | TTAGTATCTTTTTGTTTAAATTAGA | 848 | OPA1 H31 | TCTGACATAAAAACAAATGATTCTG |
| | 3UTR(+2071+2095) | | | 3UTR(+2125+2149) | |
| 831 | OPA1 H31 | TATTTAGTATCTTTTTGTTTAAATT | 849 | OPA1 H31 | AATTCTGACATAAAAACAAATGATT |
| | 3UTR(+2074+2098) | | | 3UTR(+2128+2152) | |
| 832 | OPA1 H31 | GTATATTTAGTATCTTTTTGTTTAA | 850 | OPA1 H31 | GCAAATTCTGACATAAAAACAAATG |
| | 3UTR(+2077+2101) | | | 3UTR(+2131+2155) | |
| 833 | OPA1 H31 | TCTGTATATTTAGTATCTTTTTGTT | 851 | OPA1 H31 | TTTGCAAATTCTGACATAAAAACAA |
| | 3UTR(+2080+2104) | | | 3UTR(+2134+2158) | |
| 834 | OPA1 H31 | ACTTCTGTATATTTAGTATCTTTTT | 852 | OPA1 H31 | CTCTTTGCAAATTCTGACATAAAAA |
| | 3UTR(+2083+2107) | | | 3UTR(+2137+2161) | |
| 835 | OPA1 H31 | TTAACTTCTGTATATTTAGTATCTT | 853 | OPA1 H31 | CCACTCTTTGCAAATTCTGACATAA |
| | 3UTR(+2086+2110) | | | 3UTR(+2140+2164) | |
| 836 | OPA1 H31 | AATTTAACTTCTGTATATTTAGTAT | 854 | OPA1 H31 | ACTCCACTCTTTGCAAATTCTGACA |
| | 3UTR(+2089+2113) | | | 3UTR(+2143+2167) | |
| 855 | OPA1 H31 | TCCACTCCACTCTTTGCAAATTCTG | 873 | OPA1 H31 | TTAAGTTTGGATATCATCTATTTAC |
| | 3UTR(+2146+2170) | | | 3UTR(+2200+2224) | |
| 856 | OPA1 H31 | TTGTCCACTCCACTCTTTGCAAATT | 874 | OPA1 H31 | AAATTAAGTTTGGATATCATCTATT |
| | 3UTR(+2149+2173) | | | 3UTR(+2203+2227) | |
| 857 | OPA1 H31 | GCTTTGTCCACTCCACTCTTTGCAA | 875 | OPA1 H31 | GCCAAATTAAGTTTGGATATCATCT |
| | 3UTR(+2152+2176) | | | 3UTR(+2206+2230) | |
| 858 | OPA1 H31 | AGAGCTTTGTCCACTCCACTCTTTG | 876 | OPA1 H31 | CTAGCCAAATTAAGTTTGGATATCA |
| | 3UTR(+2155+2179) | | | 3UTR(+2209+2233) | |
| 859 | OPA1 H31 | TACAGAGCTTTGTCCACTCCACTCT | 877 | OPA1 H31 | GTCCTAGCCAAATTAAGTTTGGATA |
| | 3UTR(+2158+2182) | | | 3UTR(+2212+2236) | |
| 860 | OPA1 H31 | CCATACAGAGCTTTGTCCACTCCAC | 878 | OPA1 H31 | GAAGTCCTAGCCAAATTAAGTTTGG |
| | 3UTR(+2161+2185) | | | 3UTR(+2215+2239) | |
| 861 | OPA1 H31 | CTTCCATACAGAGCTTTGTCCACTC | 879 | OPA1 H31 | ATTGAAGTCCTAGCCAAATTAAGTT |
| | 3UTR(+2164+2188) | | | 3UTR(+2218+2242) | |
| 862 | OPA1 H31 | AGTCTTCCATACAGAGCTTTGTCCA | 880 | OPA1 H31 | AAAATTGAAGTCCTAGCCAAATTAA |
| | 3UTR(+2167+2191) | | | 3UTR(+2221+2245) | |
| 863 | OPA1 H31 | TTCAGTCTTCCATACAGAGCTTTGT | 881 | OPA1 H31 | TTTAAAATTGAAGTCCTAGCCAAAT |
| | 3UTR(+2170+2194) | | | 3UTR(+2224+2248) | |
| 864 | OPA1 H31 | TTGTTCAGTCTTCCATACAGAGCTT | 882 | OPA1 H31 | ATTTTTAAAATTGAAGTCCTAGCCA |
| | 3UTR(+2173+2197) | | | 3UTR(+2227+2251) | |
| 865 | OPA1 H31 | CAGTTGTTCAGTCTTCCATACAGAG | 883 | OPA1 H31 | CTGATTTTTAAAATTGAAGTCCTAG |
| | 3UTR(+2176+2200) | | | 3UTR(+2230+2254) | |
| 866 | OPA1 H31 | TTACAGTTGTTCAGTCTTCCATACA | 884 | OPA1 H31 | ACACTGATTTTTAAAATTGAAGTCC |
| | 3UTR(+2179+2203) | | | 3UTR(+2233+2257) | |
| 867 | OPA1 H31 | TATTTACAGTTGTTCAGTCTTCCAT | 885 | OPA1 H31 | GGTACACTGATTTTTAAAATTGAAG |
| | 3UTR(+2182+2206) | | | 3UTR(+2236+2260) | |
| 868 | OPA1 H31 | ATCTATTTACAGTTGTTCAGTCTTC | 886 | OPA1 H31 | CTAGGTACACTGATTTTTAAAATTG |
| | 3UTR(+2185+2209) | | | 3UTR(+2239+2263) | |
| 869 | OPA1 H31 | ATCATCTATTTACAGTIGTTCAGTC | 887 | OPA1 H31 | TGCCTAGGTACACTGATTTTTAAAA |
| | 3UTR(+2188+2212) | | | 3UTR(+2242+2266) | |
| 870 | OPA1 H31 | GATATCATCTATTTACAGTTGTTCA | 888 | OPA1 H31 | CACTGCCTAGGTACACTGATTTTTA |
| | 3UTR(+2191+2215) | | | 3UTR(+2245+2269) | |
| 871 | OPA1 H31 | TTGGATATCATCTATTTACAGTTGT | 889 | OPA1 H31 | GTGCACTGCCTAGGTACACTGATTT |
| | 3UTR(+2194+2218) | | | 3UTR(+2248+2272) | |
| 872 | OPA1 H31 | AGTTTGGATATCATCTATTTACAGT | 890 | OPA1 H31 | GCTGTGCACTGCCTAGGTACACTGA |
| | 3UTR(+2197+2221) | | | 3UTR(+2251+2275) | |
| 891 | OPA1 H31 | CGTGCTGTGCACTGCCTAGGTACAC | 909 | OPA1 H31 | CCCGCAACTATAGCACAGTGGGTTA |
| | 3UTR(+2254+2278) | | | 3UTR(+2308+2332) | |
| 892 | OPA1 H31 | TTTCGTGCTGTGCACTGCCTAGGTA | 910 | OPA1 H31 | CCACCCGCAACTATAGCACAGTGGG |
| | 3UTR(+2257+2281) | | | 3UTR(+2311+2335) | |
| 893 | OPA1 H31 | TTATTTCGTGCTGTGCACTGCCTAG | 911 | OPA1 H31 | GTTCCACCCGCAACTATAGCACAGT |
| | 3UTR(+2260+2284) | | | 3UTR(+2314+2338) | |
| 894 | OPA1 H31 | CACTTATTTCGTGCTGTGCACTGCC | 912 | OPA1 H31 | ACTGTTCCACCCGCAACTATAGCAC |
| | 3UTR(+2263+2287) | | | 3UTR(+2317+2341) | |
| 895 | OPA1 H31 | GGCCACTTATTTCGTGCTGTGCACT | 913 | OPA1 H31 | TTGACTGTTCCACCCGCAACTATAG |
| | 3UTR(+2266+2290) | | | 3UTR(+2320+2344) | |
| 896 | OPA1 H31 | AAGGGCCACTTATTTCGTGCTGTGC | 914 | OPA1 H31 | AGGTTGACTGTTCCACCCGCAACTA |
| | 3UTR(+2269+2293) | | | 3UTR(+2323+2347) | |
| 897 | OPA1 H31 | TGCAAGGGCCACTTATTTCGTGCTG | 915 | OPA1 H31 | GAAAGGTTGACTGTTCCACCCGCAA |
| | 3UTR(+2272+2296) | | | 3UTR(+2326+2350) | |
| 898 | OPA1 H31 | AGCTGCAAGGGCCACTTATTTCGTG | 916 | OPA1 H31 | CTAGAAAGGTTGACTGTTCCACCCG |
| | 3UTR(+2275+2299) | | | 3UTR(+2329+2353) | |
| 899 | OPA1 H31 | GGAAGCTGCAAGGGCCACTTATTTC | 917 | OPA1 H31 | CTACTAGAAAGGTTGACTGTTCCAC |
| | 3UTR(+2278+2302) | | | 3UTR(+2332+2356) | |
| 900 | OPA1 H31 | CGGGGAAGCTGCAAGGGCCACTTAT | 918 | OPA1 H31 | AAACTACTAGAAAGGTTGACTGTTC |
| | 3UTR(+2281+2305) | | | 3UTR(+2335+2359) | |
| 901 | OPA1 H31 | AAACGGGGAAGCTGCAAGGGCCACT | 919 | OPA1 H31 | CATAAACTACTAGAAAGGTTGACTG |
| | 3UTR(+2284+2308) | | | 3UTR(+2338+2362) | |
| 902 | OPA1 H31 | GTTAAACGGGGAAGCTGCAAGGGCC | 920 | OPA1 H31 | TATCATAAACTACTAGAAAGGTTGA |
| | 3UTR(+2287+2311) | | | 3UTR(+2341+2365) | |
| 903 | OPA1 H31 | TGGGTTAAACGGGGAAGCTGCAAGG | 921 | OPA1 H31 | CAATATCATAAACTACTAGAAAGGT |
| | 3UTR(+2290+2314) | | | 3UTR(+2344+2368) | |
| 904 | OPA1 H31 | CAGTGGGTTAAACGGGGAAGCTGCA | 922 | OPA1 H31 | GGGCAATATCATAAACTACTAGAAA |
| | 3UTR(+2293+2317) | | | 3UTR(+2347+2371) | |
| 905 | OPA1 H31 | GCACAGTGGGTTAAACGGGGAAGCT | 923 | OPA1 H31 | AGAGGGCAATATCATAAACTACTAG |
| | 3UTR(+2296+2320) | | | 3UTR(+2350+2374) | |
| 906 | OPA1 H31 | ATAGCACAGTGGGTTAAACGGGGAA | 924 | OPA1 H31 | CAAAGAGGGCAATATCATAAACTAC |
| | 3UTR(+2299+2323) | | | 3UTR(+2353+2377) | |
| 907 | OPA1 H31 | ACTATAGCACAGTGGGTTAAACGGG | 925 | OPA1 H31 | ATACAAAGAGGGCAATATCATAAAC |
| | 3UTR(+2302+2326) | | | 3UTR(+2356+2380) | |
| 908 | OPA1 H31 | GCAACTATAGCACAGTGGGTTAAAC | 926 | OPA1 H31 | GGAATACAAAGAGGGCAATATCATA |
| | 3UTR(+2305+2329) | | | 3UTR(+2359+2383) | |
| 927 | OPA1 H31 | ATGGGAATACAAAGAGGGCAATATC | 945 | OPA1 H31 | CATTTGGAGGCTGAATAATTTGCAG |
| | 3UTR(+2362+2386) | | | 3UTR(+2416+2440) | |
| 928 | OPA1 H31 | AAAATGGGAATACAAAGAGGGCAAT | 946 | OPA1 H31 | TTGCATTTGGAGGCTGAATAATTTG |
| | 3UTR(+2365+2389) | | | 3UTR(+2419+2443) | |
| 929 | OPA1 H31 | TAGAAAATGGGAATACAAAGAGGGC | 947 | OPA1 H31 | CATTTGCATTTGGAGGCTGAATAAT |
| | 3UTR(+2368+2392) | | | 3UTR(+2422+2446) | |
| 930 | OPA1 H31 | CTGTAGAAAATGGGAATACAAAGAG | 948 | OPA1 H31 | ATTCATTTGCATTTGGAGGCTGAAT |
| | 3UTR(+2371+2395) | | | 3UTR(+2425+2449) | |
| 931 | OPA1 H31 | AAACTGTAGAAAATGGGAATACAAA | 949 | OPA1 H31 | ATCATTCATTTGCATTTGGAGGCTG |
| | 3UTR(+2374+2398) | | | 3UTR(+2428+2452) | |
| 932 | OPA1 H31 | AAAAAACTGTAGAAAATGGGAATAC | 950 | OPA1 H31 | TATATCATTCATTTGCATTTGGAGG |
| | 3UTR(+2377+2401) | | | 3UTR(+2431+2455) | |
| 933 | OPA1 H31 | CGGAAAAAACTGTAGAAAATGGGAA | 951 | OPA1 H31 | TTTTATATCATTCATTTGCATTTGG |
| | 3UTR(+2380+2404) | | | 3UTR(+2434+2458) | |
| 934 | OPA1 H31 | CTGCGGAAAAAACTGTAGAAAATGG | 952 | OPA1 H31 | TATTTTTATATCATTCATTTGCATT |
| | 3UTR(+2383+2407) | | | 3UTR(+2437+2461) | |
| 935 | OPA1 H31 | AGTCTGCGGAAAAAACTGTAGAAAA | 953 | OPA1 H31 | ACTTATTTTTATATCATTCATTTGC |
| | 3UTR(+2386+2410) | | | 3UTR(+2440+2464) | |
| 936 | OPA1 H31 | AGAAGTCTGCGGAAAAAACTGTAGA | 954 | OPA1 H31 | CCTACTTATTTTTATATCATTCATT |
| | 3UTR(+2389+2413) | | | 3UTR(+2443+2467) | |
| 937 | OPA1 H31 | GAAAGAAGTCTGCGGAAAAAACTGT | 955 | OPA1 H31 | TTCCCTACTTATTTTTATATCATTC |
| | 3UTR(+2392+2416) | | | 3UTR(+2446+2470) | |
| 938 | OPA1 H31 | GCAGAAAGAAGTCTGCGGAAAAAAC | 956 | OPA1 H31 | ATGTTCCCTACTTATTTTTATATCA |
| | 3UTR(+2395+2419) | | | 3UTR(+2449+2473) | |
| 939 | OPA1 H31 | TTTGCAGAAAGAAGTCTGCGGAAAA | 957 | OPA1 H31 | GCCATGTTCCCTACTTATTTTTATA |
| | 3UTR(+2398+2422) | | | 3UTR(+2452+2476) | |
| 940 | OPA1 H31 | TAATTTGCAGAAAGAAGTCTGCGGA | 958 | OPA1 H31 | TCTGCCATGTTCCCTACTTATTTTT |
| | 3UTR(+2401+2425) | | | 3UTR(+2455+2479) | |
| 941 | OPA1 H31 | GAATAATTTGCAGAAAGAAGTCTGC | 959 | OPA1 H31 | CTCTCTGCCATGTTCCCTACTTATT |
| | 3UTR(+2404+2428) | | | 3UTR(+2458+2482) | |
| 942 | OPA1 H31 | GCTGAATAATTTGCAGAAAGAAGTC | 960 | OPA1 H31 | CCACTCTCTGCCATGTTCCCTACTT |
| | 3UTR(+2407+2431) | | | 3UTR(+2461+2485) | |
| 943 | OPA1 H31 | GAGGCTGAATAATTTGCAGAAAGAA | 961 | OPA1 H31 | GCACCACTCTCTGCCATGTTCCCTA |
| | 3UTR(+2410+2434) | | | 3UTR(+2464+2488) | |
| 944 | OPA1 H31 | TTGGAGGCTGAATAATTTGCAGAAA | 962 | OPA1 H31 | GAAGCACCACTCTCTGCCATGTTCC |
| | 3UTR(+2413+2437) | | | 3UTR(+2467+2491) | |
| 963 | OPA1 H31 | TGGGAAGCACCACTCTCTGCCATGT | 981 | OPA1 H31 | TTTTAAACCTATACTCTGACTCTCA |
| | 3UTR(+2470+2494) | | | 3UTR(+2524+2548) | |
| 964 | OPA1 H31 | GGCTGGGAAGCACCACTCTCTGCCA | 982 | OPA1 H31 | ATCTTTTAAACCTATACTCTGACTC |
| | 3UTR(+2473+2497) | | | 3UTR(+2527+2551) | |
| 965 | OPA1 H31 | TGAGGCTGGGAAGCACCACTCTCTG | 983 | OPA1 H31 | TTTATCTTTTAAACCTATACTCTGA |
| | 3UTR(+2476+2500) | | | 3UTR(+2530+2554) | |
| 966 | OPA1 H31 | TTGTGAGGCTGGGAAGCACCACTCT | 984 | OPA1 H31 | GATTTTATCTTTTAAACCTATACTC |
| | 3UTR(+2479+2503) | | | 3UTR(+2533+2557) | |
| 967 | OPA1 H31 | ACATTGTGAGGCTGGGAAGCACCAC | 985 | OPA1 H31 | AAAGATTTTATCTTTTAAACCTATA |
| | 3UTR(+2482+2506) | | | 3UTR(+2536+2560) | |
| 968 | OPA1 H31 | CCCACATTGTGAGGCTGGGAAGCAC | 986 | OPA1 H31 | ACTAAAGATTTTATCTTTTAAACCT |
| | 3UTR(+2485+2509) | | | 3UTR(+2539+2563) | |
| 969 | OPA1 H31 | ATTCCCACATTGTGAGGCTGGGAAG | 987 | OPA1 H31 | TTAACTAAAGATTTTATCTTTTAAA |
| | 3UTR(+2488+2512) | | | 3UTR(+2542+2566) | |
| 970 | OPA1 H31 | CAAATTCCCACATTGTGAGGCTGGG | 988 | OPA1 H31 | TTATTAACTAAAGATTTTATCTTTT |
| | 3UTR(+2491+2515) | | | 3UTR(+2545+2569) | |
| 971 | OPA1 H31 | TGTCAAATTCCCACATTGTGAGGCT | 989 | OPA1 H31 | AAATTATTAACTAAAGATTTTATCT |
| | 3UTR(+2494+2518) | | | 3UTR(+2548+2572) | |
| 972 | OPA1 H31 | CTATGTCAAATTCCCACATTGTGAG | 990 | OPA1 H31 | ACAAAATTATTAACTAAAGATTTTA |
| | 3UTR(+2497+2521) | | | 3UTR(+2551+2575) | |
| 973 | OPA1 H31 | ATCCTATGTCAAATTCCCACATTGT | 991 | OPA1 H31 | AATACAAAATTATTAACTAAAGATT |
| | 3UTR(+2500+2524) | | | 3UTR(+2554+2578) | |
| 974 | OPA1 H31 | CTCATCCTATGTCAAATTCCCACAT | 992 | OPA1 H31 | ATAAATACAAAATTATTAACTAAAG |
| | 3UTR(+2503+2527) | | | 3UTR(+2557+2581) | |
| 975 | OPA1 H31 | ACTCTCATCCTATGTCAAATTCCCA | 993 | OPA1 H31 | TAAATAAATACAAAATTATTAACTA |
| | 3UTR(+2506+2530) | | | 3UTR(+2560+2584) | |
| 976 | OPA1 H31 | CTGACTCTCATCCTATGTCAAATTC | 994 | OPA1 H31 | GAATAAATAAATACAAAATTATTAA |
| | 3UTR(+2509+2533) | | | 3UTR(+2563+2587) | |
| | OPA1 H31 | ACTCTGACTCTCATCCTATGTCAAA | 995 | OPA1 H31 | CTAGAATAAATAAATACAAAATTAT |
| 977 | 3UTR(+2512+2536) | | | 3UTR(+2566+2590) | |
| 978 | OPA1 H31 | TATACTCTGACTCTCATCCTATGTC | 996 | OPA1 H31 | CATCTAGAATAAATAAATACAAAAT |
| | 3UTR(+2515+2539) | | | 3UTR(+2569+2593) | |
| 979 | OPA1 H31 | ACCTATACTCTGACTCTCATCCTAT | 997 | OPA1 H31 | ATACATCTAGAATAAATAAATACAA |
| | 3UTR(+2518+2542) | | | 3UTR(+2572+2596) | |
| 980 | OPA1 H31 | TAAACCTATACTCTGACTCTCATCC | 998 | OPA1 H31 | TACATACATCTAGAATAAATAAATA |
| | 3UTR(+2521+2545) | | | 3UTR(+2575+2599) | |
| 999 | OPA1 H31 | AGATACATACATCTAGAATAAATAA | 1017 | OPA1 H31 | ACCATTACTTTGATCCCCTTTATTG |
| | 3UTR(+2578+2602) | | | 3UTR(+2632+2656) | |
| 1000 | OPA1 H31 | CTCAGATACATACATCTAGAATAAA | 1018 | OPA1 H31 | AAAACCATTACTTTGATCCCCTTTA |
| | 3UTR(+2581+2605) | | | 3UTR(+2635+2659) | |
| 1001 | OPA1 H31 | TTCCTCAGATACATACATCTAGAAT | 1019 | OPA1 H31 | AGAAAAACCATTACTTTGATCCCCT |
| | 3UTR(+2584+2608) | | | 3UTR(+2638+2662) | |
| 1002 | OPA1 H31 | TCTTTCCTCAGATACATACATCTAG | 1020 | OPA1 H31 | GAGAGAAAAACCATTACTTTGATCC |
| | 3UTR(+2587+2611) | | | 3UTR(+2641+2665) | |
| 1003 | OPA1 H31 | ATTTCTTTCCTCAGATACATACATC | 1021 | OPA1 H31 | ACTGAGAGAAAAACCATTACTTTGA |
| | 3UTR(+2590+2614) | | | 3UTR(+2644+2668) | |
| 1004 | OPA1 H31 | CAGATTTCTTTCCTCAGATACATAC | 1022 | OPA1 H31 | AGAACTGAGAGAAAAACCATTACTT |
| | 3UTR(+2593+2617) | | | 3UTR(+2647+2671) | |
| 1005 | OPA1 H31 | TACCAGATTTCTTTCCTCAGATACA | 1023 | OPA1 H31 | TAGAGAACTGAGAGAAAAACCATTA |
| | 3UTR(+2596+2620) | | | 3UTR(+2650+2674) | |
| 1006 | OPA1 H31 | AAATACCAGATTTCTTTCCTCAGAT | 1024 | OPA1 H31 | GCTTAGAGAACTGAGAGAAAAACCA |
| | 3UTR(+2599+2623) | | | 3UTR(+2653+2677) | |
| 1007 | OPA1 H31 | CAAAAATACCAGATTTCTTTCCTCA | 1025 | OPA1 H31 | CCAGCTTAGAGAACTGAGAGAAAAA |
| | 3UTR(+2602+2626) | | | 3UTR(+2656+2680) | |
| 1008 | OPA1 H31 | AAGCAAAAATACCAGATTTCTTTCC | 1026 | OPA1 H31 | AGACCAGCTTAGAGAACTGAGAGAA |
| | 3UTR(+2605+2629) | | | 3UTR(+2659+2683) | |
| 1009 | OPA1 H31 | GGAAAGCAAAAATACCAGATTTCTT | 1027 | OPA1 H31 | CATAGACCAGCTTAGAGAACTGAGA |
| | 3UTR(+2608+2632) | | | 3UTR(+2662+2686) | |
| 1010 | OPA1 H31 | ATTGGAAAGCAAAAATACCAGATTT | 1028 | OPA1 H31 | TAACATAGACCAGCTTAGAGAACTG |
| | 3UTR(+2611+2635) | | | 3UTR(+2665+2689) | |
| 1011 | OPA1 H31 | TTTATTGGAAAGCAAAAATACCAGA | 1029 | OPA1 H31 | CTATAACATAGACCAGCTTAGAGAA |
| | 3UTR(+2614+2638) | | | 3UTR(+2668+2692) | |
| 1012 | OPA1 H31 | CCCTTTATTGGAAAGCAAAAATACC | 1030 | OPA1 H31 | GAGCTATAACATAGACCAGCTTAGA |
| | 3UTR(+2617+2641) | | | 3UTR(+2671+2695) | |
| 1013 | OPA1 H31 | ATCCCCTTTATTGGAAAGCAAAAAT | 1031 | OPA1 H31 | CTAGAGCTATAACATAGACCAGCTT |
| | 3UTR(+2620+2644) | | | 3UTR(+2674+2698) | |
| 1014 | OPA1 H31 | TTGATCCCCTTTATTGGAAAGCAAA | 1032 | OPA1 H31 | CTGCTAGAGCTATAACATAGACCAG |
| | 3UTR(+2623+2647) | | | 3UTR(+2677+2701) | |
| 1015 | OPA1 H31 | ACTTTGATCCCCTTTATTGGAAAGC | 1033 | OPA1 H31 | ATACTGCTAGAGCTATAACATAGAC |
| | 3UTR(+2626+2650) | | | 3UTR(+2680+2704) | |
| 1016 | OPA1 H31 | ATTACTTTGATCCCCTTTATTGGAA | 1034 | OPA1 H31 | TCCATACTGCTAGAGCTATAACATA |
| | 3UTR(+2629+2653) | | | 3UTR(+2683+2707) | |
| 1035 | OPA1 H31 | ATTTCCATACTGCTAGAGCTATAAC | 1053 | OPA1 H31 | ATCTCAACAACATATAGCCATGATC |
| | 3UTR(+2686+2710) | | | 3UTR(+2740+2764) | |
| 1036 | OPA1 H31 | CACATTTCCATACTGCTAGAGCTAT | 1054 | OPA1 H31 | AATATCTCAACAACATATAGCCATG |
| | 3UTR(+2689+2713) | | | 3UTR(+2743+2767) | |
| 1037 | OPA1 H31 | AAGCACATTTCCATACTGCTAGAGC | 1055 | OPA1 H31 | TCAAATATCTCAACAACATATAGCC |
| | 3UTR(+2692+2716) | | | 3UTR(+2746+2770) | |
| 1038 | OPA1 H31 | TTAAAGCACATTTCCATACTGCTAG | 1056 | OPA1 H31 | GTTTCAAATATCTCAACAACATATA |
| | 3UTR(+2695+2719) | | | 3UTR(+2749+2773) | |
| 1039 | OPA1 H31 | ATTTTAAAGCACATTTCCATACTGC | 1057 | OPA1 H31 | TAAGTTTCAAATATCTCAACAACAT |
| | 3UTR(+2698+2722) | | | 3UTR(+2752+2776) | |
| 1040 | OPA1 H31 | CATATTTTAAAGCACATTTCCATAC | 1058 | OPA1 H31 | AGGTAAGTTTCAAATATCTCAACAA |
| | 3UTR(+2701+2725) | | | 3UTR(+2755+2779) | |
| 1041 | OPA1 H31 | AAGCATATTTTAAAGCACATTTCCA | 1059 | OPA1 H31 | ACAAGGTAAGTTTCAAATATCTCAA |
| | 3UTR(+2704+2728) | | | 3UTR(+2758+2782) | |
| 1042 | OPA1 H31 | GGTAAGCATATTTTAAAGCACATTT | 1060 | OPA1 H31 | AAAACAAGGTAAGTTTCAAATATCT |
| | 3UTR(+2707+2731) | | | 3UTR(+2761+2785) | |
| 1043 | OPA1 H31 | AAAGGTAAGCATATTTTAAAGCACA | 1061 | OPA1 H31 | GTGAAAACAAGGTAAGTTTCAAATA |
| | 3UTR(+2710+2734) | | | 3UTR(+2764+2788) | |
| 1044 | OPA1 H31 | TCAAAAGGTAAGCATATTTTAAAGC | 1062 | OPA1 H31 | CAAGTGAAAACAAGGTAAGTTTCAA |
| | 3UTR(+2713+2737) | | | 3UTR(+2767+2791) | |
| 1045 | OPA1 H31 | CATTCAAAAGGTAAGCATATTTTAA | 1063 | OPA1 H31 | GCACAAGTGAAAACAAGGTAAGTTT |
| | 3UTR(+2716+2740) | | | 3UTR(+2770+2794) | |
| 1046 | OPA1 H31 | GATCATTCAAAAGGTAAGCATATTT | 1064 | OPA1 H31 | AGTGCACAAGTGAAAACAAGGTAAG |
| | 3UTR(+2719+2743) | | | 3UTR(+2773+2797) | |
| 1047 | OPA1 H31 | CATGATCATTCAAAAGGTAAGCATA | 1065 | OPA1 H31 | CACAGTGCACAAGTGAAAACAAGGT |
| | 3UTR(+2722+2746) | | | 3UTR(+2776+2800) | |
| 1048 | OPA1 H31 | AGCCATGATCATTCAAAAGGTAAGC | 1066 | OPA1 H31 | ATTCACAGTGCACAAGTGAAAACAA |
| | 3UTR(+2725+2749) | | | 3UTR(+2779+2803) | |
| 1049 | OPA1 H31 | TATAGCCATGATCATTCAAAAGGTA | 1067 | OPA1 H31 | TTCATTCACAGTGCACAAGTGAAAA |
| | 3UTR(+2728+2752) | | | 3UTR(+2782+2806) | |
| 1050 | OPA1 H31 | ACATATAGCCATGATCATTCAAAAG | 1068 | OPA1 H31 | AAGTTCATTCACAGTGCACAAGTGA |
| | 3UTR(+2731+2755) | | | 3UTR(+2785+2809) | |
| 1051 | OPA1 H31 | ACAACATATAGCCATGATCATTCAA | 1069 | OPA1 H31 | ACAAAGTTCATTCACAGTGCACAAG |
| | 3UTR(+2734+2758) | | | 3UTR(+2788+2812) | |
| 1052 | OPA1 H31 | TCAACAACATATAGCCATGATCATT | 1070 | OPA1 H31 | AATACAAAGTTCATTCACAGTGCAC |
| | 3UTR(+2737+2761) | | | 3UTR(+2791+2815) | |
| 1071 | OPA1 H31 | AATAATACAAAGTTCATTCACAGTG | 1089 | OPA1 H31 | CAGGCAAAATATAAGTTGCAATAAT |
| | 3UTR(+2794+2818) | | | 3UTR(+2848+2872) | |
| 1072 | OPA1 H31 | AAAAATAATACAAAGTTCATTCACA | 1090 | OPA1 H31 | GCTCAGGCAAAATATAAGTTGCAAT |
| | 3UTR(+2797+2821) | | | 3UTR(+2851+2875) | |
| 1073 | OPA1 H31 | TAAAAAAATAATACAAAGTTCATTC | 1091 | OPA1 H31 | CAAGCTCAGGCAAAATATAAGTTGC |
| | 3UTR(+2800+2824) | | | 3UTR(+2854+2878) | |
| 1074 | OPA1 H31 | TTTTAAAAAAATAATACAAAGTTCA | 1092 | OPA1 H31 | GATCAAGCTCAGGCAAAATATAAGT |
| | 3UTR(+2803+2827) | | | 3UTR(+2857+2881) | |
| 1075 | OPA1 H31 | AGGTTTTAAAAAAATAATACAAAGT | 1093 | OPA1 H31 | TTTGATCAAGCTCAGGCAAAATATA |
| | 3UTR(+2806+2830) | | | 3UTR(+2860+2884) | |
| 1076 | OPA1 H31 | TGAAGGTTTTAAAAAAATAATACAA | 1094 | OPA1 H31 | ACCTTTGATCAAGCTCAGGCAAAAT |
| | 3UTR(+2809+2833) | | | 3UTR(+2863+2887) | |
| 1077 | OPA1 H31 | ATGTGAAGGTTTTAAAAAAATAATA | 1095 | OPA1 H31 | ATGACCTTTGATCAAGCTCAGGCAA |
| | 3UTR(+2812+2836) | | | 3UTR(+2866+2890) | |
| 1078 | OPA1 H31 | GTAATGTGAAGGTTTTAAAAAAATA | 1096 | OPA1 H31 | CAAATGACCTTTGATCAAGCTCAGG |
| | 3UTR(+2815+2839) | | | 3UTR(+2869+2893) | |
| 1079 | OPA1 H31 | CACGTAATGTGAAGGTTTTAAAAAA | 1097 | OPA1 H31 | ACACAAATGACCTTTGATCAAGCTC |
| | 3UTR(+2818+2842) | | | 3UTR(+2872+2896) | |
| 1080 | OPA1 H31 | CTACACGTAATGTGAAGGTTTTAAA | 1098 | OPA1 H31 | TCTACACAAATGACCTTTGATCAAG |
| | 3UTR(+2821+2845) | | | 3UTR(+2875+2899) | |
| 1081 | OPA1 H31 | TATCTACACGTAATGTGAAGGTTTT | 1099 | OPA1 H31 | TCATCTACACAAATGACCTTTGATC |
| | 3UTR(+2824+2848) | | | 3UTR(+2878+2902) | |
| 1082 | OPA1 H31 | TAATATCTACACGTAATGTGAAGGT | 1100 | OPA1 H31 | TACTCATCTACACAAATGACCTTTG |
| | 3UTR(+2827+2851) | | | 3UTR(+2881+2905) | |
| 1083 | OPA1 H31 | CAATAATATCTACACGTAATGTGAA | 1101 | OPA1 H31 | AATTACTCATCTACACAAATGACCT |
| | 3UTR(+2830+2854) | | | 3UTR(+2884+2908) | |
| 1084 | OPA1 H31 | TTGCAATAATATCTACACGTAATGT | 1102 | OPA1 H31 | TTTAATTACTCATCTACACAAATGA |
| | 3UTR(+2833+2857) | | | 3UTR(+2887+2911) | |
| 1085 | OPA1 H31 | AAGTTGCAATAATATCTACACGTAA | 1103 | OPA1 H31 | TTTTTTAATTACTCATCTACACAAA |
| | 3UTR(+2836+2860) | | | 3UTR(+2890+2914) | |
| 1086 | OPA1 H31 | TATAAGTTGCAATAATATCTACACG | 1104 | OPA1 H31 | ATATTTTTTAATTACTCATCTACAC |
| | 3UTR(+2839+2863) | | | 3UTR(+2893+2917) | |
| 1087 | OPA1 H31 | AAATATAAGTTGCAATAATATCTAC | 1105 | OPA1 H31 | TAAATATTTTTTAATTACTCATCTA |
| | 3UTR(+2842+2866) | | | 3UTR(+2896+2920) | |
| 1088 | OPA1 H31 | GCAAAATATAAGTTGCAATAATATC | 1106 | OPA1 H31 | ATTTAAATATTTTTTAATTACTCAT |
| | 3UTR(+2845+2869) | | | 3UTR(+2899+2923) | |
| 1107 | OPA1 H31 | GTGATTTAAATATTTTTTAATTACT | 1125 | OPA1 H31 | CTCGTTAAAACAGAAAAAAATTTAA |
| | 3UTR(+2902+2926) | | | 3UTR(+2956+2980) | |
| 1108 | OPA1 H31 | AATGTGATTTAAATATTTTTTAATT | 1126 | OPA1 H31 | TCCCTCGTTAAAACAGAAAAAAATT |
| | 3UTR(+2905+2929) | | | 3UTR(+2959+2983) | |
| 1109 | OPA1 H31 | TATAATGTGATTTAAATATTTTTTA | 1127 | OPA1 H31 | CTTTCCCTCGTTAAAACAGAAAAAA |
| | 3UTR(+2908+2932) | | | 3UTR(+2962+2986) | |
| 1110 | OPA1 H31 | AATTATAATGTGATTTAAATATTTT | 1128 | OPA1 H31 | TCTCTTTCCCTCGTTAAAACAGAAA |
| | 3UTR(+2911+2935) | | | 3UTR(+2965+2989) | |
| 1111 | OPA1 H31 | TAGAATTATAATGTGATTTAAATAT | 1129 | OPA1 H31 | GTTTCTCTTTCCCTCGTTAAAACAG |
| | 3UTR(+2914+2938) | | | 3UTR(+2968+2992) | |
| 1112 | OPA1 H31 | TAATAGAATTATAATGTGATTTAAA | 1130 | OPA1 H31 | CAGGTTTCTCTTTCCCTCGTTAAAA |
| | 3UTR(+2917+2941) | | | 3UTR(+2971+2995) | |
| 1113 | OPA1 H31 | CAATAATAGAATTATAATGTGATTT | 1131 | OPA1 H31 | ATACAGGTTTCTCTTTCCCTCGTTA |
| | 3UTR(+2920+2944) | | | 3UTR(+2974+2998) | |
| 1114 | OPA1 H31 | CTCCAATAATAGAATTATAATGTGA | 1132 | OPA1 H31 | GGTATACAGGTTTCTCTTTCCCTCG |
| | 3UTR(+2923+2947) | | | 3UTR(+2977+3001) | |
| 1115 | OPA1 H31 | GCTCTCCAATAATAGAATTATAATG | 1133 | OPA1 H31 | CTAGGTATACAGGTTTCTCTTTCCC |
| | 3UTR(+2926+2950) | | | 3UTR(+2980+3004) | |
| 1116 | OPA1 H31 | GATGCTCTCCAATAATAGAATTATA | 1134 | OPA1 H31 | ACCCTAGGTATACAGGTTTCTCTTT |
| | 3UTR(+2929+2953) | | | 3UTR(+2983+3007) | |
| 1117 | OPA1 H31 | AAAGATGCTCTCCAATAATAGAATT | 1135 | OPA1 H31 | ATGACCCTAGGTATACAGGTTTCTC |
| | 3UTR(+2932+2956) | | | 3UTR(+2986+3010) | |
| 1118 | OPA1 H31 | TTAAAAGATGCTCTCCAATAATAGA | 1136 | OPA1 H31 | ATAATGACCCTAGGTATACAGGTTT |
| | 3UTR(+2935+2959) | | | 3UTR(+2989+3013) | |
| 1119 | OPA1 H31 | AATTTAAAAGATGCTCTCCAATAAT | 1137 | OPA1 H31 | CAAATAATGACCCTAGGTATACAGG |
| | 3UTR(+2938+2962) | | | 3UTR(+2992+3016) | |
| 1120 | OPA1 H31 | AAAAATTTAAAAGATGCTCTCCAAT | 1138 | OPA1 H31 | GGTCAAATAATGACCCTAGGTATAC |
| | 3UTR(+2941+2965) | | | 3UTR(+2995+3019) | |
| 1121 | OPA1 H31 | GAAAAAAATTTAAAAGATGCTCTCC | 1139 | OPA1 H31 | TGGGGTCAAATAATGACCCTAGGTA |
| | 3UTR(+2944+2968) | | | 3UTR(+2998+3022) | |
| 1122 | OPA1 H31 | ACAGAAAAAAATTTAAAAGATGCTC | 1140 | OPA1 H31 | CTATGGGGTCAAATAATGACCCTAG |
| | 3UTR(+2947+2971) | | | 3UTR(+3001+3025) | |
| 1123 | OPA1 H31 | AAAACAGAAAAAAATTTAAAAGATG | 1141 | OPA1 H31 | ATACTATGGGGTCAAATAATGACCC |
| | 3UTR(+2950+2974) | | | 3UTR(+3004+3028) | |
| 1124 | OPA1 H31 | GTTAAAACAGAAAAAAATTTAAAAG | 1142 | OPA1 H31 | GTTATACTATGGGGTCAAATAATGA |
| | 3UTR(+2953+2977) | | | 3UTR(+3007+3031) | |
| 1143 | OPA1 H31 | CTGGTTATACTATGGGGTCAAATAA | 1161 | OPA1 H31 | TGAAATTCAAGCATTCAGAGAAAAG |
| | 3UTR(+3010+3034) | | | 3UTR(+3064+3088) | |
| 1144 | OPA1 H31 | AATCTGGTTATACTATGGGGTCAAA | 1162 | OPA1 H31 | ATGTGAAATTCAAGCATTCAGAGAA |
| | 3UTR(+3013+3037) | | | 3UTR(+3067+3091) | |
| 1145 | OPA1 H31 | ATGAATCTGGTTATACTATGGGGTC | 1163 | OPA1 H31 | GGCATGTGAAATTCAAGCATTCAGA |
| | 3UTR(+3016+3040) | | | 3UTR(+3070+3094) | |
| 1146 | OPA1 H31 | ACCATGAATCTGGTTATACTATGGG | 1164 | OPA1 H31 | CAAGGCATGTGAAATTCAAGCATTC |
| | 3UTR(+3019+3043) | | | 3UTR(+3073+3097) | |
| 1147 | OPA1 H31 | TAGACCATGAATCTGGTTATACTAT | 1165 | OPA1 H31 | ATGCAAGGCATGTGAAATTCAAGCA |
| | 3UTR(+3022+3046) | | | 3UTR(+3076+3100) | |
| 1148 | OPA1 H31 | TGTTAGACCATGAATCTGGTTATAC | 1166 | OPA1 H31 | GAAATGCAAGGCATGTGAAATTCAA |
| | 3UTR(+3025+3049) | | | 3UTR(+3079+3103) | |
| 1149 | OPA1 H31 | GCTTGTTAGACCATGAATCTGGTTA | 1167 | OPA1 H31 | TGTGAAATGCAAGGCATGTGAAATT |
| | 3UTR(+3028+3052) | | | 3UTR(+3082+3106) | |
| 1150 | OPA1 H31 | AGAGCTTGTTAGACCATGAATCTGG | 1168 | OPA1 H31 | AACTGTGAAATGCAAGGCATGTGAA |
| | 3UTR(+3031+3055) | | | 3UTR(+3085+3109) | |
| 1151 | OPA1 H31 | CTGAGAGCTTGTTAGACCATGAATC | 1169 | OPA1 H31 | TACAACTGTGAAATGCAAGGCATGT |
| | 3UTR(+3034+3058) | | | 3UTR(+3088+3112) | |
| 1152 | OPA1 H31 | ACACTGAGAGCTTGTTAGACCATGA | 1170 | OPA1 H31 | GAGTACAACTGTGAAATGCAAGGCA |
| | 3UTR(+3037+3061) | | | 3UTR(+3091+3115) | |
| 1153 | OPA1 H31 | GCCACACTGAGAGCTTGTTAGACCA | 1171 | OPA1 H31 | ATGGAGTACAACTGTGAAATGCAAG |
| | 3UTR(+3040+3064) | | | 3UTR(+3094+3118) | |
| 1154 | OPA1 H31 | AAAGCCACACTGAGAGCTTGTTAGA | 1172 | OPA1 H31 | ACCATGGAGTACAACTGTGAAATGC |
| | 3UTR(+3043+3067) | | | 3UTR(+3097+3121) | |
| 1155 | OPA1 H31 | AGAAAAGCCACACTGAGAGCTTGTT | 1173 | OPA1 H31 | TTGACCATGGAGTACAACTGTGAAA |
| | 3UTR(+3046+3070) | | | 3UTR(+3100+3124) | |
| 1156 | OPA1 H31 | CAGAGAAAAGCCACACTGAGAGCTT | 1174 | OPA1 H31 | CGGTTGACCATGGAGTACAACTGTG |
| | 3UTR(+3049+3073) | | | 3UTR(+3103+3127) | |
| 1157 | OPA1 H31 | ATTCAGAGAAAAGCCACACTGAGAG | 1175 | OPA1 H31 | CACCGGTTGACCATGGAGTACAACT |
| | 3UTR(+3052+3076) | | | 3UTR(+3106+3130) | |
| 1158 | OPA1 H31 | AGCATTCAGAGAAAAGCCACACTGA | 1176 | OPA1 H31 | AAGCACCGGTTGACCATGGAGTACA |
| | 3UTR(+3055+3079) | | | 3UTR(+3109+3133) | |
| 1159 | OPA1 H31 | TCAAGCATTCAGAGAAAAGCCACAC | 1177 | OPA1 H31 | AAAAAGCACCGGTTGACCATGGAGT |
| | 3UTR(+3058+3082) | | | 3UTR(+3112+3136) | |
| 1160 | OPA1 H31 | AATTCAAGCATTCAGAGAAAAGCCA | 1178 | OPA1 H31 | GAAAAAAAGCACCGGTTGACCATGG |
| | 3UTR(+3061+3085) | | | 3UTR(+3115+3139) | |
| 1179 | OPA1 H31 | TGTGAAAAAAAGCACCGGTTGACCA | 1197 | OPA1 H31 | TTTTTTATATATTTTACCAAGGAAA |
| | 3UTR(+3118+3142) | | | 3UTR(+3172+3196) | |
| 1180 | OPA1 H31 | CGATGTGAAAAAAAGCACCGGTTGA | 1198 | OPA1 H31 | ACCTTTTTTATATATTTTACCAAGG |
| | 3UTR(+3121+3145) | | | 3UTR(+3175+3199) | |
| 1181 | OPA1 H31 | CCACGATGTGAAAAAAAGCACCGGT | 1199 | OPA1 H31 | AAAACCTTTTTTATATATTTTACCA |
| | 3UTR(+3124+3148) | | | 3UTR(+3178+3202) | |
| 1182 | OPA1 H31 | GTACCACGATGTGAAAAAAAGCACC | 1200 | OPA1 H31 | TAGAAAACCTTTTTTATATATTTTA |
| | 3UTR(+3127+3151) | | | 3UTR(+3181+3205) | |
| 1183 | OPA1 H31 | CAAGTACCACGATGTGAAAAAAAGC | 1201 | OPA1 H31 | AATTAGAAAACCTTTTTTATATATT |
| | 3UTR(+3130+3154) | | | 3UTR(+3184+3208) | |
| 1184 | OPA1 H31 | TGACAAGTACCACGATGTGAAAAAA | 1202 | OPA1 H31 | TGAAATTAGAAAACCTTTTTTATAT |
| | 3UTR(+3133+3157) | | | 3UTR(+3187+3211) | |
| 1185 | OPA1 H31 | TTTTGACAAGTACCACGATGTGAAA | 1203 | OPA1 H31 | AAGTGAAATTAGAAAACCTTTTTTA |
| | 3UTR(+3136+3160) | | | 3UTR(+3190+3214) | |
| 1186 | OPA1 H31 | ATGTTTTGACAAGTACCACGATGTG | 1204 | OPA1 H31 | GCAAAGTGAAATTAGAAAACCTTTT |
| | 3UTR(+3139+3163) | | | 3UTR(+3193+3217) | |
| 1187 | OPA1 H31 | AAAATGTTTTGACAAGTACCACGAT | 1205 | OPA1 H31 | GCAGCAAAGTGAAATTAGAAAACCT |
| | 3UTR(+3142+3166) | | | 3UTR(+3196+3220) | |
| 1188 | OPA1 H31 | AACAAAATGTTTTGACAAGTACCAC | 1206 | OPA1 H31 | TTGGCAGCAAAGTGAAATTAGAAAA |
| | 3UTR(+3145+3169) | | | 3UTR(+3199+3223) | |
| 1189 | OPA1 H31 | AATAACAAAATGTTTTGACAAGTAC | 1207 | OPA1 H31 | GCCTTGGCAGCAAAGTGAAATTAGA |
| | 3UTR(+3148+3172) | | | 3UTR(+3202+3226) | |
| 1190 | OPA1 H31 | GAAAATAACAAAATGTTTTGACAAG | 1208 | OPA1 H31 | ACAGCCTTGGCAGCAAAGTGAAATT |
| | 3UTR(+3151+3175) | | | 3UTR(+3205+3229) | |
| 1191 | OPA1 H31 | AAGGAAAATAACAAAATGTTTTGAC | 1209 | OPA1 H31 | ATGACAGCCTTGGCAGCAAAGTGAA |
| | 3UTR(+3154+3178) | | | 3UTR(+3208+3232) | |
| 1192 | OPA1 H31 | ACCAAGGAAAATAACAAAATGTTTT | 1210 | OPA1 H31 | AAAATGACAGCCTTGGCAGCAAAGT |
| | 3UTR(+3157+3181) | | | 3UTR (+3211−24) | |
| 1193 | OPA1 H31 | TTTACCAAGGAAAATAACAAAATGT | 1211 | OPA1 H31 | ATGAAAATGACAGCCTTGGCAGCAA |
| | 3UTR(+3160+3184) | | | 3UTR (−3−27) | |
| 1194 | OPA1 H31 | TATTTTACCAAGGAAAATAACAAAA | 1212 | OPA1 H31 | TTAATGAAAATGACAGCCTTGGCAG |
| | 3UTR(+3163+3187) | | | 3UTR (−6−30) | |
| 1195 | OPA1 H31 | ATATATTTTACCAAGGAAAATAACA | 1213 | OPA1 H31 | GCATTAATGAAAATGACAGCCTTGG |
| | 3UTR(+3166+3190) | | | 3UTR (−9−33) | |
| 1196 | OPA1 H31 | TTTATATATTTTACCAAGGAAAATA | 1214 | OPA1 H31 | GCAGCATTAATGAAAATGACAGCCT |
| | 3UTR(+3169+3193) | | | 3UTR (−12−36) | |
| 1215 | OPA1 H31 | TTGGCAGCATTAATGAAAATGACAG | — | | |
| | 3UTR(−15−39) | | | | |
| 1216 | OPA1 H31 | ATGTTGGCAGCATTAATGAAAATGA | 1237 | OPA1 H31 | GAAATTAAAGTCTCTTCCAGTTCTA |
| | 3UTR(−18−42) | | | 3UTR(−81−105) | |
| 1217 | OPA1 H31 | AACATGTTGGCAGCATTAATGAAAA | 1238 | OPA1 H31 | GATGAAATTAAAGTCTCTTCCAGTT |
| | 3UTR(−21−45) | | | 3UTR(−84−108) | |
| 1218 | OPA1 H31 | ATGAACATGTTGGCAGCATTAATGA | 1239 | OPA1 H31 | GTAGATGAAATTAAAGTCTCTTCCA |
| | 3UTR(−24−48) | | | 3UTR(−87−111) | |
| 1219 | OPA1 H31 | CATATGAACATGTTGGCAGCATTAA | 1240 | OPA1 H31 | ATGGTAGATGAAATTAAAGTCTCTT |
| | 3UTR(−27−51) | | | 3UTR(−90−114) | |
| 1220 | OPA1 H31 | CCTCATATGAACATGTTGGCAGCAT | 1241 | OPA1 H31 | CAGATGGTAGATGAAATTAAAGTCT |
| | 3UTR(−30−54) | | | 3UTR(−93−117) | |
| 1221 | OPA1 H31 | AAGCCTCATATGAACATGTTGGCAG | 1242 | OPA1 H31 | AATCAGATGGTAGATGAAATTAAAG |
| | 3UTR(−33−57) | | | 3UTR(−96−120) | |
| 1222 | OPA1 H31 | AGTAAGCCTCATATGAACATGTTGG | 1243 | OPA1 H31 | TATAATCAGATGGTAGATGAAATTA |
| | 3UTR(−36−60) | | | 3UTR(−99−123) | |
| 1223 | OPA1 H31 | CTTAGTAAGCCTCATATGAACATGT | 1244 | OPA1 H31 | GTTTATAATCAGATGGTAGATGAAA |
| | 3UTR(−39−63) | | | 3UTR(−102−126) | |
| 1224 | OPA1 H31 | CTTCTTAGTAAGCCTCATATGAACA | 1245 | OPA1 H31 | GGAGTTTATAATCAGATGGTAGATG |
| | 3UTR(−42−66) | | | 3UTR(−105−129) | |
| 1225 | OPA1 H31 | TAACTTCTTAGTAAGCCTCATATGA | 1246 | OPA1 H31 | ACAGGAGTTTATAATCAGATGGTAG |
| | 3UTR(−45−69) | | | 3UTR(−108−132) | |
| 1226 | OPA1 H31 | TTATAACTTCTTAGTAAGCCTCATA | 1247 | OPA1 H31 | GTAACAGGAGTTTATAATCAGATGG |
| | 3UTR(−48−72) | | | 3UTR(−111−135) | |
| 1227 | OPA1 H31 | TAGTTATAACTTCTTAGTAAGCCTC | 1248 | OPA1 H31 | TGAGTAACAGGAGTTTATAATCAGA |
| | 3UTR(−51−75) | | | 3UTR(−114−138) | |
| 1228 | OPA1 H31 | GCTTAGTTATAACTTCTTAGTAAGC | 1249 | OPA1 H31 | AAGTGAGTAACAGGAGTTTATAATC |
| | 3UTR(−54−78) | | | 3UTR(−117−141) | |
| 1229 | OPA1 H31 | AAAGCTTAGTTATAACTTCTTAGTA | 1250 | OPA1 H31 | ACAAAGTGAGTAACAGGAGTTTATA |
| | 3UTR(−57−81) | | | 3UTR(−120−144) | |
| 1230 | OPA1 H31 | TCTAAAGCTTAGTTATAACTTCTTA | 1251 | OPA1 H31 | ATAACAAAGTGAGTAACAGGAGTTT |
| | 3UTR(−60−84) | | | 3UTR(−123−147) | |
| 1231 | OPA1 H31 | AGTTCTAAAGCTTAGTTATAACTTC | 1252 | OPA1 H31 | GGAATAACAAAGTGAGTAACAGGAG |
| | 3UTR(−63−87) | | | 3UTR(−126−150) | |
| 1232 | OPA1 H31 | TCCAGTTCTAAAGCTTAGTTATAAC | 1253 | OPA1 H31 | GGAGGAATAACAAAGTGAGTAACAG |
| | 3UTR(−66−90) | | | 3UTR(−129−153) | |
| 1233 | OPA1 H31 | TCTTCCAGTTCTAAAGCTTAGTTAT | 1254 | OPA1 H31 | TCTGGAGGAATAACAAAGTGAGTAA |
| | 3UTR(−69−93) | | | 3UTR(−132−156) | |
| 1234 | OPA1 H31 | GTCTCTTCCAGTTCTAAAGCTTAGT | 1255 | OPA1 H31 | AGTTCTGGAGGAATAACAAAGTGAG |
| | 3UTR(−72−96) | | | 3UTR(−135−159) | |
| 1235 | OPA1 H31 | AAAGTCTCTTCCAGTTCTAAAGCTT | 1256 | OPA1 H31 | CAAAGTTCTGGAGGAATAACAAAGT |
| | 3UTR(−75−99) | | | 3UTR(−138−162) | |
| 1236 | OPA1 H31 | ATTAAAGTCTCTTCCAGTTCTAAAG | 1257 | OPA1 H31 | GAGCAAAGTTCTGGAGGAATAACAA |
| | 3UTR(−78−102) | | | 3UTR(−141−165) | |
| 1258 | OPA1 H31 | TATGAGCAAAGTTCTGGAGGAATAA | 1276 | OPA1 H31 | CTATACGCTACCTAACTTGACATAT |
| | 3UTR(−144−168) | | | 3UTR(−198−222) | |
| 1259 | OPA1 H31 | ATTTATGAGCAAAGTTCTGGAGGAA | 1277 | OPA1 H31 | CACCTATACGCTACCTAACTTGACA |
| | 3UTR(−147−171) | | | 3UTR(−201−225) | |
| 1260 | OPA1 H31 | ATCATTTATGAGCAAAGTTCTGGAG | 1278 | OPA1 H31 | GCACACCTATACGCTACCTAACTTG |
| | 3UTR(−150−174) | | | 3UTR(−204−228) | |
| 1261 | OPA1 H31 | GGTATCATTTATGAGCAAAGTTCTG | 1279 | OPA1 H31 | AAGGCACACCTATACGCTACCTAAC |
| | 3UTR(−153−177) | | | 3UTR(−207−231) | |
| 1262 | OPA1 H31 | GTAGGTATCATTTATGAGCAAAGTT | 1280 | OPA1 H31 | AAGAAGGCACACCTATACGCTACCT |
| | 3UTR(−156−180) | | | 3UTR(−210−234) | |
| 1263 | OPA1 H31 | TTAGTAGGTATCATTTATGAGCAAA | 1281 | OPA1 H31 | ATTAAGAAGGCACACCTATACGCTA |
| | 3UTR(−159−183) | | | 3UTR(−213−237) | |
| 1264 | OPA1 H31 | CAATTAGTAGGTATCATTTATGAGC | 1282 | OPA1 H31 | GGAATTAAGAAGGCACACCTATACG |
| | 3UTR(−162−186) | | | 3UTR(−216−240) | |
| 1265 | OPA1 H31 | CGACAATTAGTAGGTATCATTTATG | 1283 | OPA1 H31 | GAAGGAATTAAGAAGGCACACCTAT |
| | 3UTR(−165−189) | | | 3UTR(−219−243) | |
| 1266 | OPA1 H31 | GATCGACAATTAGTAGGTATCATTT | 1284 | OPA1 H31 | TCTGAAGGAATTAAGAAGGCACACC |
| | 3UTR(−168−192) | | | 3UTR(−222−246) | |
| 1267 | OPA1 H31 | AATGATCGACAATTAGTAGGTATCA | 1285 | OPA1 H31 | TCTTCTGAAGGAATTAAGAAGGCAC |
| | 3UTR(−171−195) | | | 3UTR(−225−249) | |
| 1268 | OPA1 H31 | TCCAATGATCGACAATTAGTAGGTA | 1286 | OPA1 H31 | GCATCTTCTGAAGGAATTAAGAAGG |
| | 3UTR(−174−198) | | | 3UTR(−228−252) | |
| 1269 | OPA1 H31 | ATATCCAATGATCGACAATTAGTAG | 1287 | OPA1 H31 | TCTGCATCTTCTGAAGGAATTAAGA |
| | 3UTR(−177−201) | | | 3UTR(−231−255) | |
| 1270 | OPA1 H31 | GACATATCCAATGATCGACAATTAG | 1288 | OPA1 H31 | GCCTCTGCATCTTCTGAAGGAATTA |
| | 3UTR(−180−204) | | | 3UTR(−234−258) | |
| 1271 | OPA1 H31 | CTTGACATATCCAATGATCGACAAT | 1289 | OPA1 H31 | CTTGCCTCTGCATCTTCTGAAGGAA |
| | 3UTR(−183−207) | | | 3UTR(−237−261) | |
| 1272 | OPA1 H31 | TAACTTGACATATCCAATGATCGAC | 1290 | OPA1 H31 | GTTCTTGCCTCTGCATCTTCTGAAG |
| | 3UTR(−186−210) | | | 3UTR(−240−264) | |
| 1273 | OPA1 H31 | ACCTAACTTGACATATCCAATGATC | 1291 | OPA1 H31 | CATGTTCTTGCCTCTGCATCTTCTG |
| | 3UTR(−189−213) | | | 3UTR(−243−267) | |
| 1274 | OPA1 H31 | GCTACCTAACTTGACATATCCAATG | 1292 | OPA1 H31 | AAACATGTTCTTGCCTCTGCATCTT |
| | 3UTR(−192−216) | | | 3UTR(−246−270) | |
| 1275 | OPA1 H31 | TACGCTACCTAACTTGACATATCCA | 1293 | OPA1 H31 | TTGAAACATGTTCTTGCCTCTGCAT |
| | 3UTR(−195−219) | | | 3UTR(−249−273) | |
| 1294 | OPA1 H31 | CGATTGAAACATGTTCTTGCCTCTG | — | | |
| | 3UTR(−252−276) | | | | |
| 1295 | OPA1 H31 | ACACGATTGAAACATGTTCTTGCCT | 1304 | OPA1 H31 | TAGCACTGATAAAGAAAACCAATGC |
| | 3UTR(−255−279) | | | 3UTR(−282−306) | |
| 1296 | OPA1 H31 | CTAACACGATTGAAACATGTTCTTG | 1305 | OPA1 H31 | TGTTAGCACTGATAAAGAAAACCAA |
| | 3UTR(−258−282) | | | 3UTR(−285−309) | |
| 1297 | OPA1 H31 | ATGCTAACACGATTGAAACATGTTC | 1306 | OPA1 H31 | GTCTGTTAGCACTGATAAAGAAAAC |
| | 3UTR(−261−285) | | | 3UTR(−288−312) | |
| 1298 | OPA1 H31 | CCAATGCTAACACGATTGAAACATG | 1307 | OPA1 H31 | TGAGTCTGTTAGCACTGATAAAGAA |
| | 3UTR(−264−288) | | | 3UTR(−291−315) | |
| 1299 | OPA1 H31 | AAACCAATGCTAACACGATTGAAAC | 1308 | OPA1 H31 | CACTGAGTCTGTTAGCACTGATAAA |
| | 3UTR(−267−291) | | | 3UTR(−294−318) | |
| 1300 | OPA1 H31 | AGAAAACCAATGCTAACACGATTGA | 1309 | OPA1 H31 | TCACACTGAGTCTGTTAGCACTGAT |
| | 3UTR(−270−294) | | | 3UTR(−297−321) | |
| 1301 | OPA1 H31 | TAAAGAAAACCAATGCTAACACGAT | 1310 | OPA1 H31 | GCCTCACACTGAGTCTGTTAGCACT |
| | 3UTR(−273−297) | | | 3UTR(−300−324) | |
| 1302 | OPA1 H31 | TGATAAAGAAAACCAATGCTAACAC | 1311 | OPA1 H31 | GGGGCCTCACACTGAGTCTGTTAGC |
| | 3UTR(−276−300) | | | 3UTR(−303−327) | |
| 1303 | OPA1 H31 | CACTGATAAAGAAAACCAATGCTAA | 1312 | OPA1 H31 | TATGGGGCCTCACACTGAGTCTGTT |
| | 3UTR(−279−303) | | | 3UTR(−306−330) | |

TABLE 5
List of antisense oligonucleotide micro-walked sequences with 17 nucleotides in that target the 3′ UTR of an OPA1 transcript.

| SEQ | | | SEQ | | |
|---|---|---|---|---|---|
| ID | ASO oordinates | Sequence 5′ > 3′ | ID | ASO oordinates | Sequence 5′ > 3′ |
| 1313 | OPA1 H30:31 | ATGAGTACGATTTTAAT | 1319 | OPA1 H31 | ATGTATGCAGAGCTGAT |
| | 3UTR(+1+17) | | | 3UTR(+19+35) | |
| 1314 | OPA1 H30:31 | ATTATGAGTACGATTTT | 1320 | OPA1 H31 | CAGATGTATGCAGAGCT |
| | 3UTR(+4+20) | | | 3UTR(+22+38) | |
| 1315 | OPA1 H31 | CTGATTATGAGTACGAT | 1321 | OPA1 H31 | CTTCAGATGTATGCAGA |
| | 3UTR(+7+23) | | | 3UTR(+25+41) | |
| 1316 | OPA1 H31 | GAGCTGATTATGAGTAC | 1322 | OPA1 H31 | GTTCTTCAGATGTATGC |
| | 3UTR(+10+26) | | | 3UTR(+28+44) | |
| 1317 | OPA1 H31 | GCAGAGCTGATTATGAG | 1323 | OPA1 H31 | TTTGTTCTTCAGATGTA |
| | 3UTR(+13+29) | | | 3UTR(+31+47) | |
| 1318 | OPA1 H31 | TATGCAGAGCTGATTAT | 1324 | OPA1 H31 | GTTTTTGTTCTTCAGAT |
| | 3UTR(+16+32) | | | 3UTR(+34+50) | |
| 1325 | OPA1 H31 | GATGTTTTTGTTCTTCA | 1343 | OPA1 H31 | GTATGTTTAGAAAGGTG |
| | 3UTR(+37+53) | | | 3UTR(+91+107) | |
| 1326 | OPA1 H31 | GTTGATGTTTTTGTTCT | 1344 | OPA1 H31 | ATTGTATGTTTAGAAAG |
| | 3UTR(+40+56) | | | 3UTR(+94+110) | |
| 1327 | OPA1 H31 | GACGTTGATGTTTTTGT | 1345 | OPA1 H31 | TTTATTGTATGTTTAGA |
| | 3UTR(+43+59) | | | 3UTR(+97+113) | |
| 1328 | OPA1 H31 | AAAGACGTTGATGTTTT | 1346 | OPA1 H31 | GACTTTATTGTATGTTT |
| | 3UTR(+46+62) | | | 3UTR(+100+116) | |
| 1329 | OPA1 H31 | ACAAAAGACGTTGATGT | 1347 | OPA1 H31 | CATGACTTTATTGTATG |
| | 3UTR(+49+65) | | | 3UTR(+103+119) | |
| 1330 | OPA1 H31 | TGGACAAAAGACGTTGA | 1348 | OPA1 H31 | TCCCATGACTTTATTGT |
| | 3UTR(+52+68) | | | 3UTR(+106+122) | |
| 1331 | OPA1 H31 | GGCTGGACAAAAGACGT | 1349 | OPA1 H31 | TTATCCCATGACTTTAT |
| | 3UTR(+55+71) | | | 3UTR(+109+125) | |
| 1332 | OPA1 H31 | AGAGGCTGGACAAAAGA | 1350 | OPA1 H31 | TTTTTATCCCATGACTT |
| | 3UTR(+58+74) | | | 3UTR(+112+128) | |
| 1333 | OPA1 H31 | AAAAGAGGCTGGACAAA | 1351 | OPA1 H31 | TTATTTTTATCCCATGA |
| | 3UTR(+61+77) | | | 3UTR(+115+131) | |
| 1334 | OPA1 H31 3UTR(+64+80) | AGAAAAAGAGGCTGGAC | 1352 | OPA1 H31 | CGATTATTTTTATCCCA |
| | | | | 3UTR(+118+134) | |
| 1335 | OPA1 H31 3UTR(+67+83) | AGAAGAAAAAGAGGCTG | 1353 | OPA1 H31 | CATCGATTATTTTTATC |
| | | | | 3UTR(+121+137) | |
| 1336 | OPA1 H31 3UTR(+70+86) | AGCAGAAGAAAAAGAGG | 1354 | OPA1 H31 | ATACATCGATTATTTTT |
| | | | | 3UTR(+124+140) | |
| 1337 | OPA1 H31 3UTR(+73+89) | AACAGCAGAAGAAAAAG | 1355 | OPA1 H31 | AACATACATCGATTATT |
| | | | | 3UTR(+127+143) | |
| 1338 | OPA1 H31 3UTR(+76+92) | TGGAACAGCAGAAGAAA | 1356 | OPA1 H31 | CGTAACATACATCGATT |
| | | | | 3UTR(+130+146) | |
| 1339 | OPA1 H31 3UTR(+79+95) | AGGTGGAACAGCAGAAG | 1357 | OPA1 H31 | GCCCGTAACATACATCG |
| | | | | 3UTR(+133+149) | |
| 1340 | OPA1 H31 3UTR(+82+98) | GAAAGGTGGAACAGCAG | 1358 | OPA1 H31 | AGCGCCCGTAACATACA |
| | | | | 3UTR(+136+152) | |
| 1341 | OPA1 H31 3UTR(+85+101) | TTAGAAAGGTGGAACAG | 1359 | OPA1 H31 | TAAAGCGCCCGTAACAT |
| | | | | 3UTR(+139+155) | |
| 1342 | OPA1 H31 3UTR(+88+104) | TGTTTAGAAAGGTGGAA | 1360 | OPA1 H31 | GGTTAAAGCGCCCGTAA |
| | | | | 3UTR(+142+158) | |
| 1361 | OPA1 H31 | GATGGTTAAAGCGCCCG | 1379 | OPA1 H31 | CAACAAAATAGGATGTT |
| | 3UTR(+145+161) | | | 3UTR(+199+215) | |
| 1362 | OPA1 H31 | GCTGATGGTTAAAGCGC | 1380 | OPA1 H31 | GTACAACAAAATAGGAT |
| | 3UTR(+148+164) | | | 3UTR(+202+218) | |
| 1363 | OPA1 H31 | GCAGCTGATGGTTAAAG | 1381 | OPA1 H31 | TTAGTACAACAAAATAG |
| | 3UTR(+151+167) | | | 3UTR(+205+221) | |
| 1364 | OPA1 H31 | GAGGCAGCTGATGGTTA | 1382 | OPA1 H31 | ACTTTAGTACAACAAAA |
| | 3UTR(+154+170) | | | 3UTR(+208+224) | |
| 1365 | OPA1 H31 | CGAGAGGCAGCTGATGG | 1383 | OPA1 H31 | GTCACTTTAGTACAACA |
| | 3UTR(+157+173) | | | 3UTR(+211+227) | |
| 1366 | OPA1 H31 | ATTCGAGAGGCAGCTGA | 1384 | OPA1 H31 | TTTGTCACTTTAGTACA |
| | 3UTR(+160+176) | | | 3UTR(+214+230) | |
| 1367 | OPA1 H31 | TCCATTCGAGAGGCAGC | 1385 | OPA1 H31 | CGATTTGTCACTTTAGT |
| | 3UTR(+163+179) | | | 3UTR(+217+233) | |
| 1368 | OPA1 H31 | TCTTCCATTCGAGAGGC | 1386 | OPA1 H31 | TTCCGATTTGTCACTTT |
| | 3UTR(+166+182) | | | 3UTR(+220+236) | |
| 1369 | OPA1 H31 | TGTTCTTCCATTCGAGA | 1387 | OPA1 H31 | TTATTCCGATTTGTCAC |
| | 3UTR(+169+185) | | | 3UTR(+223+239) | |
| 1370 | OPA1 H31 | CACTGTTCTTCCATTCG | 1388 | OPA1 H31 | ATATTATTCCGATTTGT |
| | 3UTR(+172+188) | | | 3UTR(+226+242) | |
| 1371 | OPA1 H31 | TACCACTGTTCTTCCAT | 1389 | OPA1 H31 | ATTATATTATTCCGATT |
| | 3UTR(+175+191) | | | 3UTR(+229+245) | |
| 1372 | OPA1 H31 | CATTACCACTGTTCTTC | 1390 | OPA1 H31 | CCAATTATATTATTCCG |
| | 3UTR(+178+194) | | | 3UTR(+232+248) | |
| 1373 | OPA1 H31 | ATCCATTACCACTGTTC | 1391 | OPA1 H31 | ATACCAATTATATTATT |
| | 3UTR(+181+197) | | | 3UTR(+235+251) | |
| 1374 | OPA1 H31 | TTAATCCATTACCACTG | 1392 | OPA1 H31 | GCCATACCAATTATATT |
| | 3UTR(+184+200) | | | 3UTR(+238+254) | |
| 1375 | OPA1 H31 | ATGTTAATCCATTACCA | 1393 | OPA1 H31 | ATGGCCATACCAATTAT |
| | 3UTR(+187+203) | | | 3UTR(+241+257) | |
| 1376 | OPA1 H31 | AGGATGTTAATCCATTA | 1394 | OPA1 H31 | CTAATGGCCATACCAAT |
| | 3UTR(+190+206) | | | 3UTR(+244+260) | |
| 1377 | OPA1 H31 | AATAGGATGTTAATCCA | 1395 | OPA1 H31 | AACCTAATGGCCATACC |
| | 3UTR(+193+209) | | | 3UTR(+247+263) | |
| 1378 | OPA1 H31 | CAAAATAGGATGTTAAT | 1396 | OPA1 H31 | CTGAACCTAATGGCCAT |
| | 3UTR(+196+212) | | | 3UTR(+250+266) | |
| 1397 | OPA1 H31 | GGACTGAACCTAATGGC | 1415 | OPA1 H31 | ACGAGTGCCACCACAAA |
| | 3UTR(+253+269) | | | 3UTR(+307+323) | |
| 1398 | OPA1 H31 | CAAGGACTGAACCTAAT | 1416 | OPA1 H31 | TAAACGAGTGCCACCAC |
| | 3UTR(+256+272) | | | 3UTR(+310+326) | |
| 1399 | OPA1 H31 | CTTCAAGGACTGAACCT | 1417 | OPA1 H31 | CATTAAACGAGTGCCAC |
| | 3UTR(+259+275) | | | 3UTR(+313+329) | |
| 1400 | OPA1 H31 | TATCTTCAAGGACTGAA | 1418 | OPA1 H31 | ATCCATTAAACGAGTGC |
| | 3UTR(+262+278) | | | 3UTR(+316+332) | |
| 1401 | OPA1 H31 | TCTTATCTTCAAGGACT | 1419 | OPA1 H31 | TTAATCCATTAAACGAG |
| | 3UTR(+265+281) | | | 3UTR(+319+335) | |
| 1402 | OPA1 H31 | GTTTCTTATCTTCAAGG | 1420 | OPA1 H31 | CAGTTAATCCATTAAAC |
| | 3UTR(+268+284) | | | 3UTR(+322+338) | |
| 1403 | OPA1 H31 | CAAGTTTCTTATCTTCA | 1421 | OPA1 H31 | CCTCAGTTAATCCATTA |
| | 3UTR(+271+287) | | | 3UTR(+325+341) | |
| 1404 | OPA1 H31 | GAACAAGTTTCTTATCT | 1422 | OPA1 H31 | CAACCTCAGTTAATCCA |
| | 3UTR(+274+290) | | | 3UTR(+328+344) | |
| 1405 | OPA1 H31 | AGAGAACAAGTTTCTTA | 1423 | OPA1 H31 | GAGCAACCTCAGTTAAT |
| | 3UTR(+277+293) | | | 3UTR(+331+347) | |
| 1406 | OPA1 H31 | AACAGAGAACAAGTTTC | 1424 | OPA1 H31 | ATTGAGCAACCTCAGTT |
| | 3UTR(+280+296) | | | 3UTR(+334+350) | |
| 1407 | OPA1 H31 | ACAAACAGAGAACAAGT | 1425 | OPA1 H31 | AACATTGAGCAACCTCA |
| | 3UTR(+283+299) | | | 3UTR(+337+353) | |
| 1408 | OPA1 H31 | ACAACAAACAGAGAACA | 1426 | OPA1 H31 | CTGAACATTGAGCAACC |
| | 3UTR(+286+302) | | | 3UTR(+340+356) | |
| 1409 | OPA1 H31 | AAGACAACAAACAGAGA | 1427 | OPA1 H31 | AAACTGAACATTGAGCA |
| | 3UTR(+289+305) | | | 3UTR(+343+359) | |
| 1410 | OPA1 H31 | AATAAGACAACAAACAG | 1428 | OPA1 H31 | AAGAAACTGAACATTGA |
| | 3UTR(+292+308) | | | 3UTR(+346+362) | |
| 1411 | OPA1 H31 | ACAAATAAGACAACAAA | 1429 | OPA1 H31 | GAAAAGAAACTGAACAT |
| | 3UTR(+295+311) | | | 3UTR(+349+365) | |
| 1412 | OPA1 H31 | ACCACAAATAAGACAAC | 1430 | OPA1 H31 | CTGGAAAAGAAACTGAA |
| | 3UTR(+298+314) | | | 3UTR(+352+368) | |
| 1413 | OPA1 H31 | GCCACCACAAATAAGAC | 1431 | OPA1 H31 | TTTCTGGAAAAGAAACT |
| | 3UTR(+301+317) | | | 3UTR(+355+371) | |
| 1414 | OPA1 H31 | AGTGCCACCACAAATAA | 1432 | OPA1 H31 | GTATTTCTGGAAAAGAA |
| | 3UTR(+304+320) | | | 3UTR(+358+374) | |
| 1433 | OPA1 H31 | ATTGTATTTCTGGAAAA | 1451 | OPA1 H31 | ACAATTGATAACTTTAA |
| | 3UTR(+361+377) | | | 3UTR(+415+431) | |
| 1434 | OPA1 H31 | AGCATTGTATTTCTGGA | 1452 | OPA1 H31 | TATACAATTGATAACTT |
| | 3UTR(+364+380) | | | 3UTR(+418+434) | |
| 1435 | OPA1 H31 | CCTAGCATTGTATTTCT | 1453 | OPA1 H31 | TTATATACAATTGATAA |
| | 3UTR(+367+383) | | | 3UTR(+421+437) | |
| 1436 | OPA1 H31 | ACACCTAGCATTGTATT | 1454 | OPA1 H31 | ATTTTATATACAATTGA |
| | 3UTR(+370+386) | | | 3UTR(+424+440) | |
| 1437 | OPA1 H31 | AAAACACCTAGCATTGT | 1455 | OPA1 H31 | GTGATTTTATATACAAT |
| | 3UTR(+373+389) | | | 3UTR(+427+443) | |
| 1438 | OPA1 H31 | TTCAAAACACCTAGCAT | 1456 | OPA1 H31 | ACTGTGATTTTATATAC |
| | 3UTR(+376+392) | | | 3UTR(+430+446) | |
| 1439 | OPA1 H31 | TATTTCAAAACACCTAG | 1457 | OPA1 H31 | GCTACTGTGATTTTATA |
| | 3UTR(+379+395) | | | 3UTR(+433+449) | |
| 1440 | OPA1 H31 | TTTTATTTCAAAACACC | 1458 | OPA1 H31 | CAGGCTACTGTGATTTT |
| | 3UTR(+382+398) | | | 3UTR(+436+452) | |
| 1441 | OPA1 H31 | AAGTTTTATTTCAAAAC | 1459 | OPA1 H31 | TAGCAGGCTACTGTGAT |
| | 3UTR(+385+401) | | | 3UTR(+439+455) | |
| 1442 | OPA1 H31 | TATAAGTTTTATTTCAA | 1460 | OPA1 H31 | ATTTAGCAGGCTACTGT |
| | 3UTR(+388+404) | | | 3UTR(+442+458) | |
| 1443 | OPA1 H31 | CTATATAAGTTTTATTT | 1461 | OPA1 H31 | ATGATTTAGCAGGCTAC |
| | 3UTR(+391+407) | | | 3UTR(+445+461) | |
| 1444 | OPA1 H31 | TTGCTATATAAGTTTTA | 1462 | OPA1 H31 | ACAATGATTTAGCAGGC |
| | 3UTR(+394+410) | | | 3UTR(+448+464) | |
| 1445 | OPA1 H31 | CAATTGCTATATAAGTT | 1463 | OPA1 H31 | CATACAATGATTTAGCA |
| | 3UTR(+397+413) | | | 3UTR(+451+467) | |
| 1446 | OPA1 H31 | AAACAATTGCTATATAA | 1464 | OPA1 H31 | ACACATACAATGATTTA |
| | 3UTR(+400+416) | | | 3UTR(+454+470) | |
| 1447 | OPA1 H31 | TTTAAACAATTGCTATA | 1465 | OPA1 H31 | CAGACACATACAATGAT |
| | 3UTR(+403+419) | | | 3UTR(+457+473) | |
| 1448 | OPA1 H31 | AACTTTAAACAATTGCT | 1466 | OPA1 H31 | CTACAGACACATACAAT |
| | 3UTR(+406+422) | | | 3UTR(+460+476) | |
| 1449 | OPA1 H31 | GATAACTTTAAACAATT | 1467 | OPA1 H31 | ATACTACAGACACATAC |
| | 3UTR(+409+425) | | | 3UTR(+463+479) | |
| 1450 | OPA1 H31 | ATTGATAACTTTAAACA | 1468 | OPA1 H31 | AGAATACTACAGACACA |
| | 3UTR(+412+428) | | | 3UTR(+466+482) | |
| 1469 | OPA1 H31 | AATAGAATACTACAGAC | 1487 | OPA1 H31 | TTTCTTTCATGTGGTGA |
| | 3UTR(+469+485) | | | 3UTR(+523+539) | |
| 1470 | OPA1 H31 | GGGAATAGAATACTACA | 1488 | OPA1 H31 | ATTTTTCTTTCATGTGG |
| | 3UTR(+472+488) | | | 3UTR(+526+542) | |
| 1471 | OPA1 H31 | TCTGGGAATAGAATACT | 1489 | OPA1 H31 | CCCATTTTTCTTTCATG |
| | 3UTR(+475+491) | | | 3UTR(+529+545) | |
| 1472 | OPA1 H31 | GTTTCTGGGAATAGAAT | 1490 | OPA1 H31 | TTACCCATTTTTCTTTC |
| | 3UTR(+478+494) | | | 3UTR(+532+548) | |
| 1473 | OPA1 H31 | ATAGTTTCTGGGAATAG | 1491 | OPA1 H31 | CTGTTACCCATTTTTCT |
| | 3UTR(+481+497) | | | 3UTR(+535+551) | |
| 1474 | OPA1 H31 | CAAATAGTTTCTGGGAA | 1492 | OPA1 H31 | CTTCTGTTACCCATTTT |
| | 3UTR(+484+500) | | | 3UTR(+538+554) | |
| 1475 | OPA1 H31 | GGTCAAATAGTTTCTGG | 1493 | OPA1 H31 | GTTCTTCTGTTACCCAT |
| | 3UTR(+487+503) | | | 3UTR(+541+557) | |
| 1476 | OPA1 H31 | CATGGTCAAATAGTTTC | 1494 | OPA1 H31 | AGGGTTCTTCTGTTACC |
| | 3UTR(+490+506) | | | 3UTR(+544+560) | |
| 1477 | OPA1 H31 | TATCATGGTCAAATAGT | 1495 | OPA1 H31 | TTAAGGGTTCTTCTGTT |
| | 3UTR(+493+509) | | | 3UTR(+547+563) | |
| 1478 | OPA1 H31 | AATTATCATGGTCAAAT | 1496 | OPA1 H31 | GTTTTAAGGGTTCTTCT |
| | 3UTR(+496+512) | | | 3UTR(+550+566) | |
| 1479 | OPA1 H31 | CTGAATTATCATGGTCA | 1497 | OPA1 H31 | CCTGTTTTAAGGGTTCT |
| | 3UTR(+499+515) | | | 3UTR(+553+569) | |
| 1480 | OPA1 H31 | AAACTGAATTATCATGG | 1498 | OPA1 H31 | TAACCTGTTTTAAGGGT |
| | 3UTR(+502+518) | | | 3UTR(+556+572) | |
| 1481 | OPA1 H31 | TATAAACTGAATTATCA | 1499 | OPA1 H31 | AATTAACCTGTTTTAAG |
| | 3UTR(+505+521) | | | 3UTR(+559+575) | |
| 1482 | OPA1 H31 | GAATATAAACTGAATTA | 1500 | OPA1 H31 | CCAAATTAACCTGTTTT |
| | 3UTR(+508+524) | | | 3UTR(+562+578) | |
| 1483 | OPA1 H31 | GGTGAATATAAACTGAA | 1501 | OPA1 H31 | AATCCAAATTAACCTGT |
| | 3UTR(+511+527) | | | 3UTR(+565+581) | |
| 1484 | OPA1 H31 | TGTGGTGAATATAAACT | 1502 | OPA1 H31 | TACAATCCAAATTAACC |
| | 3UTR(+514+530) | | | 3UTR(+568+584) | |
| 1485 | OPA1 H31 | TCATGTGGTGAATATAA | 1503 | OPA1 H31 | CGTTACAATCCAAATTA |
| | 3UTR(+517+533) | | | 3UTR(+571+587) | |
| 1486 | OPA1 H31 | CTTTCATGTGGTGAATA | 1504 | OPA1 H31 | GAACGTTACAATCCAAA |
| | 3UTR(+520+536) | | | 3UTR(+574+590) | |
| 1505 | OPA1 H31 | ACTGAACGTTACAATCC | 1523 | OPA1 H31 | CTTGGCTTCCAAGGCAA |
| | 3UTR(+577+593) | | | 3UTR(+631+647) | |
| 1506 | OPA1 H31 | TTCACTGAACGTTACAA | 1524 | OPA1 H31 | TGACTTGGCTTCCAAGG |
| | 3UTR(+580+596) | | | 3UTR(+634+650) | |
| 1507 | OPA1 H31 | TCTTTCACTGAACGTTA | 1525 | OPA1 H31 | TACTGACTTGGCTTCCA |
| | 3UTR(+583+599) | | | 3UTR(+637+653) | |
| 1508 | OPA1 H31 | ATTTCTTTCACTGAACG | 1526 | OPA1 H31 | TGGTACTGACTTGGCTT |
| | 3UTR(+586+602) | | | 3UTR(+640+656) | |
| 1509 | OPA1 H31 | GAAATTTCTTTCACTGA | 1527 | OPA1 H31 | AGCTGGTACTGACTTGG |
| | 3UTR(+589+605) | | | 3UTR(+643+659) | |
| 1510 | OPA1 H31 | GTTGAAATTTCTTTCAC | 1528 | OPA1 H31 | GTAAGCTGGTACTGACT |
| | 3UTR(+592+608) | | | 3UTR(+646+662) | |
| 1511 | OPA1 H31 | AGGGTTGAAATTTCTTT | 1529 | OPA1 H31 | TAGGTAAGCTGGTACTG |
| | 3UTR(+595+611) | | | 3UTR(+649+665) | |
| 1512 | OPA1 H31 | TGAAGGGTTGAAATTTC | 1530 | OPA1 H31 | AAATAGGTAAGCTGGTA |
| | 3UTR(+598+614) | | | 3UTR(+652+668) | |
| 1513 | OPA1 H31 | CTATGAAGGGTTGAAAT | 1531 | OPA1 H31 | ATCAAATAGGTAAGCTG |
| | 3UTR(+601+617) | | | 3UTR(+655+671) | |
| 1514 | OPA1 H31 | TGGCTATGAAGGGTTGA | 1532 | OPA1 H31 | TGAATCAAATAGGTAAG |
| | 3UTR(+604+620) | | | 3UTR(+658+674) | |
| 1515 | OPA1 H31 | CGCTGGCTATGAAGGGT | 1533 | OPA1 H31 | AACTGAATCAAATAGGT |
| | 3UTR(+607+623) | | | 3UTR(+661+677) | |
| 1516 | OPA1 H31 | CTTCGCTGGCTATGAAG | 1534 | OPA1 H31 | AGCAACTGAATCAAATA |
| | 3UTR(+610+626) | | | 3UTR(+664+680) | |
| 1517 | OPA1 H31 | TTTCTTCGCTGGCTATG | 1535 | OPA1 H31 | AACAGCAACTGAATCAA |
| | 3UTR(+613+629) | | | 3UTR(+667+683) | |
| 1518 | OPA1 H31 | AAATTTCTTCGCTGGCT | 1536 | OPA1 H31 | GAAAACAGCAACTGAAT |
| | 3UTR(+616+632) | | | 3UTR(+670+686) | |
| 1519 | OPA1 H31 | GGCAAATTTCTTCGCTG | 1537 | OPA1 H31 | TGAGAAAACAGCAACTG |
| | 3UTR(+619+635) | | | 3UTR(+673+689) | |
| 1520 | OPA1 H31 | CAAGGCAAATTTCTTCG | 1538 | OPA1 H31 | GAGTGAGAAAACAGCAA |
| | 3UTR(+622+638) | | | 3UTR(+676+692) | |
| 1521 | OPA1 H31 | TTCCAAGGCAAATTTCT | 1539 | OPA1 H31 | AGAGAGTGAGAAAACAG |
| | 3UTR(+625+641) | | | 3UTR(+679+695) | |
| 1522 | OPA1 H31 | GGCTTCCAAGGCAAATT | 1540 | OPA1 H31 | TATAGAGAGTGAGAAAA |
| | 3UTR(+628+644) | | | 3UTR(+682+698) | |
| 1541 | OPA1 H31 | GGATATAGAGAGTGAGA | 1559 | OPA1 H31 | TTAACAGAAAGCCTAAC |
| | 3UTR(+685+701) | | | 3UTR(+739+755) | |
| 1542 | OPA1 H31 | AATGGATATAGAGAGTG | 1560 | OPA1 H31 | CTATTAACAGAAAGCCT |
| | 3UTR(+688+704) | | | 3UTR(+742+758) | |
| 1543 | OPA1 H31 | TCAAATGGATATAGAGA | 1561 | OPA1 H31 | CCACTATTAACAGAAAG |
| | 3UTR(+691+707) | | | 3UTR(+745+761) | |
| 1544 | OPA1 H31 | ATTTCAAATGGATATAG | 1562 | OPA1 H31 | AAACCACTATTAACAGA |
| | 3UTR(+694+710) | | | 3UTR(+748+764) | |
| 1545 | OPA1 H31 | TCAATTTCAAATGGATA | 1563 | OPA1 H31 | GAAAAACCACTATTAAC |
| | 3UTR(+697+713) | | | 3UTR(+751+767) | |
| 1546 | OPA1 H31 | AAATCAATTTCAAATGG | 1564 | OPA1 H31 | GGAGAAAAACCACTATT |
| | 3UTR(+700+716) | | | 3UTR(+754+770) | |
| 1547 | OPA1 H31 | AATAAATCAATTTCAAA | 1565 | OPA1 H31 | ACAGGAGAAAAACCACT |
| | 3UTR(+703+719) | | | 3UTR(+757+773) | |
| 1548 | OPA1 H31 | TAAAATAAATCAATTTC | 1566 | OPA1 H31 | TCAACAGGAGAAAAACC |
| | 3UTR(+706+722) | | | 3UTR(+760+776) | |
| 1549 | OPA1 H31 | ATCTAAAATAAATCAAT | 1567 | OPA1 H31 | CTGTCAACAGGAGAAAA |
| | 3UTR(+709+725) | | | 3UTR(+763+779) | |
| 1550 | OPA1 H31 | AACATCTAAAATAAATC | 1568 | OPA1 H31 | GCTCTGTCAACAGGAGA |
| | 3UTR(+712+728) | | | 3UTR(+766+782) | |
| 1551 | OPA1 H31 | TACAACATCTAAAATAA | 1569 | OPA1 H31 | GTGGCTCTGTCAACAGG |
| | 3UTR(+715+731) | | | 3UTR(+769+785) | |
| 1552 | OPA1 H31 | GTATACAACATCTAAAA | 1570 | OPA1 H31 | CCGGTGGCTCTGTCAAC |
| | 3UTR(+718+734) | | | 3UTR(+772+788) | |
| 1553 | OPA1 H31 | TAAGTATACAACATCTA | 1571 | OPA1 H31 | AATCCGGTGGCTCTGTC |
| | 3UTR(+721+737) | | | 3UTR(+775+791) | |
| 1554 | OPA1 H31 | ACGTAAGTATACAACAT | 1572 | OPA1 H31 | CATAATCCGGTGGCTCT |
| | 3UTR(+724+740) | | | 3UTR(+778+794) | |
| 1555 | OPA1 H31 | CTAACGTAAGTATACAA | 1573 | OPA1 H31 | TGTCATAATCCGGTGGC |
| | 3UTR(+727+743) | | | 3UTR(+781+797) | |
| 1556 | OPA1 H31 | AGCCTAACGTAAGTATA | 1574 | OPA1 H31 | CTGTGTCATAATCCGGT |
| | 3UTR(+730+746) | | | 3UTR(+784+800) | |
| 1557 | OPA1 H31 | GAAAGCCTAACGTAAGT | 1575 | OPA1 H31 | ATCCTGTGTCATAATCC |
| | 3UTR(+733+749) | | | 3UTR(+787+803) | |
| 1558 | OPA1 H31 | ACAGAAAGCCTAACGTA | 1576 | OPA1 H31 | CTCATCCTGTGTCATAA |
| | 3UTR(+736+752) | | | 3UTR(+790+806) | |
| 1577 | OPA1 H31 | TTCCTCATCCTGTGTCA | 1595 | OPA1 H31 | TTGAAATGTTTGATAAT |
| | 3UTR(+793+809) | | | 3UTR(+847+863) | |
| 1578 | OPA1 H31 | ATCTTCCTCATCCTGTG | 1596 | OPA1 H31 | TAGTTGAAATGTTTGAT |
| | 3UTR(+796+812) | | | 3UTR(+850+866) | |
| 1579 | OPA1 H31 | TTAATCTTCCTCATCCT | 1597 | OPA1 H31 | ACCTAGTTGAAATGTTT |
| | 3UTR(+799+815) | | | 3UTR(+853+869) | |
| 1580 | OPA1 H31 | TCCTTAATCTTCCTCAT | 1598 | OPA1 H31 | GATACCTAGTTGAAATG |
| | 3UTR(+802+818) | | | 3UTR(+856+872) | |
| 1581 | OPA1 H31 | TTATCCTTAATCTTCCT | 1599 | OPA1 H31 | TCTGATACCTAGTTGAA |
| | 3UTR(+805+821) | | | 3UTR(+859+875) | |
| 1582 | OPA1 H31 | TGATTATCCTTAATCTT | 1600 | OPA1 H31 | TTTTCTGATACCTAGTT |
| | 3UTR(+808+824) | | | 3UTR(+862+878) | |
| 1583 | OPA1 H31 | AATTGATTATCCTTAAT | 1601 | OPA1 H31 | CCTTTTTCTGATACCTA |
| | 3UTR(+811+827) | | | 3UTR(+865+881) | |
| 1584 | OPA1 H31 | GTCAATTGATTATCCTT | 1602 | OPA1 H31 | AAGCCTTTTTCTGATAC |
| | 3UTR(+814+830) | | | 3UTR(+868+884) | |
| 1585 | OPA1 H31 | TTAGTCAATTGATTATC | 1603 | OPA1 H31 | AGAAAGCCTTTTTCTGA |
| | 3UTR(+817+833) | | | 3UTR(+871+887) | |
| 1586 | OPA1 H31 | AAATTAGTCAATTGATT | 1604 | OPA1 H31 | GAAAGAAAGCCTTTTTC |
| | 3UTR(+820+836) | | | 3UTR(+874+890) | |
| 1587 | OPA1 H31 | ATGAAATTAGTCAATTG | 1605 | OPA1 H31 | TATGAAAGAAAGCCTTT |
| | 3UTR(+823+839) | | | 3UTR(+877+893) | |
| 1588 | OPA1 H31 | TAAATGAAATTAGTCAA | 1606 | OPA1 H31 | TCTTATGAAAGAAAGCC |
| | 3UTR(+826+842) | | | 3UTR(+880+896) | |
| 1589 | OPA1 H31 | TTCTAAATGAAATTAGT | 1607 | OPA1 H31 | TAGTCTTATGAAAGAAA |
| | 3UTR(+829+845) | | | 3UTR(+883+899) | |
| 1590 | OPA1 H31 | ATATTCTAAATGAAATT | 1608 | OPA1 H31 | AAATAGTCTTATGAAAG |
| | 3UTR(+832+848) | | | 3UTR(+886+902) | |
| 1591 | OPA1 H31 | ATAATATTCTAAATGAA | 1609 | OPA1 H31 | TTAAAATAGTCTTATGA |
| | 3UTR(+835+851) | | | 3UTR(+889+905) | |
| 1592 | OPA1 H31 | TTGATAATATTCTAAAT | 1610 | OPA1 H31 | TATTTAAAATAGTCTTA |
| | 3UTR(+838+854) | | | 3UTR(+892+908) | |
| 1593 | OPA1 H31 | TGTTTGATAATATTCTA | 1611 | OPA1 H31 | TTCTATTTAAAATAGTC |
| | 3UTR(+841+857) | | | 3UTR(+895+911) | |
| 1594 | OPA1 H31 | AAATGTTTGATAATATT | 1612 | OPA1 H31 | AATTTCTATTTAAAATA |
| | 3UTR(+844+860) | | | 3UTR(+898+914) | |
| 1613 | OPA1 H31 | AATAATTTCTATTTAAA | 1631 | OPA1 H31 | AATTTTTCTTTATTCAG |
| | 3UTR(+901+917) | | | 3UTR(+955+971) | |
| 1614 | OPA1 H31 | TGAAATAATTTCTATTT | 1632 | OPA1 H31 | CTAAATTTTTCTTTATT |
| | 3UTR(+904+920) | | | 3UTR(+958+974) | |
| 1615 | OPA1 H31 | TGTTGAAATAATTTCTA | 1633 | OPA1 H31 | GAACTAAATTTTTCTTT |
| | 3UTR(+907+923) | | | 3UTR(+961+977) | |
| 1616 | OPA1 H31 | AATTGTTGAAATAATTT | 1634 | OPA1 H31 | ATTGAACTAAATTTTTC |
| | 3UTR(+910+926) | | | 3UTR(+964+980) | |
| 1617 | OPA1 H31 | TTTAATTGTTGAAATAA | 1635 | OPA1 H31 | TAAATTGAACTAAATTT |
| | 3UTR(+913+929) | | | 3UTR(+967+983) | |
| 1618 | OPA1 H31 | TACTTTAATTGTTGAAA | 1636 | OPA1 H31 | CAATAAATTGAACTAAA |
| | 3UTR(+916+932) | | | 3UTR(+970+986) | |
| 1619 | OPA1 H31 | CATTACTTTAATTGTTG | 1637 | OPA1 H31 | TTGCAATAAATTGAACT |
| | 3UTR(+919+935) | | | 3UTR(+973+989) | |
| 1620 | OPA1 H31 | CAACATTACTTTAATTG | 1638 | OPA1 H31 | AAATTGCAATAAATTGA |
| | 3UTR(+922+938) | | | 3UTR(+976+992) | |
| 1621 | OPA1 H31 | GGTCAACATTACTTTAA | 1639 | OPA1 H31 | ATTAAATTGCAATAAAT |
| | 3UTR(+925+941) | | | 3UTR(+979+995) | |
| 1622 | OPA1 H31 | GATGGTCAACATTACTT | 1640 | OPA1 H31 | GTAATTAAATTGCAATA |
| | 3UTR(+928+944) | | | 3UTR(+982+998) | |
| 1623 | OPA1 H31 | GGGGATGGTCAACATTA | 1641 | OPA1 H31 | ATTGTAATTAAATTGCA |
| | 3UTR(+931+947) | | | 3UTR(+985+1001) | |
| 1624 | OPA1 H31 | GAGGGGGATGGTCAACA | 1642 | OPA1 H31 | AGTATTGTAATTAAATT |
| | 3UTR(+934+950) | | | 3UTR(+988+1004) | |
| 1625 | OPA1 H31 | TGAGAGGGGGATGGTCA | 1643 | OPA1 H31 | GGTAGTATTGTAATTAA |
| | 3UTR(+937+953) | | | 3UTR(+991+1007) | |
| 1626 | OPA1 H31 | AGCTGAGAGGGGGATGG | 1644 | OPA1 H31 | GAAGGTAGTATTGTAAT |
| | 3UTR(+940+956) | | | 3UTR(+994+1010) | |
| 1627 | OPA1 H31 | TTCAGCTGAGAGGGGGA | 1645 | OPA1 H31 | TGTGAAGGTAGTATTGT |
| | 3UTR(+943+959) | | | 3UTR(+997+1013) | |
| 1628 | OPA1 H31 | TTATTCAGCTGAGAGGG | 1646 | OPA1 H31 | TGTTGTGAAGGTAGTAT |
| | 3UTR(+946+962) | | | 3UTR(+1000+1016) | |
| 1629 | OPA1 H31 | TCTTTATTCAGCTGAGA | 1647 | OPA1 H31 | AAATGTTGTGAAGGTAG |
| | 3UTR(+949+965) | | | 3UTR(+1003+1019) | |
| 1630 | OPA1 H31 | TTTTCTTTATTCAGCTG | 1648 | OPA1 H31 | TGAAAATGTTGTGAAGG |
| | 3UTR(+952+968) | | | 3UTR(+1006+1022) | |
| 1649 | OPA1 H31 | ACATGAAAATGTTGTGA | 1667 | OPA1 H31 | AGCATTTCTTTGCTTGA |
| | 3UTR(+1009+1025) | | | 3UTR(+1063+1079) | |
| 1650 | OPA1 H31 | AACACATGAAAATGTTG | 1668 | OPA1 H31 | GAAAGCATTTCTTTGCT |
| | 3UTR(+1012+1028) | | | 3UTR(+1066+1082) | |
| 1651 | OPA1 H31 | TAAAACACATGAAAATG | 1669 | OPA1 H31 | AAAGAAAGCATTTCTTT |
| | 3UTR(+1015+1031) | | | 3UTR(+1069+1085) | |
| 1652 | OPA1 H31 | ATTTAAAACACATGAAA | 1670 | OPA1 H31 | AGTAAAGAAAGCATTTC |
| | 3UTR(+1018+1034) | | | 3UTR(+1072+1088) | |
| 1653 | OPA1 H31 | TTTATTTAAAACACATG | 1671 | OPA1 H31 | TTAAGTAAAGAAAGCAT |
| | 3UTR(+1021+1037) | | | 3UTR(+1075+1091) | |
| 1654 | OPA1 H31 | ATATTTATTTAAAACAC | 1672 | OPA1 H31 | ATTTTAAGTAAAGAAAG |
| | 3UTR(+1024+1040) | | | 3UTR(+1078+1094) | |
| 1655 | OPA1 H31 | AAAATATTTATTTAAAA | 1673 | OPA1 H31 | GACATTTTAAGTAAAGA |
| | 3UTR(+1027+1043) | | | 3UTR(+1081+1097) | |
| 1656 | OPA1 H31 | TAAAAAATATTTATTTA | 1674 | OPA1 H31 | ATAGACATTTTAAGTAA |
| | 3UTR(+1030+1046) | | | 3UTR(+1084+1100) | |
| 1657 | OPA1 H31 | AATTAAAAAATATTTAT | 1675 | OPA1 H31 | GAGATAGACATTTTAAG |
| | 3UTR(+1033+1049) | | | 3UTR(+1087+1103) | |
| 1658 | OPA1 H31 | GCCAATTAAAAAATATT | 1676 | OPA1 H31 | AATGAGATAGACATTTT |
| | 3UTR(+1036+1052) | | | 3UTR(+1090+1106) | |
| 1659 | OPA1 H31 | TTAGCCAATTAAAAAAT | 1677 | OPA1 H31 | GCAAATGAGATAGACAT |
| | 3UTR(+1039+1055) | | | 3UTR(+1093+1109) | |
| 1660 | OPA1 H31 | CCTTTAGCCAATTAAAA | 1678 | OPA1 H31 | GCAGCAAATGAGATAGA |
| | 3UTR(+1042+1058) | | | 3UTR(+1096+1112) | |
| 1661 | OPA1 H31 | TGTCCTTTAGCCAATTA | 1679 | OPA1 H31 | AAGGCAGCAAATGAGAT |
| | 3UTR(+1045+1061) | | | 3UTR(+1099+1115) | |
| 1662 | OPA1 H31 | GAATGTCCTTTAGCCAA | 1680 | OPA1 H31 | GAAAAGGCAGCAAATGA |
| | 3UTR(+1048+1064) | | | 3UTR(+1102+1118) | |
| 1663 | OPA1 H31 | CTTGAATGTCCTTTAGC | 1681 | OPA1 H31 | AGTGAAAAGGCAGCAAA |
| | 3UTR(+1051+1067) | | | 3UTR(+1105+1121) | |
| 1664 | OPA1 H31 | TTGCTTGAATGTCCTTT | 1682 | OPA1 H31 | CTTAGTGAAAAGGCAGC |
| | 3UTR(+1054+1070) | | | 3UTR(+1108+1124) | |
| 1665 | OPA1 H31 | TCTTTGCTTGAATGTCC | 1683 | OPA1 H31 | AGGCTTAGTGAAAAGGC |
| | 3UTR(+1057+1073) | | | 3UTR(+1111+1127) | |
| 1666 | OPA1 H31 | ATTTCTTTGCTTGAATG | 1684 | OPA1 H31 | TAAAGGCTTAGTGAAAA |
| | 3UTR(+1060+1076) | | | 3UTR(+1114+1130) | |
| 1685 | OPA1 H31 | AAGTAAAGGCTTAGTGA | 1703 | OPA1 H31 | GATTTAGCAAACTGTAA |
| | 3UTR(+1117+1133) | | | 3UTR(+1171+1187) | |
| 1686 | OPA1 H31 | ACAAAGTAAAGGCTTAG | 1704 | OPA1 H31 | TAAGATTTAGCAAACTG |
| | 3UTR(+1120+1136) | | | 3UTR(+1174+1190) | |
| 1687 | OPA1 H31 | TTAACAAAGTAAAGGCT | 1705 | OPA1 H31 | AAATAAGATTTAGCAAA |
| | 3UTR(+1123+1139) | | | 3UTR(+1177+1193) | |
| 1688 | OPA1 H31 | TTATTAACAAAGTAAAG | 1706 | OPA1 H31 | AGAAAATAAGATTTAGC |
| | 3UTR(+1126+1142) | | | 3UTR(+1180+1196) | |
| 1689 | OPA1 H31 | CTTTTATTAACAAAGTA | 1707 | OPA1 H31 | CCAAGAAAATAAGATTT |
| | 3UTR(+1129+1145) | | | 3UTR(+1183+1199) | |
| 1690 | OPA1 H31 | ACACTTTTATTAACAAA | 1708 | OPA1 H31 | ACTCCAAGAAAATAAGA |
| | 3UTR(+1132+1148) | | | 3UTR(+1186+1202) | |
| 1691 | OPA1 H31 | TGGACACTTTTATTAAC | 1709 | OPA1 H31 | GCAACTCCAAGAAAATA |
| | 3UTR(+1135+1151) | | | 3UTR(+1189+1205) | |
| 1692 | OPA1 H31 | CAATGGACACTTTTATT | 1710 | OPA1 H31 | AAAGCAACTCCAAGAAA |
| | 3UTR(+1138+1154) | | | 3UTR(+1192+1208) | |
| 1693 | OPA1 H31 | ACACAATGGACACTTTT | 1711 | OPA1 H31 | CAAAAAGCAACTCCAAG |
| | 3UTR(+1141+1157) | | | 3UTR(+1195+1211) | |
| 1694 | OPA1 H31 | ATCACACAATGGACACT | 1712 | OPA1 H31 | TACCAAAAAGCAACTCC |
| | 3UTR(+1144+1160) | | | 3UTR(+1198+1214) | |
| 1695 | OPA1 H31 | AACATCACACAATGGAC | 1713 | OPA1 H31 | TGTTACCAAAAAGCAAC |
| | 3UTR(+1147+1163) | | | 3UTR(+1201+1217) | |
| 1696 | OPA1 H31 | AAAAACATCACACAATG | 1714 | OPA1 H31 | GGCTGTTACCAAAAAGC |
| | 3UTR(+1150+1166) | | | 3UTR(+1204+1220) | |
| 1697 | OPA1 H31 | ATCAAAAACATCACACA | 1715 | OPA1 H31 | TGGGGCTGTTACCAAAA |
| | 3UTR(+1153+1169) | | | 3UTR(+1207+1223) | |
| 1698 | OPA1 H31 | AAAATCAAAAACATCAC | 1716 | OPA1 H31 | CAATGGGGCTGTTACCA |
| | 3UTR(+1156+1172) | | | 3UTR(+1210+1226) | |
| 1699 | OPA1 H31 | TGTAAAATCAAAAACAT | 1717 | OPA1 H31 | TAGCAATGGGGCTGTTA |
| | 3UTR(+1159+1175) | | | 3UTR(+1213+1229) | |
| 1700 | OPA1 H31 | AACTGTAAAATCAAAAA | 1718 | OPA1 H31 | GAGTAGCAATGGGGCTG |
| | 3UTR(+1162+1178) | | | 3UTR(+1216+1232) | |
| 1701 | OPA1 H31 | GCAAACTGTAAAATCAA | 1719 | OPA1 H31 | GGGGAGTAGCAATGGGG |
| | 3UTR(+1165+1181) | | | 3UTR(+1219+1235) | |
| 1702 | OPA1 H31 | TTAGCAAACTGTAAAAT | 1720 | OPA1 H31 | AATGGGGAGTAGCAATG |
| | 3UTR(+1168+1184) | | | 3UTR(+1222+1238) | |
| 1721 | OPA1 H31 | TAAAATGGGGAGTAGCA | 1739 | OPA1 H31 | ACCAAGTGTTACATCTT |
| | 3UTR(+1225+1241) | | | 3UTR(+1279+1295) | |
| 1722 | OPA1 H31 | CAATAAAATGGGGAGTA | 1740 | OPA1 H31 | CATACCAAGTGTTACAT |
| | 3UTR(+1228+1244) | | | 3UTR(+1282+1298) | |
| 1723 | OPA1 H31 | AAACAATAAAATGGGGA | 1741 | OPA1 H31 | GAGCATACCAAGTGTTA |
| | 3UTR(+1231+1247) | | | 3UTR(+1285+1301) | |
| 1724 | OPA1 H31 | GTAAAACAATAAAATGG | 1742 | OPA1 H31 | ACCGAGCATACCAAGTG |
| | 3UTR(+1234+1250) | | | 3UTR(+1288+1304) | |
| 1725 | OPA1 H31 | GATGTAAAACAATAAAA | 1743 | OPA1 H31 | TCAACCGAGCATACCAA |
| | 3UTR(+1237+1253) | | | 3UTR(+1291+1307) | |
| 1726 | OPA1 H31 | ATTGATGTAAAACAATA | 1744 | OPA1 H31 | TCCTCAACCGAGCATAC |
| | 3UTR(+1240+1256) | | | 3UTR(+1294+1310) | |
| 1727 | OPA1 H31 | TGCATTGATGTAAAACA | 1745 | OPA1 H31 | ATATCCTCAACCGAGCA |
| | 3UTR(+1243+1259) | | | 3UTR(+1297+1313) | |
| 1728 | OPA1 H31 | GCATGCATTGATGTAAA | 1746 | OPA1 H31 | TTCATATCCTCAACCGA |
| | 3UTR(+1246+1262) | | | 3UTR(+1300+1316) | |
| 1729 | OPA1 H31 | GAAGCATGCATTGATGT | 1747 | OPA1 H31 | TTTTTCATATCCTCAAC |
| | 3UTR(+1249+1265) | | | 3UTR(+1303+1319) | |
| 1730 | OPA1 H31 | AACGAAGCATGCATTGA | 1748 | OPA1 H31 | TATTTTTTCATATCCTC |
| | 3UTR(+1252+1268) | | | 3UTR(+1306+1322) | |
| 1731 | OPA1 H31 | CACAACGAAGCATGCAT | 1749 | OPA1 H31 | AAGTATTTTTTCATATC |
| | 3UTR(+1255+1271) | | | 3UTR(+1309+1325) | |
| 1732 | OPA1 H31 | GATCACAACGAAGCATG | 1750 | OPA1 H31 | CGGAAGTATTTTTTCAT |
| | 3UTR(+1258+1274) | | | 3UTR(+1312+1328) | |
| 1733 | OPA1 H31 | AGGGATCACAACGAAGC | 1751 | OPA1 H31 | TTTCGGAAGTATTTTTT |
| | 3UTR(+1261+1277) | | | 3UTR(+1315+1331) | |
| 1734 | OPA1 H31 | TTGAGGGATCACAACGA | 1752 | OPA1 H31 | TGGTTTCGGAAGTATTT |
| | 3UTR(+1264+1280) | | | 3UTR(+1318+1334) | |
| 1735 | OPA1 H31 | ATCTTGAGGGATCACAA | 1753 | OPA1 H31 | TCCTGGTTTCGGAAGTA |
| | 3UTR(+1267+1283) | | | 3UTR(+1321+1337) | |
| 1736 | OPA1 H31 | TACATCTTGAGGGATCA | 1754 | OPA1 H31 | AATTCCTGGTTTCGGAA |
| | 3UTR(+1270+1286) | | | 3UTR(+1324+1340) | |
| 1737 | OPA1 H31 | TGTTACATCTTGAGGGA | 1755 | OPA1 H31 | TTGAATTCCTGGTTTCG |
| | 3UTR(+1273+1289) | | | 3UTR(+1327+1343) | |
| 1738 | OPA1 H31 | AAGTGTTACATCTTGAG | 1756 | OPA1 H31 | ACATTGAATTCCTGGTT |
| | 3UTR(+1276+1292) | | | 3UTR(+1330+1346) | |
| 1757 | OPA1 H31 | CATACATTGAATTCCTG | 1775 | OPA1 H31 | ATCTGTGCTGCTTAAGG |
| | 3UTR(+1333+1349) | | | 3UTR(+1387+1403) | |
| 1758 | OPA1 H31 | AAACATACATTGAATTC | 1776 | OPA1 H31 | CGCATCTGTGCTGCTTA |
| | 3UTR(+1336+1352) | | | 3UTR(+1390+1406) | |
| 1759 | OPA1 H31 | AACAAACATACATTGAA | 1777 | OPA1 H31 | CAGCGCATCTGTGCTGC |
| | 3UTR(+1339+1355) | | | 3UTR(+1393+1409) | |
| 1760 | OPA1 H31 | TAAAACAAACATACATT | 1778 | OPA1 H31 | TACCAGCGCATCTGTGC |
| | 3UTR(+1342+1358) | | | 3UTR(+1396+1412) | |
| 1761 | OPA1 H31 | GTATAAAACAAACATAC | 1779 | OPA1 H31 | ATCTACCAGCGCATCTG |
| | 3UTR(+1345+1361) | | | 3UTR(+1399+1415) | |
| 1762 | OPA1 H31 | ACAGTATAAAACAAACA | 1780 | OPA1 H31 | TGCATCTACCAGCGCAT |
| | 3UTR(+1348+1364) | | | 3UTR(+1402+1418) | |
| 1763 | OPA1 H31 | CAAACAGTATAAAACAA | 1781 | OPA1 H31 | CTATGCATCTACCAGCG |
| | 3UTR(+1351+1367) | | | 3UTR(+1405+1421) | |
| 1764 | OPA1 H31 | TATCAAACAGTATAAAA | 1782 | OPA1 H31 | TGACTATGCATCTACCA |
| | 3UTR(+1354+1370) | | | 3UTR(+1408+1424) | |
| 1765 | OPA1 H31 | TCTTATCAAACAGTATA | 1783 | OPA1 H31 | TCCTGACTATGCATCTA |
| | 3UTR(+1357+1373) | | | 3UTR(+1411+1427) | |
| 1766 | OPA1 H31 | TTTTCTTATCAAACAGT | 1784 | OPA1 H31 | AGTTCCTGACTATGCAT |
| | 3UTR(+1360+1376) | | | 3UTR(+1414+1430) | |
| 1767 | OPA1 H31 | TACTTTTCTTATCAAAC | 1785 | OPA1 H31 | AAAAGTTCCTGACTATG |
| | 3UTR(+1363+1379) | | | 3UTR(+1417+1433) | |
| 1768 | OPA1 H31 | ACCTACTTTTCTTATCA | 1786 | OPA1 H31 | AAAAAAAGTTCCTGACT |
| | 3UTR(+1366+1382) | | | 3UTR(+1420+1436) | |
| 1769 | OPA1 H31 | TGGACCTACTTTTCTTA | 1787 | OPA1 H31 | AATAAAAAAAGTTCCTG |
| | 3UTR(+1369+1385) | | | 3UTR(+1423+1439) | |
| 1770 | OPA1 H31 | GGCTGGACCTACTTTTC | 1788 | OPA1 H31 | AGAAATAAAAAAAGTTC |
| | 3UTR(+1372+1388) | | | 3UTR(+1426+1442) | |
| 1771 | OPA1 H31 | TAAGGCTGGACCTACTT | 1789 | OPA1 H31 | AAAAGAAATAAAAAAAG |
| | 3UTR(+1375+1391) | | | 3UTR(+1429+1445) | |
| 1772 | OPA1 H31 | GCTTAAGGCTGGACCTA | 1790 | OPA1 H31 | CCTAAAAGAAATAAAAA |
| | 3UTR(+1378+1394) | | | 3UTR(+1432+1448) | |
| 1773 | OPA1 H31 | GCTGCTTAAGGCTGGAC | 1791 | OPA1 H31 | AGACCTAAAAGAAATAA |
| | 3UTR(+1381+1397) | | | 3UTR(+1435+1451) | |
| 1774 | OPA1 H31 | TGTGCTGCTTAAGGCTG | 1792 | OPA1 H31 | CCTAGACCTAAAAGAAA |
| | 3UTR(+1384+1400) | | | 3UTR(+1438+1454) | |
| 1793 | OPA1 H31 | GTCCCTAGACCTAAAAG | 1811 | OPA1 H31 | TCCTAATCTGTGTATAG |
| | 3UTR(+1441+1457) | | | 3UTR(+1495+1511) | |
| 1794 | OPA1 H31 | CCTGTCCCTAGACCTAA | 1812 | OPA1 H31 | ATCTCCTAATCTGTGTA |
| | 3UTR(+1444+1460) | | | 3UTR(+1498+1514) | |
| 1795 | OPA1 H31 | ACTCCTGTCCCTAGACC | 1813 | OPA1 H31 | GTCATCTCCTAATCTGT |
| | 3UTR(+1447+1463) | | | 3UTR(+1501+1517) | |
| 1796 | OPA1 H31 | TTCACTCCTGTCCCTAG | 1814 | OPA1 H31 | AAGGTCATCTCCTAATC |
| | 3UTR(+1450+1466) | | | 3UTR(+1504+1520) | |
| 1797 | OPA1 H31 | CTATTCACTCCTGTCCC | 1815 | OPA1 H31 | AGTAAGGTCATCTCCTA |
| | 3UTR(+1453+1469) | | | 3UTR(+1507+1523) | |
| 1798 | OPA1 H31 | TTTCTATTCACTCCTGT | 1816 | OPA1 H31 | CCCAGTAAGGTCATCTC |
| | 3UTR(+1456+1472) | | | 3UTR(+1510+1526) | |
| 1799 | OPA1 H31 | CCCTTTCTATTCACTCC | 1817 | OPA1 H31 | GTACCCAGTAAGGTCAT |
| | 3UTR(+1459+1475) | | | 3UTR(+1513+1529) | |
| 1800 | OPA1 H31 | CCTCCCTTTCTATTCAC | 1818 | OPA1 H31 | GGTGTACCCAGTAAGGT |
| | 3UTR(+1462+1478) | | | 3UTR(+1516+1532) | |
| 1801 | OPA1 H31 | TCTCCTCCCTTTCTATT | 1819 | OPA1 H31 | AGGGGTGTACCCAGTAA |
| | 3UTR(+1465+1481) | | | 3UTR(+1519+1535) | |
| 1802 | OPA1 H31 | AGCTCTCCTCCCTTTCT | 1820 | OPA1 H31 | TAGAGGGGTGTACCCAG |
| | 3UTR(+1468+1484) | | | 3UTR(+1522+1538) | |
| 1803 | OPA1 H31 | TAGAGCTCTCCTCCCTT | 1821 | OPA1 H31 | GGTTAGAGGGGTGTACC |
| | 3UTR(+1471+1487) | | | 3UTR(+1525+1541) | |
| 1804 | OPA1 H31 | TAATAGAGCTCTCCTCC | 1822 | OPA1 H31 | ACTGGTTAGAGGGGTGT |
| | 3UTR(+1474+1490) | | | 3UTR(+1528+1544) | |
| 1805 | OPA1 H31 | ACATAATAGAGCTCTCC | 1823 | OPA1 H31 | AGCACTGGTTAGAGGGG |
| | 3UTR(+1477+1493) | | | 3UTR(+1531+1547) | |
| 1806 | OPA1 H31 | AGAACATAATAGAGCTC | 1824 | OPA1 H31 | GTAAGCACTGGTTAGAG |
| | 3UTR(+1480+1496) | | | 3UTR(+1534+1550) | |
| 1807 | OPA1 H31 | TATAGAACATAATAGAG | 1825 | OPA1 H31 | CCTGTAAGCACTGGTTA |
| | 3UTR(+1483+1499) | | | 3UTR(+1537+1553) | |
| 1808 | OPA1 H31 | GTGTATAGAACATAATA | 1826 | OPA1 H31 | TAACCTGTAAGCACTGG |
| | 3UTR(+1486+1502) | | | 3UTR(+1540+1556) | |
| 1809 | OPA1 H31 | TCTGTGTATAGAACATA | 1827 | OPA1 H31 | CATTAACCTGTAAGCAC |
| | 3UTR(+1489+1505) | | | 3UTR(+1543+1559) | |
| 1810 | OPA1 H31 | TAATCTGTGTATAGAAC | 1828 | OPA1 H31 | ATGCATTAACCTGTAAG |
| | 3UTR(+1492+1508) | | | 3UTR(+1546+1562) | |
| 1829 | OPA1 H31 | AACATGCATTAACCTGT | 1847 | OPA1 H31 | GAAATAGATGTGTAGTT |
| | 3UTR(+1549+1565) | | | 3UTR(+1603+1619) | |
| 1830 | OPA1 H31 | ATTAACATGCATTAACC | 1848 | OPA1 H31 | TTAGAAATAGATGTGTA |
| | 3UTR(+1552+1568) | | | 3UTR(+1606+1622) | |
| 1831 | OPA1 H31 | TTCATTAACATGCATTA | 1849 | OPA1 H31 | TCTTTAGAAATAGATGT |
| | 3UTR(+1555+1571) | | | 3UTR(+1609+1625) | |
| 1832 | OPA1 H31 | ATATTCATTAACATGCA | 1850 | OPA1 H31 | TATTCTTTAGAAATAGA |
| | 3UTR(+1558+1574) | | | 3UTR(+1612+1628) | |
| 1833 | OPA1 H31 | AAAATATTCATTAACAT | 1851 | OPA1 H31 | TTTTATTCTTTAGAAAT |
| | 3UTR(+1561+1577) | | | 3UTR(+1615+1631) | |
| 1834 | OPA1 H31 | GCAAAAATATTCATTAA | 1852 | OPA1 H31 | CTGTTTTATTCTTTAGA |
| | 3UTR(+1564+1580) | | | 3UTR(+1618+1634) | |
| 1835 | OPA1 H31 | ACTGCAAAAATATTCAT | 1853 | OPA1 H31 | GTCCTGTTTTATTCTTT |
| | 3UTR(+1567+1583) | | | 3UTR(+1621+1637) | |
| 1836 | OPA1 H31 | ACAACTGCAAAAATATT | 1854 | OPA1 H31 | ATGGTCCTGTTTTATTC |
| | 3UTR(+1570+1586) | | | 3UTR(+1624+1640) | |
| 1837 | OPA1 H31 | TTTACAACTGCAAAAAT | 1855 | OPA1 H31 | AATATGGTCCTGTTTTA |
| | 3UTR(+1573+1589) | | | 3UTR(+1627+1643) | |
| 1838 | OPA1 H31 | TGCTTTACAACTGCAAA | 1856 | OPA1 H31 | ATAAATATGGTCCTGTT |
| | 3UTR(+1576+1592) | | | 3UTR(+1630+1646) | |
| 1839 | OPA1 H31 | TTATGCTTTACAACTGC | 1857 | OPA1 H31 | TAAATAAATATGGTCCT |
| | 3UTR(+1579+1595) | | | 3UTR(+1633+1649) | |
| 1840 | OPA1 H31 | TTGTTATGCTTTACAAC | 1858 | OPA1 H31 | AAGTAAATAAATATGGT |
| | 3UTR(+1582+1598) | | | 3UTR(+1636+1652) | |
| 1841 | OPA1 H31 | TAATTGTTATGCTTTAC | 1859 | OPA1 H31 | CAGAAGTAAATAAATAT |
| | 3UTR(+1585+1601) | | | 3UTR(+1639+1655) | |
| 1842 | OPA1 H31 | TTGTAATTGTTATGCTT | 1860 | OPA1 H31 | TGACAGAAGTAAATAAA |
| | 3UTR(+1588+1604) | | | 3UTR(+1642+1658) | |
| 1843 | OPA1 H31 | TAGTTGTAATTGTTATG | 1861 | OPA1 H31 | AGTTGACAGAAGTAAAT |
| | 3UTR(+1591+1607) | | | 3UTR(+1645+1661) | |
| 1844 | OPA1 H31 | GTGTAGTTGTAATTGTT | 1862 | OPA1 H31 | TATAGTTGACAGAAGTA |
| | 3UTR(+1594+1610) | | | 3UTR(+1648+1664) | |
| 1845 | OPA1 H31 | GATGTGTAGTTGTAATT | 1863 | OPA1 H31 | TTCTATAGTTGACAGAA |
| | 3UTR(+1597+1613) | | | 3UTR(+1651+1667) | |
| 1846 | OPA1 H31 | ATAGATGTGTAGTTGTA | 1864 | OPA1 H31 | TCTTTCTATAGTTGACA |
| | 3UTR(+1600+1616) | | | 3UTR(+1654+1670) | |
| 1865 | OPA1 H31 | CTTTCTTTCTATAGTTG | 1883 | OPA1 H31 | TGACTAATATTAGCCTT |
| | 3UTR(+1657+1673) | | | 3UTR(+1711+1727) | |
| 1866 | OPA1 H31 | GGTCTTTCTTTCTATAG | 1884 | OPA1 H31 | TAGTGACTAATATTAGC |
| | 3UTR(+1660+1676) | | | 3UTR(+1714+1730) | |
| 1867 | OPA1 H31 | GAAGGTCTTTCTTTCTA | 1885 | OPA1 H31 | CAGTAGTGACTAATATT |
| | 3UTR(+1663+1679) | | | 3UTR(+1717+1733) | |
| 1868 | OPA1 H31 | GCTGAAGGTCTTTCTTT | 1886 | OPA1 H31 | TAACAGTAGTGACTAAT |
| | 3UTR(+1666+1682) | | | 3UTR(+1720+1736) | |
| 1869 | OPA1 H31 | ACAGCTGAAGGTCTTTC | 1887 | OPA1 H31 | TGATAACAGTAGTGACT |
| | 3UTR(+1669+1685) | | | 3UTR(+1723+1739) | |
| 1870 | OPA1 H31 | AATACAGCTGAAGGTCT | 1888 | OPA1 H31 | ATGTGATAACAGTAGTG |
| | 3UTR(+1672+1688) | | | 3UTR(+1726+1742) | |
| 1871 | OPA1 H31 | GGAAATACAGCTGAAGG | 1889 | OPA1 H31 | GGGATGTGATAACAGTA |
| | 3UTR(+1675+1691) | | | 3UTR(+1729+1745) | |
| 1872 | OPA1 H31 | TGTGGAAATACAGCTGA | 1890 | OPA1 H31 | AAAGGGATGTGATAACA |
| | 3UTR(+1678+1694) | | | 3UTR(+1732+1748) | |
| 1873 | OPA1 H31 | ATCTGTGGAAATACAGC | 1891 | OPA1 H31 | TACAAAGGGATGTGATA |
| | 3UTR(+1681+1697) | | | 3UTR(+1735+1751) | |
| 1874 | OPA1 H31 | GAAATCTGTGGAAATAC | 1892 | OPA1 H31 | TTATACAAAGGGATGTG |
| | 3UTR(+1684+1700) | | | 3UTR(+1738+1754) | |
| 1875 | OPA1 H31 | GGAGAAATCTGTGGAAA | 1893 | OPA1 H31 | AACTTATACAAAGGGAT |
| | 3UTR(+1687+1703) | | | 3UTR(+1741+1757) | |
| 1876 | OPA1 H31 | TTGGGAGAAATCTGTGG | 1894 | OPA1 H31 | TAAAACTTATACAAAGG |
| | 3UTR(+1690+1706) | | | 3UTR(+1744+1760) | |
| 1877 | OPA1 H31 | TCCTTGGGAGAAATCTG | 1895 | OPA1 H31 | TTTTAAAACTTATACAA |
| | 3UTR(+1693+1709) | | | 3UTR(+1747+1763) | |
| 1878 | OPA1 H31 | TTTTCCTTGGGAGAAAT | 1896 | OPA1 H31 | TCTTTTTAAAACTTATA |
| | 3UTR(+1696+1712) | | | 3UTR(+1750+1766) | |
| 1879 | OPA1 H31 | GCCTTTTCCTTGGGAGA | 1897 | OPA1 H31 | ATCTCTTTTTAAAACTT |
| | 3UTR(+1699+1715) | | | 3UTR(+1753+1769) | |
| 1880 | OPA1 H31 | TTAGCCTTTTCCTTGGG | 1898 | OPA1 H31 | TCCATCTCTTTTTAAAA |
| | 3UTR(+1702+1718) | | | 3UTR(+1756+1772) | |
| 1881 | OPA1 H31 | ATATTAGCCTTTTCCTT | 1899 | OPA1 H31 | CCCTCCATCTCTTTTTA |
| | 3UTR(+1705+1721) | | | 3UTR(+1759+1775) | |
| 1882 | OPA1 H31 | CTAATATTAGCCTTTTC | 1900 | OPA1 H31 | TCTCCCTCCATCTCTTT |
| | 3UTR(+1708+1724) | | | 3UTR(+1762+1778) | |
| 1901 | OPA1 H31 | AGATCTCCCTCCATCTC | 1919 | OPA1 H31 | AATTGTTATCATCTATT |
| | 3UTR(+1765+1781) | | | 3UTR(+1819+1835) | |
| 1902 | OPA1 H31 | TGAAGATCTCCCTCCAT | 1920 | OPA1 H31 | ATTAATTGTTATCATCT |
| | 3UTR(+1768+1784) | | | 3UTR(+1822+1838) | |
| 1903 | OPA1 H31 | AAATGAAGATCTCCCTC | 1921 | OPA1 H31 | AATATTAATTGTTATCA |
| | 3UTR(+1771+1787) | | | 3UTR(+1825+1841) | |
| 1904 | OPA1 H31 | AAGAAATGAAGATCTCC | 1922 | OPA1 H31 | AGTAATATTAATTGTTA |
| | 3UTR(+1774+1790) | | | 3UTR(+1828+1844) | |
| 1905 | OPA1 H31 | TCAAAGAAATGAAGATC | 1923 | OPA1 H31 | TTTAGTAATATTAATTG |
| | 3UTR(+1777+1793) | | | 3UTR(+1831+1847) | |
| 1906 | OPA1 H31 | TCCTCAAAGAAATGAAG | 1924 | OPA1 H31 | ACTTTTAGTAATATTAA |
| | 3UTR(+1780+1796) | | | 3UTR(+1834+1850) | |
| 1907 | OPA1 H31 | ATCTCCTCAAAGAAATG | 1925 | OPA1 H31 | GGGACTTTTAGTAATAT |
| | 3UTR(+1783+1799) | | | 3UTR(+1837+1853) | |
| 1908 | OPA1 H31 | CTGATCTCCTCAAAGAA | 1926 | OPA1 H31 | TGTGGGACTTTTAGTAA |
| | 3UTR(+1786+1802) | | | 3UTR(+1840+1856) | |
| 1909 | OPA1 H31 | ATACTGATCTCCTCAAA | 1927 | OPA1 H31 | TCATGTGGGACTTTTAG |
| | 3UTR(+1789+1805) | | | 3UTR(+1843+1859) | |
| 1910 | OPA1 H31 | ACAATACTGATCTCCTC | 1928 | OPA1 H31 | CTCTCATGTGGGACTTT |
| | 3UTR(+1792+1808) | | | 3UTR(+1846+1862) | |
| 1911 | OPA1 H31 | GTTACAATACTGATCTC | 1929 | OPA1 H31 | GGACTCTCATGTGGGAC |
| | 3UTR(+1795+1811) | | | 3UTR(+1849+1865) | |
| 1912 | OPA1 H31 | TACGTTACAATACTGAT | 1930 | OPA1 H31 | TCAGGACTCTCATGTGG |
| | 3UTR(+1798+1814) | | | 3UTR(+1852+1868) | |
| 1913 | OPA1 H31 | ACATACGTTACAATACT | 1931 | OPA1 H31 | GCGTCAGGACTCTCATG |
| | 3UTR(+1801+1817) | | | 3UTR(+1855+1871) | |
| 1914 | OPA1 H31 | TTCACATACGTTACAAT | 1932 | OPA1 H31 | AGGGCGTCAGGACTCTC |
| | 3UTR(+1804+1820) | | | 3UTR(+1858+1874) | |
| 1915 | OPA1 H31 | CTATTCACATACGTTAC | 1933 | OPA1 H31 | GAGAGGGCGTCAGGACT |
| | 3UTR(+1807+1823) | | | 3UTR(+1861+1877) | |
| 1916 | OPA1 H31 | CATCTATTCACATACGT | 1934 | OPA1 H31 | ATGGAGAGGGCGTCAGG |
| | 3UTR(+1810+1826) | | | 3UTR(+1864+1880) | |
| 1917 | OPA1 H31 | TATCATCTATTCACATA | 1935 | OPA1 H31 | GGCATGGAGAGGGCGTC |
| | 3UTR(+1813+1829) | | | 3UTR(+1867+1883) | |
| 1918 | OPA1 H31 | TGTTATCATCTATTCAC | 1936 | OPA1 H31 | TGGGGCATGGAGAGGGC |
| | 3UTR(+1816+1832) | | | 3UTR(+1870+1886) | |
| 1937 | OPA1 H31 | CTGTGGGGCATGGAGAG | 1955 | OPA1 H31 | AGGATGAGATCAGCTAA |
| | 3UTR(+1873+1889) | | | 3UTR(+1927+1943) | |
| 1938 | OPA1 H31 | TTACTGTGGGGCATGGA | 1956 | OPA1 H31 | CTTAGGATGAGATCAGC |
| | 3UTR(+1876+1892) | | | 3UTR(+1930+1946) | |
| 1939 | OPA1 H31 | ACATTACTGTGGGGCAT | 1957 | OPA1 H31 | ATGCTTAGGATGAGATC |
| | 3UTR(+1879+1895) | | | 3UTR(+1933+1949) | |
| 1940 | OPA1 H31 | GCCACATTACTGTGGGG | 1958 | OPA1 H31 | AGCATGCTTAGGATGAG |
| | 3UTR(+1882+1898) | | | 3UTR(+1936+1952) | |
| 1941 | OPA1 H31 | GAAGCCACATTACTGTG | 1959 | OPA1 H31 | TAAAGCATGCTTAGGAT |
| | 3UTR(+1885+1901) | | | 3UTR(+1939+1955) | |
| 1942 | OPA1 H31 | AAAGAAGCCACATTACT | 1960 | OPA1 H31 | AAATAAAGCATGCTTAG |
| | 3UTR(+1888+1904) | | | 3UTR(+1942+1958) | |
| 1943 | OPA1 H31 | ATGAAAGAAGCCACATT | 1961 | OPA1 H31 | GAAAAATAAAGCATGCT |
| | 3UTR(+1891+1907) | | | 3UTR(+1945+1961) | |
| 1944 | OPA1 H31 | CCCATGAAAGAAGCCAC | 1962 | OPA1 H31 | AAGGAAAAATAAAGCAT |
| | 3UTR(+1894+1910) | | | 3UTR(+1948+1964) | |
| 1945 | OPA1 H31 | AAACCCATGAAAGAAGC | 1963 | OPA1 H31 | TTCAAGGAAAAATAAAG |
| | 3UTR(+1897+1913) | | | 3UTR(+1951+1967) | |
| 1946 | OPA1 H31 | AAAAAACCCATGAAAGA | 1964 | OPA1 H31 | GCTTTCAAGGAAAAATA |
| | 3UTR(+1900+1916) | | | 3UTR(+1954+1970) | |
| 1947 | OPA1 H31 | AAAAAAAAACCCATGAA | 1965 | OPA1 H31 | CTAGCTTTCAAGGAAAA |
| | 3UTR(+1903+1919) | | | 3UTR(+1957+1973) | |
| 1948 | OPA1 H31 | AAGAAAAAAAAACCCAT | 1966 | OPA1 H31 | TACCTAGCTTTCAAGGA |
| | 3UTR(+1906+1922) | | | 3UTR(+1960+1976) | |
| 1949 | OPA1 H31 | AAGAAGAAAAAAAAACC | 1967 | OPA1 H31 | AAATACCTAGCTTTCAA |
| | 3UTR(+1909+1925) | | | 3UTR(+1963+1979) | |
| 1950 | OPA1 H31 | AAAAAGAAGAAAAAAAA | 1968 | OPA1 H31 | GATAAATACCTAGCTTT |
| | 3UTR(+1912+1928) | | | 3UTR(+1966+1982) | |
| 1951 | OPA1 H31 | GCTAAAAAGAAGAAAAA | 1969 | OPA1 H31 | GTTGATAAATACCTAGC |
| | 3UTR(+1915+1931) | | | 3UTR(+1969+1985) | |
| 1952 | OPA1 H31 | TCAGCTAAAAAGAAGAA | 1970 | OPA1 H31 | GCAGTTGATAAATACCT |
| | 3UTR(+1918+1934) | | | 3UTR(+1972+1988) | |
| 1953 | OPA1 H31 | AGATCAGCTAAAAAGAA | 1971 | OPA1 H31 | TCTGCAGTTGATAAATA |
| | 3UTR(+1921+1937) | | | 3UTR(+1975+1991) | |
| 1954 | OPA1 H31 | ATGAGATCAGCTAAAAA | 1972 | OPA1 H31 | ACATCTGCAGTTGATAA |
| | 3UTR(+1924+1940) | | | 3UTR(+1978+1994) | |
| 1973 | OPA1 H31 | ATAACATCTGCAGTTGA | 1991 | OPA1 H31 | CTACAGACACAAGACAC |
| | 3UTR(+1981+1997) | | | 3UTR(+2035+2051) | |
| 1974 | OPA1 H31 | TCAATAACATCTGCAGT | 1992 | OPA1 H31 | GAACTACAGACACAAGA |
| | 3UTR(+1984+2000) | | | 3UTR(+2038+2054) | |
| 1975 | OPA1 H31 | CTTTCAATAACATCTGC | 1993 | OPA1 H31 | TTTGAACTACAGACACA |
| | 3UTR(+1987+2003) | | | 3UTR(+2041+2057) | |
| 1976 | OPA1 H31 | TTTCTTTCAATAACATC | 1994 | OPA1 H31 | ACTTTTGAACTACAGAC |
| | 3UTR(+1990+2006) | | | 3UTR(+2044+2060) | |
| 1977 | OPA1 H31 | TATTTTCTTTCAATAAC | 1995 | OPA1 H31 | CTGACTTTTGAACTACA |
| | 3UTR(+1993+2009) | | | 3UTR(+2047+2063) | |
| 1978 | OPA1 H31 | TTTTATTTTCTTTCAAT | 1996 | OPA1 H31 | TTTCTGACTTTTGAACT |
| | 3UTR(+1996+2012) | | | 3UTR(+2050+2066) | |
| 1979 | OPA1 H31 | GAATTTTATTTTCTTTC | 1997 | OPA1 H31 | TCATTTCTGACTTTTGA |
| | 3UTR(+1999+2015) | | | 3UTR(+2053+2069) | |
| 1980 | OPA1 H31 | ACTGAATTTTATTTTCT | 1998 | OPA1 H31 | GAATCATTTCTGACTTT |
| | 3UTR(+2002+2018) | | | 3UTR(+2056+2072) | |
| 1981 | OPA1 H31 | GAGACTGAATTTTATTT | 1999 | OPA1 H31 | TTAGAATCATTTCTGAC |
| | 3UTR(+2005+2021) | | | 3UTR(+2059+2075) | |
| 1982 | OPA1 H31 | CTTGAGACTGAATTTTA | 2000 | OPA1 H31 | AAATTAGAATCATTTCT |
| | 3UTR(+2008+2024) | | | 3UTR(+2062+2078) | |
| 1983 | OPA1 H31 | ACTCTTGAGACTGAATT | 2001 | OPA1 H31 | TTTAAATTAGAATCATT |
| | 3UTR(+2011+2027) | | | 3UTR(+2065+2081) | |
| 1984 | OPA1 H31 | TTTACTCTTGAGACTGA | 2002 | OPA1 H31 | TTGTTTAAATTAGAATC |
| | 3UTR(+2014+2030) | | | 3UTR(+2068+2084) | |
| 1985 | OPA1 H31 | GGGTTTACTCTTGAGAC | 2003 | OPA1 H31 | TTTTTGTTTAAATTAGA |
| | 3UTR(+2017+2033) | | | 3UTR(+2071+2087) | |
| 1986 | OPA1 H31 | ACAGGGTTTACTCTTGA | 2004 | OPA1 H31 | ATCTTTTTGTTTAAATT |
| | 3UTR(+2020+2036) | | | 3UTR(+2074+2090) | |
| 1987 | OPA1 H31 | GACACAGGGTTTACTCT | 2005 | OPA1 H31 | AGTATCTTTTTGTTTAA |
| | 3UTR(+2023+2039) | | | 3UTR(+2077+2093) | |
| 1988 | OPA1 H31 | CAAGACACAGGGTTTAC | 2006 | OPA1 H31 | TTTAGTATCTTTTTGTT |
| | 3UTR(+2026+2042) | | | 3UTR(+2080+2096) | |
| 1989 | OPA1 H31 | ACACAAGACACAGGGTT | 2007 | OPA1 H31 | ATATTTAGTATCTTTTT |
| | 3UTR(+2029+2045) | | | 3UTR(+2083+2099) | |
| 1990 | OPA1 H31 | CAGACACAAGACACAGG | 2008 | OPA1 H31 | TGTATATTTAGTATCTT |
| | 3UTR(+2032+2048) | | | 3UTR(+2086+2102) | |
| 2009 | OPA1 H31 | TTCTGTATATTTAGTAT | 2027 | OPA1 H31 | CTTTGCAAATTCTGACA |
| | 3UTR(+2089+2105) | | | 3UTR(+2143+2159) | |
| 2010 | OPA1 H31 | AACTTCTGTATATTTAG | 2028 | OPA1 H31 | ACTCTTTGCAAATTCTG |
| | 3UTR(+2092+2108) | | | 3UTR(+2146+2162) | |
| 2011 | OPA1 H31 | TTTAACTTCTGTATATT | 2029 | OPA1 H31 | TCCACTCTTTGCAAATT |
| | 3UTR(+2095+2111) | | | 3UTR(+2149+2165) | |
| 2012 | OPA1 H31 | GAATTTAACTTCTGTAT | 2030 | OPA1 H31 | CACTCCACTCTTTGCAA |
| | 3UTR(+2098+2114) | | | 3UTR(+2152+2168) | |
| 2013 | OPA1 H31 | TTCGAATTTAACTTCTG | 2031 | OPA1 H31 | GTCCACTCCACTCTTTG |
| | 3UTR(+2101+2117) | | | 3UTR(+2155+2171) | |
| 2014 | OPA1 H31 | TAGTTCGAATTTAACTT | 2032 | OPA1 H31 | TTTGTCCACTCCACTCT |
| | 3UTR(+2104+2120) | | | 3UTR(+2158+2174) | |
| 2015 | OPA1 H31 | GGCTAGTTCGAATTTAA | 2033 | OPA1 H31 | AGCTTTGTCCACTCCAC |
| | 3UTR(+2107+2123) | | | 3UTR(+2161+2177) | |
| 2016 | OPA1 H31 | TGTGGCTAGTTCGAATT | 2034 | OPA1 H31 | CAGAGCTTTGTCCACTC |
| | 3UTR(+2110+2126) | | | 3UTR(+2164+2180) | |
| 2017 | OPA1 H31 | TTCTGTGGCTAGTTCGA | 2035 | OPA1 H31 | ATACAGAGCTTTGTCCA |
| | 3UTR(+2113+2129) | | | 3UTR(+2167+2183) | |
| 2018 | OPA1 H31 | TGATTCTGTGGCTAGTT | 2036 | OPA1 H31 | TCCATACAGAGCTTTGT |
| | 3UTR(+2116+2132) | | | 3UTR(+2170+2186) | |
| 2019 | OPA1 H31 | AAATGATTCTGTGGCTA | 2037 | OPA1 H31 | TCTTCCATACAGAGCTT |
| | 3UTR(+2119+2135) | | | 3UTR(+2173+2189) | |
| 2020 | OPA1 H31 | AACAAATGATTCTGTGG | 2038 | OPA1 H31 | CAGTCTTCCATACAGAG |
| | 3UTR(+2122+2138) | | | 3UTR(+2176+2192) | |
| 2021 | OPA1 H31 | AAAAACAAATGATTCTG | 2039 | OPA1 H31 | GTTCAGTCTTCCATACA |
| | 3UTR(+2125+2141) | | | 3UTR(+2179+2195) | |
| 2022 | OPA1 H31 | CATAAAAACAAATGATT | 2040 | OPA1 H31 | GTTGTTCAGTCTTCCAT |
| | 3UTR(+2128+2144) | | | 3UTR(+2182+2198) | |
| 2023 | OPA1 H31 | TGACATAAAAACAAATG | 2041 | OPA1 H31 | ACAGTTGTTCAGTCTTC |
| | 3UTR(+2131+2147) | | | 3UTR(+2185+2201) | |
| 2024 | OPA1 H31 | TTCTGACATAAAAACAA | 2042 | OPA1 H31 | TTTACAGTTGTTCAGTC |
| | 3UTR(+2134+2150) | | | 3UTR(+2188+2204) | |
| 2025 | OPA1 H31 | AAATTCTGACATAAAAA | 2043 | OPA1 H31 | CTATTTACAGTTGTTCA |
| | 3UTR(+2137+2153) | | | 3UTR(+2191+2207) | |
| 2026 | OPA1 H31 | TGCAAATTCTGACATAA | 2044 | OPA1 H31 | CATCTATTTACAGTTGT |
| | 3UTR(+2140+2156) | | | 3UTR(+2194+2210) | |
| 2045 | OPA1 H31 | TATCATCTATTTACAGT | 2063 | OPA1 H31 | CTGCCTAGGTACACTGA |
| | 3UTR(+2197+2213) | | | 3UTR(+2251+2267) | |
| 2046 | OPA1 H31 | GGATATCATCTATTTAC | 2064 | OPA1 H31 | GCACTGCCTAGGTACAC |
| | 3UTR(+2200+2216) | | | 3UTR(+2254+2270) | |
| 2047 | OPA1 H31 | TTTGGATATCATCTATT | 2065 | OPA1 H31 | TGTGCACTGCCTAGGTA |
| | 3UTR(+2203+2219) | | | 3UTR(+2257+2273) | |
| 2048 | OPA1 H31 | AAGTTTGGATATCATCT | 2066 | OPA1 H31 | TGCTGTGCACTGCCTAG |
| | 3UTR(+2206+2222) | | | 3UTR(+2260+2276) | |
| 2049 | OPA1 H31 | ATTAAGTTTGGATATCA | 2067 | OPA1 H31 | TCGTGCTGTGCACTGCC |
| | 3UTR(+2209+2225) | | | 3UTR(+2263+2279) | |
| 2050 | OPA1 H31 | CAAATTAAGTTTGGATA | 2068 | OPA1 H31 | ATTTCGTGCTGTGCACT |
| | 3UTR(+2212+2228) | | | 3UTR(+2266+2282) | |
| 2051 | OPA1 H31 | AGCCAAATTAAGTTTGG | 2069 | OPA1 H31 | CTTATTTCGTGCTGTGC |
| | 3UTR(+2215+2231) | | | 3UTR(+2269+2285) | |
| 2052 | OPA1 H31 | CCTAGCCAAATTAAGTT | 2070 | OPA1 H31 | CCACTTATTTCGTGCTG |
| | 3UTR(+2218+2234) | | | 3UTR(+2272+2288) | |
| 2053 | OPA1 H31 | AGTCCTAGCCAAATTAA | 2071 | OPA1 H31 | GGGCCACTTATTTCGTG |
| | 3UTR(+2221+2237) | | | 3UTR(+2275+2291) | |
| 2054 | OPA1 H31 | TGAAGTCCTAGCCAAAT | 2072 | OPA1 H31 | CAAGGGCCACTTATTTC |
| | 3UTR(+2224+2240) | | | 3UTR(+2278+2294) | |
| 2055 | OPA1 H31 | AATTGAAGTCCTAGCCA | 2073 | OPA1 H31 | CTGCAAGGGCCACTTAT |
| | 3UTR(+2227+2243) | | | 3UTR(+2281+2297) | |
| 2056 | OPA1 H31 | TAAAATTGAAGTCCTAG | 2074 | OPA1 H31 | AAGCTGCAAGGGCCACT |
| | 3UTR(+2230+2246) | | | 3UTR(+2284+2300) | |
| 2057 | OPA1 H31 | TTTTAAAATTGAAGTCC | 2075 | OPA1 H31 | GGGAAGCTGCAAGGGCC |
| | 3UTR(+2233+2249) | | | 3UTR(+2287+2303) | |
| 2058 | OPA1 H31 | GATTTTTAAAATTGAAG | 2076 | OPA1 H31 | ACGGGGAAGCTGCAAGG |
| | 3UTR(+2236+2252) | | | 3UTR(+2290+2306) | |
| 2059 | OPA1 H31 | ACTGATTTTTAAAATTG | 2077 | OPA1 H31 | TAAACGGGGAAGCTGCA |
| | 3UTR(+2239+2255) | | | 3UTR(+2293+2309) | |
| 2060 | OPA1 H31 | TACACTGATTTTTAAAA | 2078 | OPA1 H31 | GGTTAAACGGGGAAGCT |
| | 3UTR(+2242+2258) | | | 3UTR(+2296+2312) | |
| 2061 | OPA1 H31 | AGGTACACTGATTTTTA | 2079 | OPA1 H31 | GTGGGTTAAACGGGGAA |
| | 3UTR(+2245+2261) | | | 3UTR(+2299+2315) | |
| 2062 | OPA1 H31 | CCTAGGTACACTGATTT | 2080 | OPA1 H31 | ACAGTGGGTTAAACGGG |
| | 3UTR(+2248+2264) | | | 3UTR(+2302+2318) | |
| 2081 | OPA1 H31 | AGCACAGTGGGTTAAAC | 2099 | OPA1 H31 | AAGAGGGCAATATCATA |
| | 3UTR(+2305+2321) | | | 3UTR(+2359+2375) | |
| 2082 | OPA1 H31 | TATAGCACAGTGGGTTA | 2100 | OPA1 H31 | ACAAAGAGGGCAATATC |
| | 3UTR(+2308+2324) | | | 3UTR(+2362+2378) | |
| 2083 | OPA1 H31 | AACTATAGCACAGTGGG | 2101 | OPA1 H31 | AATACAAAGAGGGCAAT |
| | 3UTR(+2311+2327) | | | 3UTR(+2365+2381) | |
| 2084 | OPA1 H31 | CGCAACTATAGCACAGT | 2102 | OPA1 H31 | GGGAATACAAAGAGGGC |
| | 3UTR(+2314+2330) | | | 3UTR(+2368+2384) | |
| 2085 | OPA1 H31 | ACCCGCAACTATAGCAC | 2103 | OPA1 H31 | AATGGGAATACAAAGAG |
| | 3UTR(+2317+2333) | | | 3UTR(+2371+2387) | |
| 2086 | OPA1 H31 | TCCACCCGCAACTATAG | 2104 | OPA1 H31 | GAAAATGGGAATACAAA |
| | 3UTR(+2320+2336) | | | 3UTR(+2374+2390) | |
| 2087 | OPA1 H31 | TGTTCCACCCGCAACTA | 2105 | OPA1 H31 | GTAGAAAATGGGAATAC |
| | 3UTR(+2323+2339) | | | 3UTR(+2377+2393) | |
| 2088 | OPA1 H31 | GACTGTTCCACCCGCAA | 2106 | OPA1 H31 | ACTGTAGAAAATGGGAA |
| | 3UTR(+2326+2342) | | | 3UTR(+2380+2396) | |
| 2089 | OPA1 H31 | GTTGACTGTTCCACCCG | 2107 | OPA1 H31 | AAAACTGTAGAAAATGG |
| | 3UTR(+2329+2345) | | | 3UTR(+2383+2399) | |
| 2090 | OPA1 H31 | AAGGTTGACTGTTCCAC | 2108 | OPA1 H31 | GAAAAAACTGTAGAAAA |
| | 3UTR(+2332+2348) | | | 3UTR(+2386+2402) | |
| 2091 | OPA1 H31 | AGAAAGGTTGACTGTTC | 2109 | OPA1 H31 | GCGGAAAAAACTGTAGA |
| | 3UTR(+2335+2351) | | | 3UTR(+2389+2405) | |
| 2092 | OPA1 H31 | ACTAGAAAGGTTGACTG | 2110 | OPA1 H31 | TCTGCGGAAAAAACTGT |
| | 3UTR(+2338+2354) | | | 3UTR(+2392+2408) | |
| 2093 | OPA1 H31 | ACTACTAGAAAGGTTGA | 2111 | OPA1 H31 | AAGTCTGCGGAAAAAAC |
| | 3UTR(+2341+2357) | | | 3UTR(+2395+2411) | |
| 2094 | OPA1 H31 | TAAACTACTAGAAAGGT | 2112 | OPA1 H31 | AAGAAGTCTGCGGAAAA |
| | 3UTR(+2344+2360) | | | 3UTR(+2398+2414) | |
| 2095 | OPA1 H31 | TCATAAACTACTAGAAA | 2113 | OPA1 H31 | AGAAAGAAGTCTGCGGA |
| | 3UTR(+2347+2363) | | | 3UTR(+2401+2417) | |
| 2096 | OPA1 H31 | ATATCATAAACTACTAG | 2114 | OPA1 H31 | TGCAGAAAGAAGTCTGC |
| | 3UTR(+2350+2366) | | | 3UTR(+2404+2420) | |
| 2097 | OPA1 H31 | GCAATATCATAAACTAC | 2115 | OPA1 H31 | ATTTGCAGAAAGAAGTC |
| | 3UTR(+2353+2369) | | | 3UTR(+2407+2423) | |
| 2098 | OPA1 H31 | AGGGCAATATCATAAAC | 2116 | OPA1 H31 | ATAATTTGCAGAAAGAA |
| | 3UTR(+2356+2372) | | | 3UTR(+2410+2426) | |
| 2117 | OPA1 H31 | TGAATAATTTGCAGAAA | 2135 | OPA1 H31 | ACTCTCTGCCATGTTCC |
| | 3UTR(+2413+2429) | | | 3UTR(+2467+2483) | |
| 2118 | OPA1 H31 | GGCTGAATAATTTGCAG | 2136 | OPA1 H31 | ACCACTCTCTGCCATGT |
| | 3UTR(+2416+2432) | | | 3UTR(+2470+2486) | |
| 2119 | OPA1 H31 | GGAGGCTGAATAATTTG | 2137 | OPA1 H31 | AGCACCACTCTCTGCCA |
| | 3UTR(+2419+2435) | | | 3UTR(+2473+2489) | |
| 2120 | OPA1 H31 | TTTGGAGGCTGAATAAT | 2138 | OPA1 H31 | GGAAGCACCACTCTCTG |
| | 3UTR(+2422+2438) | | | 3UTR(+2476+2492) | |
| 2121 | OPA1 H31 | GCATTTGGAGGCTGAAT | 2139 | OPA1 H31 | CTGGGAAGCACCACTCT |
| | 3UTR(+2425+2441) | | | 3UTR(+2479+2495) | |
| 2122 | OPA1 H31 | TTTGCATTTGGAGGCTG | 2140 | OPA1 H31 | AGGCTGGGAAGCACCAC |
| | 3UTR(+2428+2444) | | | 3UTR(+2482+2498) | |
| 2123 | OPA1 H31 | TCATTTGCATTTGGAGG | 2141 | OPA1 H31 | GTGAGGCTGGGAAGCAC |
| | 3UTR(+2431+2447) | | | 3UTR(+2485+2501) | |
| 2124 | OPA1 H31 | CATTCATTTGCATTTGG | 2142 | OPA1 H31 | ATTGTGAGGCTGGGAAG |
| | 3UTR(+2434+2450) | | | 3UTR(+2488+2504) | |
| 2125 | OPA1 H31 | TATCATTCATTTGCATT | 2143 | OPA1 H31 | CACATTGTGAGGCTGGG |
| | 3UTR(+2437+2453) | | | 3UTR(+2491+2507) | |
| 2126 | OPA1 H31 | TTATATCATTCATTTGC | 2144 | OPA1 H31 | TCCCACATTGTGAGGCT |
| | 3UTR(+2440+2456) | | | 3UTR(+2494+2510) | |
| 2127 | OPA1 H31 | TTTTTATATCATTCATT | 2145 | OPA1 H31 | AATTCCCACATTGTGAG |
| | 3UTR(+2443+2459) | | | 3UTR(+2497+2513) | |
| 2128 | OPA1 H31 | TTATTTTTATATCATTC | 2146 | OPA1 H31 | TCAAATTCCCACATTGT |
| | 3UTR(+2446+2462) | | | 3UTR(+2500+2516) | |
| 2129 | OPA1 H31 | TACTTATTTTTATATCA | 2147 | OPA1 H31 | ATGTCAAATTCCCACAT |
| | 3UTR(+2449+2465) | | | 3UTR(+2503+2519) | |
| 2130 | OPA1 H31 | CCCTACTTATTTTTATA | 2148 | OPA1 H31 | CCTATGTCAAATTCCCA |
| | 3UTR(+2452+2468) | | | 3UTR(+2506+2522) | |
| 2131 | OPA1 H31 | GTTCCCTACTTATTTTT | 2149 | OPA1 H31 | CATCCTATGTCAAATTC |
| | 3UTR(+2455+2471) | | | 3UTR(+2509+2525) | |
| 2132 | OPA1 H31 | CATGTTCCCTACTTATT | 2150 | OPA1 H31 | TCTCATCCTATGTCAAA |
| | 3UTR(+2458+2474) | | | 3UTR(+2512+2528) | |
| 2133 | OPA1 H31 | TGCCATGTTCCCTACTT | 2151 | OPA1 H31 | GACTCTCATCCTATGTC |
| | 3UTR(+2461+2477) | | | 3UTR(+2515+2531) | |
| 2134 | OPA1 H31 | CTCTGCCATGTTCCCTA | 2152 | OPA1 H31 | TCTGACTCTCATCCTAT |
| | 3UTR(+2464+2480) | | | 3UTR(+2518+2534) | |
| 2153 | OPA1 H31 | TACTCTGACTCTCATCC | 2171 | OPA1 H31 | TCTAGAATAAATAAATA |
| | 3UTR(+2521+2537) | | | 3UTR(+2575+2591) | |
| 2154 | OPA1 H31 | CTATACTCTGACTCTCA | 2172 | OPA1 H31 | ACATCTAGAATAAATAA |
| | 3UTR(+2524+2540) | | | 3UTR(+2578+2594) | |
| 2155 | OPA1 H31 | AACCTATACTCTGACTC | 2173 | OPA1 H31 | CATACATCTAGAATAAA |
| | 3UTR(+2527+2543) | | | 3UTR(+2581+2597) | |
| 2156 | OPA1 H31 | TTAAACCTATACTCTGA | 2174 | OPA1 H31 | ATACATACATCTAGAAT |
| | 3UTR(+2530+2546) | | | 3UTR(+2584+2600) | |
| 2157 | OPA1 H31 | CTTTTAAACCTATACTC | 2175 | OPA1 H31 | CAGATACATACATCTAG |
| | 3UTR(+2533+2549) | | | 3UTR(+2587+2603) | |
| 2158 | OPA1 H31 | TATCTTTTAAACCTATA | 2176 | OPA1 H31 | CCTCAGATACATACATC |
| | 3UTR(+2536+2552) | | | 3UTR(+2590+2606) | |
| 2159 | OPA1 H31 | TTTTATCTTTTAAACCT | 2177 | OPA1 H31 | TTTCCTCAGATACATAC |
| | 3UTR(+2539+2555) | | | 3UTR(+2593+2609) | |
| 2160 | OPA1 H31 | AGATTTTATCTTTTAAA | 2178 | OPA1 H31 | TTCTTTCCTCAGATACA |
| | 3UTR(+2542+2558) | | | 3UTR(+2596+2612) | |
| 2161 | OPA1 H31 | TAAAGATTTTATCTTTT | 2179 | OPA1 H31 | GATTTCTTTCCTCAGAT |
| | 3UTR(+2545+2561) | | | 3UTR(+2599+2615) | |
| 2162 | OPA1 H31 | AACTAAAGATTTTATCT | 2180 | OPA1 H31 | CCAGATTTCTTTCCTCA |
| | 3UTR(+2548+2564) | | | 3UTR(+2602+2618) | |
| 2163 | OPA1 H31 | ATTAACTAAAGATTTTA | 2181 | OPA1 H31 | ATACCAGATTTCTTTCC |
| | 3UTR(+2551+2567) | | | 3UTR(+2605+2621) | |
| 2164 | OPA1 H31 | ATTATTAACTAAAGATT | 2182 | OPA1 H31 | AAAATACCAGATTTCTT |
| | 3UTR(+2554+2570) | | | 3UTR(+2608+2624) | |
| 2165 | OPA1 H31 | AAAATTATTAACTAAAG | 2183 | OPA1 H31 | GCAAAAATACCAGATTT |
| | 3UTR(+2557+2573) | | | 3UTR(+2611+2627) | |
| 2166 | OPA1 H31 | TACAAAATTATTAACTA | 2184 | OPA1 H31 | AAAGCAAAAATACCAGA |
| | 3UTR(+2560+2576) | | | 3UTR(+2614+2630) | |
| 2167 | OPA1 H31 | AAATACAAAATTATTAA | 2185 | OPA1 H31 | TGGAAAGCAAAAATACC |
| | 3UTR(+2563+2579) | | | 3UTR(+2617+2633) | |
| 2168 | OPA1 H31 | AATAAATACAAAATTAT | 2186 | OPA1 H31 | TATTGGAAAGCAAAAAT |
| | 3UTR(+2566+2582) | | | 3UTR(+2620+2636) | |
| 2169 | OPA1 H31 | ATAAATAAATACAAAAT | 2187 | OPA1 H31 | CTTTATTGGAAAGCAAA |
| | 3UTR(+2569+2585) | | | 3UTR(+2623+2639) | |
| 2170 | OPA1 H31 | AGAATAAATAAATACAA | 2188 | OPA1 H31 | CCCCTTTATTGGAAAGC |
| | 3UTR(+2572+2588) | | | 3UTR(+2626+2642) | |
| 2189 | OPA1 H31 | GATCCCCTTTATTGGAA | 2207 | OPA1 H31 | GCTAGAGCTATAACATA |
| | 3UTR(+2629+2645) | | | 3UTR(+2683+2699) | |
| 2190 | OPA1 H31 | TTTGATCCCCTTTATTG | 2208 | OPA1 H31 | ACTGCTAGAGCTATAAC |
| | 3UTR(+2632+2648) | | | 3UTR(+2686+2702) | |
| 2191 | OPA1 H31 | TACTTTGATCCCCTTTA | 2209 | OPA1 H31 | CATACTGCTAGAGCTAT |
| | 3UTR(+2635+2651) | | | 3UTR(+2689+2705) | |
| 2192 | OPA1 H31 | CATTACTTTGATCCCCT | 2210 | OPA1 H31 | TTCCATACTGCTAGAGC |
| | 3UTR(+2638+2654) | | | 3UTR(+2692+2708) | |
| 2193 | OPA1 H31 | AACCATTACTTTGATCC | 2211 | OPA1 H31 | CATTTCCATACTGCTAG |
| | 3UTR(+2641+2657) | | | 3UTR(+2695+2711) | |
| 2194 | OPA1 H31 | AAAAACCATTACTTTGA | 2212 | OPA1 H31 | GCACATTTCCATACTGC |
| | 3UTR(+2644+2660) | | | 3UTR(+2698+2714) | |
| 2195 | OPA1 H31 | GAGAAAAACCATTACTT | 2213 | OPA1 H31 | AAAGCACATTTCCATAC |
| | 3UTR(+2647+2663) | | | 3UTR(+2701+2717) | |
| 2196 | OPA1 H31 | TGAGAGAAAAACCATTA | 2214 | OPA1 H31 | TTTAAAGCACATTTCCA |
| | 3UTR(+2650+2666) | | | 3UTR(+2704+2720) | |
| 2197 | OPA1 H31 | AACTGAGAGAAAAACCA | 2215 | OPA1 H31 | TATTTTAAAGCACATTT |
| | 3UTR(+2653+2669) | | | 3UTR(+2707+2723) | |
| 2198 | OPA1 H31 | GAGAACTGAGAGAAAAA | 2216 | OPA1 H31 | GCATATTTTAAAGCACA |
| | 3UTR(+2656+2672) | | | 3UTR(+2710+2726) | |
| 2199 | OPA1 H31 | TTAGAGAACTGAGAGAA | 2217 | OPA1 H31 | TAAGCATATTTTAAAGC |
| | 3UTR(+2659+2675) | | | 3UTR(+2713+2729) | |
| 2200 | OPA1 H31 | AGCTTAGAGAACTGAGA | 2218 | OPA1 H31 | AGGTAAGCATATTTTAA |
| | 3UTR(+2662+2678) | | | 3UTR(+2716+2732) | |
| 2201 | OPA1 H31 | ACCAGCTTAGAGAACTG | 2219 | OPA1 H31 | AAAAGGTAAGCATATTT |
| | 3UTR(+2665+2681) | | | 3UTR(+2719+2735) | |
| 2202 | OPA1 H31 | TAGACCAGCTTAGAGAA | 2220 | OPA1 H31 | TTCAAAAGGTAAGCATA |
| | 3UTR(+2668+2684) | | | 3UTR(+2722+2738) | |
| 2203 | OPA1 H31 | ACATAGACCAGCTTAGA | 2221 | OPA1 H31 | TCATTCAAAAGGTAAGC |
| | 3UTR(+2671+2687) | | | 3UTR(+2725+2741) | |
| 2204 | OPA1 H31 | ATAACATAGACCAGCTT | 2222 | OPA1 H31 | TGATCATTCAAAAGGTA |
| | 3UTR(+2674+2690) | | | 3UTR(+2728+2744) | |
| 2205 | OPA1 H31 | GCTATAACATAGACCAG | 2223 | OPA1 H31 | CCATGATCATTCAAAAG |
| | 3UTR(+2677+2693) | | | 3UTR(+2731+2747) | |
| 2206 | OPA1 H31 | AGAGCTATAACATAGAC | 2224 | OPA1 H31 | TAGCCATGATCATTCAA |
| | 3UTR(+2680+2696) | | | 3UTR(+2734+2750) | |
| 2225 | OPA1 H31 | ATATAGCCATGATCATT | 2243 | OPA1 H31 | GTTCATTCACAGTGCAC |
| | 3UTR(+2737+2753) | | | 3UTR(+2791+2807) | |
| 2226 | OPA1 H31 | AACATATAGCCATGATC | 2244 | OPA1 H31 | AAAGTTCATTCACAGTG |
| | 3UTR(+2740+2756) | | | 3UTR(+2794+2810) | |
| 2227 | OPA1 H31 | AACAACATATAGCCATG | 2245 | OPA1 H31 | TACAAAGTTCATTCACA |
| | 3UTR(+2743+2759) | | | 3UTR(+2797+2813) | |
| 2228 | OPA1 H31 | CTCAACAACATATAGCC | 2246 | OPA1 H31 | TAATACAAAGTTCATTC |
| | 3UTR(+2746+2762) | | | 3UTR(+2800+2816) | |
| 2229 | OPA1 H31 | TATCTCAACAACATATA | 2247 | OPA1 H31 | AAATAATACAAAGTTCA |
| | 3UTR(+2749+2765) | | | 3UTR(+2803+2819) | |
| 2230 | OPA1 H31 | AAATATCTCAACAACAT | 2248 | OPA1 H31 | AAAAAATAATACAAAGT |
| | 3UTR(+2752+2768) | | | 3UTR(+2806+2822) | |
| 2231 | OPA1 H31 | TTCAAATATCTCAACAA | 2249 | OPA1 H31 | TTAAAAAAATAATACAA |
| | 3UTR(+2755+2771) | | | 3UTR(+2809+2825) | |
| 2232 | OPA1 H31 | AGTTTCAAATATCTCAA | 2250 | OPA1 H31 | GTTTTAAAAAAATAATA |
| | 3UTR(+2758+2774) | | | 3UTR(+2812+2828) | |
| 2233 | OPA1 H31 | GTAAGTTTCAAATATCT | 2251 | OPA1 H31 | AAGGTTTTAAAAAAATA |
| | 3UTR(+2761+2777) | | | 3UTR(+2815+2831) | |
| 2234 | OPA1 H31 | AAGGTAAGTTTCAAATA | 2252 | OPA1 H31 | GTGAAGGTTTTAAAAAA |
| | 3UTR(+2764+2780) | | | 3UTR(+2818+2834) | |
| 2235 | OPA1 H31 | AACAAGGTAAGTTTCAA | 2253 | OPA1 H31 | AATGTGAAGGTTTTAAA |
| | 3UTR(+2767+2783) | | | 3UTR(+2821+2837) | |
| 2236 | OPA1 H31 | GAAAACAAGGTAAGTTT | 2254 | OPA1 H31 | CGTAATGTGAAGGTTTT |
| | 3UTR(+2770+2786) | | | 3UTR(+2824+2840) | |
| 2237 | OPA1 H31 | AGTGAAAACAAGGTAAG | 2255 | OPA1 H31 | ACACGTAATGTGAAGGT |
| | 3UTR(+2773+2789) | | | 3UTR(+2827+2843) | |
| 2238 | OPA1 H31 | ACAAGTGAAAACAAGGT | 2256 | OPA1 H31 | TCTACACGTAATGTGAA |
| | 3UTR(+2776+2792) | | | 3UTR(+2830+2846) | |
| 2239 | OPA1 H31 | TGCACAAGTGAAAACAA | 2257 | OPA1 H31 | ATATCTACACGTAATGT |
| | 3UTR(+2779+2795) | | | 3UTR(+2833+2849) | |
| 2240 | OPA1 H31 | CAGTGCACAAGTGAAAA | 2258 | OPA1 H31 | ATAATATCTACACGTAA |
| | 3UTR(+2782+2798) | | | 3UTR(+2836+2852) | |
| 2241 | OPA1 H31 | TCACAGTGCACAAGTGA | 2259 | OPA1 H31 | GCAATAATATCTACACG |
| | 3UTR(+2785+2801) | | | 3UTR(+2839+2855) | |
| 2242 | OPA1 H31 | CATTCACAGTGCACAAG | 2260 | OPA1 H31 | GTTGCAATAATATCTAC |
| | 3UTR(+2788+2804) | | | 3UTR(+2842+2858) | |
| 2261 | OPA1 H31 | TAAGTTGCAATAATATC | 2279 | OPA1 H31 | ATTTTTTAATTACTCAT |
| | 3UTR(+2845+2861) | | | 3UTR(+2899+2915) | |
| 2262 | OPA1 H31 | ATATAAGTTGCAATAAT | 2280 | OPA1 H31 | AATATTTTTTAATTACT |
| | 3UTR(+2848+2864) | | | 3UTR(+2902+2918) | |
| 2263 | OPA1 H31 | AAAATATAAGTTGCAAT | 2281 | OPA1 H31 | TTAAATATTTTTTAATT |
| | 3UTR(+2851+2867) | | | 3UTR(+2905+2921) | |
| 2264 | OPA1 H31 | GGCAAAATATAAGTTGC | 2282 | OPA1 H31 | GATTTAAATATTTTTTA |
| | 3UTR(+2854+2870) | | | 3UTR(+2908+2924) | |
| 2265 | OPA1 H31 | TCAGGCAAAATATAAGT | 2283 | OPA1 H31 | TGTGATTTAAATATTTT |
| | 3UTR(+2857+2873) | | | 3UTR(+2911+2927) | |
| 2266 | OPA1 H31 | AGCTCAGGCAAAATATA | 2284 | OPA1 H31 | TAATGTGATTTAAATAT |
| | 3UTR(+2860+2876) | | | 3UTR(+2914+2930) | |
| 2267 | OPA1 H31 | TCAAGCTCAGGCAAAAT | 2285 | OPA1 H31 | TTATAATGTGATTTAAA |
| | 3UTR(+2863+2879) | | | 3UTR(+2917+2933) | |
| 2268 | OPA1 H31 | TGATCAAGCTCAGGCAA | 2286 | OPA1 H31 | GAATTATAATGTGATTT |
| | 3UTR(+2866+2882) | | | 3UTR(+2920+2936) | |
| 2269 | OPA1 H31 | CTTTGATCAAGCTCAGG | 2287 | OPA1 H31 | ATAGAATTATAATGTGA |
| | 3UTR(+2869+2885) | | | 3UTR(+2923+2939) | |
| 2270 | OPA1 H31 | GACCTTTGATCAAGCTC | 2288 | OPA1 H31 | ATAATAGAATTATAATG |
| | 3UTR(+2872+2888) | | | 3UTR(+2926+2942) | |
| 2271 | OPA1 H31 | AATGACCTTTGATCAAG | 2289 | OPA1 H31 | CCAATAATAGAATTATA |
| | 3UTR(+2875+2891) | | | 3UTR(+2929+2945) | |
| 2272 | OPA1 H31 | ACAAATGACCTTTGATC | 2290 | OPA1 H31 | TCTCCAATAATAGAATT |
| | 3UTR(+2878+2894) | | | 3UTR(+2932+2948) | |
| 2273 | OPA1 H31 | TACACAAATGACCTTTG | 2291 | OPA1 H31 | TGCTCTCCAATAATAGA |
| | 3UTR(+2881+2897) | | | 3UTR(+2935+2951) | |
| 2274 | OPA1 H31 | ATCTACACAAATGACCT | 2292 | OPA1 H31 | AGATGCTCTCCAATAAT |
| | 3UTR(+2884+2900) | | | 3UTR(+2938+2954) | |
| 2275 | OPA1 H31 | CTCATCTACACAAATGA | 2293 | OPA1 H31 | AAAAGATGCTCTCCAAT |
| | 3UTR(+2887+2903) | | | 3UTR(+2941+2957) | |
| 2276 | OPA1 H31 | TTACTCATCTACACAAA | 2294 | OPA1 H31 | TTTAAAAGATGCTCTCC |
| | 3UTR(+2890+2906) | | | 3UTR(+2944+2960) | |
| 2277 | OPA1 H31 | TAATTACTCATCTACAC | 2295 | OPA1 H31 | AAATTTAAAAGATGCTC |
| | 3UTR(+2893+2909) | | | 3UTR(+2947+2963) | |
| 2278 | OPA1 H31 | TTTTAATTACTCATCTA | 2296 | OPA1 H31 | AAAAAATTTAAAAGATG |
| | 3UTR(+2896+2912) | | | 3UTR(+2950+2966) | |
| 2297 | OPA1 H31 | AGAAAAAAATTTAAAAG | 2315 | OPA1 H31 | ATGGGGTCAAATAATGA |
| | 3UTR(+2953+2969) | | | 3UTR(+3007+3023) | |
| 2298 | OPA1 H31 | AACAGAAAAAAATTTAA | 2316 | OPA1 H31 | ACTATGGGGTCAAATAA |
| | 3UTR(+2956+2972) | | | 3UTR(+3010+3026) | |
| 2299 | OPA1 H31 | TAAAACAGAAAAAAATT | 2317 | OPA1 H31 | TATACTATGGGGTCAAA |
| | 3UTR(+2959+2975) | | | 3UTR(+3013+3029) | |
| 2300 | OPA1 H31 | CGTTAAAACAGAAAAAA | 2318 | OPA1 H31 | GGTTATACTATGGGGTC |
| | 3UTR(+2962+2978) | | | 3UTR(+3016+3032) | |
| 2301 | OPA1 H31 | CCTCGTTAAAACAGAAA | 2319 | OPA1 H31 | TCTGGTTATACTATGGG |
| | 3UTR(+2965+2981) | | | 3UTR(+3019+3035) | |
| 2302 | OPA1 H31 | TTCCCTCGTTAAAACAG | 2320 | OPA1 H31 | GAATCTGGTTATACTAT |
| | 3UTR(+2968+2984) | | | 3UTR(+3022+3038) | |
| 2303 | OPA1 H31 | TCTTTCCCTCGTTAAAA | 2321 | OPA1 H31 | CATGAATCTGGTTATAC |
| | 3UTR(+2971+2987) | | | 3UTR(+3025+3041) | |
| 2304 | OPA1 H31 | TTCTCTTTCCCTCGTTA | 2322 | OPA1 H31 | GACCATGAATCTGGTTA |
| | 3UTR(+2974+2990) | | | 3UTR(+3028+3044) | |
| 2305 | OPA1 H31 | GGTTTCTCTTTCCCTCG | 2323 | OPA1 H31 | TTAGACCATGAATCTGG |
| | 3UTR(+2977+2993) | | | 3UTR(+3031+3047) | |
| 2306 | OPA1 H31 | ACAGGTTTCTCTTTCCC | 2324 | OPA1 H31 | TTGTTAGACCATGAATC |
| | 3UTR(+2980+2996) | | | 3UTR(+3034+3050) | |
| 2307 | OPA1 H31 | TATACAGGTTTCTCTTT | 2325 | OPA1 H31 | AGCTTGTTAGACCATGA |
| | 3UTR(+2983+2999) | | | 3UTR(+3037+3053) | |
| 2308 | OPA1 H31 | AGGTATACAGGTTTCTC | 2326 | OPA1 H31 | GAGAGCTTGTTAGACCA |
| | 3UTR(+2986+3002) | | | 3UTR(+3040+3056) | |
| 2309 | OPA1 H31 | CCTAGGTATACAGGTTT | 2327 | OPA1 H31 | ACTGAGAGCTTGTTAGA |
| | 3UTR(+2989+3005) | | | 3UTR(+3043+3059) | |
| 2310 | OPA1 H31 | GACCCTAGGTATACAGG | 2328 | OPA1 H31 | CACACTGAGAGCTTGTT |
| | 3UTR(+2992+3008) | | | 3UTR(+3046+3062) | |
| 2311 | OPA1 H31 | AATGACCCTAGGTATAC | 2329 | OPA1 H31 | AGCCACACTGAGAGCTT |
| | 3UTR(+2995+3011) | | | 3UTR(+3049+3065) | |
| 2312 | OPA1 H31 | AATAATGACCCTAGGTA | 2330 | OPA1 H31 | AAAAGCCACACTGAGAG |
| | 3UTR(+2998+3014) | | | 3UTR(+3052+3068) | |
| 2313 | OPA1 H31 | TCAAATAATGACCCTAG | 2331 | OPA1 H31 | GAGAAAAGCCACACTGA |
| | 3UTR(+3001+3017) | | | 3UTR(+3055+3071) | |
| 2314 | OPA1 H31 | GGGTCAAATAATGACCC | 2332 | OPA1 H31 | TCAGAGAAAAGCCACAC |
| | 3UTR(+3004+3020) | | | 3UTR(+3058+3074) | |
| 2333 | OPA1 H31 | CATTCAGAGAAAAGCCA | 2351 | OPA1 H31 | GCACCGGTTGACCATGG |
| | 3UTR(+3061+3077) | | | 3UTR(+3115+3131) | |
| 2334 | OPA1 H31 | AAGCATTCAGAGAAAAG | 2352 | OPA1 H31 | AAAGCACCGGTTGACCA |
| | 3UTR(+3064+3080) | | | 3UTR(+3118+3134) | |
| 2335 | OPA1 H31 | TTCAAGCATTCAGAGAA | 2353 | OPA1 H31 | AAAAAAGCACCGGTTGA |
| | 3UTR(+3067+3083) | | | 3UTR(+3121+3137) | |
| 2336 | OPA1 H31 | AAATTCAAGCATTCAGA | 2354 | OPA1 H31 | TGAAAAAAAGCACCGGT |
| | 3UTR(+3070+3086) | | | 3UTR(+3124+3140) | |
| 2337 | OPA1 H31 | GTGAAATTCAAGCATTC | 2355 | OPA1 H31 | ATGTGAAAAAAAGCACC |
| | 3UTR(+3073+3089) | | | 3UTR(+3127+3143) | |
| 2338 | OPA1 H31 | CATGTGAAATTCAAGCA | 2356 | OPA1 H31 | ACGATGTGAAAAAAAGC |
| | 3UTR(+3076+3092) | | | 3UTR(+3130+3146) | |
| 2339 | OPA1 H31 | AGGCATGTGAAATTCAA | 2357 | OPA1 H31 | ACCACGATGTGAAAAAA |
| | 3UTR(+3079+3095) | | | 3UTR(+3133+3149) | |
| 2340 | OPA1 H31 | GCAAGGCATGTGAAATT | 2358 | OPA1 H31 | AGTACCACGATGTGAAA |
| | 3UTR(+3082+3098) | | | 3UTR(+3136+3152) | |
| 2341 | OPA1 H31 | AATGCAAGGCATGTGAA | 2359 | OPA1 H31 | ACAAGTACCACGATGTG |
| | 3UTR(+3085+3101) | | | 3UTR(+3139+3155) | |
| 2342 | OPA1 H31 | TGAAATGCAAGGCATGT | 2360 | OPA1 H31 | TTGACAAGTACCACGAT |
| | 3UTR(+3088+3104) | | | 3UTR(+3142+3158) | |
| 2343 | OPA1 H31 | CTGTGAAATGCAAGGCA | 2361 | OPA1 H31 | GTTTTGACAAGTACCAC |
| | 3UTR(+3091+3107) | | | 3UTR(+3145+3161) | |
| 2344 | OPA1 H31 | CAACTGTGAAATGCAAG | 2362 | OPA1 H31 | AATGTTTTGACAAGTAC |
| | 3UTR(+3094+3110) | | | 3UTR(+3148+3164) | |
| 2345 | OPA1 H31 | GTACAACTGTGAAATGC | 2363 | OPA1 H31 | CAAAATGTTTTGACAAG |
| | 3UTR(+3097+3113) | | | 3UTR(+3151+3167) | |
| 2346 | OPA1 H31 | GGAGTACAACTGTGAAA | 2364 | OPA1 H31 | TAACAAAATGTTTTGAC |
| | 3UTR(+3100+3116) | | | 3UTR(+3154+3170) | |
| 2347 | OPA1 H31 | CATGGAGTACAACTGTG | 2365 | OPA1 H31 | AAATAACAAAATGTTTT |
| | 3UTR(+3103+3119) | | | 3UTR(+3157+3173) | |
| 2348 | OPA1 H31 | GACCATGGAGTACAACT | 2366 | OPA1 H31 | GGAAAATAACAAAATGT |
| | 3UTR(+3106+3122) | | | 3UTR(+3160+3176) | |
| 2349 | OPA1 H31 | GTTGACCATGGAGTACA | 2367 | OPA1 H31 | CAAGGAAAATAACAAAA |
| | 3UTR(+3109+3125) | | | 3UTR(+3163+3179) | |
| 2350 | OPA1 H31 | CCGGTTGACCATGGAGT | 2368 | OPA1 H31 | TACCAAGGAAAATAACA |
| | 3UTR(+3112+3128) | | | 3UTR(+3166+3182) | |
| 2369 | OPA1 H31 | TTTTACCAAGGAAAATA | 2388 | OPA1 H31 3UTR(−15−31) | ATTAATGAAAATGACAG |
| | 3UTR(+3169+3185) | | | | |
| 2370 | OPA1 H31 | ATATTTTACCAAGGAAA | 2389 | OPA1 H31 3UTR(−18−34) | AGCATTAATGAAAATGA |
| | 3UTR(+3172+3188) | | | | |
| 2371 | OPA1 H31 | TATATATTTTACCAAGG | 2390 | OPA1 H31 3UTR(−21−37) | GGCAGCATTAATGAAAA |
| | 3UTR(+3175+3191) | | | | |
| 2372 | OPA1 H31 | TTTTATATATTTTACCA | 2391 | OPA1 H31 3UTR(−24−40) | GTTGGCAGCATTAATGA |
| | 3UTR(+3178+3194) | | | | |
| 2373 | OPA1 H31 | CTTTTTTATATATTTTA | 2392 | OPA1 H31 3UTR(−27−43) | CATGTTGGCAGCATTAA |
| | 3UTR(+3181+3197) | | | | |
| 2374 | OPA1 H31 | AACCTTTTTTATATATT | 2393 | OPA1 H31 3UTR(−30−46) | GAACATGTTGGCAGCAT |
| | 3UTR(+3184+3200) | | | | |
| 2375 | OPA1 H31 | GAAAACCTTTTTTATAT | 2394 | OPA1 H31 3UTR(−33−49) | TATGAACATGTTGGCAG |
| | 3UTR(+3187+3203) | | | | |
| 2376 | OPA1 H31 | TTAGAAAACCTTTTTTA | 2395 | OPA1 H31 3UTR(−36−52) | TCATATGAACATGTTGG |
| | 3UTR(+3190+3206) | | | | |
| 2377 | OPA1 H31 | AAATTAGAAAACCTTTT | 2396 | OPA1 H31 3UTR(−39−55) | GCCTCATATGAACATGT |
| | 3UTR(+3193+3209) | | | | |
| 2378 | OPA1 H31 | GTGAAATTAGAAAACCT | 2397 | OPA1 H31 3UTR(−42−58) | TAAGCCTCATATGAACA |
| | 3UTR(+3196+3212) | | | | |
| 2379 | OPA1 H31 | AAAGTGAAATTAGAAAA | 2398 | OPA1 H31 3UTR(−45−61) | TAGTAAGCCTCATATGA |
| | 3UTR(+3199+3215) | | | | |
| 2380 | OPA1 H31 | AGCAAAGTGAAATTAGA | 2399 | OPA1 H31 3UTR(−48−64) | TCTTAGTAAGCCTCATA |
| | 3UTR(+3202+3218) | | | | |
| 2381 | OPA1 H31 | GGCAGCAAAGTGAAATT | 2400 | OPA1 H31 3UTR(−51−67) | ACTTCTTAGTAAGCCTC |
| | 3UTR(+3205+3221) | | | | |
| 2382 | OPA1 H31 | CTTGGCAGCAAAGTGAA | 2401 | OPA1 H31 3UTR(−54−70) | ATAACTTCTTAGTAAGC |
| | 3UTR(+3208+3224) | | | | |
| 2383 | OPA1 H31 3UTR | AGCCTTGGCAGCAAAGT | 2402 | OPA1 H31 3UTR(−57−73) | GTTATAACTTCTTAGTA |
| | (+3211−16) | | | | |
| 2384 | OPA1 H31 3UTR(−3−19) | GACAGCCTTGGCAGCAA | 2403 | OPA1 H31 3UTR(−60−76) | TTAGTTATAACTTCTTA |
| 2385 | OPA1 H31 3UTR(−6−22) | AATGACAGCCTTGGCAG | 2404 | OPA1 H31 3UTR(−63−79) | AGCTTAGTTATAACTTC |
| 2386 | OPA1 H31 3UTR(−9−25) | GAAAATGACAGCCTTGG | 2405 | OPA1 H31 3UTR(−66−82) | TAAAGCTTAGTTATAAC |
| 2387 | OPA1 H31 3UTR(−12−28) | AATGAAAATGACAGCCT | 2406 | OPA1 H31 3UTR(−69−85) | TTCTAAAGCTTAGTTAT |
| 2407 | OPA1 H31 3UTR(−72−88) | CAGTTCTAAAGCTTAGT | 2425 | OPA1 H31 3UTR | AAAGTGAGTAACAGGAG |
| | | | | (−126−142) | |
| 2408 | OPA1 H31 3UTR(−75−91) | TTCCAGTTCTAAAGCTT | 2426 | OPA1 H31 3UTR | AACAAAGTGAGTAACAG |
| | | | | (−129−145) | |
| 2409 | OPA1 H31 3UTR(−78−94) | CTCTTCCAGTTCTAAAG | 2427 | OPA1 H31 3UTR | AATAACAAAGTGAGTAA |
| | | | | (−132−148) | |
| 2410 | OPA1 H31 3UTR(−81−97) | AGTCTCTTCCAGTTCTA | 2428 | OPA1 H31 3UTR | AGGAATAACAAAGTGAG |
| | | | | (−135−151) | |
| 2411 | OPA1 H31 3UTR(−84−100) | TAAAGTCTCTTCCAGTT | 2429 | OPA1 H31 3UTR | TGGAGGAATAACAAAGT |
| | | | | (−138−154) | |
| 2412 | OPA1 H31 3UTR(−87−103) | AATTAAAGTCTCTTCCA | 2430 | OPA1 H31 3UTR | TTCTGGAGGAATAACAA |
| | | | | (−141−157) | |
| 2413 | OPA1 H31 3UTR(−90−106) | TGAAATTAAAGTCTCTT | 2431 | OPA1 H31 3UTR | AAGTTCTGGAGGAATAA |
| | | | | (−144−160) | |
| 2414 | OPA1 H31 3UTR(−93−109) | AGATGAAATTAAAGTCT | 2432 | OPA1 H31 3UTR | GCAAAGTTCTGGAGGAA |
| | | | | (−147−163) | |
| 2415 | OPA1 H31 3UTR(−96−112) | GGTAGATGAAATTAAAG | 2433 | OPA1 H31 3UTR | TGAGCAAAGTTCTGGAG |
| | | | | (−150−166) | |
| 2416 | OPA1 H31 3UTR(−99−115) | GATGGTAGATGAAATTA | 2434 | OPA1 H31 3UTR | TTATGAGCAAAGTTCTG |
| | | | | (−153−169) | |
| 2417 | OPA1 H31 3UTR(−102−118) | TCAGATGGTAGATGAAA | 2435 | OPA1 H31 3UTR | CATTTATGAGCAAAGTT |
| | | | | (−156−172) | |
| 2418 | OPA1 H31 3UTR(−105−121) | TAATCAGATGGTAGATG | 2436 | OPA1 H31 3UTR | TATCATTTATGAGCAAA |
| | | | | (−159−175) | |
| 2419 | OPA1 H31 3UTR(−108−124) | TTATAATCAGATGGTAG | 2437 | OPA1 H31 3UTR | AGGTATCATTTATGAGC |
| | | | | (−162−178) | |
| 2420 | OPA1 H31 3UTR(−111−127) | AGTTTATAATCAGATGG | 2438 | OPA1 H31 3UTR | AGTAGGTATCATTTATG |
| | | | | (−165−181) | |
| 2421 | OPA1 H31 3UTR(−114−130) | AGGAGTTTATAATCAGA | 2439 | OPA1 H31 3UTR | ATTAGTAGGTATCATTT |
| | | | | (−168−184) | |
| 2422 | OPA1 H31 3UTR(−117−133) | AACAGGAGTTTATAATC | 2440 | OPA1 H31 3UTR | ACAATTAGTAGGTATCA |
| | | | | (−171−187) | |
| 2423 | OPA1 H31 3UTR(−120−136) | AGTAACAGGAGTTTATA | 2441 | OPA1 H31 3UTR | TCGACAATTAGTAGGTA |
| | | | | (−174−190) | |
| 2424 | OPA1 H31 3UTR(−123−139) | GTGAGTAACAGGAGTTT | 2442 | OPA1 H31 3UTR | TGATCGACAATTAGTAG |
| | | | | (−177−193) | |
| 2443 | OPA1 H31 3UTR(−180−196) | CAATGATCGACAATTAG | 2461 | OPA1 H31 3UTR | ATCTTCTGAAGGAATTA |
| | | | | (−234−250) | |
| 2444 | OPA1 H31 3UTR(−183−199) | ATCCAATGATCGACAAT | 2462 | OPA1 H31 3UTR | TGCATCTTCTGAAGGAA |
| | | | | (−237−253) | |
| 2445 | OPA1 H31 3UTR(−186−202) | CATATCCAATGATCGAC | 2463 | OPA1 H31 3UTR | CTCTGCATCTTCTGAAG |
| | | | | (−240−256) | |
| 2446 | OPA1 H31 3UTR(−189−205) | TGACATATCCAATGATC | 2464 | OPA1 H31 3UTR | TGCCTCTGCATCTTCTG |
| | | | | (−243−259) | |
| 2447 | OPA1 H31 3UTR(−192−208) | ACTTGACATATCCAATG | 2465 | OPA1 H31 3UTR | TCTTGCCTCTGCATCTT |
| | | | | (−246−262) | |
| 2448 | OPA1 H31 3UTR(−195−211) | CTAACTTGACATATCCA | 2466 | OPA1 H31 3UTR | TGTTCTTGCCTCTGCAT |
| | | | | (−249−265) | |
| 2449 | OPA1 H31 3UTR(−198−214) | TACCTAACTTGACATAT | 2467 | OPA1 H31 3UTR | ACATGTTCTTGCCTCTG |
| | | | | (−252−268) | |
| 2450 | OPA1 H31 3UTR(−201−217) | CGCTACCTAACTTGACA | 2468 | OPA1 H31 3UTR | GAAACATGTTCTTGCCT |
| | | | | (−255−271) | |
| 2451 | OPA1 H31 3UTR(−204−220) | ATACGCTACCTAACTTG | 2469 | OPA1 H31 3UTR | ATTGAAACATGTTCTTG |
| | | | | (−258−274) | |
| 2452 | OPA1 H31 3UTR(−207−223) | CCTATACGCTACCTAAC | 2470 | OPA1 H31 3UTR | ACGATTGAAACATGTTC |
| | | | | (−261−277) | |
| 2453 | OPA1 H31 3UTR(−210−226) | ACACCTATACGCTACCT | 2471 | OPA1 H31 3UTR | AACACGATTGAAACATG |
| | | | | (−264−280) | |
| 2454 | OPA1 H31 3UTR(−213−229) | GGCACACCTATACGCTA | 2472 | OPA1 H31 3UTR | GCTAACACGATTGAAAC |
| | | | | (−267−283) | |
| 2455 | OPA1 H31 3UTR(−216−232) | GAAGGCACACCTATACG | 2473 | OPA1 H31 3UTR | AATGCTAACACGATTGA |
| | | | | (−270−286) | |
| 2456 | OPA1 H31 3UTR(−219−235) | TAAGAAGGCACACCTAT | 2474 | OPA1 H31 3UTR | ACCAATGCTAACACGAT |
| | | | | (−273−289) | |
| 2457 | OPA1 H31 3UTR(−222−238) | AATTAAGAAGGCACACC | 2475 | OPA1 H31 3UTR | AAAACCAATGCTAACAC |
| | | | | (−276−292) | |
| 2458 | OPA1 H31 3UTR(−225−241) | AGGAATTAAGAAGGCAC | 2476 | OPA1 H31 3UTR | AAGAAAACCAATGCTAA |
| | | | | (−279−295) | |
| 2459 | OPA1 H31 3UTR(−228−244) | TGAAGGAATTAAGAAGG | 2477 | OPA1 H31 3UTR | ATAAAGAAAACCAATGC |
| | | | | (−282−298) | |
| 2460 | OPA1 H31 3UTR(−231−247) | TTCTGAAGGAATTAAGA | 2478 | OPA1 H31 3UTR | CTGATAAAGAAAACCAA |
| | | | | (−285−301) | |
| 2479 | OPA1 H31 3UTR(−288−304) | GCACTGATAAAGAAAAC | 2484 | OPA1 H31 3UTR | ACACTGAGTCTGTTAGC |
| | | | | (−303−319) | |
| 2480 | OPA1 H31 3UTR(−291−307) | TTAGCACTGATAAAGAA | 2485 | OPA1 H31 3UTR | CTCACACTGAGTCTGTT |
| | | | | (−306−322) | |
| 2481 | OPA1 H31 3UTR(−294−310) | CTGTTAGCACTGATAAA | 2486 | OPA1 H31 3UTR | GGCCTCACACTGAGTCT |
| | | | | (−309−325) | |
| 2482 | OPA1 H31 3UTR(−297−313) | AGTCTGTTAGCACTGAT | 2487 | OPA1 H31 3UTR | TGGGGCCTCACACTGAG |
| | | | | (−312−328) | |
| 2483 | OPA1 H31 3UTR(−300−316) | CTGAGTCTGTTAGCACT | 2488 | OPA1 H31 3UTR | GTATGGGGCCTCACACT |
| | | | | (−315−331) | |

SEQ ID: 2489: OPA1 transcript cDNA sequence (NM_130837)
AGGCTCTTGCGGAAGTCCATGCGCCATTGGGAGGGCCTCGGCCGCGGCTCTGTGCCCTTGCTGCTGAGGGCCACTTCCTGGGTCATTCCTGGACCGGGAGCC
GGGCTGGGGCTCACACGGGGGCTCCCGCGTGGCCGTCTCGGCGCCTGCGTGACCTCCCCGCCGGCGGGATGTGGCGACTACGTCGGGCCGCTGTGGCCTGTG
AGGTCTGCCAGTCTTTAGTGAAACACAGCTCTGGAATAAAAGGAAGTTTACCACTACAAAAACTACATCTGGTTTCACGAAGCATTTATCATTCACATCATC
CTACCTTAAAGCTTCAACGACCCCAATTAAGGACATCCTTTCAGCAGTTCTCTTCTCTGACAAACCTTCCTTTACGTAAACTGAAATTCTCTCCAATTAAATAT
GGCTACCAGCCTCGCAGGAATTTTTGGCCAGCAAGATTAGCTACGAGACTCTTAAAACTTCGCTATCTCATACTAGGATCGGCTGTTGGGGGTGGCTACACA
GCCAAAAAGACTTTTGATCAGTGGAAAGATATGATACCGGACCTTAGTGAATATAAATGGATTGTGCCTGACATTGTGTGGGAAATTGATGAGTATATCGAT
TTTGAGAAAATTAGAAAAGCCCTTCCTAGTTCAGAAGACCTTGTAAAGTTAGCACCAGACTTTGACAAGATTGTTGAAAGCCTTAGCTTATTGAAGGACTTT
TTTACCTCAGGTCACAAATTGGTTAGTGAAGTCATAGGAGCTTCTGACCTACTTCTCTTGTTAGGTTCTCCGGAAGAAACGGCGTTTAGAGCAACAGATCGTG
GATCTGAAAGTGACAAGCATTTTAGAAAGGGTCTGCTTGGTGAGCTCATTCTCTTACAACAACAAATTCAAGAGCATGAAGAGGAAGCGCGCAGAGCCGCT
GGCCAATATAGCACGAGCTATGCCCAACAGAAGCGCAAGGTGTCAGACAAAGAGAAAATTGACCAACTTCAGGAAGAACTTCTGCACACTCAGTTGAAGTA
TCAGAGAATCTTGGAACGATTAGAAAAGGAGAACAAAGAATTGAGAAAATTAGTATTGCAGAAAGATGACAAAGGCATTCATCATAGAAAGCTTAAGAAA
TCTTTGATTGACATGTATTCTGAAGTTCTTGATGTTCTCTCTGATTATGATGCCAGTTATAATACGCAAGATCATCTGCCACGGGTTGTTGTGGTTGGAGATCA
GAGTGCTGGAAAGACTAGTGTGTTGGAAATGATTGCCCAAGCTCGAATATTCCCAAGAGGATCTGGGGAGATGATGACACGTTCTCCAGTTAAGGTGACTCT
GAGTGAAGGTCCTCACCATGTGGCCCTATTTAAAGATAGTTCTCGGGAGTTTGATCTTACCAAAGAAGAAGATCTTGCAGCATTAAGACATGAAATAGAACT
TCGAATGAGGAAAAATGTGAAAGAAGGCTGTACCGTTAGCCCTGAGACCATATCCTTAAATGTAAAAGGCCCTGGACTACAGAGGATGGTGCTTGTTGACT
TACCAGGTGTGATTAATACTGTGACATCAGGCATGGCTCCTGACACAAAGGAAACTATTTTCAGTATCAGCAAAGCTTACATGCAGAATCCTAATGCCATCA
TACTGTGTATTCAAGATGGATCTGTGGATGCTGAACGCAGTATTGTTACAGACTTGGTCAGTCAAATGGACCCTCATGGAAGGAGAACCATATTCGTTTTGA
CCAAAGTAGACCTGGCAGAGAAAAATGTAGCCAGTCCAAGCAGGATTCAGCAGATAATTGAAGGAAAGCTCTTCCCAATGAAAGCTTTAGGTTATTTTGCT
GTTGTAACAGGAAAAGGGAACAGCTCTGAAAGCATTGAAGCTATAAGAGAATATGAAGAAGAGTTTTTTCAGAATTCAAAGCTCCTAAAGACAAGCATGCT
AAAGGCACACCAAGTGACTACAAGAAATTTAAGCCTTGCAGTATCAGACTGCTTTTGGAAAATGGTACGAGAGTCTGTTGAACAACAGGCTGATAGTTTCA
AAGCAACACGTTTTAACCTTGAAACTGAATGGAAGAATAACTATCCTCGCCTGCGGGAACTTGACCGGAATGAACTATTTGAAAAAGCTAAAAATGAAATC
CTTGATGAAGTTATCAGTCTGAGCCAGGTTACACCAAAACATTGGGAGGAAATCCTTCAACAATCTTTGTGGGAAAGAGTATCAACTCATGTGATTGAAAAC
ATCTACCTTCCAGCTGCGCAGACCATGAATTCAGGAACTTTTAACACCACAGTGGATATCAAGCTTAAACAGTGGACTGATAAACAACTTCCTAATAAAGCA
GTAGAGGTTGCTTGGGAGACCCTACAAGAAGAATTTTCCCGCTTTATGACAGAACCGAAAGGGAAAGAGCATGATGACATATTTGATAAACTTAAAGAGGC
TGTTAAGGAAGAAAGTATTAAACGACACAAGTGGAATGACTTTGCGGAGGACAGCTTGAGGGTTATTCAACACAATGCTTTGGAAGACCGATCCATATCTG
ATAAACAGCAATGGGATGCAGCTATTTATTTTATGGAAGAGGCTCTGCAGGCTCGTCTCAAGGATACTGAAAATGCAATTGAAAACATGGTGGGTCCAGAC
TGGAAAAAGAGGTGGTTATACTGGAAGAATCGGACCCAAGAACAGTGTGTTCACAATGAAACCAAGAATGAATTGGAGAAGATGTTGAAATGTAATGAGG
AGCACCCAGCTTATCTTGCAAGTGATGAAATAACCACAGTCCGGAAGAACCTTGAATCCCGAGGAGTAGAAGTAGATCCAAGCTTGATTAAGGATACTTGG
CATCAAGTTTATAGAAGACATTTTTTAAAAACAGCTCTAAACCATTGTAACCTTTGTCGAAGAGGTTTTTATTACTACCAAAGGCATTTTGTAGATTCTGAGT
TGGAATGCAATGATGTGGTCTTGTTTTGGCGTATACAGCGCATGCTTGCTATCACCGCAAATACTTTAAGGCAACAACTTACAAATACTGAAGTTAGGCGAT
TAGAGAAAAATGTTAAAGAGGTATTGGAAGATTTTGCTGAAGATGGTGAGAAGAAGATTAAATTGCTTACTGGTAAACGCGTTCAACTGGCGGAAGACCTC
AAGAAAGTTAGAGAAATTCAAGAAAAACTTGATGCTTTCATTGAAGCTCTTCATCAGGAGAAATAAATTAAAATCGTACTCATAATCAGCTCTGCATACATC
TGAAGAACAAAAACATCAACGTCTTTTGTCCAGCCTCTTTTTCTTCTGCTGTTCCACCTTTCTAAACATACAATAAAGTCATGGGATAAAAATAATCGATGTA
TGTTACGGGCGCTTTAACCATCAGCTGCCTCTCGAATGGAAGAACAGTGGTAATGGATTAACATCCTATTTTGTTGTACTAAAGTGACAAATCGGAATAATA
TAATTGGTATGGCCATTAGGTTCAGTCCTTGAAGATAAGAAACTTGTTCTCTGTTTGTTGTCTTATTTGTGGTGGCACTCGTTTAATGGATTAACTGAGGTTGC
TCAATGTTCAGTTTCTTTTCCAGAAATACAATGCTAGGTGTTTTGAAATAAAACTTATATAGCAATTGTTTAAAGTTATCAATTGTATATAAAATCACAGTAG
CCTGCTAAATCATTGTATGTGTCTGTAGTATTCTATTCCCAGAAACTATTTGACCATGATAATTCAGTTTATATTCACCACATGAAAGAAAAATGGGTAACAG
AAGAACCCTTAAAACAGGTTAATTTGGATTGTAACGTTCAGTGAAAGAAATTTCAACCCTTCATAGCCAGCGAAGAAATTTGCCTTGGAAGCCAAGTCAGTA
CCAGCTTACCTATTTGATTCAGTTGCTGTTTTCTCACTCTCTATATCCATTTGAAATTGATTTATTTTAGATGTTGTATACTTACGTTAGGCTTTCTGTTAATAG
TGGTTTTTCTCCTGTTGACAGAGCCACCGGATTATGACACAGGATGAGGAAGATTAAGGATAATCAATTGACTAATTTCATTTAGAATATTATCAAACATTTC
AACTAGGTATCAGAAAAAGGCTTTCTTTCATAAGACTATTTTAAATAGAAATTATTTCAACAATTAAAGTAATGTTGACCATCCCCCTCTCAGCTGAATAAA
GAAAAATTTAGTTCAATTTATTGCAATTTAATTACAATACTACCTTCACAACATTTTCATGTGTTTTAAATAAATATTTTTTAATTGGCTAAAGGACATTCAAG
CAAAGAAATGCTTTCTTTACTTAAAATGTCTATCTCATTTGCTGCCTTTTCACTAAGCCTTTACTTTGTTAATAAAAGTGTCCATTGTGTGATGTTTTTGATTTT
ACAGTTTGCTAAATCTTATTTTCTTGGAGTTGCTTTTTGGTAACAGCCCCATTGCTACTCCCCATTTTATTGTTTTACATCAATGCATGCTTCGTTGTGATCCCT
CAAGATGTAACACTTGGTATGCTCGGTTGAGGATATGAAAAAATACTTCCGAAACCAGGAATTCAATGTATGTTTGTTTTATACTGTTTGATAAGAAAAGTA
GGTCCAGCCTTAAGCAGCACAGATGCGCTGGTAGATGCATAGTCAGGAACTTTTTTTATTTCTTTTAGGTCTAGGGACAGGAGTGAATAGAAAGGGAGGAG
AGCTCTATTATGTTCTATACACAGATTAGGAGATGACCTTACTGGGTACACCCCTCTAACCAGTGCTTACAGGTTAATGCATGTTAATGAATATTTTTGCAGT
TGTAAAGCATAACAATTACAACTACACATCTATTTCTAAAGAATAAAACAGGACCATATTTATTTACTTCTGTCAACTATAGAAAGAAAGACCTTCAGCTGT
ATTTCCACAGATTTCTCCCAAGGAAAAGGCTAATATTAGTCACTACTGTTATCACATCCCTTTGTATAAGTTTTAAAAAGAGATGGAGGGAGATCTTCATTTC
TTTGAGGAGATCAGTATTGTAACGTATGTGAATAGATGATAACAATTAATATTACTAAAAGTCCCACATGAGAGTCCTGACGCCCTCTCCATGCCCCACAGT
AATGTGGCTTCTTTCATGGGTTTTTTTTTCTTCTTTTTAGCTGATCTCATCCTAAGCATGCTTTATTTTTCCTTGAAAGCTAGGTATTTATCAACTGCAGATGTT
ATTGAAAGAAAATAAAATTCAGTCTCAAGAGTAAACCCTGTGTCTTGTGTCTGTAGTTCAAAAGTCAGAAATGATTCTAATTTAAACAAAAAGATACTAAAT
ATACAGAAGTTAAATTCGAACTAGCCACAGAATCATTTGTTTTTATGTCAGAATTTGCAAAGAGTGGAGTGGACAAAGCTCTGTATGGAAGACTGAACAACT
GTAAATAGATGATATCCAAACTTAATTTGGCTAGGACTTCAATTTTAAAAATCAGTGTACCTAGGCAGTGCACAGCACGAAATAAGTGGCCCTTGCAGCTTC
CCCGTTTAACCCACTGTGCTATAGTTGCGGGTGGAACAGTCAACCTTTCTAGTAGTTTATGATATTGCCCTCTTTGTATTCCCATTTTCTACAGTTTTTTCCGC
AGACTTCTTTCTGCAAATTATTCAGCCTCCAAATGCAAATGAATGATATAAAAATAAGTAGGGAACATGGCAGAGAGTGGTGCTTCCCAGCCTCACAATGTG
GGAATTTGACATAGGATGAGAGTCAGAGTATAGGTTTAAAAGATAAAATCTTTAGTTAATAATTTTGTATTTATTTATTCTAGATGTATGTATCTGAGGAAAG
AAATCTGGTATTTTTGCTTTCCAATAAAGGGGATCAAAGTAATGGTTTTTCTCTCAGTTCTCTAAGCTGGTCTATGTTATAGCTCTAGCAGTATGGAAATGTG
CTTTAAAATATGCTTACCTTTTGAATGATCATGGCTATATGTTGTTGAGATATTTGAAACTTACCTTGTTTTCACTTGTGCACTGTGAATGAACTTTGTATTAT
TTTTTTAAAACCTTCACATTACGTGTAGATATTATTGCAACTTATATTTTGCCTGAGCTTGATCAAAGGTCTTTGTGTAGATGAGTAATTAAAAAATATTTAAA
TCACATTATAATTCTATTATTGGAGAGCATCTTTTAAATTTTTTTCTGTTTTAACGAGGGAAAGAGAAACCTGTATACCTAGGGTCATTATTTGACCCCATAGT
ATAACCAGATTCATGGTCTAACAAGCTCTCAGTGTGGCTTTTCTCTGAATGCTTGAATTTCACATGCCTTGCATTTCACAGTTGTACTCCATGGTCAACCGGT
GCTTTTTTTCACATCGTGGTACTTGTCAAAACATTTTGTTATTTTCCTTGGTAAAATATATAAAAAAGGTTTTCTAATTTCA
SEQ ID: 2490: OPA1 protein sequence (UniProt ID 060313)
MWRLRRAAVACEVCQSLVKHSSGIKGSLPLQKLHLVSRSIYHSHHPTLKLQRPQLRTSFQQFSSL
TNLPLRKLKFSPIKYGYQPRRNFWPARLATRLLKLRYLILGSAVGGGYTAKKTFDQWKDMIPDL
SEYKWIVPDIVWEIDEYIDFEKIRKALPSSEDLVKLAPDFDKIVESLSLLKDFFTSGSPEETAFRAT
DRGSESDKHFRKVSDKEKIDQLQEELLHTQLKYQRILERLEKENKELRKLVLQKDDKGIHHRKL
KKSLIDMYSEVLDVLSDYDASYNTQDHLPRVVVVGDQSAGKTSVLEMIAQARIFPRGSGEMMT
RSPVKVTLSEGPHHVALFKDSSREFDLTKEEDLAALRHEIELRMRKNVKEGCTVSPETISLNVKG
PGLQRMVLVDLPGVINTVTSGMAPDTKETIFSISKAYMQNPNAIILCIQDGSVDAERSIVTDLVSQ
MDPHGRRTIFVLTKVDLAEKNVASPSRIQQIIEGKLFPMKALGYFAVVTGKGNSSESIEAIREYEE
EFFQNSKLLKTSMLKAHQVTTRNLSLAVSDCFWKMVRESVEQQADSFKATRFNLETEWKNNY
PRLRELDRNELFEKAKNEILDEVISLSQVTPKHWEEILQQSLWERVSTHVIENIYLPAAQTMNSG
TFNTTVDIKLKQWTDKQLPNKAVEVAWETLQEEFSRFMTEPKGKEHDDIFDKLKEAVKEESIKR
HKWNDFAEDSLRVIQHNALEDRSISDKQQWDAAIYFMEEALQARLKDTENAIENMVGPDWKK
RWLYWKNRTQEQCVHNETKNELEKMLKCNEEHPAYLASDEITTVRKNLESRGVEVDPSLIKDT
WHQVYRRHFLKTALNHCNLCRRGFYYYQRHFVDSELECNDVVLFWRIQRMLAITANTLRQQL
TNTEVRRLEKNVKEVLEDFAEDGEKKIKLLTGKRVQLAEDLKKVREIQEKLDAFIEALHQEK

TABLE 6
PMO refinement to target intron 7
of an OPA1 transcript

| SEQ | | |
|---|---|---|
| ID | ASO Coordinates | Sequence 5′>3′ |
| 2491 | OPA1_H7xA(-134-105) | AAATACATATATAAAAAGGTCATTGTAAAA |
| 2492 | OPA1_H7xA(−133−104) | GAAATACATATATAAAAAGGTCATTGTAAA |
| 2493 | OPA1_H7xA(−132−108) | TACATATATAAAAAGGTCATTGTAA |
| 2494 | OPA1_H7xA(−132−103) | TGAAATACATATATAAAAAGGTCATTGTAA |
| 2495 | OPA1_H7xA(−131−102) | CTGAAATACATATATAAAAAGGTCATTGTA |
| 2496 | OPA1_H7xA(−127−98) | AATTCTGAAATACATATATAAAAAGGTCAT |
| 2497 | OPA1_H7xA(−126−97) | AAATTCTGAAATACATATATAAAAAGGTCA |
| 2498 | OPA1_H7xA(−126−102) | CTGAAATACATATATAAAAAGGTCA |
| 2499 | OPA1_H7xA(−125−96) | GAAATTCTGAAATACATATATAAAAAGGTC |

TABLE 7
PMO refinement to target the
5′UTR of an OPA1 transcript

| SEQ ID | ASO Coordinates | Sequence 5′>3′ |
|---|---|---|
| 2500 | hOPA1_H1A(-45-24) | CATGCGCACAG |
| | 2MM | TGCGTCGGAAT |
| 2501 | hOPA1_H1A(−2+21) | CCTTTCTAGGC |
| | 2MM | GTGCAGCACTA |
| 2502 | hOPA1_H1A(+64+3085) | CATGGACTTCT |
| | 2MM | |
| | | GCAAGAGCCTA |
| 2503 | hOPA1_H1A(+66+87) | CGCATGGACTT |
| | 2MM | CCGCAAGAGAT |

SEQ ID NO: 2504: CPP Sequence
RRSRTARAGRPGRNSSRPSAPRGASGGASG

1. A method of treating, preventing and/or delaying progression of glaucoma in a subject, the method comprising administering an antisense oligonucleotide that modulates mRNA productive transcript, stability and/or translation of OPA1 gene transcript or part thereof.

2. The method of claim 1, wherein the antisense oligonucleotide increases the level of OPA1 mRNA or the amount of functional OPA1 protein in a cell and/or a tissue of the subject.

3. The method of claim 2, wherein the amount of functional OPA1 protein in the cell and/or the tissue is increased by about 1.1 to about 10-fold.

4. The method of claim 2, wherein the tissue is selected from the group consisting of the retina, retinal pigment epithelium and combinations thereof.

5. The method of claim 1, wherein the antisense oligonucleotide binds to a targeted portion of:
(i) an OPA1 gene pre-mRNA in a cell to promote exclusion of a nonsense-mediated RNA decay-inducing (NMD) exon during splicing of the OPA1 pre-mRNA to increase the level of OPA1 mRNA transcripts encoding full length, functional OPA1;
(ii) the 5′ untranslated region (UTR) of an OPA1 gene transcript in a cell to increase translation efficiency of an OPA1 mRNA;
(iii) the 5′ UTR of an OPA1 gene transcript in a cell to increase transcript stability; and/or
(iv) the 3′ UTR of an OPA1 gene transcript in a cell to increase transcript stability.

6. The method of claim 5, wherein the antisense oligonucleotide binds to intron 7 of an OPA1 gene pre-mRNA in a cell and increases the level of OPA1 gene transcripts encoding full length, functional OPA1 by exclusion of NMD exon 7×.

7. The method of claim 1, wherein:
a) the antisense oligonucleotide binds within a targeted portion of the OPA1 pre-mRNA nucleotide sequence corresponding to SEQ ID NOs: 1, 55, 139, or
b) the antisense oligonucleotide binds within a targeted portion of the 5′ UTR of OPA1 mRNA, wherein the targeted portion is within the nucleotide sequence corresponding to SEQ ID NO: 55; or
c) the antisense oligonucleotide binds within a targeted portion of the 3′ UTR of OPA1 mRNA, wherein the targeted portion is within the nucleotide sequence corresponding to SEQ ID NO: 139.

8. (canceled)

9. (canceled)

10. (canceled)

11. The method of claim 10, wherein the antisense oligonucleotide comprises;
a) a backbone modification comprising a phosphorothioate linkage or a phosphorodiamidate linkage; and/or
b) a phosphorodiamidate morpholino, a locked nucleic acid, a peptide nucleic acid, a 2′-O-methyl, a 2′-Fluoro, or a 2′-O-methoxyethyl moiety; and/or
c) at least one modified sugar moiety; and/or
d) a 2′-O-methoxyethyl moiety.

12. (canceled)

13. (canceled)

14. The method of claim 11, wherein:
a) each sugar moiety in the antisense oligonucleotide is a modified sugar moiety; and/or
b) each nucleotide of the antisense oligonucleotide comprises a 2′-O-methoxyethyl moiety.

15. (canceled)

16. (canceled)

17. The method of claim 1, wherein the nucleotide sequence of the antisense oligonucleotide consists of 10 to 50 nucleotides, 15 to 40 nucleotides, 18 to 40 nucleotides, 17 to 25 nucleotides, 20 to 35 nucleotides, 20 to 30 nucleotides, 22 to 30 nucleotides, 22 to 28 nucleotides, 24 to 30 nucleotides, 25 to 30 nucleotides, or 26 to 30 nucleotides.

18. (canceled)

19. The method of claim 17, wherein the antisense oligonucleotide comprises one or more phosphorodiamidate morpholino moieties.

20. The method of claim 1, wherein the antisense oligonucleotide is linked to a functional moiety.

21. The method of claim 20, wherein the functional moiety;
a) comprises or consists of a delivery moiety or a stabilising moiety;
b) is covalently or non-covalently linked to the antisense oligonucleotide; and/or
c) is linked to the 5′ end of the antisense oligonucleotide or is linked to the 3′ end of the antisense oligonucleotide.

22. The method of claim 21, wherein the delivery moiety;
a) is selected from the group consisting of lipids, peptides, carbohydrates, and antibodies; and/or
b) comprises a cell-penetrating peptide (CPP) or a N-acetylgalactosamine (GalNAc) moiety.

23. (canceled)

24. (canceled)

25. (canceled)

26. The method of claim 1, wherein the nucleotide sequence of the antisense oligonucleotide;
a) is at least 60%, 65%, 70%, 75%, 80%, 85%, 90%, 95%, or 100% complementary to the nucleotide sequence of the targeted portion over the length of the antisense oligonucleotide; and/or
b) corresponds to any one of SEQ ID NOs: 2-54, 56-138, 140-2488 or 2491-2503.

27. (canceled)

28. The method of claim 1, wherein the antisense oligonucleotide is complexed with a delivery nanocarrier.

29. The method of claim 28, wherein the delivery nanocarrier;
a) is selected from the group consisting of: lipoplexes, liposomes, exosomes, inorganic nanoparticles, and DNA nanostructures; and/or
b) comprises a lipid nanoparticle (LNP) encapsulating the antisense oligonucleotide.

30. (canceled)

31. The method of claim 1, wherein the antisense oligonucleotide is formulated for a route of administration selected from the group consisting of intravitreal, suprachoroidal, subretinal, ciliary intramuscular, intravenous, intra-arterial, subcutaneous, and topical routes.

32. (canceled)

33. An antisense oligonucleotide that:
a) binds to a targeted portion of the intron 7× of an OPA1 gene transcript in a cell and increases the level of OPA1 gene transcripts encoding full length, functional OPA1 by exclusion of NMD exon 7×, wherein the antisense oligonucleotide comprises or consists of any one of SEQ ID NOs: 2-54 or SEQ ID NOs: 2491-2503; or
b) binds to a targeted portion of the 5′ UTR of an OPA1 gene transcript in a cell and increases transcript stability of an OPA1 mRNA, wherein the targeted portion is within the nucleotide sequence corresponding to SEQ ID NO: 55, wherein the antisense oligonucleotide comprises or consists of any one of SEQ ID NOs: 56-138; or
c) binds to a targeted portion of the 3′ UTR of an OPA1 gene transcript in a cell and increases transcript stability of an OPA1 mRNA, wherein the antisense oligonucleotide binds within a targeted portion of the 3′ UTR of OPA1 mRNA, wherein the targeted portion is within the nucleotide sequence corresponding to SEQ ID NO: 139, and wherein the antisense oligonucleotide comprises or consists of any one of SEQ ID NOs: 140-2488.

34. (canceled)

35. (canceled)

36. (canceled)

37. (canceled)

38. (canceled)

39. (canceled)
